SEQUENCE LISTING (22 sequences)

SEQ ID NO: 1 - DNA, 7229 bp, synthetic construct
  source 1..7229: mol_type = other DNA; note = Lymphocytic choriomeningitis virus clone 13 segment L (GenBank: DQ361066.1)
gcgcaccggggatcctaggcgtttagttgcgctgtttggttgcacaactttcttcgtgaggctgtcagaagtggacctggctgatagcgatgggtcaaggcaagtccagagaggagaaaggcaccaatagtacaaacagggccgaaatcctaccagataccacctatcttggccctttaagctgcaaatcttgctggcagaaatttgacagcttggtaagatgccatgaccactacctttgcaggcactgtttaaaccttctgctgtcagtatccgacaggtgtcctctttgtaaatatccattaccaaccagattgaagatatcaacagccccaagctctccacctccctacgaagagtaacaccgtccggccccggccccgacaaacagcccagcacaagggaaccgcacgtcacccaacgcacacagacacagcacccaacacagaacacgcacacacacacacacacacacccacacgcacgcgcccccaccaccggggggcgcccccccccggggggcggccccccgggagcccgggcggagccccacggagatgcccatcagtcgatgtcctcggccaccgacccgcccagccaatcgtcgcaggacctccccttgagtctaaacctgccccccactgtttcatacatcaaagtgctcctagatttgctaaaacaaagtctgcaatccttaaaggcgaaccagtctggcaaaagcgacagtggaatcagcagaatagatctgtctatacatagttcctggaggattacacttatctctgaacccaacaaatgttcaccagttctgaatcgatgcaggaagaggttcccaaggacatcactaatcttttcatagccctcaagtcctgctagaaagactttcatgtccttggtctccagcttcacaatgatattttggacaaggtttcttccttcaaaaagggcacccatctttacagtcagtggcacaggctcccactcaggtccaactctctcaaagtcaatagatctaatcccatccagtattcttttggagcccaacaactcaagctcaagagaatcaccaagtatcaagggatcttccatgtaatcctcaaactcttcagatctgatatcaaagacaccatcgttcaccttgaagacagagtctgtcctcagtaagtggaggcattcatccaacattcttctatctatctcacccttaaagaggtgagagcatgataaaagttcagccacacctggattctgtaattggcacctaaccaagaatatcaatgaaaatttccttaaacagtcagtattattctgattgtgcgtaaagtccactgaaattgaaaactccaataccccttttgtgtagttgagcatgtagtcccacagatcctttaaggatttaaatgcctttgggtttgtcaggccctgcctaatcaacatggcagcattacacacaacatctcccattcggtaagagaaccacccaaaaccaaactgcaaatcattcctaaacataggcctctccacatttttgttcaccacctttgagacaaatgattgaaaggggcccagtgcctcagcaccatcttcagatggcatcatttctttatgagggaaccatgaaaaattgcctaatgtcctggttgttgcaacaaattctcgaacaaatgattcaaaatacacctgttttaagaagttcttgcagacatccctcgtgctaacaacaaattcatcaaccagactggagtcagatcgctgatgagaattggcaaggtcagaaaacagaacagtgtaatgttcatcccttttccacttaacaacatgagaaatgagtgacaaggattctgagttaatatcaattaaaacacagaggtcaaggaatttaattctgggactccacctcatgttttttgagctcatgtcagacataaatggaagaagctgatcctcaaagatcttgggatatagccgcctcacagattgaatcacttggttcaaattcactttgtcctccagtagccttgagctctcaggctttcttgctacataatcacatgggtttaagtgcttaagagttaggttctcactgttattcttccctttggtcggttctgctaggacccaaacacccaactcaaaagagttgctcaatgaaatacaaatgtagtcccaaagaagaggccttaaaaggcatatatgatcacggtgggcttctggatgagactgtttgtcacaaatgtacagcgttataccatcccgattgcaaactcttgtcacatgatcatctgtggttagatcctcaagcagctttttgatatacagattttccctatttttgtttctcacacacctgcttcctagagttttgcaaaggcctataaagccagatgagatacaactctggaaagctgacttgttgattgcttctgacagcagcttctgtgcaccccttgtgaatttactacaaagtttgttctggagtgtcttgatcaatgatgggattctttcctcttggaaagtcatcactgatggataaaccaccttttgtcttaaaaccatccttaatgggaacatttcattcaaattcaaccagttaacatctgctaactgattcagatcttcttcaagaccgaggaggtctcccaattgaagaatggcctcctttttatctctgttaaataggtctaagaaaaattcttcattaaattcaccatttttgagcttatgatgcagtttccttacaagctttcttacaacctttgtttcattaggacacagttcctcaatgagtctttgtattctgtaacctctagaaccatccagccaatctttcacatcagtgttggtattcagtagaaatggatccaaagggaaattggcatactttaggaggtccagtgttctcctttggatactattaactagggagactgggacgccatttgcgatggcttgatctgcaattgtatctattgtttcacaaagttgatgtggctctttacacttgacattgtgtagcgctgcagatacaaactttgtgagaagagggacttcctccccccatacatagaatctagatttaaattctgcagcgaacctcccagccacactttttgggctgataaatttgtttaacaagccgctcagatgagattggaattccaacaggacaaggacttcctccggatcacttacaaccaggtcactcagcctcctatcaaataaagtgatctgatcatcacttgatgtgtaagcctctggtctttcgccaaagataacaccaatgcagtagttgatgaacctctcgctaagcaaaccatagaagtcagaagcattatgcaagattccctgccccatatcaataaggctggatatatgggatggcactatccccatttcaaaatattgtctgaaaattctctcagtaacagttgtttctgaacccctgagaagttttagcttcgacttgacatatgatttcatcattgcattcacaacaggaaaggggacctcgacaagcttatgcatgtgccaagttaacaaagtgctaacatgatctttcccggaacgcacatactggtcatcacctagtttgagattttgtagaaacattaagaacaaaaatgggcacatcattggtccccatttgctgtgatccatactatagtttaagaacccttcccgcacattgatagtcattgacaagattgcattttcaaattccttatcattgtttaaacaggagcctgaaaagaaacttgaaaaagactcaaaataatcttctattaaccttgtgaacatttttgtcctcaaatctccaatatagagttctctatttcccccaacctgctctttataagatagtgcaaatttcagccttccagagtcaggacctactgaggtgtatgatgttggtgattcttctgagtagaagcacagatttttcaaagcagcactcatacattgtgtcaacgacagagctttactaagggactcagaattactttccctctcactgattctcacgtcttcttccagtttgtcccagtcaaatttgaaattcaagccttgcctttgcatatgcctgtatttccctgagtacgcatttgcattcatttgcaacagaatcatcttcatgcaagaaaaccaatcattctcagaaaagaactttctacaaaggttttttgccatctcatcgaggccacactgatctttaatgactgaggtgaaatacaaaggtgacagctctgtggaaccctcaacagcctcacagataaatttcatgtcatcattggttagacatgatgggtcaaagtcttctactaaatggaaagatatttctgacaagataacttttcttaagtgagccatcttccctgttagaataagctgtaaatgatgtagtccttttgtatttgtaagtttttctccatctcctttgtcattggccctcctacctcttctgtaccgtgctattgtggtgttgaccttttcttcgagacttttgaagaagcttgtctcttcttctccatcaaaacatatttctgccaggttgtcttccgatctccctgtctcttctcccttggaaccgatgaccaatctagagactaacttggaaactttatattcatagtctgagtggctcaacttatacttttgttttcttacgaaactctccgtaatttgactcacagcactaacaagcaatttgttaaagtcatattccagaagtcgttctccatttagatgcttattaaccaccacacttttgttactagcaagatctaatgctgtcgcacatccagagttagtcatgggatctaggctgtttagcttcttctctcctttgaaaattaaagtgccgttgttaaatgaagacaccattaggctaaaggcttccagattaacacctggagttgtatgctgacagtcaatttctttactagtgaatctcttcatttgctcatagaacacacattcttcctcaggagtgattgcttccttggggttgacaaaaaaaccaaattgacttttgggctcaaagaacttttcaaaacattttatctgatctgttagcctgtcaggggtctcctttgtgatcaaatgacacaggtatgacacattcaacataaatttaaattttgcactcaacaacaccttctcaccagtaccaaaaatagtttttattaggaatctaagcagcttatacaccaccttctcagcaggtgtgatcagatcctccctcaacttatccattaatgatgtagatgaaaaatctgacactattgccatcaccaaatatctgacactctgtacctgcttttgatttctctttgttgggttggtgagcattagcaacaatagggtcctcagtgcaacctcaatgtcggtgagacagtctttcaaatcaggacatgatctaatccatgaaatcatgatgtctatcatattgtataagacctcatctgaaaaaattggtaaaaagaaccttttaggatctgcatagaaggaaattaaatgaccatccgggccttgtatggagtagcaccttgaagattctccagtcttctggtataataggtggtattcttcagagtccagttttattacttggcaaaacacttctttgcattctaccacttgatatctcacagaccctatttgattttgccttagtctagcaactgagctagttttcatactgtttgttaaggccagacaaacagatgataatcttctcaggctctgtatgttcttcagctgctctgtgctgggttggaaattgtaatcttcaaacttcgtataatacattatcgggtgagctccaattttcataaagttctcaaattcagtgaatggtatgtggcattcttgctcaaggtgttcagacagtccgtaatgctcgaaactcagtcccaccactaacaggcatttttgaatttttgcaatgaactcactaatagatgccctaaacaattcctcaaaagacacctttctaaacacctttgacttttttctattcctcaaaagtctaatgaactcctctttagtgctgtgaaagcttaccagcctatcattcacactactatagcaacaacccacccagtgtttatcattttttaaccctttgaatttcgactgttttatcaatgaggaaagacacaaaacatccagatttaacaactgtctccttctagtattcaacagtttcaaactcttgactttgtttaacatagagaggagcctctcatattcagtgctagtctcacttcccctttcgtgcccatgggtctctgcagttatgaatctcatcaaaggacaggattcgactgcctccctgcttaatgttaagatatcatcactatcagcaaggttttcatagagctcagagaattccttgatcaagccttcagggtttactttctgaaagtttctctttaatttcccactttctaaatctcttctaaacctgctgaaaagagagtttattccaaaaaccacatcatcacagctcatgttggggttgatgccttcgtggcacatcctcataatttcatcattgtgagttgacctcgcatctttcagaattttcatagagtccataccggagcgcttgtcgatagtagtcttcagggactcacagagtctaaaatattcagactcttcaaagactttctcattttggttagaatactccaaaagtttgaataaaaggtctctaaatttgaagtttgcccactctggcataaaactattatcataatcacaacgaccatctactattggaactaatgtgacacccgcaacagcaaggtcttccctgatgcatgccaatttgttagtgtcctctataaatttcttctcaaaactggctggagtgctcctaacaaaacactcaagaagaatgagagaattgtctatcagcttgtaaccatcaggaatgataagtggtagtcctgggcatacaattccagactccaccaaaattgtttccacagacttatcgtcgtggttgtgtgtgcagccactcttgtctgcactgtctatttcaatgcagcgtgacagcaacttgagtccctcaatcagaaccattctgggttccctttgtcccagaaagttgagtttctgccttgacaacctctcatcctgttctatatagtttaaacataactctctcaattctgagatgatttcatccattgcgcatcaaaaagcctaggatcctcggtgcg

SEQ ID NO: 2 - DNA, 3376 bp, synthetic construct
  source 1..3376: mol_type = other DNA; note = Lymphocytic choriomeningitis virus segment S
cgcaccggggatcctaggctttttggattgcgctttcctctagatcaactgggtgtcaggccctatcctacagaaggatgggtcagattgtgacaatgtttgaggctctgcctcacatcatcgatgaggtgatcaacattgtcattattgtgcttatcgtgatcacgggtatcaaggctgtctacaattttgccacctgtgggatattcgcattgatcagtttcctacttctggctggcaggtcctgtggcatgtacggtcttaagggacccgacatttacaaaggagtttaccaatttaagtcagtggagtttgatatgtcacatctgaacctgaccatgcccaacgcatgttcagccaacaactcccaccattacatcagtatggggacttctggactagaattgaccttcaccaatgattccatcatcagtcacaacttttgcaatctgacctctgccttcaacaaaaagacctttgaccacacactcatgagtatagtttcgagcctacacctcagtatcagagggaactccaactataaggcagtatcctgcgacttcaacaatggcataaccatccaatacaacttgacattctcagatcgacaaagtgctcagagccagtgtagaaccttcagaggtagagtcctagatatgtttagaactgccttcggggggaaatacatgaggagtggctggggctggacaggctcagatggcaagaccacctggtgtagccagacgagttaccaatacctgattatacaaaatagaacctgggaaaaccactgcacatatgcaggtccttttgggatgtccaggattctcctttcccaagagaagactaagttcttcactaggagactagcgggcacattcacctggactttgtcagactcttcaggggtggagaatccaggtggttattgcctgaccaaatggatgattcttgctgcagagcttaagtgtttcgggaacacagcagttgcgaaatgcaatgtaaatcatgatgccgaattctgtgacatgctgcgactaattgactacaacaaggctgctttgagtaagttcaaagaggacgtagaatctgccttgcacttattcaaaacaacagtgaattctttgatttcagatcaactactgatgaggaaccacttgagagatctgatgggggtgccatattgcaattactcaaagttttggtacctagaacatgcaaagaccggcgaaactagtgtccccaagtgctggcttgtcaccaatggttcttacttaaatgagacccacttcagtgatcaaatcgaacaggaagccgataacatgattacagagatgttgaggaaggattacataaagaggcaggggagtacccccctagcattgatggaccttctgatgttttccacatctgcatatctagtcagcatcttcctgcaccttgtcaaaataccaacacacaggcacataaaaggtggctcatgtccaaagccacaccgattaaccaacaaaggaatttgtagttgtggtgcatttaaggtgcctggtgtaaaaaccgtctggaaaagacgctgaagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttgtcttcatactccctcgaagcttccctggtcatttcaacatcgataagcttaatgtccttcctattctgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgaattgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagaggtcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

SEQ ID NO: 3 - DNA, 3377 bp, synthetic construct
  source 1..3377: mol_type = other DNA; note = Lymphocytic choriomeningitis virus clone 13 segment S (GenBank: DQ361065.2)
gcgcaccggggatcctaggctttttggattgcgctttcctctagatcaactgggtgtcaggccctatcctacagaaggatgggtcagattgtgacaatgtttgaggctctgcctcacatcatcgatgaggtgatcaacattgtcattattgtgcttatcgtgatcacgggtatcaaggctgtctacaattttgccacctgtgggatattcgcattgatcagtttcctacttctggctggcaggtcctgtggcatgtacggtcttaagggacccgacatttacaaaggagtttaccaatttaagtcagtggagtttgatatgtcacatctgaacctgaccatgcccaacgcatgttcagccaacaactcccaccattacatcagtatggggacttctggactagaattgaccttcaccaatgattccatcatcagtcacaacttttgcaatctgacctctgccttcaacaaaaagacctttgaccacacactcatgagtatagtttcgagcctacacctcagtatcagagggaactccaactataaggcagtatcctgcgacttcaacaatggcataaccatccaatacaacttgacattctcagatgcacaaagtgctcagagccagtgtagaaccttcagaggtagagtcctagatatgtttagaactgccttcggggggaaatacatgaggagtggctggggctggacaggctcagatggcaagaccacctggtgtagccagacgagttaccaatacctgattatacaaaatagaacctgggaaaaccactgcacatatgcaggtccttttgggatgtccaggattctcctttcccaagagaagactaagttcctcactaggagactagcgggcacattcacctggactttgtcagactcttcaggggtggagaatccaggtggttattgcctgaccaaatggatgattcttgctgcagagcttaagtgtttcgggaacacagcagttgcgaaatgcaatgtaaatcatgatgaagaattctgtgacatgctgcgactaattgactacaacaaggctgctttgagtaagttcaaagaggacgtagaatctgccttgcacttattcaaaacaacagtgaattctttgatttcagatcaactactgatgaggaaccacttgagagatctgatgggggtgccatattgcaattactcaaagttttggtacctagaacatgcaaagaccggcgaaactagtgtccccaagtgctggcttgtcaccaatggttcttacttaaatgagacccacttcagtgaccaaatcgaacaggaagccgataacatgattacagagatgttgaggaaggattacataaagaggcaggggagtacccccctagcattgatggaccttctgatgttttccacatctgcatatctagtcagcatcttcctgcaccttgtcaaaataccaacacacaggcacataaaaggtggctcatgtccaaagccacaccgattaaccaacaaaggaatttgtagttgtggtgcatttaaggtgcctggtgtaaaaaccgtctggaaaagacgctgaagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttgtcttcatactccctcgaagcttccctggtcatttcaacatcgataagcttaatgtccttcctattctgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgaattgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagaggtcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

SEQ ID NO: 4 - DNA, 7205 bp, synthetic construct
  source 1..7205: mol_type = other DNA; note = Lymphocytic choriomeningitis strain MP segment L
gcgcaccggggatcctaggcatttttgttgcgcattttgttgtgttatttgttgcacagcccttcatcgtgggaccttcacaaacaaaccaaaccaccagccatgggccaaggcaagtccaaagagggaagggatgccagcaatacgagcagagctgaaattctgccagacaccacctatctcggacctctgaactgcaagtcatgctggcagagatttgacagtttagtcagatgccatgaccactatctctgcagacactgcctgaacctcctgctgtcagtctccgacaggtgccctctctgcaaacatccattgccaaccaaactgaaaatatccacggccccaagctctccacccccttacgaggagtgacgccccgagccccaacaccgacacaaggaggccaccaacacaacgcccaacacggaacacacacacacacacccacacacacatccacacacacgcgcccccacaacgggggcgcccccccgggggtggccccccgggtgctcgggcggagccccacggagaggccaattagtcgatctcctcgaccaccgacttggtcagccagtcatcacaggacttgcccttaagtctgtacttgcccacaactgtttcatacatcaccgtgttctttgacttactgaaacatagcctacagtctttgaaagtgaaccagtcaggcacaagtgacagcggtaccagtagaatggatctatctatacacaactcttggagaattgtgctaatttccgacccctgtagatgctcaccagttctgaatcgatgtagaagaaggctcccaaggacgtcatcaaaatttccataaccctcgagctctgccaagaaaactctcatatccttggtctccagtttcacaacgatgttctgaacaaggcttcttccctcaaaaagagcacccattctcacagtcaagggcacaggctcccattcaggcccaatcctctcaaaatcaagggatctgatcccgtccagtattttccttgagcctatcagctcaagctcaagagagtcaccgagtatcagggggtcctccatatagtcctcaaactcttcagacctaatgtcaaaaacaccatcgttcaccttgaagatagagtctgatctcaacaggtggaggcattcgtccaagaaccttctgtccacctcacctttaaagaggtgagagcatgataggaactcagctacacctggaccttgtaactggcacttcactaaaaagatcaatgaaaacttcctcaaacaatcagtgttattctggttgtgagtgaaatctactgtaattgagaactctagcactccctctgtattatttatcatgtaatcccacaagtttctcaaagacttgaatgcctttggatttgtcaagccttgtttgattagcatggcagcattgcacacaatatctcccaatcggtaagagaaccatccaaatccaaattgcaagtcattcctaaacatgggcctctccatatttttgttcactacttttaagatgaatgattggaaaggccccaatgcttcagcgccatcttcagatggcatcatgtctttatgagggaaccatgaaaaacttcctagagttctgcttgttgctacaaattctcgtacaaatgactcaaaatacacttgttttaaaaagtttttgcagacatcccttgtactaacgacaaattcatcaacaaggcttgagtcagagcgctgatgggaatttacaagatcagaaaatagaacagtgtagtgttcgtccctcttccacttaactacatgagaaatgagcgataaagattctgaattgatatcgatcaatacgcaaaggtcaaggaatttgattctgggactccatctcatgttttttgagctcatatcagacatgaagggaagcagctgatcttcatagattttagggtacaatcgcctcacagattggattacatggtttaaacttatcttgtcctccagtagccttgaactctcaggcttccttgctacataatcacatgggttcaagtgcttgaggcttgagcttccctcattcttccctttcacaggttcagctaagacccaaacacccaactcaaaggaattactcagtgagatgcaaatatagtcccaaaggaggggcctcaagagactgatgtggtcgcagtgagcttctggatgactttgcctgtcacaaatgtacaacattatgccatcatgtctgtggattgctgtcacatgcgcatccatagctagatcctcaagcacttttctaatgtatagattgtccctatttttatttctcacacatctacttcccaaagttttgcaaagacctataaagcctgatgagatgcaactttgaaaggctgacttattgattgcttctgacagcaacttctgtgcacctcttgtgaacttactgcagagcttgttctggagtgtcttgattaatgatgggattctttcctcttggaaagtcattactgatggataaaccactttctgcctcaagaccattcttaatgggaacaactcattcaaattcagccaatttatgtttgccaattgacttagatcctcttcgaggccaaggatgtttcccaactgaagaatggcttcctttttatccctattgaagaggtctaagaagaattcttcattgaactcaccattcttgagcttatgatgtagtctccttacaagccttctcatgaccttcgtttcactaggacacaattcttcaataagcctttggattctgtaacctctagagccatccaaccaatccttgacatcagtattagtgttaagcaaaaatgggtccaagggaaagttggcatattttaagaggtctaatgttctcttctggatgcagtttaccaatgaaactggaacaccatttgcaacagcttgatcggcaattgtatctattgtttcacagagttggtgtggctctttacacttaacgttgtgtaatgctgctgacacaaattttgttaaaagtgggacctcttccccccacacataaaatctggatttaaattctgcagcaaatcgccccaccacacttttcggactgatgaacttgttaagcaagccactcaaatgagaatgaaattccagcaatacaaggacttcctcagggtcactatcaaccagttcactcaatctcctatcaaataaggtgatctgatcatcacttgatgtgtaagattctggtctctcaccaaaaatgacaccgatacaataattaatgaatctctcactgattaagccgtaaaagtcagaggcattatgtaagattccctgtcccatgtcaatgagactgcttatatgggaaggcactattcctaattcaaaatattctcgaaagattctttcagtcacagttgtctctgaacccctaagaagtttcagctttgatttgatatatgatttcatcattgcattcacaacaggaaaagggacctcaacaagtttgtgcatgtgccaagttaataaggtgctgatatgatcctttccggaacgcacatactggtcatcacccagtttgagattttgaaggagcattaaaaacaaaaatgggcacatcattggcccccatttgctatgatccatactgtagttcaacaacccctctcgcacattgatggtcattgatagaattgcattttcaaattctttgtcattgtttaagcatgaacctgagaagaagctagaaaaagactcaaaataatcctctatcaatcttgtaaacatttttgttctcaaatccccaatataaagttctctgtttcctccaacctgctctttgtatgataacgcaaacttcaaccttccggaatcaggaccaactgaagtgtatgacgttggtgactcctctgagtaaaaacataaattctttaaagcagcactcatgcattttgtcaatgatagagccttacttagagactcagaattactttccctttcactaattctaacatcttcttctagtttgtcccagtcaaacttgaaattcagaccttgtctttgcatgtgcctgtatttccctgagtatgcatttgcattcatttgcagtagaatcattttcatacacgaaaaccaatcaccctctgaaaaaaacttcctgcagaggttttttgccatttcatccagaccacattgttctttgacagctgaagtgaaatacaatggtgacagttctgtagaagtttcaatagcctcacagataaatttcatgtcatcattggtgagacaagatgggtcaaaatcttccacaagatgaaaagaaatttctgataagatgaccttccttaaatatgccattttacctgacaatatagtctgaaggtgatgcaatccttttgtattttcaaaccccacctcattttccccttcattggtcttcttgcttctttcataccgctttattgtggagttgaccttatcttctaaattcttgaagaaacttgtctcttcttccccatcaaagcatatgtctgctgagtcaccttctagtttcccagcttctgtttctttagagccgataaccaatctagagaccaactttgaaaccttgtactcgtaatctgagtggttcaatttgtacttctgctttctcatgaagctctctgtgatctgactcacagcactaacaagcaatttgttaaaatcatactctaggagccgttccccatttaaatgtttgttaacaaccacacttttgttgctggcaaggtctaatgctgttgcacacccagagttagtcatgggatccaagctattgagcctcttctcccctttgaaaatcaaagtgccattgttgaatgaggacaccatcatgctaaaggcctccagattgacacctggggttgtgcgctgacagtcaacttctttcccagtgaacttcttcatttggtcataaaaaacacactcttcctcaggggtgattgactctttagggttaacaaagaagccaaactcacttttaggctcaaagaatttctcaaagcatttaatttgatctgtcagcctatcaggggtttcctttgtgattaaatgacacaggtatgacacattcaacatgaacttgaactttgcgctcaacagtaccttttcaccagtcccaaaaacagttttgatcaaaaatctgagcaatttgtacactactttctcagcaggtgtgatcaaatcctccttcaacttgtccatcaatgatgtggatgagaagtctgagacaatggccatcactaaatacctaatgttttgaacctgtttttgattcctctttgttgggttggtgagcatgagtaataatagggttctcaatgcaatctcaacatcatcaatgctgtccttcaagtcaggacatgatctgatccatgagatcatggtgtcaatcatgttgtgcaacacttcatctgagaagattggtaaaaagaacctttttgggtctgcataaaaagagattagatggccattgggaccttgtatagaataacaccttgaggattctccagtcttttgatacagcaggtgatattcctcagagtccaattttatcacttggcaaaatacctctttacattccaccacttgataccttacagagcccaattggttttgtcttaatctagcaactgaacttgttttcatactgtttgtcaaagctagacagacagatgacaatcttttcaaactatgcatgttccttaattgttccgtattaggctggaaatcataatcttcaaactttgtataatacattataggatgagttccggacctcatgaaattctcaaactcaataaatggtatgtggcactcatgctcaagatgttcagacagaccatagtgcccaaaactaagtcccaccactgacaagcacctttgaacttttaaaatgaactcatttatggatgttctaaacaaatcctcaagagatacctttctatacgcctttgactttctcctgttccttagaagtctgatgaactcttccttggtgctatgaaagctcaccaacctatcattcacactcccatagcaacaaccaacccagtgcttatcattttttgaccctttgagtttagactgtttgatcaacgaagagagacacaagacatccaaattcagtaactgtctccttctggtgttcaataattttaaacttttaactttgttcaacatagagaggagcctctcatactcagtgctagtctcacttcctctctcataaccatgggtatctgctgtgataaatctcatcaaaggacaggattcaactgcctccttgcttagtgctgaaatgtcatcactgtcagcaagagtctcataaagctcagagaattccttaattaaatttccggggttgattttctgaaaactcctcttgagcttcccagtttccaagtctcttctaaacctgctgtaaagggagtttatgccaagaaccacatcatcgcagttcatgtttgggttgacaccatcatggcacattttcataatttcatcattgtgaaatgatcttgcatctttcaagattttcatagagtctataccggaacgcttatcaacagtggtcttgagagattcgcaaagtctgaagtactcagattcctcaaagactttctcatcttggctagaatactctaaaagtttaaacagaaggtctctgaacttgaaattcacccactctggcataaagctgttatcataatcacaccgaccatccactattgggaccaatgtgatacccgcaatggcaaggtcttctttgatacaggctagtttattggtgtcctctataaatttcttctcaaaactagctggtgtgcttctaacgaagcactcaagaagaatgagggaattgtcaatcagtttataaccatcaggaatgatcaaaggcagtcccgggcacacaatcccagactctattagaattgcctcaacagatttatcatcatggttgtgtatgcagccgctcttgtcagcactgtctatctctatacaacgcgacaaaagtttgagtccctctatcaataccattctgggttctctttgccctaaaaagttgagcttctgccttgacaacctctcatcttgttctatgtggtttaagcacaactctctcaactccgaaatagcctcatccattgcgcatcaaaaagcctaggatcctcggtgcg

SEQ ID NO: 5 - DNA, 3359 bp, synthetic construct
  source 1..3359: mol_type = other DNA; note = Lymphocytic choriomeningitis strain MP segment S
cgcaccggggatcctaggctttttggattgcgctttcctcagctccgtcttgtgggagaatgggtcaaattgtgacgatgtttgaggctctgcctcacatcattgatgaggtcattaacattgtcattatcgtgcttattatcatcacgagcatcaaagctgtgtacaatttcgccacctgcgggatacttgcattgatcagctttctttttctggctggcaggtcctgtggaatgtatggtcttgatgggcctgacatttacaaaggggtttaccgattcaagtcagtggagtttgacatgtcttaccttaacctgacgatgcccaatgcatgttcggcaaacaactcccatcattatataagtatggggacttctggattggagttaaccttcacaaatgactccatcatcacccacaacttttgtaatctgacttccgccctcaacaagaggacttttgaccacacacttatgagtatagtctcaagtctgcacctcagcattagaggggtccccagctacaaagcagtgtcctgtgattttaacaatggcatcactattcaatacaacctgtcattttctaatgcacagagcgctctgagtcaatgtaagaccttcagggggagagtcctggatatgttcagaactgcttttggaggaaagtacatgaggagtggctggggctggacaggttcagatggcaagactacttggtgcagccagacaaactaccaatatctgattatacaaaacaggacttgggaaaaccactgcaggtacgcaggccctttcggaatgtctagaattctcttcgctcaagaaaagacaaggtttctaactagaaggcttgcaggcacattcacttggactttatcagactcatcaggagtggagaatccaggtggttactgcttgaccaagtggatgatcctcgctgcagagctcaagtgttttgggaacacagctgttgcaaagtgcaatgtaaatcatgatgaagagttctgtgatatgctacgactgattgattacaacaaggctgctttgagtaaattcaaagaagatgtagaatccgctctacatctgttcaagacaacagtgaattctttgatttctgatcagcttttgatgagaaatcacctaagagacttgatgggagtgccatactgcaattactcgaaattctggtatctagagcatgcaaagactggtgagactagtgtccccaagtgctggcttgtcagcaatggttcttatttgaatgaaacccatttcagcgaccaaattgagcaggaagcagataatatgatcacagaaatgctgagaaaggactacataaaaaggcaagggagtacccctctagccttgatggatctattgatgttttctacatcagcatatttgatcagcatctttctgcatcttgtgaggataccaacacacagacacataaagggcggctcatgcccaaaaccacatcggttaaccagcaagggaatctgtagttgtggtgcatttaaagtaccaggtgtggaaaccacctggaaaagacgctgaacagcagcgcctccctgactcaccacctcgaaagaggtggtgagtcagggaggcccagagggtcttagagtgttacgacatttggacctctgaagattaggtcatgtggtaggatattgtggacagttttcaggtcggggagccttgccttggaggcgctttcaaagatgatacagtccatgagtgcacagtgtggggtgacctctttctttttcttgtccctcactattccagtgtgcatcttgcatagccagccatatttgtcccagactttgtcctcatattctcttgaagcttctttagtcatctcaacatcgatgagcttaatgtctcttctgttttgtgaatctaggagtttcctgatgtcatcagatccctgacaacttaggaccattccctgtggaagagcacctattactgaagatgtcagcccaggttgtgcattgaagaggtcagcaaggtccatgccatgtgagtatttggagtcctgcttgaattgtttttgatcagtgggttctctatagaaatgtatgtactgcccattctgtggctgaaatattgctatttctaccgggtcattaaatctgccctcaatgtcaatccatgtaggagcgttagggtcaatacctcccatgaggtccttcagcaacattgtttggctgtagcttaagcccacctgaggtgggcccgctgccccaggcgctggtttgggtgagttggccataggcctctcatttgtcagatcaattgttgtgttctcccatgctctccctacaactgatgttctacaagctatgtatggccacccctcccctgaaagacagactttgtagaggatgttctcgtaaggattcctgtctccaacctgatcagaaacaaacatgttgagtttcttcttggccccaagaactgctttcaggagatcctcactgttgcttggcttaattaagatggattccaacatgttacccccatctaacaaggctgcccctgctttcacagcagcaccgagactgaaattgtagccagatatgttgatgctagactgctgctcagtgatgactcccaagactgggtgcttgtctttcagcctttcaaggtcacttaggttcgggtacttgactgtgtaaagcagcccaaggtctgtgagtgcttgcacaacgtcattgagtgaggtttgtgattgtttggccatacaagccattgttaagcttggcattgtgccgaattgattgttcagaagtgatgagtccttcacatcccagaccctcaccacaccatttgcactctgctgaggtctcctcattccaaccatttgcagaatctgagatctttggtcaagctgttgtgctgttaagttccccatgtagactccagaagttagaggcctttcagacctcatgattttagccttcagtttttcaaggtcagctgcaagggacatcagttcttctgcactaagcctccctacttttagaacattcttttttgatgttgactttaggtccacaagggaatacacagtttggttgaggcttctgagtctctgtaaatctttgtcatccctcttctctttcctcatgatcctctgaacattgctcacctcagagaagtctaatccattcagaaggctggtggcatccttgatcacagcagctttcacatctgatgtgaagccttgaagctctctcctcaatgcctgggtccattgaaagcttttaacttctttggacagagacattttgtcactcagtggatttccaagtcaaatgcgcaatcaaaatgcctaggatccactgtgcg

SEQ ID NO: 6 - protein, 558 aa, synthetic construct
  source 1..558: note = NP protein of the MP strain of LCMV
MSLSKEVKSFQWTQALRRELQGFTSDVKAAVIKDATSLLNGLDFSEVSNVQRIMRKEKRDDKDLQRLRSLNQTVYSLVDLKSTSKKNVLKVGRLSAEELMSLAADLEKLKAKIMRSERPLTSGVYMGNLTAQQLDQRSQILQMVGMRRPQQSANGVVRVWDVKDSSLLNNQFGTMPSLTMACMAKQSQTSLNDVVQALTDLGLLYTVKYPNLSDLERLKDKHPVLGVITEQQSSINISGYNFSLGAAVKAGAALLDGGNMLESILIKPSNSEDLLKAVLGAKKKLNMFVSDQVGDRNPYENILYKVCLSGEGWPYIACRTSVVGRAWENTTIDLTNERPMANSPKPAPGAAGPPQVGLSYSQTMLLKDLMGGIDPNAPTWIDIEGRFNDPVEIAIFQPQNGQYIHFYREPTDQKQFKQDSKYSHGMDLADLFNAQPGLTSSVIGALPQGMVLSCQGSDDIRKLLDSQNRRDIKLIDVEMTKEASREYEDKVWDKYGWLCKMHTGIVRDKKKKEVTPHCALMDCIIFESASKARLPDLKTVHNILPHDLIFRGPNVVTL

SEQ ID NO: 7 - protein, 498 aa, synthetic construct
  source 1..498: note = GP protein of the MP strain of LCMV
MGQIVTMFEALPHIIDEVINIVIIVLIIITSIKAVYNFATCGILALISFLFLAGRSCGMYGLDGPDIYKGVYRFKSVEFDMSYLNLTMPNACSANNSHHYISMGTSGLELTFTNDSIITHNFCNLTSALNKRTFDHTLMSIVSSLHLSIRGVPSYKAVSCDFNNGITIQYNLSFSNAQSALSQCKTFRGRVLDMFRTAFGGKYMRSGWGWTGSDGKTTWCSQTNYQYLIIQNRTWENHCRYAGPFGMSRILFAQEKTRFLTRRLAGTFTWTLSDSSGVENPGGYCLTKWMILAAELKCFGNTAVAKCNVNHDEEFCDMLRLIDYNKAALSKFKEDVESALHLFKTTVNSLISDQLLMRNHLRDLMGVPYCNYSKFWYLEHAKTGETSVPKCWLVSNGSYLNETHFSDQIEQEADNMITEMLRKDYIKRQGSTPLALMDLLMFSTSAYLISIFLHLVRIPTHRHIKGGSCPKPHRLTSKGICSCGAFKVPGVETTWKRR

SEQ ID NO: 8 - protein, 2209 aa, synthetic construct
  source 1..2209: note = L protein of the MP strain of LCMV
MDEAISELRELCLNHIEQDERLSRQKLNFLGQREPRMVLIEGLKLLSRCIEIDSADKSGCIHNHDDKSVEAILIESGIVCPGLPLIIPDGYKLIDNSLILLECFVRSTPASFEKKFIEDTNKLACIKEDLAIAGITLVPIVDGRCDYDNSFMPEWVNFKFRDLLFKLLEYSSQDEKVFEESEYFRLCESLKTTVDKRSGIDSMKILKDARSFHNDEIMKMCHDGVNPNMNCDDVVLGINSLYSRFRRDLETGKLKRSFQKINPGNLIKEFSELYETLADSDDISALSKEAVESCPLMRFITADTHGYERGSETSTEYERLLSMLNKVKSLKLLNTRRRQLLNLDVLCLSSLIKQSKLKGSKNDKHWVGCCYGSVNDRLVSFHSTKEEFIRLLRNRRKSKAYRKVSLEDLFRTSINEFILKVQRCLSVVGLSFGHYGLSEHLEHECHIPFIEFENFMRSGTHPIMYYTKFEDYDFQPNTEQLRNMHSLKRLSSVCLALTNSMKTSSVARLRQNQLGSVRYQVVECKEVFCQVIKLDSEEYHLLYQKTGESSRCYSIQGPNGHLISFYADPKRFFLPIFSDEVLHNMIDTMISWIRSCPDLKDSIDDVEIALRTLLLLMLTNPTKRNQKQVQNIRYLVMAIVSDFSSTSLMDKLKEDLITPAEKVVYKLLRFLIKTVFGTGEKVLLSAKFKFMLNVSYLCHLITKETPDRLTDQIKCFEKFFEPKSEFGFFVNPKESITPEEECVFYDQMKKFTGKEVDCQRTTPGVNLEAFSMMVSSFNNGTLIFKGEKRLNSLDPMTNSGCATALDLASNKSVVVNKHLNGERLLEYDFNKLLVSAVSQITESFMRKQKYKLNHSDYEYKVSKLVSRLVIGSKETEAGKLEGDSADICFDGEEETSFFKNLEDKVNSTIKRYERSKKTNEGENEVGFENTKGLHHLQTILSGKMAYLRKVILSEISFHLVEDFDPSCLTNDDMKFICEAIETSTELSPLYFTSAVKEQCGLDEMAKNLCRKFFSEGDWFSCMKMILLQMNANAYSGKYRHMQRQGLNFKFDWDKLEEDVRISERESNSESLSKALSLTKCMSAALKNLCFYSEESPTSYTSVGPDSGRLKFALSYKEQVGGNRELYIGDLRTKMFTRLIEDYFESFSSFFSGSCLNNDKEFENAILSMTINVREGLLNYSMDHSKWGPMMCPFLFLMLLQNLKLGDDQYVRSGKDHISTLLTWHMHKLVEVPFPVVNAMMKSYIKSKLKLLRGSETTVTERIFREYFELGIVPSHISSLIDMGQGILHNASDFYGLISERFINYCIGVIFGERPESYTSSDDQITLFDRRLSELVDSDPEEVLVLLEFHSHLSGLLNKFISPKSVVGRFAAEFKSRFYVWGEEVPLLTKFVSAALHNVKCKEPHQLCETIDTIADQAVANGVPVSLVNCIQKRTLDLLKYANFPLDPFLLNTNTDVKDWLDGSRGYRIQRLIEELCPSETKVMRRLVRRLHHKLKNGEFNEEFFLDLFNRDKKEAILQLGNILGLEEDLSQLANINWLNLNELFPLRMVLRQKVVYPSVMTFQEERIPSLIKTLQNKLCSKFTRGAQKLLSEAINKSAFQSCISSGFIGLCKTLGSRCVRNKNRDNLYIRKVLEDLAMDAHVTAIHRHDGIMLYICDRQSHPEAHCDHISLLRPLLWDYICISLSNSFELGVWVLAEPVKGKNEGSSSLKHLNPCDYVARKPESSRLLEDKISLNHVIQSVRRLYPKIYEDQLLPFMSDMSSKNMRWSPRIKFLDLCVLIDINSESLSLISHVVKWKRDEHYTVLFSDLVNSHQRSDSSLVDEFVVSTRDVCKNFLKQVYFESFVREFVATSRTLGSFSWFPHKDMMPSEDGAEALGPFQSFILKVVNKNMERPMFRNDLQFGFGWFSYRLGDIVCNAAMLIKQGLTNPKAFKSLRNLWDYMINNTEGVLEFSITVDFTHNQNNTDCLRKFSLIFLVKCQLQGPGVAEFLSCSHLFKGEVDRRFLDECLHLLRSDSIFKVNDGVFDIRSEEFEDYMEDPLILGDSLELELIGSRKILDGIRSLDFERIGPEWEPVPLTVRMGALFEGRSLVQNIVVKLETKDMRVFLAELEGYGNFDDVLGSLLLHRFRTGEHLQGSEISTILQELCIDRSILLVPLSLVPDWFTFKDCRLCFSKSKNTVMYETVVGKYRLKGKSCDDWLTKSVVEEID

SEQ ID NO: 9 - protein, 90 aa, synthetic construct
  source 1..90: note = Z protein of the MP strain of LCMV
MGQGKSKEGRDASNTSRAEILPDTTYLGPLNCKSCWQRFDSLVRCHDHYLCRHCLNLLLSVSDRCPLCKHPLPTKLKISTAPSSPPPYEE

SEQ ID NO: 10 - DNA, 7115 bp, synthetic construct
  source 1..7115: mol_type = other DNA; note = Junin virus Candid#1 L segment
gcgcaccggggatcctaggcgtaacttcatcattaaaatctcagattctgctctgagtgtgacttactgcgaagaggcagacaaatgggcaactgcaacggggcatccaagtctaaccagccagactcctcaagagccacacagccagccgcagaatttaggagggtagctcacagcagtctatatggtagatataactgtaagtgctgctggtttgctgataccaatttgataacctgtaatgatcactacctttgtttaaggtgccatcagggtatgttaaggaattcagatctctgcaatatctgctggaagcccctgcccaccacaatcacagtaccggtggagccaacagcaccaccaccataggcagactgcacagggtcagacccgaccccccggggggcccccatggggaccccccgtgggggaaccccgggggtgatgcgccattagtcaatgtctttgatctcgactttgtgcttcagtggcctgcatgtcacccctttcaatctgaactgcccttggggatctgatatcagcaggtcatttaaagatctgctgaatgccaccttgaaatttgagaattccaaccagtcaccaaatttatcaagtgaacggatcaactgctctttgtgtagatcataaacgaggacaaagtcctcttgctgaaataatattgtttgtgatgttgtttttagataaggccatagttggcttaataaggtttccacactatcaatgtcctctagtgctccaattgccttgactatgacatccccagacaactcaactctatatgttgacaacctttcattacctctgtaaaagataccctctttcaagacaagaggttctcctgggttatctggcccaatgaggtcatatgcatacttgttacttagttcagaataaaagtcaccaaagttgaacttaacatggctcagaatattgtcatcatttgtcgcagcgtagcctgcatcaataaacaagccagctaggtcaaagctctcatggcctgtgaacaatggtaggctagcgataaccagtgcaccatccaacaatgagtggcttccctcagacccagaaacacattgactcattgcatccacattcagctctaattcaggggtaccgacatcatccactcctagtgaactgacaatggtgtaactgtacaccatctttcttctaagtttaaattttgtcgaaactcgtgtgtgttctacttgaatgatcaattttagtttcacagcttcttggcaagcaacattgcgcaacacagtgtgcaggtccatcatgtcttcctgaggcaacaaggagatgttgtcaacagagacaccctcaaggaaaaccttgatattatcaaagctagaaactacataacccattgcaatgtcttcaacaaacattgctcttgatactttattattcctaactgacaaggtaaaatctgtgagttcagctagatctacttgactgtcatcttctagatctagaacttcattgaaccaaaagaaggatttgagacacgatgttgacatgactagtgggtttatcatcgaagataagacaacttgcaccatgaagttcctgcaaacttgctgtgggctgatgccaacttcccaatttgtatactctgactgtctaacatgggctgaagcgcaatcactctgtttcacaatataaacattattatctcttactttcaataagtgacttataatccctaagttttcattcatcatgtctagagccacacagacatctagaaacttgagtcttccactatccaaagatctgttcacttgaagatcattcataaagggtgccaaatgttcttcaaatagtttggggtaatttcttcgtatagaatgcaatacatggttcatgcctaattggtcttctatctgtcgtactgctttgggtttaacagcccagaagaaattcttattacataagaccagaggggcctgtggactcttaatagcagaaaacacccactcccctaactcacaggcatttgtcagcaccaaagagaagtaatcccacaaaattggtttagaaaattggttaacttctttaagtgatttttgacagtaaataactttaggctttctctcacaaattccacaaagacatggcattattcgagtaaatatgtcctttatatacagaaatccgcctttaccatccctaacacacttactccccatactcttacaaaacccaatgaagcctgaggcaacagaagactgaaatgcagatttgttgattgactctgccaagatcttcttcacgccttttgtgaaatttcttgacagcctggactgtattgtccttatcaatgttggcatctcttctttctctaacactcttcgacttgtcatgagtttggtcctcaagaccaacctcaagtccccaaagctcgctaaattgacccatctgtagtctagagtttgtctgatttcatcttcactacacccggcatattgcaggaatccggataaagcctcatcccctcccctgcttatcaagttgataaggttttcctcaaagattttgcctctcttaatgtcattgaacactttcctcgcgcagttccttataaacattgtctccttatcatcagaaaaaatagcttcaattttcctctgtagacggtaccctctagacccatcaacccagtctttgacatcttgttcttcaatagctccaaacggagtctctctgtatccagagtatctaatcaattggttgactctaatggaaatctttgacactatatgagtgctaaccccattagcaatacattgatcacaaattgtgtctatggtctctgacagttgtgttggagttttacacttaacgttgtgtagagcagcagacacaaacttggtgagtaaaggagtctcttcacccatgacaaaaaatcttgacttaaactcagcaacaaaagttcctatcacactctttgggctgataaacttgtttaatttagaagataagaattcatggaagcacaccatttccagcagttctgtcctgtcttgaaacttttcatcactaaggcaaggaatttttataaggctaacctggtcatcgctggaggtataagtgacaggtatcacatcatacaataagtcaagtgcataacacagaaattgttcagtaattagcccatataaatctgatgtgttgtgcaagattccctggcccatgtccaagacagacattatatggctggggacctggtcccttgactgcagatactggtgaaaaaactcttcaccaacactagtacagtcacaacccattaaacctaaagatctcttcaatttccctacacagtaggcttctgcaacattaattggaacttcaacgaccttatgaagatgccatttgagaatgttcattactggttcaagattcacctttgttctatctctgggattcttcaattctaatgtgtacaaaaaagaaaggaaaagtgctgggctcatagttggtccccatttggagtggtcatatgaacaggacaagtcaccattgttaacagccattttcatatcacagattgcacgttcgaattccttttctgaattcaagcatgtgtatttcattgaactacccacagcttctgagaagtcttcaactaacctggtcatcagcttagtgttgaggtctcccacatacagttctctatttgagccaacctgctccttataacttagtccaaatttcaagttccctgtatttgagctgatgcttgtgaactctgtaggagagtcgtctgaatagaaacataaattccgtagggctgcatttgtaaaataacttttgtctagcttatcagcaatggcttcagaattgctttccctggtactaagccgaacctcatcctttagtctcagaacttcactggaaaagcccaatctagatctacttctatgctcataactacccaatttctgatcataatgtccttgaattaaaagatacttgaagcattcaaagaattcatcttcttggtaggctattgttgtcaaattttttaataacaaacccaaagggcagatgtcctgcggtgcttcaagaaaataagtcaatttaaatggagatagataaacagcatcacataactctttatacacatcagacctgagcacatctggatcaaaatccttcacctcatgcattgacacctctgctttaatctctctcaacactccaaaaggggcccacaatgactcaagagactctcgctcatcaacagatggattttttgatttcaacttggtgatctcaacttttgtcccctcactattagccatcttggctagtgtcatttgtacgtcatttctaataccctcaaaggcccttacttgatcctctgttaaactctcatacatcactgataattcttcttgattggttctggttcttgaaccggtgctcacaagacctgttagattttttaatattaagtagtccatggaatcaggatcaagattatacctgccttttgttttaaacctctcagccatagtagaaacgcatgttgaaacaagtttctccttatcataaacagaaagaatatttccaagttcgtcgagcttggggattaccacacttttattgcttgacagatccagagctgtgctagtgatgttaggcctgtagggattgcttttcagttcacctgtaactttaagtcttcctctattgaagagagaaatgcagaaggacaaaatctctttacacactcctggaatttgagtatctgaggaagtcttagcctctttggaaaagaatctgtccaatcctcttatcatggtgtcctcttgttccagtgttagactcccacttagaggggggtttacaacaacacaatcaaacttgactttgggctcaataaacttctcaaaacactttatttgatctgtcaggcgatcaggtgtctctttggttaccaagtgacacagataactaacatttaatagatatttaaaccttcttgcaaagtaaagatctgcatcttccccttcacccaaaattgtctggaaaagttccacagccatcctctgaatcagcacctctgatccagacatgcagtcgacccttaactttgacatcaaatccacatgatggatttgatttgcatatgccatcaagaaatatcttagaccttgtaaaaatgtctggttccttttggaaggggaacagagtacagctaacactaacaatcttaatattggccttgtcattgtcatgagttcgtggctaaaatccaaccagctggtcatttcctcacacatttcaattaacacatcctccgaaaatataggcaggaaaaatctctttggatcacagtaaaaagagccttgttcttccaataccccattgatggatagatagatagaatagcaccttgacttctcacctgttttttggtaaaacaagagaccaaatgtattctttgtcagatgaaatctttgtacataacactctcttagtctaacattcccaaaatatctagaatactctctttcattgattaacaatcgggaggaaaatgatgtcttcatcgagttgaccaatgcaagggaaatggaggacaaaatcctaaataatttcttctgctcaccttccactaagctgctgaatggctgatgtctacagattttctcaaattccttgttaatagtatatctcatcactggtctgtcagaaacaagtgcctgagctaaaatcatcaagctatccatatcagggtgttttattagtttttccagctgtgaccagagatcttgatgagagttcttcaatgttctggaacacgcttgaacccacttggggctggtcatcaatttcttccttattagtttaatcgcctccagaatatctagaagtctgtcattgactaacattaacatttgtccaacaactattcccgcatttcttaaccttacaattgcatcatcatgcgttttgaaaagatcacaaagtaaattgagtaaaactaagtccagaaacagtaaagtgtttctcctggtgttgaaaacttttagacctttcactttgttacacacggaaagggcttgaagataacacctctctacagcatcaatagatatagaattctcatctgactggctttccatgttgacttcatctattggatgcaatgcgatagagtagactacatccatcaacttgtttgcacaaaaagggcagctgggcacatcactgtctttgtggcttcctaataagatcaagtcatttataagcttagacttttgtgaaaatttgaatttccccaactgcttgtcaaaaatctccttcttaaaccaaaaccttaactttatgagttcttctcttatgacagattctctaatgtctcctctaaccccaacaaagagggattcatttaacctctcatcataacccaaagaattctttttcaagcattcgatgttttctaatcccaagctctggttttttgtgttggacaaactatggatcaatcgctggtattcttgttcttcaatattaatctcttgcataaattttgatttctttaggatgtcgatcagcaaccaccgaactctttcaacaacccaatcagcaaggaatctattgctgtagctagatctgccatcaaccacaggaaccaacgtaatccctgcccttagtaggtcggactttaggtttaagagctttgacatgtcactcttccattttctctcaaactcatcaggattgaccctaacaaaggtttccaataggatgagtgttttccctgtgagtttgaagccatccggaatgacttttggaagggtgggacatagtatgccatagtcagacaggatcacatcaacaaacttctgatctgaattgatctgacaggcgtgtgcctcacaggactcaagctctactaaacttgacagaagtttgaacccttccaacaacagagagctggggtgatgttgagataaaaagatgtccctttggtatgctagctcctgtctttctggaaaatgctttctaataaggctttttatttcatttactgattcctccatgctcaagtgccgcctaggatcctcggtgcg

SEQ ID NO: 11 - DNA, 3411 bp, synthetic construct
  source 1..3411: mol_type = other DNA; note = Junin virus Candid#1 S segment
gcgcaccggggatcctaggcgattttggttacgctataattgtaactgttttctgtttggacaacatcaaaaacatccattgcacaatggggcagttcattagcttcatgcaagaaataccaacctttttgcaggaggctctgaacattgctcttgttgcagtcagtctcattgccatcattaagggtatagtgaacttgtacaaaagtggtttattccaattctttgtattcctagcgcttgcaggaagatcctgcacagaagaagctttcaaaatcggactgcacactgagttccagactgtgtccttctcaatggtgggtctcttttccaacaatccacatgacctacctttgttgtgtaccttaaacaagagccatctttacattaaggggggcaatgcttcatttcagatcagctttgatgatattgcagtattgttgccacagtatgatgttataatacaacatccagcagatatgagctggtgttccaaaagtgatgatcaaatttggttgtctcagtggttcatgaatgctgtgggacatgattggcatctagacccaccatttctgtgtaggaaccgtgcaaagacagaaggcttcatctttcaagtcaacacctccaagactggtgtcaatggaaattatgctaagaagtttaagactggcatgcatcatttatatagagaatatcctgacccttgcttgaatggcaaactgtgcttaatgaaggcacaacctaccagttggcctctccaatgtccactcgaccacgttaacacattacacttccttacaagaggtaaaaacattcaacttccaaggaggtccttgaaagcattcttctcctggtctttgacagactcatccggcaaggatacccctggaggctattgtctagaagagtggatgctcgtagcagccaaaatgaagtgttttggcaatactgctgtagcaaaatgcaatttgaatcatgactctgaattctgtgacatgttgaggctctttgattacaacaaaaatgctatcaaaaccctaaatgatgaaactaagaaacaagtaaatctgatggggcagacaatcaatgccctgatatctgacaatttattgatgaaaaacaaaattagggaactgatgagtgtcccttactgcaattacacaaaattttggtatgtcaaccacacactttcaggacaacactcattaccaaggtgctggttaataaaaaacaacagctatttgaacatctctgacttccgtaatgactggatattagaaagtgacttcttaatttctgaaatgctaagcaaagagtattcggacaggcagggtaaaactcctttgactttagttgacatctgtatttggagcacagtattcttcacagcgtcactcttccttcacttggtgggtataccctcccacagacacatcaggggcgaagcatgccctttgccacacaggttgaacagcttgggtggttgcagatgtggtaagtaccccaatctaaagaaaccaacagtttggcgtagaggacactaagacctcctgagggtccccaccagcccgggcactgcccgggctggtgtggccccccagtccgcggcctggccgcggactggggaggcactgcttacagtgcataggctgccttcgggaggaacagcaagctcggtggtaatagaggtgtaggttcctcctcatagagcttcccatctagcactgactgaaacattatgcagtctagcagagcacagtgtggttcactggaggccaacttgaagggagtatccttttccctctttttcttattgacaaccactccattgtgatatttgcataagtgaccatatttctcccagacctgttgatcaaactgcctggcttgttcagatgtgagcttaacatcaaccagtttaagatctcttcttccatggaggtcaaacaacttcctgatgtcatcggatccttgagtagtcacaaccatgtctggaggcagcaagccgatcacgtaactaagaactcctggcattgcatcttctatgtccttcattaagatgccgtgagagtgtctgctaccatttttaaaccctttctcatcatgtggttttctgaagcagtgaatgtactgcttacctgcaggttggaataatgccatctcaacagggtcagtggctggtccttcaatgtcgagccaaagggtgttggtggggtcgagtttccccactgcctctctgatgacagcttcttgtatctctgtcaagttagccaatctcaaattctgaccgtttttttccggctgtctaggaccagcaactggtttccttgtcagatcaatacttgtgttgtcccatgacctgcctgtgatttgtgatctagaaccaatataaggccaaccatcgccagaaagacaaagtttgtacaaaaggttttcataaggatttctattgcctggtttctcatcaataaacatgccttctcttcgtttaacctgaatggttgattttatgagggaagagaagttttctggggtgactctgattgtttccaacatgtttccaccatcaagaatagatgctccagcctttactgcagctgaaagactgaagttgtaaccagaaatattgatggagctttcatctttagtcacaatctgaaggcagtcatgttcctgagtcagtctgtcaaggtcacttaagtttggatacttcacagtgtatagaagcccaagtgaggttaaagcttgtatgacactgttcattgtctcacctccttgaacagtcatgcatgcaattgtcaatgcaggaacagagccaaactgattgtttagctttgaagggtctttaacatcccatatcctcaccacaccatttcccccagtcccttgctgttgaaatcccagtgttctcaatatctctgatcttttagcaagttgtgactgggacaagttacccatgtaaaccccctgagagcctgtctctgctcttcttatcttgttttttaatttctcaaggtcagacgccaactccatcagttcatccctccccagatctcccaccttgaaaactgtgtttcgttgaacactcctcatggacatgagtctgtcaacctctttattcaggtccctcaacttgttgaggtcttcttccccctttttagtctttctgagtgcccgctgcacctgtgccacttggttgaagtcgatgctgtcagcaattagcttggcgtccttcaaaacatctgacttgacagtctgagtgaattggctcaaacctctccttaaggactgagtccatctaaagcttggaacctccttggagtgtgccatgccagaagttctggtgattttgatctagaatagagttgctcagtgaaagtgttagacactatgcctaggatccactgtgcg

SEQ ID NO: 12 - protein, 558 aa, synthetic construct
  source 1..558: note = NP protein of the Clone 13 strain of LCMV (GenBank Accession No. ABC96002.1; GI:86440166)
MSLSKEVKSFQWTQALRRELQSFTSDVKAAVIKDATNLLNGLDFSEVSNVQRIMRKEKRDDKDLQRLRSLNQTVHSLVDLKSTSKKNVLKVGRLSAEELMSLAADLEKLKAKIMRSERPQASGVYMGNLTTQQLDQRSQILQIVGMRKPQQGASGVVRVWDVKDSSLLNNQFGTMPSLTMACMAKQSQTPLNDVVQALTDLGLLYTVKYPNLNDLERLKDKHPVLGVITEQQSSINISGYNFSLGAAVKAGAALLDGGNMLESILIKPSNSEDLLKAVLGAKRKLNMFVSDQVGDRNPYENILYKVCLSGEGWPYIACRTSIVGRAWENTTIDLTSEKPAVNSPRPAPGAAGPPQVGLSYSQTMLLKDLMGGIDPNAPTWIDIEGRFNDPVEIAIFQPQNGQFIHFYREPVDQKQFKQDSKYSHGMDLADLFNAQPGLTSSVIGALPQGMVLSCQGSDDIRKLLDSQNRKDIKLIDVEMTREASREYEDKVWDKYGWLCKMHTGIVRDKKKKEITPHCALMDCIIFESASKARLPDLKTVHNILPHDLIFRGPNVVTL

SEQ ID NO: 13 - protein, 498 aa, synthetic construct
  source 1..498: note = GP protein of the Clone 13 strain of LCMV (GenBank Accession No. ABC96001.2; GI:116563462)
MGQIVTMFEALPHIIDEVINIVIIVLIVITGIKAVYNFATCGIFALISFLLLAGRSCGMYGLKGPDIYKGVYQFKSVEFDMSHLNLTMPNACSANNSHHYISMGTSGLELTFTNDSIISHNFCNLTSAFNKKTFDHTLMSIVSSLHLSIRGNSNYKAVSCDFNNGITIQYNLTFSDAQSAQSQCRTFRGRVLDMFRTAFGGKYMRSGWGWTGSDGKTTWCSQTSYQYLIIQNRTWENHCTYAGPFGMSRILLSQEKTKFLTRRLAGTFTWTLSDSSGVENPGGYCLTKWMILAAELKCFGNTAVAKCNVNHDEEFCDMLRLIDYNKAALSKFKEDVESALHLFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLVSIFLHLVKIPTHRHIKGGSCPKPHRLTNKGICSCGAFKVPGVKTVWKRR

SEQ ID NO: 14 - protein, 2210 aa, synthetic construct
  source 1..2210: note = L protein of the Clone 13 strain of LCMV (GenBank Accession No. ABC96004.1; GI:86440169)
MDEIISELRELCLNYIEQDERLSRQKLNFLGQREPRMVLIEGLKLLSRCIEIDSADKSGCTHNHDDKSVETILVESGIVCPGLPLIIPDGYKLIDNSLILLECFVRSTPASFEKKFIEDTNKLACIREDLAVAGVTLVPIVDGRCDYDNSFMPEWANFKFRDLLFKLLEYSNQNEKVFEESEYFRLCESLKTTIDKRSGMDSMKILKDARSTHNDEIMRMCHEGINPNMSCDDVVFGINSLFSRFRRDLESGKLKRNFQKVNPEGLIKEFSELYENLADSDDILTLSREAVESCPLMRFITAETHGHERGSETSTEYERLLSMLNKVKSLKLLNTRRRQLLNLDVLCLSSLIKQSKFKGLKNDKHWVGCCYSSVNDRLVSFHSTKEEFIRLLRNRKKSKVFRKVSFEELFRASISEFIAKIQKCLLVVGLSFEHYGLSEHLEQECHIPFTEFENFMKIGAHPIMYYTKFEDYNFQPSTEQLKNIQSLRRLSSVCLALTNSMKTSSVARLRQNQIGSVRYQVVECKEVFCQVIKLDSEEYHLLYQKTGESSRCYSIQGPDGHLISFYADPKRFFLPIFSDEVLYNMIDIMISWIRSCPDLKDCLTDIEVALRTLLLLMLTNPTKRNQKQVQSVRYLVMAIVSDFSSTSLMDKLREDLITPAEKVVYKLLRFLIKTIFGTGEKVLLSAKFKFMLNVSYLCHLITKETPDRLTDQIKCFEKFFEPKSQFGFFVNPKEAITPEEECVFYEQMKRFTSKEIDCQHTTPGVNLEAFSLMVSSFNNGTLIFKGEKKLNSLDPMTNSGCATALDLASNKSVVVNKHLNGERLLEYDFNKLLVSAVSQITESFVRKQKYKLSHSDYEYKVSKLVSRLVIGSKGEETGRSEDNLAEICFDGEEETSFFKSLEEKVNTTIARYRRGRRANDKGDGEKLTNTKGLHHLQLILTGKMAHLRKVILSEISFHLVEDFDPSCLTNDDMKFICEAVEGSTELSPLYFTSVIKDQCGLDEMAKNLCRKFFSENDWFSCMKMILLQMNANAYSGKYRHMQRQGLNFKFDWDKLEEDVRISERESNSESLSKALSLTQCMSAALKNLCFYSEESPTSYTSVGPDSGRLKFALSYKEQVGGNRELYIGDLRTKMFTRLIEDYFESFSSFFSGSCLNNDKEFENAILSMTINVREGFLNYSMDHSKWGPMMCPFLFLMFLQNLKLGDDQYVRSGKDHVSTLLTWHMHKLVEVPFPVVNAMMKSYVKSKLKLLRGSETTVTERIFRQYFEMGIVPSHISSLIDMGQGILHNASDFYGLLSERFINYCIGVIFGERPEAYTSSDDQITLFDRRLSDLVVSDPEEVLVLLEFQSHLSGLLNKFISPKSVAGRFAAEFKSRFYVWGEEVPLLTKFVSAALHNVKCKEPHQLCETIDTIADQAIANGVPVSLVNSIQRRTLDLLKYANFPLDPFLLNTNTDVKDWLDGSRGYRIQRLIEELCPNETKVVRKLVRKLHHKLKNGEFNEEFFLDLFNRDKKEAILQLGDLLGLEEDLNQLADVNWLNLNEMFPLRMVLRQKVVYPSVMTFQEERIPSLIKTLQNKLCSKFTRGAQKLLSEAINKSAFQSCISSGFIGLCKTLGSRCVRNKNRENLYIKKLLEDLTTDDHVTRVCNRDGITLYICDKQSHPEAHRDHICLLRPLLWDYICISLSNSFELGVWVLAEPTKGKNNSENLTLKHLNPCDYVARKPESSRLLEDKVNLNQVIQSVRRLYPKIFEDQLLPFMSDMSSKNMRWSPRIKFLDLCVLIDINSESLSLISHVVKWKRDEHYTVLFSDLANSHQRSDSSLVDEFVVSTRDVCKNFLKQVYFESFVREFVATTRTLGNFSWFPHKEMMPSEDGAEALGPFQSFVSKVVNKNVERPMFRNDLQFGFGWFSYRMGDVVCNAAMLIRQGLTNPKAFKSLKDLWDYMLNYTKGVLEFSISVDFTHNQNNTDCLRKFSLIFLVRCQLQNPGVAELLSCSHLFKGEIDRRMLDECLHLLRTDSVFKVNDGVFDIRSEEFEDYMEDPLILGDSLELELLGSKRILDGIRSIDFERVGPEWEPVPLTVKMGALFEGRNLVQNIIVKLETKDMKVFLAGLEGYEKISDVLGNLFLHRFRTGEHLLGSEISVILQELCIDRSILLIPLSLLPDWFAFKDCRLCFSKSRSTLMYETVGGRFRLKGRSCDDWLGGSVAEDID

SEQ ID NO: 15 - protein, 90 aa, synthetic construct
  source 1..90: note = Z protein of the Clone 13 strain of LCMV (GenBank Accession No. ABC96003.1; GI:86440168)
MGQGKSREEKGTNSTNRAEILPDTTYLGPLSCKSCWQKFDSLVRCHDHYLCRHCLNLLLSVSDRCPLCKYPLPTRLKISTAPSSPPPYEE

SEQ ID NO: 16 - protein, 498 aa, synthetic construct
  source 1..498: note = GP protein of the WE strain of LCMV
MGQIVTMFEALPHIIDEVINIVIIVLIIITSIKAVYNFATCGILALVSFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAISQCRTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 17 - DNA, 35 bp, synthetic construct
  source 1..35: mol_type = other DNA; note = WE specific primer
aatcgtctctaaggatgggtcagattgtgacaatg

SEQ ID NO: 18 - DNA, 35 bp, synthetic construct; identical to SEQ ID NO: 17
  source 1..35: mol_type = other DNA; note = WE specific fusion-primer carrying an overhang complementary to the WET-specific primer

SEQ ID NO: 19 - DNA, 37 bp, synthetic construct
  source 1..37: mol_type = other DNA; note = WE specific primer
ctcggtgatcatgttatctgcttcttgttcgatttga

SEQ ID NO: 20 - DNA, 34 bp, synthetic construct
  source 1..34: mol_type = other DNA; note = WE specific fusion-primer complementary to the WE-sequence
aatcgtctctttctttatctcctcttccagatgg

SEQ ID NO: 21 - DNA, 23 bp, synthetic construct
  source 1..23: mol_type = other DNA; note = Primer specific for LCMV NP
ggctcccagatctgaaaactgtt

SEQ ID NO: 22 - DNA, 22 bp, synthetic construct
  source 1..22: mol_type = other DNA; note = NP- and GP-specific primers
gctggcttgtcactaatggctc

(19) United States
(12) Patent Application Publication
(10) Pub. No.: US 2025/0009860 A1
(43) Pub. Date: Jan. 9, 2025

(54) REPLICATION-DEFICIENT ARENAVIRUS PARTICLES AND TRI-SEGMENTED ARENAVIRUS PARTICLES AS CANCER VACCINES

(71) Applicant: Hookipa Biotech GmbH, Vienna (AT)

(72) Inventors: Sarah Schmidt, Vienna (AT); Klaus Orlinger, Wien (AT); Sandra Stephanie Ring, Unterföhring (DE); Lukas Roland Flatz, Schaan (CH)

(21) Appl. No.: 18/658,403

(22) Filed: May 8, 2024

Related U.S. Application Data
(63) Continuation of application No. 16/347,501, filed on May 3, 2019, now abandoned.
(60) Provisional application No. 62/417,865, filed on Nov. 4, 2016.
(60) Provisional application No. 62/417,891, filed on Nov. 4, 2016.

(51) Int. Cl. A61K 39/00; A61K 39/12; A61P 31/00; A61P 35/00

(52) U.S. Cl. CPC A61K 39/001156; A61K 39/001192; A61K 39/12; A61P 31/00; A61P 35/00; A61K 2039/572; A61K 2039/585

(57) ABSTRACT

The present application relates generally to genetically modified arenaviruses that are suitable vaccines against neoplastic diseases, such as cancer. The arenaviruses described herein may be suitable as vaccines and/or for treatment of neoplastic diseases and/or for the use in immunotherapies. In particular, provided herein are methods and compositions for treating a neoplastic disease by administering a genetically modified arenavirus in combination with a chemotherapeutic agent, wherein the arenavirus has been engineered to include a nucleotide sequence encoding a tumor antigen, tumor associated antigen or antigenic fragment thereof.

This application is a continuation application of U.S. patent application Ser. No. 16/347,501, which is a U.S. National Stage Application under 35 U.S.C. § 371 of International Patent Application No. PCT/EP2017/078149, filed Nov. 3, 2017, which claims the priority of and the benefit of the filing date of U.S. Provisional Application No. 62/417,865, filed Nov. 4, 2016, and U.S. Provisional Application No. 62/417,891, filed Nov. 4, 2016, the entire contents of each of which are herein incorporated by reference in their entireties.

### REFERENCE TO SEQUENCE LISTING SUBMITTED ELECTRONICALLY

This application contains a computer readable Sequence Listing which has been submitted in XML file format via Patent Center, the entire content of which is incorporated by reference herein in its entirety. The Sequence Listing XML file submitted via Patent Center is entitled “13194-099-999_SUB_SEQ_LISTING.xml”, was created on Sep. 12, 2024, and is 67,195 bytes in size.

### 1. INTRODUCTION

The present application relates generally to genetically modified arenaviruses that are suitable vaccines against neoplastic diseases, such as cancer. The arenaviruses described herein may be suitable as vaccines and/or for treatment of neoplastic diseases and/or for the use in immunotherapies. In particular, provided herein are methods and compositions for treating a neoplastic disease by administering a genetically modified arenavirus in combination with a chemotherapeutic agent, wherein the arenavirus has been engineered to include a nucleotide sequence encoding a tumor antigen, tumor associated antigen or antigenic fragment thereof.

### 2. BACKGROUND

The generation of recombinant negative-stranded RNA viruses expressing foreign genes of interest has been pursued for a long time. Different strategies have been published for other viruses (Garcia-Sastre et al., 1994, J Virol 68(10): 6254-6261; Percy et al., 1994, J Virol 68(7): 4486-4492; Flick and Hobom, 1999, Virology 262(1): 93-103; Machado et al., 2003, Virology 313(1): 235-249). In the past it has been shown that it is possible to introduce additional foreign genes into the genome of bi-segmented LCMV particles (Emonet et al., 2009, PNAS, 106(9):3473-3478). Two foreign genes of interest were inserted into the bi-segmented genome of LCMV, resulting in tri-segmented LCMV particles (r3LCMV) with two S segments and one L segment. In the tri-segmented virus, published by Emonet et al., (2009), both NP and GP were kept in their respective natural position in the S segment and thus were expressed under their natural promoters in the flanking UTR.

### 2.1 Replication-Deficient Arenavirus Vectors Expressing Genes of Interest

The use of infectious, replication-deficient arenaviruses as vectors for the expression of antigens has been reported (see Flatz et. al., 2010, Nat. Med., 16(3):339-345; Flatz et al., 2012, J. Virol., 86(15), 7760-7770). These infectious, replication-deficient arenaviruses can infect a host cell, i.e., attach to a host cell and release their genetic material into the host cell. However, they are replication-deficient, i.e., the arenavirus is unable to produce further infectious progeny particles in a non-complementing cell, due to a deletion or functional inactivation of an open reading frame (ORF) encoding a viral protein, such as the GP protein. Instead, the ORF is substituted with a nucleotide sequence of an antigen of interest. In Flatz et al. 2010, the authors used infectious, replication-deficient arenaviruses as vectors to express OVA (SIINFEKL epitope). In Flatz et al. 2012, the authors used replication deficient arenaviruses as vectors to express HIV/SIV Env.

### 2.2 Recombinant LCMV Expressing Genes of Interest

Recently, it has been shown that an infectious arenavirus particle can be engineered to contain a genome with the ability to amplify and express its genetic material in infected cells but unable to produce further progeny in normal, not genetically engineered cells (i.e., an infectious, replication-deficient arenavirus particle) (International Publication No.: WO 2009/083210 A1 and International Publication No.: WO 2014/140301 A1).

Recently published International Publication No.: WO 2016/075250 A1 shows that arenavirus genomic segments may be engineered to form tri-segmented arenavirus particles with rearrangements of their open reading frames (“ORF”), wherein the arenavirus genomic segment carries a viral ORF in a position other than the wild-type position of the ORF, comprising one L segment and two S segments or two L segments and one S segment that do not recombine into a replication-competent bi-segmented arenavirus particle.

### 2.3 Cancer and Chemotherapy

Chemotherapeutics are widely used to treat cancer, and traditionally act in the direct killing of tumor cells, such as through interference with DNA synthesis and replication. However, chemotherapeutics also are known for their severe side effects and are not always efficacious. Better treatment options are needed to more effectively treat cancer.

### 3. SUMMARY OF THE INVENTION

Provided herein are methods and compositions for treating a neoplastic disease using an arenavirus particle comprising a nucleotide sequence encoding a tumor antigen, tumor associated antigen or antigenic fragment thereof. Also provided herein are methods and compositions for treating a neoplastic disease using a chemotherapeutic agent. Thus, in certain embodiments, provided herein are methods for treating a neoplastic disease using an arenavirus particle comprising a nucleotide sequence encoding a tumor antigen, tumor associated antigen or antigenic fragment thereof, and a chemotherapeutic agent. Also, in certain embodiments, provided herein are compositions comprising an arenavirus particle comprising a nucleotide sequence encoding a tumor antigen, tumor associated antigen or antigenic fragment thereof, and a chemotherapeutic agent. In certain embodiments, the arenavirus particle provided herein is an infectious, replication deficient arenavirus particle.

In certain embodiments, the arenavirus particle provided herein is engineered to contain an arenavirus genomic segment having a nucleotide sequence encoding a tumor antigen, tumor associated antigen or antigenic fragment thereof and at least one arenavirus open reading frame (“ORF”) in a position other than the wild-type position of the ORF. In certain embodiments, the arenavirus particle provided herein is an infectious, replication deficient arenavirus particle. In other embodiments, the arenavirus particle provided herein is a tri-segmented arenavirus particle, which can be replication-deficient or replication-competent. In still other embodiments, the tri-segmented arenavirus particle provided herein, when propagated, does not result in a replication-competent bi-segmented viral particle.

### 3.1 Infectious, Replication Deficient Arenavirus Particle

In certain embodiments, an arenavirus particle provided herein is infectious, i.e., it is capable of entering into or injecting its genetic material into a host cell. In certain more specific embodiments, an arenavirus particle as provided herein is infectious, i.e., is capable of entering into or injecting its genetic material into a host cell followed by amplification and expression of its genetic information inside the host cell. In certain embodiments, the arenavirus particle provided herein is engineered to be an infectious, replication-deficient arenavirus particle, i.e., it contains a genome with the ability to amplify and express its genetic information in infected cells but unable to produce further infectious progeny particles in non-complementing cells.

In certain embodiments, provided herein is an arenavirus particle engineered to contain a genome comprising: a nucleotide sequence encoding a tumor antigen, tumor associated antigen or an antigenic fragment thereof; and the ability to amplify and express its genetic information in infected cells but unable to produce further infectious progeny particles in non-complementing cells. In certain embodiments, the arenavirus particle is infectious and replication-deficient.

The tumor antigen or tumor associated antigen encoded by the nucleotide sequence included within an arenavirus particle provided herein can be one or more of the tumor antigens or tumor associated antigens selected from the group consisting of oncogenic viral antigens, cancer-testis antigens, oncofetal antigens, tissue differentiation antigens, mutant protein antigens, Adipophilin, AIM-2, ALDH1AI, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUCI, MUC5AC, p53 (non-mutant), PAX5, PBF, PRAME, PSMA, RAGE, RAGE-1, RGS5, RhoC, RNF43, RU2AS, secernin 1, SOXI0, STEAPI (six-transmembrane epithelial antigen of the prostate 1), survivin, Telomerase, VEGF, WTl, EGF-R, CEA, CD20, CD33, CD52, MELANA/MART1, MART2,NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSXl or SSX2 fusion protein, TGF-betaRII, Triosephosphate isomerase, ormdm-2, LMP2, HPV E6/E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP-1, GP100, CA-125, CA19-9, Calretinin, Epithelial membrane antigen (EMA), Epithelial tumor antigen (ETA), CD19, CD34, CD99, CD 117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29BCAA), CA 195, CA 242, CA 50, CAM 43, CEA, EBNA, EF2, Epstein-Barr virus antigen, HLA-A2, HLA-All, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, pi80erbB-3, c-met, nm-23H1, TAG-72, TAG-72-4, CA-72-4, CAM 17.1, NuMa, 13-catenin, P16, TAGE, CT7, 43-9F,5T4, 791Tgp72, 13HCG, BCA225, BTAA, CD68KP1, CO-029, HTgp-175, M344, MG7-Ag, MOV18, NB70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin avP3 (CD61), galactin, or Ral-B, CD123, CLL-1, CD38, CS-1, CD138, and ROR1. In certain embodiments, the nucleotide sequence encodes two, three, four, five, six, seven, eight, nine, ten or more tumor antigens, tumor associated antigens or antigenic fragments thereof. In certain embodiments, an antigenic fragment of a tumor antigen or tumor assocaited provided herein is encoded by the nucleotide sequence included within the arenavirus.

In certain embodiments, an infectious, replication-deficient arenavirus particle provided herein comprises at least one arenavirus open reading frame (“ORF”) that is at least partially removed or functionally inactivated. The ORF can encode the glycoprotein (“GP”), the nucleoprotein (“NP”), the matrix protein Z (“Z protein”) or the RNA dependent RNA polymerase L (“L protein”) of the arenavirus particle. Additoinally, in certain embodiments, at least one ORF encoding the GP, NP, Z protein, or L protein is removed and replaced with a nucleotide sequence encoding a tumor antigen, tumor associated antigen or an antigenic fragment thereof provided herein. In certain embodiments, only one of the four ORFs encoding GP, NP, Z protein and L protein is removed. Thus, in certain embodiments, the ORF encoding GP is removed. In certain embodiments, the ORF encoding NP is removed. In certain embodiments, the ORF encoding Z protein is removed. In certain embodiments, the ORF encoding L protein is removed.

In certain embodiments, an infectious, replication-deficient arenavirus particle comprising a nucleotide sequence encoding a tumor antigen, tumor associated antigen or antigenic fragment thereof as provided herein further comprises at least one nucleotide sequence encoding at least one immunomodulatory peptide, polypeptide or protein. In certain embodiments, the immunomodulatory peptide, polypeptide or protein is Calreticulin (CRT), or a fragment thereof, Ubiquitin or a fragment thereof, Granulocyte-Macrophage Colony-Stimulating Factor (GM-CSF), or a fragment thereof, Invariant chain (CD74) or an antigenic fragment thereof; Mycobacterium tuberculosis Heat shock protein 70 or an antigenic fragment thereof; Herpes simplex virus 1 protein VP22 or an antigenic fragment thereof, CD40 ligand or an antigenic fragment thereof, or Fms-related tyrosine kinase 3 (Flt3) ligand or an antigenic fragment thereof.

In certain embodiments, an infectious, replication-deficient arenavirus particle provided herein is derived from a specific arenavirus species, such as lymphocytic choriomeningitis virus (“LCMV”), Junin virus (“JUNV”), or Pichinde virus (“PICV”). In other words, the genomic information encoding the infectious, replication-deficient arenavirus particle is derived from a specific species of arenavirus. Thus, in certain embodiments, the infectious, replication-deficient arenavirus particle is derived from LCMV. In other embodiments, the infectious, replication-deficient arenavirus particle is derived from JUNV. In other embodiments, the infectious, replication-deficient arenavirus particle is derived from PICV. Additionally, is specific embodiments, the LCMV is MP strain, WE strain, Armstrong strain, or Armstrong Clone 13 strain. In other specific embodiments, the JUNV is JUNV vaccine Candid #1 strain, or JUNV vaccine XJ Clone 3 strain. In other specific embodiments, the PICV is strain Munchique CoAn4763 isolate P18, or P2 strain.
(a) Methods for Treating a Neoplastic Disease

In certain embodiments, provided herein are methods of treating a neoplastic disease in a subject. Such methods can include administering to a subject in need thereof an arenavirus particle provided herein in combinatoin with a chemotherapeutic agent provided herein. In certain embodiments, the arenavirus particle used in the methods is an infectious, replication-deficient arenavirus particle. Thus, in certain embodiments, the infectious, replication-deficient arenavirus particle used in the methods is engineered to contain a genome comprising (1) a nucleotide sequence encoding a tumor antigen, tumor associated antigen or an antigenic fragment thereof, and (2) the ability to amplify and express its genetic information in infected cells but unable to produce further infectious progeny particles in non-complementing cells.

In certain embodiments, the tumor antigen or tumor associated antigen encoded by the nucleotide sequence included within an arenavirus particle provided herein can be one or more of the tumor antigens or tumor associated antiegens selected from the group consistintg of oncogenic viral antigens, cancer-testis antigens, oncofetal antigens, tissue differentiation antigens, mutant protein antigens, Adipophilin, AIM-2, ALDH1AI, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUCI, MUC5AC, p53 (non-mutant), PAX5, PBF, PRAME, PSMA, RAGE, RAGE-1, RGS5, RhoC, RNF43, RU2AS, secernin 1, SOXI0, STEAPI (six-transmembrane epithelial antigen of the prostate 1), survivin, Telomerase, VEGF, WTi, EGF-R, CEA, CD20, CD33, CD52, MELANA/MART1, MART2,NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSXl or SSX2 fusion protein, TGF-betaRII, Triosephosphate isomerase, ormdm-2, LMP2, HPV E6/E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP-1, GP100, CA-125, CA19-9, Calretinin, Epithelial membrane antigen (EMA), Epithelial tumor antigen (ETA), CD19, CD34, CD99, CD 117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29BCAA), CA 195, CA 242, CA 50, CAM 43, CEA, EBNA, EF2, Epstein-Barr virus antigen, HLA-A2, HLA-Ai i, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F,5T4, 791Tgp72, 13HCG, BCA225, BTAA, CD68KP1, CO-029, HTgp-175, M344, MG7-Ag, MOV18, NB70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin avP3 (CD61), galactin, or Ral-B, CD123, CLL-1, CD38, CS-1, CD138, and ROR1. In certain embodiments, the nucleotide sequence encodes two, three, four, five, six, seven, eight, nine, ten or more tumor antigen, tumor associated antigens or antigenic fragments thereof. In certain embodiments, an antigenic fragment of a tumor antigen, tumor associated antigen provided herein is encoded by the nucleotide sequence included within the arenavirus. In specific embodiments, the tumor antigen is selected from the group consisting of GP100, Trp1, Trp2, and a combination thereof. In specific embodiments, the tumor antigen is GP100. In specific embodiments, the tumor antigen is Trp1. In specific embodiments, the tumor antigen is Trp2.

In certain embodiments, provided herein are methods for treating a neoplastic disease in a subject by administering a chemotherapeutic agent in combination with a replication-deficient arenavirus particle. In certain embodiments, the chemotherapeutic agent is an alkylating agent (e.g., cyclophosphamide), a platinum-based therapeutic, an antimetabolite, a topoisomerase inhibitor, a cytotoxic antibiotic, an intercalating agent, a mitosis inhibitor, a taxane, or a combination of two or more thereof. In certain embodiments, the alkylating agent is a nitrogen mustard, a nitrosourea, an alkyl sulfonate, a non-classical alkylating agent, or a triazene. In certain embodiments, the chemotherapeutic agent comprises one or more of cyclophosphamide, thiotepa, mechlorethamine (chlormethine/mustine), uramustine, melphalan, chlorambucil, ifosfamide, chlornaphazine, cholophosphamide, estramustine, novembichin, phenesterine, prednimustine, trofosfamide, uracil mustard, bendamustine, busulfan, improsulfan, piposulfan, carmustine, lomustine, chlorozotocin, fotemustine, nimustine, ranimustine, streptozucin, cisplatin, carboplatin, nedaplatin, oxaliplatin, satraplatin, triplatin tetranitrate, procarbazine, altretamine, dacarbazine, mitozolomide, temozolomide, paclitaxel, docetaxel, vinblastine, vincristine, vinorelbine, cabazitaxel, dactinomycin (actinomycin D), calicheamicin, dynemicin, amsacrine, doxarubicin, daunorubicin, epirubicin, mitoxantrone, idarubicin, pirarubicin, benzodopa, carboquone, meturedopa, uredopa, altretamine, triethylenemelamine, trietylenephosphoramide, triethiylenethiophosphoramide, trimethylolomelamine, bullatacin, bullatacinone, camptothecin, topotecan, bryostatin, callystatin, CC-1065, adozelesin, carzelesin, bizelesin, cryptophycin, dolastatin, duocarmycin, KW-2189, CB1-TM1, eleutherobin, pancratistatin, sarcodictyin, spongistatin, clodronate, esperamicin, neocarzinostatin chromophore, aclacinomysin, anthramycin, azaserine, bleomycin, cactinomycin, carabicin, carminomycin, carzinophilin, chromomycinis, detorubicin, 6-diazo-5-oxo-L-norleucine, esorubicin, idarubicin, marcellomycin, mitomycin, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, mitotane, trilostane, frolinic acid, aceglatone, aldophosphamide glycoside, aminolevulinic acid, eniluracil, bestrabucil, bisantrene, edatraxate, defofamine, demecolcine, diaziquone, elformithine, elliptinium acetate, etoglucid, gallium nitrate, hydroxyurea, lentinan, lonidainine, maytansine, ansamitocins, mitoguazone, mopidanmol, nitraerine, pentostatin, phenamet, pirarubicin, losoxantrone, podophyllinic acid, 2-ethylhydrazide, PSK polysaccharide complex, razoxane, rhizoxin, sizofiran, spirogermanium, tenuazonic acid, triaziquone, 2,2′,2″-trichlorotriethylamine; T-2 toxin, verracurin A, roridin A and anguidine, urethan, vindesine, mannomustine, mitobronitol, mitolactol, pipobroman, gacytosine, arabinoside (“Ara-C”), etoposide (VP-16), vinorelbine, novantrone, teniposide, edatrexate, aminopterin, xeloda, ibandronate, irinotecan (e.g., CPT-11), topoisomerase inhibitor RFS 2000, difluorometlhylornithine (DMFO), retinoic acid, capecitabine, plicomycin, gemcitabine, navelbine, transplatinum, and pharmaceutically acceptable salts, acids, or derivatives of any of the above. In specific embodiments, the chemotherapeutic agent comprises cyclophosphamide. In certain embodiments, the nitrogen mustard is mechlorethamine, cyclophosphamide, melphalan, chlorambucil, ifosfamide, or busulfan. In certain embodiments, the chemotherapeutic agent alkylates DNA. In certain embodiments, the chemotherapeutic agent alkylates DNA, resulting in the formation of interstrand cross-links (“ICLs”).

In certain embodiments, provided herein are methods for treating a neoplastic disease in a subject by administering a chemotherapeutic agent in combination with a replication-deficient arenavirus particle and an immune checkpoint inhibitor that inhibits, decreases or interferes with the activity of a negative checkpoint regulator. In certain embodiments, the negative checkpoint regulator is selected from the group consisting of Cytotoxic T-lymphocyte antigen-4 (CTLA-4), CD80, CD86, Programmed cell death 1 (PD-1), Programmed cell death ligand 1 (PD-L1), Programmed cell death ligand 2 (PD-L2), Lymphocyte activation gene-3 (LAG-3; also known as CD223), Galectin-3, B and T lymphocyte attenuator (BTLA), T-cell membrane protein 3 (TIM3), Galectin-9 (GAL9), B7-H1, B7-H3, B7-H4, T-Cell immunoreceptor with Ig and ITIM domains (TIGIT/Vstm3/WUCAM/VSIG9), V-domain Ig suppressor of T-Cell activation (VISTA), Glucocorticoid-induced tumor necrosis factor receptor-related (GITR) protein, Herpes Virus Entry Mediator (HVEM), OX40, CD27, CD28, CD137. CGEN-15001T, CGEN-15022, CGEN-15027, CGEN-15049, CGEN-15052, and CGEN-15092. In certain embodiments, the immune checkpoint inhibitor is an anti-PD-1 antibody.

In certain embodiments, the subject that is treated using the methods provided herein is suffering from, is susceptible to, or is at risk for a neoplastic disease. Thus, in some embodiments, the subject is suffering from a neoplastic disease. In some embodiments, the subject is susceptible to a neoplastic disease. In some embodiments, the subject is at risk for a neoplastic disease. In certain embodiments, the neoplastic disease that a subject treatable by the methods provided herein is selected from the group consisting of acute lymphoblastic leukemia; acute lymphoblastic lymphoma; acute lymphocytic leukaemia; acute myelogenous leukemia; acute myeloid leukemia (adult/childhood); adrenocortical carcinoma; AIDS-related cancers; AIDS-related lymphoma; anal cancer; appendix cancer; astrocytomas; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; ewing's sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; hodgkin lymphoma; hypopharyngeal cancer; hypothalamic and visual pathway glioma; intraocular melanoma; islet cell carcinoma (endocrine pancreas); Kaposi Sarcoma; kidney cancer (renal cell cancer); langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); merkel cell cancer; merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-hodgkin lymophoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; respiratory tract carcinoma involving the NUT gene on chromosome 15; retinoblastoma; rhabdomyosarcoma, childhood; salivary gland cancer; sarcoma, Ewing family of tumors; Sezary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sezary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor. In certain embodiments, the neoplastic disease of a subject treatable by the methods provided herein is melanoma. In specific embodiments, the neoplastic disease is melanoma and the chemotherapeutic agent is cyclophosphamide. In specific embodiments, the neoplastic disease is melanoma, the tumor antigen is selected from the group consisting of GP100, Trp1, Trp2, and a combination thereof, and the chemotherapeutic agent is cyclophosphamide. In specific embodiments, the neoplastic disease is melanoma, the tumor antigen is GP100, and the chemotherapeutic agent is cyclophosphamide. In specific embodiments, the neoplastic disease is melanoma, the tumor antigen is Trp2, and the chemotherapeutic agent is cyclophosphamide. In specific embodiments, the neoplastic disease is melanoma, the tumor antigen is Trp1, and the chemotherapeutic agent is cyclophosphamide. In more specific embodiments, the neoplastic disease is melanoma, the tumor antigen is Trp1, the chemotherapeutic agent is cyclophosphamide, and the method further comprises administering an anti-PD-1 antibody.

In certain embodiments, the arenavirus particle provided herein and chemotherapeutic agent provided herein, which are used in the methods provided herein, can be administered in a variety of different combinations. Thus, in certain embodiments, the arenavirus particle and the chemotherapeutic agent are co-administered simultaneously. In other embodiments, the arenavirus particle is administered prior to administration of the chemotherapeutic agent. In still other embodiments, the arenavirus particle is administered after administration of the chemotherapeutic agent. The interval between administration of the arenavirus particle and the chemotherapeutic agent be hours, days, weeks or months. Thus, in some embodiments, interval is about 1 hour, about 2 hours, about 3 hours, about 4 hours, about 5 hours, about 6 hours, about 7 hours, about 8 hours, about 9 hours, about 10 hours, about 11 hours, about 12 hours, about 1 day, about 2 days, about 3 days, about 4 days, about 5 days, about 6 days, about 1 week, about 8 days, about 9 days, about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, or more.

In certain embodiments, the method provided here includes administering an arenavirus particle provided herein and a chemotherapeutic agent provided herein in a therapeutically effective amount. Thus, in certain embodiments, provided herein is a method for treating a neoplastic disease in a subject comprising, administering to a subject in need thereof a therapeutically effective amount of an infectious, replication-deficient arenavirus particle and a therapeutically effective amount of a chemotherapeutic agent, wherein the arenavirus particle is engineered to contain a genome comprising: a nucleotide sequence encoding a tumor antigen, tumor associated antigen or an antigenic fragment thereof; and the ability to amplify and express its genetic information in infected cells but unable to produce further infectious progeny particles in non-complementing cells.

In certain embodiments, provided herein are methods of treating a neoplastic disease in a subject comprising, administering to the subject two or more arenaviruses expressing a tumor antigen, tumor associated antigen or antigenic fragment thereof. In a more specific embodiment, the method provided herein includes administering to the subject a first infectious, replication-deficient arenavirus particle, and administering to the subject, after a period of time, a second infectious, replication-deficient arenavirus particle. In still another embodiment, the first infectious, replication-deficient arenavirus particle and the second infectious, replication-deficient arenavirus particle are derived from different arenavirus species and/or comprise nucleotide sequences encoding different tumor antigen, tumor associated antigens or antigenic fragments thereof.

In certain embodiments, the methods and compositions provided herein are used in combination with personalized medicine. Personalized medicine seeks to benefit patients by using information from a patient's unique genetic and/or epigenetic profile to predict a patient's response to different therapies and identify which therapies are more likely to be effective. Techniques that can be used in combination with the methods and compositions provided herein to obtain a patient's unique genetic and/or epigenetic profile include, but are not limited to, genome sequencing, RNA sequencing, gene expression analysis and identification of a tumor antigen (e.g., neoantigen), tumor associated antigen or an antigenic fragment thereof. In certain embodiments, the selection of an arenavirus tumor antigen or tumor associated antigen for use in the methods and compositions provided herein is performed based on the genetic profile of the patient. In certain embodiments, the selection of an arenavirus tumor antigen or tumor associated antigen for use in the methods and compositions provided herein is performed based on the genetic profile of a tumor or tumor cell. In certain embodiments, the selection of a chemotherapeutic for use in the methods and compositions provided herein is performed based on the genetic profile of a tumor or tumor cell. In certain embodiments, the selection of an arenavirus tumor antigen or tumor associated antigen and the selection of a chemotherapeutic for use in the methods and compositions provided herein are performed based on the genetic profile of a tumor or tumor cell.

### (b) Pharmaceutical Compositions and Kits

In certain embodiments, provided herein are compositions, e.g., pharmaceutical, immunogenic or vaccine compositions, comprising an arenavirus particle provided herein, a chemotherapeutic agent provided herein, and a pharmaceutically acceptable carrier. Thus, in some embodiments, provided herein is a pharmaceutical composition comprising an infectious, replication-deficient arenavirus particle as provided herein, a chemotherapeutic agent as provided herein and a pharmaceutically acceptable carrier. In specific certain embodiments, the arenavirus particle is engineered to contain a genome comprising: (1) a nucleotide sequence encoding a tumor antigen, tumor associated antigen or an antigenic fragment thereof, and (2) the ability to amplify and express its genetic information in infected cells but unable to produce further infectious progeny particles in non-complementing cells.

In certain embodiments, the tumor antigen or tumor associated antigen encoded by the nucleotide sequence included within an arenavirus particle provided herein can be one or more of the tumor antigens or tumor associated antigens selected from the group consistintg of oncogenic viral antigens, cancer-testis antigens, oncofetal antigens, tissue differentiation antigens, mutant protein antigens, Adipophilin, AIM-2, ALDH1AI, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUCI, MUC5AC, p53 (non-mutant), PAX5, PBF, PRAME, PSMA, RAGE, RAGE-1, RGS5, RhoC, RNF43, RU2AS, secernin 1, SOXI0, STEAPI (six-transmembrane epithelial antigen of the prostate 1), survivin, Telomerase, VEGF, WT1, EGF-R, CEA, CD20, CD33, CD52, MELANA/MART1, MART2,NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSXl or SSX2 fusion protein, TGF-betaRII, Triosephosphate isomerase, ormdm-2, LMP2, HPV E6/E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP-1, GP100, CA-125, CA19-9, Calretinin, Epithelial membrane antigen (EMA), Epithelial tumor antigen (ETA), CD19, CD34, CD99, CD 117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29BCAA), CA 195, CA 242, CA 50, CAM 43, CEA, EBNA, EF2, Epstein-Barr virus antigen, HLA-A2, HLA-A11, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F,5T4, 791Tgp72, 13HCG, BCA225, BTAA, CD68KP1, CO-029, HTgp-175, M344, MG7-Ag, MOV18, NB70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin avP3 (CD61), galactin, or Ral-B, CD123, CLL-1, CD38, CS-1, CD138, and ROR1. In certain embodiments, the nucleotide sequence encodes two, three, four, five, six, seven, eight, nine, ten or more tumor antigen, tumor associated antigens or antigenic fragments thereof. In certain embodiments, an antigenic fragment of a tumor antigen or tumor associated antigen provided herein is encoded by the nucleotide sequence included within the arenavirus.

In certain embodiments, a composition provided herein, including a pharmaceutical, immunogenic or vaccine composition, includes a chemotherapeutic agent in combination with a replication-deficient arenavirus particle. In certain embodiments, the chemotherapeutic agent is an alkylating agent (e.g., cyclophosphamide), a platinum-based therapeutic, an antimetabolite, a topoisomerase inhibitor, a cytotoxic antibiotic, an intercalating agent, a mitosis inhibitor, a taxane, or a combination of two or more thereof. In certain embodiments, the alkylating agent is a nitrogen mustard, a nitrosourea, an alkyl sulfonate, a non-classical alkylating agent, or a triazene. In certain embodiments, the chemotherapeutic agent comprises one or more of cyclophosphamide, thiotepa, mechlorethamine (chlormethine/mustine), uramustine, melphalan, chlorambucil, ifosfamide, chlornaphazine, cholophosphamide, estramustine, novembichin, phenesterine, prednimustine, trofosfamide, uracil mustard, bendamustine, busulfan, improsulfan, piposulfan, carmustine, lomustine, chlorozotocin, fotemustine, nimustine, ranimustine, streptozucin, cisplatin, carboplatin, nedaplatin, oxaliplatin, satraplatin, triplatin tetranitrate, procarbazine, altretamine, dacarbazine, mitozolomide, temozolomide, paclitaxel, docetaxel, vinblastine, vincristine, vinorelbine, cabazitaxel, dactinomycin (actinomycin D), calicheamicin, dynemicin, amsacrine, doxarubicin, daunorubicin, epirubicin, mitoxantrone, idarubicin, pirarubicin, benzodopa, carboquone, meturedopa, uredopa, altretamine, triethylenemelamine, trietylenephosphoramide, triethiylenethiophosphoramide, trimethylolomelamine, bullatacin, bullatacinone, camptothecin, topotecan, bryostatin, callystatin, CC-1065, adozelesin, carzelesin, bizelesin, cryptophycin, dolastatin, duocarmycin, KW-2189, CB1-TM1, eleutherobin, pancratistatin, sarcodictyin, spongistatin, clodronate, esperamicin, neocarzinostatin chromophore, aclacinomysin, anthramycin, azaserine, bleomycin, cactinomycin, carabicin, carminomycin, carzinophilin, chromomycinis, detorubicin, 6-diazo-5-oxo-L-norleucine, esorubicin, idarubicin, marcellomycin, mitomycin, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, mitotane, trilostane, frolinic acid, aceglatone, aldophosphamide glycoside, aminolevulinic acid, eniluracil, bestrabucil, bisantrene, edatraxate, defofamine, demecolcine, diaziquone, elformithine, elliptinium acetate, etoglucid, gallium nitrate, hydroxyurea, lentinan, lonidainine, maytansine, ansamitocins, mitoguazone, mopidanmol, nitraerine, pentostatin, phenamet, pirarubicin, losoxantrone, podophyllinic acid, 2-ethylhydrazide, PSK polysaccharide complex, razoxane, rhizoxin, sizofiran, spirogermanium, tenuazonic acid, triaziquone, 2,2′,2″-trichlorotriethylamine; T-2 toxin, verracurin A, roridin A and anguidine, urethan, vindesine, mannomustine, mitobronitol, mitolactol, pipobroman, gacytosine, arabinoside (“Ara-C”), etoposide (VP-16), vinorelbine, novantrone, teniposide, edatrexate, aminopterin, xeloda, ibandronate, irinotecan (e.g., CPT-11), topoisomerase inhibitor RFS 2000, difluorometlhylornithine (DMFO), retinoic acid, capecitabine, plicomycin, gemcitabine, navelbine, transplatinum, and pharmaceutically acceptable salts, acids, or derivatives of any of the above. In specific embodiments, the chemotherapeutic agent comprises cyclophosphamide. In certain embodiments, the nitrogen mustard is mechlorethamine, cyclophosphamide, melphalan, chlorambucil, ifosfamide, or busulfan. In certain embodiments, the chemotherapeutic agent alkylates DNA. In certain embodiments, the chemotherapeutic agent alkylates DNA, resulting in the formation of interstrand cross-links (“ICLs”).

In certain embodiments, the composition provided herein, including a pharmaceutical, immunogenic or vaccine composition, includes a chemotherapeutic agent and an immune checkpoint inhibitor that inhibits, decreases or interferes with the activity of a negative checkpoint regulator. In certain embodiments, the negative checkpoint regulator is selected from the group consisting of Cytotoxic T-lymphocyte antigen-4 (CTLA-4), CD80, CD86, Programmed cell death 1 (PD-1), Programmed cell death ligand 1 (PD-L1), Programmed cell death ligand 2 (PD-L2), Lymphocyte activation gene-3 (LAG-3; also known as CD223), Galectin-3, B and T lymphocyte attenuator (BTLA), T-cell membrane protein 3 (TIM3), Galectin-9 (GAL9), B7-H1, B7-H3, B7-H4, T-Cell immunoreceptor with Ig and ITIM domains (TIGIT/Vstm3/WUCAM/VSIG9), V-domain Ig suppressor of T-Cell activation (VISTA), Glucocorticoid-induced tumor necrosis factor receptor-related (GITR) protein, Herpes Virus Entry Mediator (HVEM), OX40, CD27, CD28, CD137. CGEN-15001T, CGEN-15022, CGEN-15027, CGEN-15049, CGEN-15052, and CGEN-15092. In certain embodiments, the immune checkpoint inhibitor is an anti-PD-1 antibody.

In certain embodiments, the compostions provided herein, including a pharmaceutical, immunogenic or vaccine composition, can be used in the methods described herein. Thus, in certain embodiments, the compositions can be used for the treatment of a neoplastic disease. In specific certain embodiments, the compositions provided herein can be used for the treatment of a neoplastic disease selected from the group consisting of acute lymphoblastic leukemia; acute lymphoblastic lymphoma; acute lymphocytic leukaemia; acute myelogenous leukemia; acute myeloid leukemia (adult/childhood); adrenocortical carcinoma; AIDS-related cancers; AIDS-related lymphoma; anal cancer; appendix cancer; astrocytomas; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; ewing's sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; hodgkin lymphoma; hypopharyngeal cancer; hypothalamic and visual pathway glioma; intraocular melanoma; islet cell carcinoma (endocrine pancreas); Kaposi Sarcoma; kidney cancer (renal cell cancer); langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); merkel cell cancer; merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-hodgkin lymophoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; respiratory tract carcinoma involving the NUT gene on chromosome 15; retinoblastoma; rhabdomyosarcoma, childhood; salivary gland cancer; sarcoma, Ewing family of tumors; Sezary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sezary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor.

Also provided herein are kits that can be used to perform the methods described herein. Thus, in certain embodiments, the kit provided herein includes one or more containers and instructions for use, wherein the one or more containers comprise a composition (e.g., pharmaceutical, immunogenic or vaccine composition) provided herein. In other certain embodiments, a kit provided herein includes containers that each contain the active ingrediates for performing the methods described herein. Thus, in certain embodiments, the kit provided herein includes two or more containers and instructions for use, wherein one of the containers comprises an infectious, replication-deficient arenavirus particle provided herein and another container that comprises a chemotherapeutic agent provided herein. In a specific embodiment, a kit provided herein includes two or more containers and instructions for use, wherein one of the containers comprises an infectious, replication-deficient arenavirus particle provided herein and another container that comprises a chemotherapeutic agent provided herein, wherein the arenavirus particle is engineered to contain a genome comprising: a nucleotide sequence encoding a tumor antigen, tumor associated antigen or an antigenic fragment thereof, and the ability to amplify and express its genetic information in infected cells but unable to produce further infectious progeny particles in non-complementing cells.

### 3.2 Arenavirus Particles Having Non-Natural Open Reading Frame

In certain embodiments, arenaviruses with rearrangements of their ORFs in their genomes and a nucleotide sequence encoding a tumor antigen, tumor associated antigen or an antigenic fragment thereof can be used with the methods and compositions provided herein, such as combinations with a chemotherapeutic agent. In a particular embodiment, an arenavirus particle provided herein includes an arenavirus genomic segment that has been engineered to carry an arenavirus ORF in a position other than the wild-type position. Thus, in certain particular embodiments, provided herein is an arenavirus genomic segment comprising: a nucleotide sequence encoding a tumor antigen, tumor associated antigen or an antigenic fragment thereof; and at least one arenavirus ORF in a position other than the wild-type position of said ORF, wherein the ORF encodes the glycoprotein (“GP”), the nucleoprotein (“NP”), the matrix protein Z (“Z protein”) or the RNA dependent RNA polymerase L (“L protein”) of an arenavirus particle. Also provided herein is an arenavirus particle that has been engineered to contain such an arenavirus genomic segment.

In certain embodiments, an arenavirus particle provided herein is infectious, i.e., it is capable of entering into or injecting its genetic material into a host cell. In certain more specific embodiments, an arenavirus particle as provided herein is infectious, i.e., is capable of entering into or injecting its genetic material into a host cell followed by amplification and expression of its genetic information inside the host cell. In certain embodiments, the arenavirus particle provided herein is engineered to be an infectious, replication-deficient arenavirus particle, i.e., it contains a genome with the ability to amplify and express its genetic information in infected cells but unable to produce further infectious progeny particles in non-complementing cells.

The tumor antigen or tumor associated antigen encoded by the nucleotide sequence included within an arenavirus genomic segment or arenavirus particle provided herein can be one or more of the tumor antigens or tumor associated antigens selected from the group consisting of oncogenic viral antigens, cancer-testis antigens, oncofetal antigens, tissue differentiation antigens, mutant protein antigens, Adipophilin, AIM-2, ALDH1AI, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUCI, MUC5AC, p53 (non-mutant), PAX5, PBF, PRAME, PSMA, RAGE, RAGE-1, RGS5, RhoC, RNF43, RU2AS, secernin 1, SOXI0, STEAPI (six-transmembrane epithelial antigen of the prostate 1), survivin, Telomerase, VEGF, WT1, EGF-R, CEA, CD20, CD33, CD52, MELANA/MART1, MART2,NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSXl or SSX2 fusion protein, TGF-betaRII, Triosephosphate isomerase, ormdm-2, LMP2, HPV E6/E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP-1, GP100, CA-125, CA19-9, Calretinin, Epithelial membrane antigen (EMA), Epithelial tumor antigen (ETA), CD19, CD34, CD99, CD 117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29BCAA), CA 195, CA 242, CA 50, CAM 43, CEA, EBNA, EF2, Epstein-Barr virus antigen, HLA-A2, HLA-A11, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F,5T4, 791Tgp72, 13HCG, BCA225, BTAA, CD68KP1, CO-029, HTgp-175, M344, MG7-Ag, MOV18, NB70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin avP3 (CD61), galactin, or Ral-B, CD123, CLL-1, CD38, CS-1, CD138, and ROR1. In certain embodiments, the nucleotide sequence encodes two, three, four, five, six, seven, eight, nine, ten or more tumor antigens, tumor associated antigens or antigenic fragments thereof. In certain embodiments, an antigenic fragment of a tumor antigen or tumor associated antigen provided herein is encoded by the nucleotide sequence included within the arenavirus.

Accordingly, in certain embodiments, provided herein is an arenavirus genomic segment comprising a nucleotide sequence encoding a tumor antigen, tumor associated antigen or an antigenic fragment thereof provided herein. In certain embodiments, the genomic segment is engineered to carry an arenavirus ORF in a position other than the wild-type position of the ORF. In some embodiments, the arenavirus genomic segment is selected from the group consisting of:
(i) an S segment, wherein the ORF encoding the NP is under control of an arenavirus 5′ UTR;
(ii) an S segment, wherein the ORF encoding the Z protein is under control of an arenavirus 5′ UTR;
(iii) an S segment, wherein the ORF encoding the L protein is under control of an arenavirus 5′ UTR;
(iv) an S segment, wherein the ORF encoding the GP is under control of an arenavirus 3′ UTR;
(v) an S segment, wherein the ORF encoding the L protein is under control of an arenavirus 3′ UTR;
(vi) an S segment, wherein the ORF encoding the Z protein is under control of an arenavirus 3′ UTR;
(vii) an L segment, wherein the ORF encoding the GP is under control of an arenavirus 5′ UTR;
(viii) an L segment, wherein the ORF encoding the NP is under control of an arenavirus 5′ UTR;
(ix) an L segment, wherein the ORF encoding the L protein is under control of an arenavirus 5′ UTR;
(x) an L segment, wherein the ORF encoding the GP is under control of an arenavirus 3′ UTR;
(xi) an L segment, wherein the ORF encoding the NP is under control of an arenavirus 3′ UTR; and
(xii) an L segment, wherein the ORF encoding the Z protein is under control of an arenavirus 3′ UTR.

In certain embodiments, the arenavirus 3′ UTR is the 3′ UTR of the arenavirus S segment or the arenavirus L segment. In certain embodiments, the arenavirus 5′ UTR is the 5′ UTR of the arenavirus S segment or the arenavirus L segment.

In certain embodiments, the arenavirus particle provided herein comprises a second arenavirus genomic segment so that the arenavirus particle comprises an S segment and an L segment.

In certain embodiments, an arenavirus particle provided herein is infectious, i.e., it is capable of entering into or injecting its genetic material into a host cell. In certain more specific embodiments, an arenavirus particle as provided herein is infectious, i.e., is capable of entering into or injecting its genetic material into a host cell followed by amplification and expression of its genetic information inside the host cell. In certain embodiments, the arenavirus particle is an infectious, replication-deficient arenavirus particle engineered to contain a genome with the ability to amplify and express its genetic information in infected cells but unable to produce further infectious progeny particles in non-complementing cells. In certain embodiments, the arenavirus particle is replication-competent and able to produce further infectious progeny particles in normal, not genetically engineered cells. In certain more specific embodiments, such a replication-competent particle is attenuated relative to the wild type virus from which the replication-competent particle is derived.

In certain embodiments, an arenavirus genomic segment provided herein, including an arenavirus particle comprising the arenavirus genomic segment, comprises at least one arenavirus ORF that is at least partially removed or functionally inactivated. The ORF can encode the GP, NP, Z protein, or L protein of an arenavirus particle. Additionally, in certain embodiments, at least one ORF encoding the GP, NP, Z protein, or L protein is removed and replaced with a nucleotide sequence encoding a tumor antigen, tumor associated antigen or an antigenic fragment thereof provided herein. In certain embodiments, only one of the four ORFs encoding GP, NP, Z protein, and L protein is removed. Thus, in certain embodiments, the ORF encoding GP is removed. In certain embodiments, the ORF encoding NP is removed. In certain embodiments, the ORF encoding Z protein is removed. In certain embodiments, the ORF encoding L protein is removed.

In certain embodiments, an arenavirus particle provided herein is derived from a specific arenavirus species, such as lymphocytic choriomeningitis virus (“LCMV”), Junin virus (“JUNV”), or Pichinde virus (“PICV”). In other words, the genomic information encoding the arenavirus particle is derived from a specific species of arenavirus. Thus, in certain embodiments, the arenavirus particle is derived from LCMV. In other embodiments, the arenavirus particle is derived from JUNV. In other embodiments, the arenavirus particle is derived from PICV. Additionally, is specific embodiments, the LCMV is MP strain, WE strain, Armstrong strain, or Armstrong Clone 13 strain. In other specific embodiments, the JUNV is JUNV vaccine Candid #1 strain, or JUNV vaccine XJ Clone 3 strain. In other specific embodiments, the PICV is strain Munchique CoAn4763 isolate P18, or P2 strain.

### (a) Tri-segmented Arenaviruses

In certain embodiments, tri-segmented arenavirus particles comprising a nucleotide sequence encoding a tumor antigen, tumor associated antigen or an antigenic fragment thereof can be used with the methods and compositions provided herein, such as combinations with a chemotherapeutic agent. Thus, in certain embodiments, an arenavirus particle provided herein can comprise one L segment and two S segments or two L segments and one S segment. In certain embodiments, the tri-segmented arenavirus particle provided herein does not recombine into a replication-competent bi-segmented arenavirus particle. Accordingly, in certain embodiments, propagation of the tri-segmented arenavirus particle does not result in a replication-competent bi-segmented particle after 70 days of persistent infection in mice lacking type I interferon receptor, type II interferon receptor and recombination activating gene 1 (RAG1) and having been infected with 10⁴ PFU of the tri-segmented arenavirus particle. The tri-segmented arenavirus particles provided herein, in certain embodiments, can be engineered to improve genetic stability and ensure lasting transgene expression. Moreover, in certain embodiments, inter-segmental recombination of the two S segments or two L segments, uniting two arenavirus ORFs on only one instead of two separate segments, abrogates viral promoter activity.

In certain embodiments, a tri-segmented arenavirus particle, as provided herein, is infectious, i.e., it is capable of entering into or injecting its genetic material into a host cell. In certain more specific embodiments, a tri-segmented arenavirus particle as provided herein is infectious, i.e., is capable of entering into or injecting its genetic material into a host cell followed by amplification and expression of its genetic information inside the host cell. In certain embodiments, the tri-segmented arenavirus particle is an infectious, replication-deficient arenavirus particle engineered to contain a genome with the ability to amplify and express its genetic information in infected cells but unable to produce further infectious progeny particles in non-complementing cells. In certain embodiments, the tri-segmented arenavirus particle is replication-competent and able to produce further infectious progeny particles in normal, not genetically engineered cells. In certain more specific embodiments, such a replication-competent particle is attenuated relative to the wild type virus from which the replication-competent particle is derived.

The tumor antigen or tumor associated antigen encoded by the nucleotide sequence included within a tri-segmented arenavirus particle provided herein can be one or more of the tumor antigens or tumor associated antigens selected from the group consisting of oncogenic viral antigens, cancer-testis antigens, oncofetal antigens, tissue differentiation antigens, mutant protein antigens, Adipophilin, AIM-2, ALDH1AI, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUCI, MUC5AC, p53 (non-mutant), PAX5, PBF, PRAME, PSMA, RAGE, RAGE-1, RGS5, RhoC, RNF43, RU2AS, secernin 1, SOXI0, STEAPI (six-transmembrane epithelial antigen of the prostate 1), survivin, Telomerase, VEGF, WT1, EGF-R, CEA, CD20, CD33, CD52, MELANA/MART1, MART2,NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSXl or-SSX2 fusion protein, TGF-betaRII, Triosephosphate isomerase, ormdm-2, LMP2, HPV E6/E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP-1, GP100, CA-125, CA19-9, Calretinin, Epithelial membrane antigen (EMA), Epithelial tumor antigen (ETA), CD19, CD34, CD99, CD 117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29BCAA), CA 195, CA 242, CA 50, CAM 43, CEA, EBNA, EF2, Epstein-Barr virus antigen, HLA-A2, HLA-A11, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F,5T4, 791Tgp72, 13HCG, BCA225, BTAA, CD68KP1, CO-029, HTgp-175, M344, MG7-Ag, MOV18, NB70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin avP3 (CD61), galactin, or Ral-B, CD123, CLL-1, CD38, CS-1, CD138, and ROR1. In certain embodiments, the nucleotide sequence encodes two, three, four, five, six, seven, eight, nine, ten or more tumor antigens, tumor associated antigens or antigenic fragments thereof. In certain embodiments, an antigenic fragment of a tumor antigen or tumor associated antigen provided herein is encoded by the nucleotide sequence included within the tri-segmented arenavirus.

In certain embodiments, provided herein are tri-segmented arenaviruses with rearrangements of their ORFs in their genomes and a nucleotide sequence encoding a tumor antigen, tumor associated antigen or an antigenic fragment thereof provided herein. In a particular embodiment, a tri-segmented arenavirus particle provided herein has been engineered to carry an arenavirus ORF in a position other than the wild-type position. Thus, in certain particular embodiments, provided herein is a tri-segmented arenavirus comprising: a nucleotide sequence encoding a tumor antigen, tumor associated antigen or an antigenic fragment thereof, and at least one arenavirus ORF in a position other than the wild-type position of said ORF, wherein the ORF encodes the GP, NP, Z protein or L protein of an arenavirus particle.

In certain embodiments, one of the two S segments included in the tri-segmented arenavirus particle provided herein is selected from the group consisting of:
(i) an S segment, wherein the ORF encoding the NP is under control of an arenavirus 5′ UTR
(ii) an S segment, wherein the ORF encoding the Z protein is under control of an arenavirus 5′ UTR;
(iii) an S segment, wherein the ORF encoding the L protein is under control of an arenavirus 5′ UTR;
(iv) an S segment, wherein the ORF encoding the GP is under control of an arenavirus 3′ UTR;
(v) an S segment, wherein the ORF encoding the L protein is under control of an arenavirus 3′ UTR; and
(vi) an S segment, wherein the ORF encoding the Z protein is under control of an arenavirus 3′ UTR.

In certain embodiments, one of the two L segments included in the tri-segmented arenavirus particle provided herein is selected from the group consisting of:
(xiii) an L segment, wherein the ORF encoding the GP is under control of an arenavirus 5′ UTR;
(xiv) an L segment, wherein the ORF encoding the NP is under control of an arenavirus 5′ UTR;
(xv) an L segment, wherein the ORF encoding the L protein is under control of an arenavirus 5′ UTR;
(xvi) an L segment, wherein the ORF encoding the GP is under control of an arenavirus 3′ UTR;
(xvii) an L segment, wherein the ORF encoding the NP is under control of an arenavirus 3′ UTR; and
(xviii) an L segment, wherein the ORF encoding the Z protein is under control of an arenavirus 3′ UTR.

In certain embodiments, the tri-segmented arenavirus particle 3′ UTR is the 3′ UTR of the arenavirus S segment or the arenavirus L segment. In other embodiments, the tri-segmented arenavirus particle 5′ UTR is the 5′ UTR of the arenavirus S segment or the arenavirus L segment.

In certain embodiments, the two S segments comprise: (i) one or two nucleotide sequences each encoding a tumor antigen, tumor associated antigen or an antigenic fragment thereof; or (ii) one or two duplicated arenavirus ORFs; or (iii) one nucleotide sequence encoding a tumor antigen, tumor associated antigen or an antigenic fragment thereof and one duplicated arenavirus ORF.

In certain embodiments, the two L segments comprise (i) one or two nucleotide sequences each encoding a tumor antigen, tumor associated antigen or an antigenic fragment thereof; or (ii) one or two duplicated arenavirus ORFs; or (iii) one nucleotide sequence encoding a tumor antigen, tumor associated antigen or an antigenic fragment thereof and one duplicated arenavirus ORF.

In certain embodiments, a tri-segmented arenavirus particle provided herein, comprises at least one arenavirus ORF that is at least partially removed or functionally inactivated. The ORF can encode the GP, NP, Z protein, or L protein of an arenavirus particle. Additionally, in certain embodiments, at least one ORF encoding the GP, NP, Z protein, or L protein is removed and replaced with a nucleotide sequence encoding a tumor antigen, tumor associated antigen or an antigenic fragment thereof provided herein. In certain embodiments, only one of the four ORFs encoding GP, NP, Z protein, and L protein is removed. Thus, in certain embodiments, the ORF encoding GP is removed. In certain embodiments, the ORF encoding NP is removed. In certain embodiments, the ORF encoding Z protein is removed. In certain embodiments, the ORF encoding L protein is removed.

In certain embodiments, an arenavirus particle provided herein is derived from a specific arenavirus species, such as lymphocytic choriomeningitis virus (“LCMV”), Junin virus (“JUNV”), or Pichinde virus (“PICV”). In other words, the genomic information encoding the arenavirus particle is derived from a specific species of arenavirus. Thus, in certain embodiments, the arenavirus particle is derived from LCMV. In other embodiments, the arenavirus particle is derived from JUNV. In other embodiments, the arenavirus particle is derived from PICV. Additionally, is specific embodiments, the LCMV is MP strain, WE strain, Armstrong strain, or Armstrong Clone 13 strain. In other specific embodiments, the JUNV is JUNV vaccine Candid #1 strain, or JUNV vaccine XJ Clone 3 strain. In other specific embodiments, the PICV is strain Munchique CoAn4763 isolate P18, or P2 strain.

### (b) Methods for Treating a Neoplastic Disease

In certain embodiments, provided herein are methods of treating a neoplastic disease in a subject. Such methods can include administering to a subject in need thereof an arenavirus particle, including a tri-segmented arenavirus particle, provided herein in combination with a chemotherapeutic agent provided herein.

In certain embodiments, the arenavirus particle used in the methods is an infectious, replication-deficient arenavirus particle provided herein. In certain embodiments, the arenavirus particle used in the methods is a tri-segmented arenavirus particle provided herein, including an infectious, replication-deficient tri-segmented arenavirus particle or a replication-competent tri-segmented arenavirus particle. Thus, in certain embodiments, the arenavirus particle, including a tri-segmented arenavirus particle, used in the methods is replication deficient, wherein the tri-segmented arenavirus particle is engineered to contain a genome comprising: (1) a nucleotide sequence encoding a tumor antigen, tumor associated antigen or an antigenic fragment thereof, and (2) the ability to amplify and express its genetic information in infected cells but unable to produce further infectious progeny particles in non-complementing cells. Moreover, in certain embodiments, a tri-segmented arenavirus particle used in the methods is replication-competent, wherein the tri-segmented arenavirus particle is engineered to contain a genome comprising: (1) a nucleotide sequence encoding a tumor antigen, tumor associated antigen or an antigenic fragment thereof; (2) the ability to amplify and express its genetic information in infected cells; and (3) the ability to produce further infectious progeny particles in normal, not genetically engineered cells.

In certain embodiments, the tumor antigen or tumor associated antigen encoded by the nucleotide sequence included within an arenavirus particle, including a tri-segmented arenavirus particle, provided herein can be one or more of the tumor antigens or tumor associated antigens selected from the group consisting of oncogenic viral antigens, cancer-testis antigens, oncofetal antigens, tissue differentiation antigens, mutant protein antigens, Adipophilin, AIM-2, ALDH1AI, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUCl, MUC5AC, p53 (non-mutant), PAX5, PBF, PRAME, PSMA, RAGE, RAGE-1, RGS5, RhoC, RNF43, RU2AS, secernin 1, SOXI0, STEAPI (six-transmembrane epithelial antigen of the prostate 1), survivin, Telomerase, VEGF, WT1, EGF-R, CEA, CD20, CD33, CD52, MELANA/MART1, MART2,NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSXl or-SSX2 fusion protein, TGF-betaRII, Triosephosphate isomerase, ormdm-2, LMP2, HPV E6/E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP-1, GP100, CA-125, CA19-9, Calretinin, Epithelial membrane antigen (EMA), Epithelial tumor antigen (ETA), CD19, CD34, CD99, CD 117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXBI, SPA17, SSX, SYCP1, TPTE, Carbohydrate/ganglioside GM2 (oncofetal antigen-immunogenic-1 OFA-I-1), GM3, CA 15-3 (CA 27.29BCAA), CA 195, CA 242, CA 50, CAM 43, CEA, EBNA, EF2, Epstein-Barr virus antigen, HLA-A2, HLA-All, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, pi80erbB-3, c-met, nm-23H1, TAG-72, TAG-72-4, CA-72-4, CAM 17.1, NuMa, 13-catenin, P16, TAGE, CT7, 43-9F,5T4, 791Tgp72, 13HCG, BCA225, BTAA, CD68KP1, CO-029, HTgp-175, M344, MG7-Ag, MOV18, NB70K, NY-CO-1, RCAS1, SDCCAGi6, TA-90, TAAL6, TLP, TPS, CD22, CD27, CD30, CD70, prostein, TARP (T cell receptor gamma alternate reading frame protein), Trp-p8, integrin avP3 (CD61), galactin, or Ral-B, CD123, CLL-1, CD38, CS-1, CD138, and ROR1. In certain embodiments, the nucleotide sequence encodes two, three, four, five, six, seven, eight, nine, ten or more tumor antigen, tumor associated antigens or antigenic fragments thereof. In certain embodiments, an antigenic fragment of a tumor antigen, tumor associated antigen provided herein is encoded by the nucleotide sequence included within the arenavirus, including a tri-segmented arenavirus. In specific embodiments, the tumor antigen is selected from the group consisting of GP100, Trp1, Trp2, and a combination thereof. In specific embodiments, the tumor antigen is GP100. In specific embodiments, the tumor antigen is Trp1. In specific embodiments, the tumor antigen is Trp2.

In certain embodiments, provided herein are methods for treating a neoplastic disease in a subject by administering a chemotherapeutic agent in combination with a tri-segmented arenavirus particle. In certain embodiments, the chemotherapeutic agent is an alkylating agent (e.g., cyclophosphamide), a platinum-based therapeutic, an antimetabolite, a topoisomerase inhibitor, a cytotoxic antibiotic, an intercalating agent, a mitosis inhibitor, a taxane, or a combination of two or more thereof. In certain embodiments, the alkylating agent is a nitrogen mustard, a nitrosourea, an alkyl sulfonate, a non-classical alkylating agent, or a triazene. In certain embodiments, the chemotherapeutic agent comprises one or more of cyclophosphamide, thiotepa, mechlorethamine (chlormethine/mustine), uramustine, melphalan, chlorambucil, ifosfamide, chlornaphazine, cholophosphamide, estramustine, novembichin, phenesterine, prednimustine, trofosfamide, uracil mustard, bendamustine, busulfan, improsulfan, piposulfan, carmustine, lomustine, chlorozotocin, fotemustine, nimustine, ranimustine, streptozucin, cisplatin, carboplatin, nedaplatin, oxaliplatin, satraplatin, triplatin tetranitrate, procarbazine, altretamine, dacarbazine, mitozolomide, temozolomide, paclitaxel, docetaxel, vinblastine, vincristine, vinorelbine, cabazitaxel, dactinomycin (actinomycin D), calicheamicin, dynemicin, amsacrine, doxarubicin, daunorubicin, epirubicin, mitoxantrone, idarubicin, pirarubicin, benzodopa, carboquone, meturedopa, uredopa, altretamine, triethylenemelamine, trietylenephosphoramide, triethiylenethiophosphoramide, trimethylolomelamine, bullatacin, bullatacinone, camptothecin, topotecan, bryostatin, callystatin, CC-1065, adozelesin, carzelesin, bizelesin, cryptophycin, dolastatin, duocarmycin, KW-2189, CB1-TM1, eleutherobin, pancratistatin, sarcodictyin, spongistatin, clodronate, esperamicin, neocarzinostatin chromophore, aclacinomysin, anthramycin, azaserine, bleomycin, cactinomycin, carabicin, carminomycin, carzinophilin, chromomycinis, detorubicin, 6-diazo-5-oxo-L-norleucine, esorubicin, idarubicin, marcellomycin, mitomycin, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, mitotane, trilostane, frolinic acid, aceglatone, aldophosphamide glycoside, aminolevulinic acid, eniluracil, bestrabucil, bisantrene, edatraxate, defofamine, demecolcine, diaziquone, elformithine, elliptinium acetate, etoglucid, gallium nitrate, hydroxyurea, lentinan, lonidainine, maytansine, ansamitocins, mitoguazone, mopidanmol, nitraerine, pentostatin, phenamet, pirarubicin, losoxantrone, podophyllinic acid, 2-ethylhydrazide, PSK polysaccharide complex, razoxane, rhizoxin, sizofiran, spirogermanium, tenuazonic acid, triaziquone, 2,2′,2″-trichlorotriethylamine; T-2 toxin, verracurin A, roridin A and anguidine, urethan, vindesine, mannomustine, mitobronitol, mitolactol, pipobroman, gacytosine, arabinoside (“Ara-C”), etoposide (VP-16), vinorelbine, novantrone, teniposide, edatrexate, aminopterin, xeloda, ibandronate, irinotecan (e.g., CPT-11), topoisomerase inhibitor RFS 2000, difluorometlhylornithine (DMFO), retinoic acid, capecitabine, plicomycin, gemcitabine, navelbine, transplatinum, and pharmaceutically acceptable salts, acids, or derivatives of any of the above. In specific embodiments, the chemotherapeutic agent comprises cyclophosphamide. In certain embodiments, the nitrogen mustard is mechlorethamine, cyclophosphamide, melphalan, chlorambucil, ifosfamide, or busulfan. In certain embodiments, the chemotherapeutic agent alkylates DNA. In certain embodiments, the chemotherapeutic agent alkylates DNA, resulting in the formation of interstrand cross-links (“ICLs”).

In certain embodiments, provided herein are methods for treating a neoplastic disease in a subject by administering a chemotherapeutic agent in combination with a tri-segmented arenavirus particle and an immune checkpoint inhibitor that inhibits, decreases or interferes with the activity of a negative checkpoint regulator. In certain embodiments, the negative checkpoint regulator is selected from the group consisting of Cytotoxic T-lymphocyte antigen-4 (CTLA-4), CD80, CD86, Programmed cell death 1 (PD-1), Programmed cell death ligand 1 (PD-L1), Programmed cell death ligand 2 (PD-L2), Lymphocyte activation gene-3 (LAG-3; also known as CD223), Galectin-3, B and T lymphocyte attenuator (BTLA), T-cell membrane protein 3 (TIM3), Galectin-9 (GAL9), B7-H1, B7-H3, B7-H4, T-Cell immunoreceptor with Ig and ITIM domains (TIGIT/Vstm3/WUCAM/VSIG9), V-domain Ig suppressor of T-Cell activation (VISTA), Glucocorticoid-induced tumor necrosis factor receptor-related (GITR) protein, Herpes Virus Entry Mediator (HVEM), OX40, CD27, CD28, CD137. CGEN-15001T, CGEN-15022, CGEN-15027, CGEN-15049, CGEN-15052, and CGEN-15092. In certain embodiments, the immune checkpoint inhibitor is an anti-PD-1 antibody.

In certain embodiments, the subject that is treated using the methods provided herein is suffering from, is susceptible to, or is at risk for a neoplastic disease. Thus, in some embodiments, the subject is suffering from a neoplastic disease. In some embodiments, the subject is susceptible to a neoplastic disease. In some embodiments, the subject is at risk for a neoplastic disease. In certain embodiments, the neoplastic disease of a subject treatable by the methods provided herein is selected from the group consisting of acute lymphoblastic leukemia; acute lymphoblastic lymphoma; acute lymphocytic leukaemia; acute myelogenous leukemia; acute myeloid leukemia (adult/childhood); adrenocortical carcinoma; AIDS-related cancers; AIDS-related lymphoma; anal cancer; appendix cancer; astrocytomas; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; ewing's sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; hodgkin lymphoma; hypopharyngeal cancer; hypothalamic and visual pathway glioma; intraocular melanoma; islet cell carcinoma (endocrine pancreas); Kaposi Sarcoma; kidney cancer (renal cell cancer); langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); merkel cell cancer; merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-hodgkin lymophoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; respiratory tract carcinoma involving the NUT gene on chromosome 15; retinoblastoma; rhabdomyosarcoma, childhood; salivary gland cancer; sarcoma, Ewing family of tumors; Sezary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sezary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor. In certain embodiments, the neoplastic disease of a subject treatable by the methods provided herein is melanoma. In specific embodiments, the neoplastic disease is melanoma and the chemotherapeutic agent is cyclophosphamide. In specific embodiments, the neoplastic disease is melanoma, the tumor antigen is selected from the group consisting of GP100, Trp1, Trp2, and a combination thereof, and the chemotherapeutic agent is cyclophosphamide. In specific embodiments, the neoplastic disease is melanoma, the tumor antigen is GP100, and the chemotherapeutic agent is cyclophosphamide. In specific embodiments, the neoplastic disease is melanoma, the tumor antigen is Trp2, and the chemotherapeutic agent is cyclophosphamide. In specific embodiments, the neoplastic disease is melanoma, the tumor antigen is Trp1, and the chemotherapeutic agent is cyclophosphamide. In more specific embodiments, the neoplastic disease is melanoma, the tumor antigen is Trp1, the chemotherapeutic agent is cyclophosphamide, and the method further comprises administering an anti-PD-1 antibody.

In certain embodiments, the arenavirus particle, including a tri-segmented arenavirus, provided herein and chemotherapeutic agents, which are used in the methods provided herein, can be administered in a variety of different combinations. Thus, in certain embodiments, the arenavirus particle and the chemotherapeutic agent are co-administered simultaneously. In other embodiments, the arenavirus particle is administered prior to administration of the chemotherapeutic agent. In still other embodiments, the arenavirus particle is administered after administration of the chemotherapeutic agent. The interval between administration of the arenavirus particle and the chemotherapeutic agent can be hours, days, weeks or months. Thus, in some embodiments, the interval is about 1 hour, about 2 hours, about 3 hours, about 4 hours, about 5 hours, about 6 hours, about 7 hours, about 8 hours, about 9 hours, about 10 hours, about 11 hours, about 12 hours, about 1 day, about 2 days, about 3 days, about 4 days, about 5 days, about 6 days, about 1 week, about 8 days, about 9 days, about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, or more.

In certain embodiments, the method provided here includes administering an arenavirus particle, including a tri-segmented arena virus, provided herein and the chemotherapeutic agent provided herein in a therapeutically effective amount. Thus, in certain embodiments, provided herein is a method for treating a neoplastic disease in a subject comprising, administering to a subject in need thereof a therapeutically effective amount of an arenavirus particle and a therapeutically effective amount of a chemotherapeutic agent, wherein the arenavirus particle is engineered to contain a genomic segment comprising: a nucleotide sequence encoding a tumor antigen, tumor associated antigen or an antigenic fragment thereof, and at least one arenavirus ORF in a position other than the wild-type position of the ORF, wherein the ORF encodes the GP, NP, Z protein or L protein of the arenavirus particle.

In certain embodiments, provided herein are methods of treating a neoplastic disease in a subject comprising, administering to the subject two or more arenaviruses, including a tri-segmented arenavirus, provided herein expressing a tumor antigen, tumor associated antigen or antigenic fragment thereof. In a more specific embodiment, the method provided herein includes administering to the subject a first arenavirus particle, and administering to the subject, after a period of time, a second arenavirus particle. In still another embodiment, the first arenavirus particle and the second arenavirus particle are derived from different arenavirus species and/or comprise nucleotide sequences encoding different tumor antigen, tumor associated antigens or antigenic fragments thereof.

In certain embodiments, the methods and compositions provided herein are used in combination with personalized medicine. Personalized medicine seeks to benefit patients by using information from a patient's unique genetic and/or epigenetic profile to predict a patient's response to different therapies and identify which therapies are more likely to be effective. Techniques that can be used in combination with the methods and compositions provided herein to obtain a patient's unique genetic and/or epigenetic profile include, but are not limited to, genome sequencing, RNA sequencing, gene expression analysis and identification of a tumor antigen (e.g., neoantigen), tumor associated antigen or an antigenic fragment thereof. In certain embodiments, the selection of an arenavirus tumor antigen or tumor associated antigen for use in the methods and compositions provided herein is performed based on the genetic profile of the patient. In certain embodiments, the selection of an arenavirus tumor antigen or tumor associated antigen for use in the methods and compositions provided herein is performed based on the genetic profile of a tumor or tumor cell. In certain embodiments, the selection of a chemotherapeutic for use in the methods and compositions provided herein is performed based on the genetic profile of a tumor or tumor cell. In certain embodiments, the selection of an arenavirus tumor antigen or tumor associated antigen and the selection of a chemotherapeutic for use in the methods and compositions provided herein are performed based on the genetic profile of a tumor or tumor cell.

In one embodiment, disclosed herein is a method for treating a neoplastic disease in a subject comprising administering to a subject in need thereof an arenavirus particle and a chemotherapeutic agent, wherein said arenavirus particle is engineered to contain an arenavirus genomic segment comprising: (i) a nucleotide sequence encoding a tumor antigen, tumor associated antigen or an antigenic fragment thereof; and (ii) at least one arenavirus open reading frame (“ORF”) in a position other than the wild-type position of said ORF, wherein said ORF encodes the glycoprotein (“GP”), the nucleoprotein (“NP”), the matrix protein Z (“Z protein”) or the RNA dependent RNA polymerase L (“L protein”) of said arenavirus particle. In certain embodiments, said tumor antigen or tumor associated antigen is selected from the group consisting of GP100, Trp1, and Trp2. In certain embodiments, said chemotherapeutic agent is cyclophosphamide. In certain embodiments, said subject is suffering from, is susceptible to, or is at risk for melanoma. In certain embodiments, the arenavirus particle is a tri-segmented arenavirus particle comprising one L segment and two S segments. In certain embodiments, one of said two S segments is an S segment, wherein the ORF encoding the GP is under control of an arenavirus 3′ UTR. In certain embodiments, each of the two S segments comprise a nucleotide sequence encoding a tumor antigen, tumor associated antigen or an antigenic fragment thereof. In certain embodiments, said arenavirus particle is derived from LCMV. In specific embodiments, said arenavirus particle is derived from LCMV Clone 13. In specific embodiments, said arenavirus particle is derived from LCMV strain WE. In specific embodiments, said arenavirus particle is derived from LCMV Clone 13 and strain WE.

In one embodiment, disclosed herein is a method for treating melanoma in a subject comprising administering to a subject in need thereof an arenavirus particle and a chemotherapeutic agent, wherein said arenavirus particle is engineered to contain an arenavirus genomic segment comprising: (i) a nucleotide sequence encoding a tumor antigen, tumor associated antigen or an antigenic fragment thereof; and (ii) at least one arenavirus open reading frame (“ORF”) in a position other than the wild-type position of said ORF, wherein said ORF encodes the glycoprotein (“GP”), the nucleoprotein (“NP”), the matrix protein Z (“Z protein”) or the RNA dependent RNA polymerase L (“L protein”) of said arenavirus particle, wherein said tumor antigen or tumor associated antigen is selected from the group consisting of GP100, Trp1, and Trp2, said chemotherapeutic agent is cyclophosphamide, said arenavirus particle is derived from LCMV and is a tri-segmented arenavirus particle comprising one L segment and two S segments, and wherein, in one of said two S segments the ORF encoding the GP is under control of an arenavirus 3′ UTR, and each of the two S segments comprise a nucleotide sequence encoding said tumor antigen, tumor associated antigen or antigenic fragment thereof. (c) Pharmaceutical Compositions and Kits

In certain embodiments, provided herein are compositions, e.g., pharmaceutical, immunogenic or vaccine compositions, comprising an arenavirus particle, including a tri-segmented arenavirus particle, provided herein, a chemotherapeutic agent provided herein, and a pharmaceutically acceptable carrier. Thus, in some embodiments, provided herein is a pharmaceutical composition comprising an arenavirus particle as provided herein, a chemotherapeutic agent as provided herein and a pharmaceutically acceptable carrier.

In certain embodiments, the arenavirus particle contained within the compositions is an infectious, replication-deficient arenavirus particle provided herein. In certain embodiments, the arenavirus particle contained within the compositions is a tri-segmented arenavirus particle provided herein, including an infectious, replication-deficient tri-segmented arenavirus particle or a replication-competent tri-segmented arenavirus particle. Thus, in certain embodiments, the compositions providing herein, including a pharmaceutical, immunogenic or vaccine composition, comprise an arenavirus particle, including a tri-segmented arenavirus particle, that is replication-deficient, wherein the arenavirus particle is engineered to contain a genome comprising: (1) a nucleotide sequence encoding a tumor antigen, tumor associated antigen or an antigenic fragment thereof; and (2) the ability to amplify and express its genetic information in infected cells but unable to produce further infectious progeny particles in non-complementing cells. Moreover, in certain embodiments, the compositions providing herein, including a pharmaceutical, immunogenic or vaccine composition, comprise a tri-segmented arenavirus particle, that is replication-competent, wherein the arenavirus particle is engineered to contain a genome comprising: (1) a nucleotide sequence encoding a tumor antigen, tumor associated antigen or an antigenic fragment thereof; (2) the ability to amplify and express its genetic information in infected cells; and (3) the ability to produce further infectious progeny particles in normal, not genetically engineered cells.

In certain embodiments, the tumor antigen or tumor associated antigen encoded by the nucleotide sequence included within an arenavirus particle provided herein can be one or more of the tumor antigens or tumor associated antigens selected from the group consisting of oncogenic viral antigens, cancer-testis antigens, oncofetal antigens, tissue differentiation antigens, mutant protein antigens, Adipophilin, AIM-2, ALDH1AI, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUCI, MUC5AC, p53 (non-mutant), PAX5, PBF, PRAME, PSMA, RAGE, RAGE-1, RGS5, RhoC, RNF43, RU2AS, secernin 1, SOXI0, STEAPI (six-transmembrane epithelial antigen of the prostate 1), survivin, Telomerase, VEGF, WT1, EGF-R, CEA, CD20, CD33, CD52, MELANA/MART1, MART2,NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSXl or-SSX2 fusion protein, TGF-betaRII, Triosephosphate isomerase, ormdm-2, LMP2, HPV E6/E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP-1, GP100, CA-125, CA19-9, Calretinin, Epithelial membrane antigen (EMA), Epithelial tumor antigen (ETA), CD19, CD34, CD99, CD 117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29BCAA), CA 195, CA 242, CA 50, CAM 43, CEA, EBNA, EF2, Epstein-Barr virus antigen, HLA-A2, HLA-Ai i, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, pi80erbB-3, c-met, nm-23H1, TAG-72, TAG-72-4, CA-72-4, CAM 17.1, NuMa, 13-catenin, P16, TAGE, CT7, 43-9F,5T4, 791Tgp72, 13HCG, BCA225, BTAA, CD68KP1, CO-029, HTgp-175, M344, MG7-Ag, MOV18, NB70K, NY-CO-1, RCAS1, SDCCAGi6, TA-90, TAAL6, TLP, TPS, CD22, CD27, CD30, CD70, prostein, TARP (T cell receptor gamma alternate reading frame protein), Trp-p8, integrin avP3 (CD61), galactin, or Ral-B, CD123, CLL-1, CD38, CS-1, CD138, and ROR1. In certain embodiments, the nucleotide sequence encodes two, three, four, five, six, seven, eight, nine, ten or more tumor antigen, tumor associated antigens or antigenic fragments thereof. In certain embodiments, an antigenic fragment of a tumor antigen or tumor associated antigen provided herein is encoded by the nucleotide sequence included within the arenavirus.

In certain embodiments, the composition provided herein, including a pharmaceutical, immunogenic or vaccine composition, includes a chemotherapeutic agent. In certain embodiments, the chemotherapeutic agent is an alkylating agent (e.g., cyclophosphamide), a platinum-based therapeutic, an antimetabolite, a topoisomerase inhibitor, a cytotoxic antibiotic, an intercalating agent, a mitosis inhibitor, a taxane, or a combination of two or more thereof. In certain embodiments, the alkylating agent is a nitrogen mustard, a nitrosourea, an alkyl sulfonate, a non-classical alkylating agent, or a triazene. In certain embodiments, the chemotherapeutic agent comprises one or more of cyclophosphamide, thiotepa, mechlorethamine (chlormethine/mustine), uramustine, melphalan, chlorambucil, ifosfamide, chlornaphazine, cholophosphamide, estramustine, novembichin, phenesterine, prednimustine, trofosfamide, uracil mustard, bendamustine, busulfan, improsulfan, piposulfan, carmustine, lomustine, chlorozotocin, fotemustine, nimustine, ranimustine, streptozucin, cisplatin, carboplatin, nedaplatin, oxaliplatin, satraplatin, triplatin tetranitrate, procarbazine, altretamine, dacarbazine, mitozolomide, temozolomide, paclitaxel, docetaxel, vinblastine, vincristine, vinorelbine, cabazitaxel, dactinomycin (actinomycin D), calicheamicin, dynemicin, amsacrine, doxarubicin, daunorubicin, epirubicin, mitoxantrone, idarubicin, pirarubicin, benzodopa, carboquone, meturedopa, uredopa, altretamine, triethylenemelamine, trietylenephosphoramide, triethiylenethiophosphoramide, trimethylolomelamine, bullatacin, bullatacinone, camptothecin, topotecan, bryostatin, callystatin, CC-1065, adozelesin, carzelesin, bizelesin, cryptophycin, dolastatin, duocarmycin, KW-2189, CB1-TM1, eleutherobin, pancratistatin, sarcodictyin, spongistatin, clodronate, esperamicin, neocarzinostatin chromophore, aclacinomysin, anthramycin, azaserine, bleomycin, cactinomycin, carabicin, carminomycin, carzinophilin, chromomycinis, detorubicin, 6-diazo-5-oxo-L-norleucine, esorubicin, idarubicin, marcellomycin, mitomycin, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, mitotane, trilostane, frolinic acid, aceglatone, aldophosphamide glycoside, aminolevulinic acid, eniluracil, bestrabucil, bisantrene, edatraxate, defofamine, demecolcine, diaziquone, elformithine, elliptinium acetate, etoglucid, gallium nitrate, hydroxyurea, lentinan, lonidainine, maytansine, ansamitocins, mitoguazone, mopidanmol, nitraerine, pentostatin, phenamet, pirarubicin, losoxantrone, podophyllinic acid, 2-ethylhydrazide, PSK polysaccharide complex, razoxane, rhizoxin, sizofiran, spirogermanium, tenuazonic acid, triaziquone, 2,2′,2″-trichlorotriethylamine; T-2 toxin, verracurin A, roridin A and anguidine, urethan, vindesine, mannomustine, mitobronitol, mitolactol, pipobroman, gacytosine, arabinoside (“Ara-C”), etoposide (VP-16), vinorelbine, novantrone, teniposide, edatrexate, aminopterin, xeloda, ibandronate, irinotecan (e.g., CPT-11), topoisomerase inhibitor RFS 2000, difluorometlhylornithine (DMFO), retinoic acid, capecitabine, plicomycin, gemcitabine, navelbine, transplatinum, and pharmaceutically acceptable salts, acids, or derivatives of any of the above. In specific embodiments, the chemotherapeutic agent comprises cyclophosphamide. In certain embodiments, the nitrogen mustard is mechlorethamine, cyclophosphamide, melphalan, chlorambucil, ifosfamide, or busulfan. In certain embodiments, the chemotherapeutic agent alkylates DNA. In certain embodiments, the chemotherapeutic agent alkylates DNA, resulting in the formation of interstrand cross-links (“ICLs”).

In certain embodiments, the composition provided herein, including a pharmaceutical, immunogenic or vaccine composition, includes a chemotherapeutic agent and an immune checkpoint inhibitor that inhibits, decreases or interferes with the activity of a negative checkpoint regulator. In certain embodiments, the negative checkpoint regulator is selected from the group consisting of Cytotoxic T-lymphocyte antigen-4 (CTLA-4), CD80, CD86, Programmed cell death 1 (PD-1), Programmed cell death ligand 1 (PD-L1), Programmed cell death ligand 2 (PD-L2), Lymphocyte activation gene-3 (LAG-3; also known as CD223), Galectin-3, B and T lymphocyte attenuator (BTLA), T-cell membrane protein 3 (TIM3), Galectin-9 (GAL9), B7-H1, B7-H3, B7-H4, T-Cell immunoreceptor with Ig and ITIM domains (TIGIT/Vstm3/WUCAM/VSIG9), V-domain Ig suppressor of T-Cell activation (VISTA), Glucocorticoid-induced tumor necrosis factor receptor-related (GITR) protein, Herpes Virus Entry Mediator (HVEM), OX40, CD27, CD28, CD137. CGEN-15001T, CGEN-15022, CGEN-15027, CGEN-15049, CGEN-15052, and CGEN-15092. In certain embodiments, the immune checkpoint inhibitor is an anti-PD-1 antibody.

In certain embodiments, the compositions provided herein, a pharmaceutical, immunogenic or vaccine composition, can be used in the methods described herein. Thus, in certain embodiments, the compositions can be used for the treatment of a neoplastic disease. In specific certain embodiments, the compositions provided herein can be used for the treatment of a neoplastic disease selected from the group consisting of acute lymphoblastic leukemia; acute lymphoblastic lymphoma; acute lymphocytic leukaemia; acute myelogenous leukemia; acute myeloid leukemia (adult/childhood); adrenocortical carcinoma; AIDS-related cancers; AIDS-related lymphoma; anal cancer; appendix cancer; astrocytomas; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; ewing's sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; hodgkin lymphoma; hypopharyngeal cancer; hypothalamic and visual pathway glioma; intraocular melanoma; islet cell carcinoma (endocrine pancreas); Kaposi Sarcoma; kidney cancer (renal cell cancer); langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); merkel cell cancer; merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-hodgkin lymophoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; respiratory tract carcinoma involving the NUT gene on chromosome 15; retinoblastoma; rhabdomyosarcoma, childhood; salivary gland cancer; sarcoma, Ewing family of tumors; Sezary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sezary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor.

Also provided herein are kits that can be used to perform the methods described herein. Thus, in certain embodiments, the kit provided herein includes one or more containers and instructions for use, wherein the one or more containers comprise a composition (e.g., pharmaceutical, immunogenic or vaccine composition) provided herein. In other certain embodiments, a kit provided herein includes containers that each contains the active ingredients for performing the methods described herein. Thus, in certain embodiments, the kit provided herein includes two or more containers and instructions for use, wherein one of the containers comprises an arenavirus particle, including a tri-segmented arenavirus particle, provided herein and another container comprises a chemotherapeutic agent provided herein. In a specific embodiment, a kit provided herein includes two or more containers and instructions for use, wherein one of the containers comprises an arenavirus particle, including a tri-segmented arenavirus particle, provided herein and another container comprises a chemotherapeutic agent provided herein, wherein the arenavirus particle is engineered to contain a genome comprising: a nucleotide sequence encoding a tumor antigen, tumor associated antigen or an antigenic fragment thereof; and the ability to amplify and express its genetic information in infected cells but unable to produce further infectious progeny particles in non-complementing cells. Moreover, in certain embodiments, one of the containers comprises a tri-segmented arenavirus particle that is engineered to contain a genome comprising: a nucleotide sequence encoding a tumor antigen, tumor associated antigen or an antigenic fragment thereof; the ability to amplify and express its genetic information in infected cells; and the ability to produce further infectious progeny particles in normal, not genetically engineered cells.

### 3.3 Conventions and Abbreviations

| Abbreviation | Convention |
|---|---|
| APC | Antigen presenting cells |
| C-cell | Complementing cell line |
| CD4 | Cluster of Differentiation 4 |
| CD8 | Cluster of Differentiation 8 |
| CMI | Cell-mediated immunity |
| GP | Glycoprotein |
| GS-plasmid | Plasmid expressing genome segments |
| IGR | Intergenic region |
| JUNV | Junin virus |
| L protein | RNA-dependent RNA polymerase |
| L segment | Long segment |
| LCMV | Lymphocytic choriomeningitis virus |
| MHC | Major Histocompatibility Complex |
| NP | Nucleoprotein |
| ORF | Open reading frame |
| S segment | Short segment |
| TF-plasmid | Plasmid expressing transacting factors |
| UTR | Untranslated region |
| Z protein | Matrix Protein Z |

### 4. BRIEF DESCRIPTION OF THE FIGURES

FIG. 1. The genome of wild type arenaviruses consists of a short (1; ˜3.4 kb) and a large (2; ˜7.2 kb) RNA segment. The short segment carries open reading frames encoding the nucleoprotein (3) and glycoprotein (4). The large segment encodes the RNA-dependent RNA polymerase L (5) and the matrix protein Z (6). Wild type arenaviruses can be rendered replication-deficient vaccine vectors by deleting the glycoprotein gene and inserting, instead of the glycoprotein gene, a tumor antigen, tumor associated antigen, or antigenic fragment thereof described herein (7) against which immune responses are to be induced.

FIG. 2. Schematic representation of the genomic organization of bi- and tri-segmented LCMV. The bi-segmented genome of wild-type LCMV consists of one S segment encoding the GP and NP and one L segment encoding the Z protein and the L protein (i). Both segments are flanked by the respective 5′ and 3′ UTRs. The genome of recombinant tri-segmented LCMV (r3LCMV) consists of one L and two S segments with one position where to insert a gene of interest (here GFP, which can alternatively be a tumor antigen, tumor associated antigen or antigenic fragment thereof as described herein) into each one of the S segments. r3LCMV-GFP^{natural} (nat) has all viral genes in their natural position (ii), whereas the GP ORF in r3LCMV-GFP^{arificial} (art) is artificially juxtaposed to and expressed under control of the 3′ UTR (iii).

FIG. 3A-C. Tumor growth in C57BL/6 mice after tumor challenge with B16F10 tumor cells (A) as well as animal survival (B and C) were monitored. Results are shown for C57BL/6 mice left untreated (group 1), treated with cyclophosphamide (group 2), treated with vector mix (each of r3LCMV-GP100, r3LCMV-Trp1 and r3LCMV-Trp2) (group 3), or treated with a combination of cyclophosphamide and vector mix (group 4). Symbols represent the mean±SEM of three mice (groups 1-3) or four mice (group 4) per group.

FIG. 4A-B. Relative (left panel) and absolute (right panel) numbers of (A) Trp2-specific CD8+ T cells or (B) GP100-specific CD8+ T cells induced in mice treated with a combination of cyclophosphamide and r3LCMV-vectors compared to animals treated with r3LCMV vectors only.

FIG. 5A-C. C57BL/6 mice (5 mice per group) were immunized intravenously on day 0 with 10⁵ RCV FFU of r3LCMV-E7E6 (group 1) or 10⁵ RCV FFU of r3PICV-E7E6 (group 2) or were left untreated (group 3). On day 13 mice in groups 1 and 2 were boosted with 10⁵ RCV FFU of r3LCMV-E7E6. Mice of group 3 were again left untreated. E7-specific CD8+ T cell frequencies were subsequently analyzed by tetramer staining (Db-E7 (49-57)-Tetramer) on days 20 (A) and 42 (B) in the blood, and on day 51 in the spleen (C) of test animals.

FIG. 6. On day 0 of the experiment female C57BL/6 mice (n=5 or n=3 animals per group for experimental groups and buffer group, respectively) were challenged subcutaneously with 1×10⁵ TC-1 tumor cells, derived from mouse primary epithelial cells, co-transformed with HPV16 E6 and E7 and c-Ha-ras oncogenes. Ten days later (day 10 of the experiment) mice were immunized intravenously with either buffer (group 1) or 10⁵ RCV FFU r3LCMV-E7E6 (group 2) or 10⁵ RCV FFU r3PICV-E7E6 (group 3). 14 days post prime (day 24 of the experiment) mice in groups 2 and 3 received a boost administration of 105 RCV FFU r3LCMV-E7E6. Tumor growth was subsequently monitored over time. Arithmetic means+/−SEM are shown. Arrows indicate time points of vaccination.

FIG. 7A-B. 1×10⁵ B16F10 tumor cells were implanted subcutaneously into C57BL/6 mice on day 0. Mice were subsequently left untreated (group 1), treated intraperitoneally with 2 mg cyclophosphamide (CTX) on day 6 and 200 μg each of anti PD-1 and anti-CTLA-4 on days 10, 13, 16, 19 and 22 (group 2), treated intraperitoneally with 2 mg cyclophosphamide on day 6 and injected intravenously with 1.2×10⁵ FFU (in total) of a r3LCMV vector mix (r3LCMV-GP100, r3LCMV-Trp1 and r3LCMV-Trp2) on day 7 (group 3), or treated with cyclophosphamide on day 6, an r3LCMV-vector mix on day 7 and anti PD-1 and anti-CTLA-4 on days 10, 13, 16, 19 and 22 (group 4). Tumor size (A) and percent animal survival (B) were monitored.

### 5. DETAILED DESCRIPTION OF THE INVENTION

### 5.1 Replication-Deficient Arenavirus Particles

In certain embodiments, replication-deficient arenavirus particles comprising a nucleotide sequence encoding a tumor antigen, tumor associated antigen or an antigenic fragment thereof in combination with chemotherapeutic agent, can be used as immunotherapies for treating a neoplastic disease, such as cancer. The term “neoplastic” or “neoplasm” refers to an abnormal new growth of cells or tissue. This abnormal new growth can form a mass, also known as a tumor or neoplasia. A neoplasm includes a benign neoplasm, an in situ neoplasm, a malignant neoplasm, and a neoplasm of uncertain or unknown behavior. In certain embodiments, the neoplastic disease treated using the methods and compositions described herein is cancer.

Provided herein are combination treatments for the treatment and/or prevention of a neoplastic disease, such as cancer. Specifically, such combination treatments comprise administering arenavirus particles or viral vectors that comprise a nucleotide sequence encoding one or more tumor antigens, tumor associated antigens or antigenic fragments thereof in combination with one or more chemotherapeutic agents. These genetically modified viruses can be administered to a subject for the treatment of a neoplastic disease, such as cancer. Detailed descriptions of the arenaviruses provided herein, including the nucleotide sequences encoding a tumor antigen, tumor associated antigen or antigenic fragment thereof can be found in Sections 5.1.(a) and 5.1.(b). Additionally, methods for generation of arenavirus particles or viral vectors for use in the methods and compositions described herein are described in more detail in Section 5.1.(c).

In addition to administering arenavirus particles or viral vectors to a subject, the immunotherapies for treating a neoplastic disease provided herein can include a chemotherapeutic agent. “Chemotherapeutic agents” are cytotoxic anti-cancer agents, and can be categorized by their mode of activity within a cell, for example, at what stage they affect the cell cycle (e.g., a mitosis inhibitor). Alternatively, chemotherapeutic agents can be characterized based on ability to cross-link DNA, to intercalate into DNA, or to induce chromosomal aberrations by affecting nucleic acid synthesis (e.g., alkylating agents), among other mechanisms of action. Chemotherapeutic agents can also be characterized based on chemical components or structure (e.g., platinum-based therapeutics). Thus, in certain embodiments, provided herein are methods and compositions for treating a neoplastic disease using an arenavirus particle or viral vector comprising a nucleotide sequence encoding a tumor antigen, tumor associated antigen or antigenic fragment thereof and a chemotherapeutic agent.

Thus, in certain embodiments, provided herein are methods and compositions for treating a neoplastic disease using an arenavirus particle or viral vector comprising a nucleotide sequence encoding a tumor antigen, tumor associated antigen or antigenic fragment thereof and a chemotherapeutic agent. Also, in certain embodiments, provided herein are compositions comprising an arenavirus particle or viral vector comprising a nucleotide sequence encoding a tumor antigen, tumor associated antigen or antigenic fragment thereof, and a chemotherapeutic agent. In certain embodiments, the arenavirus particle provided herein is an infectious, replication deficient arenavirus particle.

Methods of using arenavirus particles for viral vectors for the treatment of a neoplastic disease, e.g., non-malignant neoplasm or cancer, are provided herein. Specifically, provided herein are methods for treating a neoplastic disease, such as cancer, in a subject comprising administering to the subject one or more arenaviruses expressing a tumor antigen, tumor associated antigen or an antigenic fragment thereof. In a specific embodiment, provided herein are methods for treating cancer in a subject comprising administering to the subject one or more arenaviruses expressing a tumor antigen, tumor associated antigen or an antigenic fragment thereof, alone or in combination with one or more chemotherapeutic agents. In certain embodiments, immunization with an arenavirus that expresses a tumor antigen, tumor associated antigen or an antigenic fragment thereof, as described herein provides a cytotoxic T-cell response, which can be enhanced by the administration of a chemotherapeutic agent. Methods and compositions for using an arenavirus particle or viral vector and a chemotherapeutic agent provided herein are described in more detail in Sections 5.1.(e) and 5.1.(f).

In addition to administering arenavirus particles or viral vectors to a subject in combination with a chemotherapeutic agent, the immunotherapies for treating a neoplastic disease provided herein can also include an immune checkpoint modulator. The term “immune checkpoint modulator” (also referred to as “checkpoint modulator” or as “checkpoint regulator”) refers to a molecule or to a compound that modulates (e.g., totally or partially reduces, inhibits, interferes with, activates, stimulates, increases, reinforces or supports) the function of one or more checkpoint molecules. Thus, an immune checkpoint modulator may be an immune checkpoint inhibitor or an immune checkpoint activator.

An “immune checkpoint inhibitor” refers to a molecule that inhibits, decreases or interferes with the activity of a negative checkpoint regulator. In certain embodiments, immune checkpoint inhibitors for use with the methods and compositions disclosed herein can inhibit the activity of a negative checkpoint regulator directly, or decrease the expression of a negative checkpoint regulator, or interfere with the interaction of a negative checkpoint regulator and a binding partner (e.g., a ligand). Immune checkpoint inhibitors for use with the methods and compositions disclosed herein include a protein, a polypeptide, a peptide, an antisense oligonucleotide, an antibody, an antibody fragment, or an inhibitory RNA molecule that targets the expression of a negative checkpoint regulator.

A “negative checkpoint regulator” refers to a molecule that down-regulates immune responses (e.g., T-cell activation) by delivery of a negative signal to T-cells following their engagement by ligands or counter-receptors. Exemplary functions of a negative-checkpoint regulator are to prevent out-of-proportion immune activation, minimize collateral damage, and/or maintain peripheral self-tolerance. In certain embodiments, a negative checkpoint regulator is a ligand or receptor expressed by an antigen presenting cell. In certain embodiments, a negative checkpoint regulator is a ligand or receptor expressed by a T-cell. In certain embodiments, a negative checkpoint regulator is a ligand or receptor expressed by both an antigen presenting cell and a T-cell.
(a) Infectious, Replication-Deficient Arenavirus Particles

In certain embodiments, a genetically modified arenavirus provided herein, where the arenavirus:
is infectious;
cannot form infectious progeny virus in a non-complementary cell (i.e., a cell that does not express the functionality that is missing from the replication-deficient arenavirus and causes it to be replication-deficient);
is capable of replicating its genome and expressing its genetic information; and
encodes a tumor antigen, tumor associated antigen or an antigenic fragment thereof, can be used with the methods and compositions provided herein, such as combinations with a chemotherapeutic agent.

A genetically modified arenavirus described herein is infectious, i.e., it can attach to a host cell and release its genetic material into the host cell. A genetically modified arenavirus described herein is replication-deficient, i.e., the arenavirus is unable to produce further infectious progeny particles in a non-complementing cell. In particular, the genome of the arenavirus is modified (e.g., by removal or functional inactivation of an ORF) such that a virus carrying the modified genome can no longer produce infectious progeny viruses. A non-complementing cell is a cell that does not provide the functionality that has been eliminated from the replication-deficient arenavirus by modification of the virus genome (e.g., if the ORF encoding the GP protein is removed or functionally inactivated, a non-complementing cell does not provide the GP protein). However, a genetically modified arenavirus provided herein is capable of producing infectious progeny viruses in complementing cells. Complementing cells are cells that provide (in trans) the functionality that has been eliminated from the replication-deficient arenavirus by modification of the virus genome (e.g., if the ORF encoding the GP protein is removed or functionally inactivated, a complementing cell does provide the GP protein). Expression of the complementing functionality (e.g., the GP protein) can be accomplished by any method known to the skilled artisan (e.g., transient or stable expression). A genetically modified arenavirus described herein can amplify and express its genetic information in a cell that has been infected by the virus. A genetically modified arenavirus provided herein can comprise a nucleotide sequence that encodes a tumor antigen, tumor associated antigen or an antigenic fragment thereof such as, but not limited to, the tumor antigen, tumor associated antigen or an antigenic fragment thereof described in Section 5.1.(b).

In certain embodiments, provided herein is a genetically modified arenavirus in which an ORF of the arenavirus genome is removed or functionally inactivated such that the resulting virus cannot produce further infectious progeny virus particles in non-complementing cells. An arenavirus particle comprising a genetically modified genome in which an ORF is removed or functionally inactivated can be produced in complementing cells (i.e., in cells that express the arenaviral ORF that has been removed or functionally inactivated). The genetic material of the resulting arenavirus particles can be transferred upon infection of a host cell into the host cell, wherein the genetic material can be expressed and amplified. In addition, the genome of the genetically modified arenavirus particles provided herein encodes a tumor antigen, tumor associated antigen or antigenic fragment thereof that can be expressed in the host cell.

In certain embodiments, an ORF of the arenavirus is deleted or functionally inactivated and replaced with a nucleotide encoding a tumor antigen or tumor associated antigen as described herein. In a specific embodiment, the ORF that encodes the glycoprotein GP of the arenavirus is deleted or functionally inactivated. In certain embodiments, functional inactivation of a gene eliminates any translation product. In certain embodiments, functional inactivation refers to a genetic alteration that allows some translation, the translation product, however, is not longer functional and cannot replace the wild type protein.

In certain embodiments, the ORF that encodes the glycoprotein (GP) of the arenavirus is deleted to generate a replication-deficient arenavirus for use in the methods and compositions provided herein. In a specific embodiment, the replication-deficient arenavirus comprises a genomic segment comprising a nucleotide sequence encoding a tumor antigen, tumor associated antigen or antigenic fragment thereof. Thus, in certain embodiments, a genetically modified arenavirus particle provided herein comprises a genomic segment that a) has a deletion or functional inactivation of an ORF that is present in the wild type form of the genomic segment; and b) encodes (either in sense or antisense) a tumor antigen, tumor associated antigen or antigenic fragment thereof.

In certain embodiments, the antigen encoded by the nucleotide that is inserted into the genome of replication-deficient arenavirus can encode, for example, a tumor antigen, tumor associated antigen or antigenic fragment thereof or combinations of tumor antigens, tumor associated antigens or antigenic fragments thereof including, but not limited to, oncogenic viral antigens, cancer-testis antigens, oncofetal antigens, tissue differentiation antigens, mutant protein antigens, Adipophilin, AIM-2, ALDH1AI, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUCI, MUC5AC, p53 (non-mutant), PAX5, PBF, PRAME, PSMA, RAGE, RAGE-1, RGS5, RhoC, RNF43, RU2AS, secernin 1, SOXI0, STEAPI (six-transmembrane epithelial antigen of the prostate 1), survivinn, Telomerase, VEGF, WTi, EGF-R, CEA, CD20, CD33, CD52, gp 100 protein, MELANA/MART1, MART2,NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSXl or-SSX2 fusion protein, TGF-betaRII, Triosephosphate isomerase, ormdm-2, LMP2, HPV E6/E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP-1, CA-125, CA19-9, Calretinin, Epithelial membrane antigen (EMA), Epithelial tumor antigen (ETA), CD19, CD34, CD99, CD 117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXBI, SPA17, SSX, SYCP1, TPTE, Carbohydrate/ganglioside GM2 (oncofetal antigen-immunogenic-1 OFA-I-1), GM3, CA 15-3 (CA 27.29BCAA), CA 195, CA 242, CA 50, CAM 43, CEA, EBNA, EF2, Epstein-Barr virus antigen, HLA-A2, HLA-All, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, pi80erbB-3, c-met, nm-23H1, TAG-72, TAG-72-4, CA-72-4, CAM 17.1, NuMa, 13-catenin, P16, TAGE, CT7, 43-9F,5T4, 791Tgp72, 13HCG, BCA225, BTAA, CD68KP1, CO-029, HTgp-175, M344, MG7-Ag, MOV18, NB70K, NY-CO-1, RCAS1, SDCCAGi6, TA-90, TAAL6, TLP, TPS, CD22, CD27, CD30, CD70, prostein, TARP (T cell receptor gamma alternate reading frame protein), Trp-p8, integrin avP3 (CD61), galactin, or Ral-B, CD123, CLL-1, CD38, CS-1, CD138, and ROR1. A detailed description of the antigens described herein is provided in Section 5.1.(b).

Arenaviruses for use with the methods and compositions provided herein can be Old World viruses, for example Lassa virus, Lymphocytic choriomeningitis virus (LCMV), Mobala virus, Mopeia virus, or Ippy virus, or New World viruses, for example Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Bear Canyon virus, or Whitewater Arroyo virus.

The wild type arenavirus genome consists of a short (˜3.4 kb) and a large (˜7.2 kb) RNA segment. The short segment carries the ORFs encoding the nucleoprotein NP and glycoprotein GP genes. The large segment comprises the RNA-dependent RNA polymerase L and the matrix protein Z genes. Wild type arenaviruses can be rendered replication-deficient to generate vaccine vectors by substituting the glycoprotein gene for one or more tumor antigens, tumor associated antigens or antigenic fragments thereof, against which immune responses are to be induced.

Infectious, replication-deficient arenavirus particles expressing a tumor antigen, tumor associated antigen, or antigenic fragment thereof, or a combination of tumor antigens, tumor associated antigens or antigenic fragments thereof as described herein, can be used to treat (in an immunotherapeutic manner) subjects having a neoplastic disease described herein.

Arenavirus disease and immunosuppression in wild type arenavirus infection are known to result from unchecked viral replication. By abolishing replication, i.e., the ability to produce infectious progeny virus particles, of arenavirus particles by deleting from their genome, e.g., the Z gene which is required for particle release, or the GP gene which is required for infection of target cells, the total number of infected cells can be limited by the inoculum administered, e.g., to a vaccine recipient, or accidentally transmitted to personnel involved in medical or biotechnological applications, or to animals. Therefore, abolishing replication of arenavirus particles prevents pathogenesis as a result of intentional or accidental transmission of vector particles. In this invention, one important aspect consists in exploiting the above necessity of abolishment of replication in a beneficial way for the purpose of expressing tumor antigens, tumor associated antigens or antigenic fragments thereof. In certain embodiments, an arenavirus particle is rendered replication deficient by genetic modification of its genome. Such modifications to the genome can include:
deletion of an ORF (e.g., the ORF encoding the GP, NP, L, or Z protein);
functional inactivation of an ORF (e.g., the ORF encoding the GP, NP, L, or Z protein). For example, this can be achieved by introducing a missense or a nonsense mutation;
change of the sequence of the ORF (e.g., the exchange of an SiP cleavage site with the cleavage site of another protease);
mutagenesis of one of the 5′ or 3′ termini of one of the genomic segments;
mutagenesis of an intergenic region (i.e., of the L or the S genomic segment).

In certain embodiments, an infectious, replication-deficient arenavirus expressing a tumor antigen, tumor associated antigen or antigenic fragment thereof described herein is a Lymphocytic choriomeningitis virus (LCMV) wherein the S segment of the virus is modified by substituting the ORF encoding the GP protein with an ORF encoding a tumor antigen, tumor associated antigen or antigenic fragment thereof.

In certain embodiments, a wild type arenavirus vector genome can be designed to retain at least the essential regulatory elements on the 5′ and 3′ untranslated regions (UTRs) of both segments, and/or also the intergenic regions (IGRs). Without being bound by theory, the minimal transacting factors for gene expression in infected cells remain in the vector genome as ORFs that can be expressed, yet they can be placed differently in the genome and can be placed under control of a different promoter than naturally, or can be expressed from internal ribosome entry sites. In certain embodiments, the nucleic acid encoding a tumor antigen, tumor associated antigen or antigenic fragment thereof is transcribed from one of the endogenous arenavirus promoters (i.e., 5′ UTR, 3′ UTR of the S segment, 5′ UTR, 3′ UTR of the L segment). In other embodiments, the nucleic acid encoding a tumor antigen, tumor associated antigen or antigenic fragment thereof is expressed from a heterologous introduced promoter sequences that can be read by the viral RNA-dependent RNA polymerase, by cellular RNA polymerase I, RNA polymerase II or RNA polymerase III, such as duplications of viral promoter sequences that are naturally found in the viral UTRs, the 28S ribosomal RNA promoter, the beta-actin promoter or the 5S ribosomal RNA promoter, respectively. In certain embodiments, ribonucleic acids coding for a tumor antigen, tumor associated antigen or antigenic fragment thereof are transcribed and translated either by themselves or as read-through by fusion to arenavirus protein ORFs, and expression of proteins in the host cell may be enhanced by introducing in the viral transcript sequence at the appropriate place(s) one or more, e.g., two, three or four, internal ribosome entry sites.

In certain embodiments, the vector generated to encode one or more tumor antigens, tumor associated antigens or antigenic fragments thereof may be based on a specific strain of LCMV. Strains of LCMV include Clone 13, MP strain, Arm CA 1371, Arm E-250, WE, UBC, Traub, Pasteur, 810885, CH-5692, Marseille #12, HP65-2009, 200501927, 810362, 811316, 810316, 810366, 20112714, Douglas, GRO1, SN05, CABN and their derivatives. In certain embodiments, the vector generated to encode one or more tumor antigens, tumor associated antigens or antigenic fragments thereof may be based on LCMV Clone 13. In other embodiments, the vector generated to encode one or more tumor antigens, tumor associated antigens or antigenic fragments thereof may be based on LCMV MP strain.

In certain embodiments, the vector generated to encode one or more tumor antigens, tumor associated antigens or antigenic fragments thereof may be based on a specific strain of Junin virus. Strains of Junin virus include vaccine strains XJ 13, XJ #44, and Candid #1 as well as IV4454, a human isolate. In certain embodiments, the vector generated to encode one or more tumor antigens, tumor associated antigens or antigenic fragments thereof is based on Junin virus Candid #1 strain.

### (b) Tumor Antigens, Tumor Associated Antigens and Antigenic Fragments

In certain embodiments, arenavirus particles with a nucleotide sequence encoding a tumor antigen, tumor associated antigen, or an antigenic fragment thereof provided herein can be used with the methods and compositions provided herein, such as combinations with a chemotherapeutic agent. In certain embodiments, a tumor antigen or tumor associated antigen for use with the methods and compositions described herein is an immunogenic protein expressed in or on a neoplastic cell or tumor, such as a cancer cell or malignant tumor. In certain embodiments, a tumor antigen or tumor associated antigen for use with the methods and compositions described herein is a non-specific, mutant, overexpressed or abnormally expressed protein, which can be present on both a neoplastic cell or tumor and a normal cell or tissue. In certain embodiments, a tumor antigen or tumor associated antigen for use with the methods and compositions described herein is a tumor-specific antigen which is restricted to tumor cells. In certain embodiments, a tumor antigen for use with the methods and compositions described herein is a cancer-specific antigen which is restricted to cancer cells.

In certain embodiments, a tumor antigen or tumor associated antigen can exhibit one, two, three, or more, including all, of the following characteristics: overexpressed/accumulated (i.e., expressed by both normal and neoplastic tissue, but highly expressed in neoplasia), oncofetal (i.e., usually only expressed in fetal tissues and in cancerous somatic cells), oncoviral or oncogenic viral (i.e., encoded by tumorigenic transforming viruses), cancer-testis (i.e., expressed only by cancer cells and adult reproductive tissues, e.g., the testis), lineage-restricted (i.e., expressed largely by a single cancer histotype), mutated (i.e., only expressed in neoplastic tissue as a result of genetic mutation or alteration in transcription), post-translationally altered (e.g., tumor-associated alterations in glycosylation), or idiotypic (i.e., developed from malignant clonal expansions of B or T lymphocytes).

In certain embodiments, the tumor antigen or tumor associated antigen for use with the methods and compositions described herein includes antigens from neoplastic diseases including acute lymphoblastic leukemia; acute lymphoblastic lymphoma; acute lymphocytic leukaemia; acute myelogenous leukemia; acute myeloid leukemia (adult/childhood); adrenocortical carcinoma; AIDS-related cancers; AIDS-related lymphoma; anal cancer; appendix cancer; astrocytomas; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; ewing's sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; hodgkin lymphoma; hypopharyngeal cancer; hypothalamic and visual pathway glioma; intraocular melanoma; islet cell carcinoma (endocrine pancreas); Kaposi Sarcoma; kidney cancer (renal cell cancer); langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); merkel cell cancer; merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-hodgkin lymophoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; respiratory tract carcinoma involving the NUT gene on chromosome 15; retinoblastoma; rhabdomyosarcoma, childhood; salivary gland cancer; sarcoma, Ewing family of tumors; Sezary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sezary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor.

In certain embodiments, the tumor antigen or tumor associated antigen for use with the methods and compositions disclosed herein includes oncogenic viral antigens, cancer-testis antigens, oncofetal antigens, tissue differentiation antigens, mutant protein antigens, Adipophilin, AIM-2, ALDH1AI, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUCI, MUC5AC, p53 (non-mutant), PAX5, PBF, PRAME, PSMA, RAGE, RAGE-1, RGS5, RhoC, RNF43, RU2AS, secernin 1, SOXI0, STEAPI (six-transmembrane epithelial antigen of the prostate 1), survivin, Telomerase, VEGF, WT1, EGF-R, CEA, CD20, CD33, CD52, MELANA/MART1, MART2,NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSXl or-SSX2 fusion protein, TGF-betaRII, Triosephosphate isomerase, ormdm-2, LMP2, HPV E6/E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP-1, GP100, CA-125, CA19-9, Calretinin, Epithelial membrane antigen (EMA), Epithelial tumor antigen (ETA), CD19, CD34, CD99, CD 117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29BCAA), CA 195, CA 242, CA 50, CAM 43, CEA, EBNA, EF2, Epstein-Barr virus antigen, HLA-A2, HLA-All, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F,5T4, 791Tgp72, 13HCG, BCA225, BTAA, CD68KP1, CO-029, HTgp-175, M344, MG7-Ag, MOV18, NB70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin avP3 (CD61), galactin, or Ral-B, CD123, CLL-1, CD38, CS-1, CD138, and ROR1.

In certain embodiments, the tumor antigen or tumor associated antigen is a neoantigen. A “neoantigen,” as used herein, means an antigen that arises by mutation in a tumor cell and such an antigen is not generally expressed in normal cells or tissue. Without being bound by theory, because healthy tissues generally do not posses these antigens, neoantigens represent a preferred target. Additionally, without being bound by theory, in the context of the present invention, since the T cells that recognize the neoantigen may not have undergone negative thymic selection, such cells can have high avidity to the antigen and mount a strong immune response against tumors, while lacking the risk to induce destruction of normal tissue and autoimmune damage. In certain embodiments, the neoantigen is an MHC class I-restricted neoantigen. In certain embodiments, the neoantigen is an MHC class II-restricted neoantigen. In certain embodiments, a mutation in a tumor cell of the patient results in a novel protein that produces the neoantigen.

In certain embodiments, the tumor antigen or tumor associated antigen can be an antigen ortholog, e.g., a mammalian (i.e., non-human primate, pig, dog, cat, or horse) to a human tumor antigen or tumor associated antigen.

In certain embodiments, an antigenic fragment of a tumor antigen or tumor associated antigen described herein is encoded by the nucleotide sequence included within the arenavirus. In certain embodiments, a fragment is antigenic when it is capable of (i) eliciting an antibody immune response in a host (e.g., mouse, rabbit, goat, donkey or human) wherein the resulting antibodies bind specifically to an immunogenic protein expressed in or on a neoplastic cell (e.g., a cancer cell); and/or (ii) eliciting a specific T cell immune response.

In certain embodiments, the nucleotide sequence encoding antigenic fragment of a tumor antigen or tumor associated antigen is 8 to 100 nucleotides in length, 15 to 100 nucleotides in length, 25 to 100 nucleotides in length, 50 to 200 nucleotide in length, 50 to 400 nucleotide in length, 200 to 500 nucleotide in length, or 400 to 600 nucleotides in length, 500 to 800 nucleotide in length. In other embodiments, the nucleotide sequence is 750 to 900 nucleotides in length, 800 to 100 nucleotides in length, 850 to 1000 nucleotides in length, 900 to 1200 nucleotides in length, 1000 to 1200 nucleotides in length, 1000 to 1500 nucleotides or 10 to 1500 nucleotides in length, 1500 to 2000 nucleotides in length, 1700 to 2000 nucleotides in length, 2000 to 2300 nucleotides in length, 2200 to 2500 nucleotides in length, 2500 to 3000 nucleotides in length, 3000 to 3200 nucleotides in length, 3000 to 3500 nucleotides in length, 3200 to 3600 nucleotides in length, 3300 to 3800 nucleotides in length, 4000 nucleotides to 4400 nucleotides in length, 4200 to 4700 nucleotides in length, 4800 to 5000 nucleotides in length, 5000 to 5200 nucleotides in length, 5200 to 5500 nucleotides in length, 5500 to 5800 nucleotides in length, 5800 to 6000 nucleotides in length, 6000 to 6400 nucleotides in length, 6200 to 6800 nucleotides in length, 6600 to 7000 nucleotides in length, 7000 to 7200 nucleotides in lengths, 7200 to 7500 nucleotides in length, or 7500 nucleotides in length. In some embodiments, the nucleotide sequence encodes a peptide or polypeptide that is 5 to 10 amino acids in length, 10 to 25 amino acids in length, 25 to 50 amino acids in length, 50 to 100 amino acids in length, 100 to 150 amino acids in length, 150 to 200 amino acids in length, 200 to 250 amino acids in length, 250 to 300 amino acids in length, 300 to 400 amino acids in length, 400 to 500 amino acids in length, 500 to 750 amino acids in length, 750 to 1000 amino acids in length, 1000 to 1250 amino acids in length, 1250 to 1500 amino acids in length, 1500 to 1750 amino acids in length, 1750 to 2000 amino acids in length, 2000 to 2500 amino acids in length, or more than 2500 or more amino acids in length. In some embodiments, the nucleotide sequence encodes a polypeptide that does not exceed 2500 amino acids in length. In specific embodiments the nucleotide sequence does not contain a stop codon. In certain embodiments, the nucleotide sequence is codon-optimized. In certain embodiments the nucleotide composition, nucleotide pair composition or both can be optimized. Techniques for such optimizations are known in the art and can be applied to optimize a nucleotide sequence of a tumor antigen or tumor associated antigen.

Nucleic acid sequences encoding a tumor antigen, tumor associated antigen, or antigenic fragment thereof can be introduced in the genome of an infectious, replication-deficient arenavirus by substitution of the nucleic acid sequence of the ORF of glycoprotein GP, the matrix protein Z, the nucleoprotein NP, or the polymerase protein L. In other embodiments, the nucleic acid sequence encoding the a tumor antigen, tumor associated antigen, or antigenic fragment thereof is fused to the ORF of glycoprotein GP, the matrix protein Z, the nucleoprotein NP, or the polymerase protein L. The nucleotide sequence encoding the a tumor antigen, tumor associated antigen, or antigenic fragment thereof, once inserted into the genome of an infectious, replication-deficient arenavirus, can be transcribed and/or expressed under control of the four arenavirus promoters (5′ UTR and 3′ UTR of the S segment, and 5′ UTR and 3′ UTR of the L segment), as well as ribonucleic acids that can be inserted with regulatory elements that can be read by the viral RNA-dependent RNA polymerase, cellular RNA polymerase I, RNA polymerase II or RNA polymerase III, such as duplications of viral promoter sequences that are naturally found in the viral UTRs, the 28S ribosomal RNA promoter, the beta-actin promoter or the 5S ribosomal RNA promoter, respectively. The nucleic acids encoding the a tumor antigen, tumor associated antigen, or antigenic fragment thereof can be transcribed and/or expressed either by themselves or as read-through by fusion to arenavirus ORFs and genes, respectively, and/or in combination with one or more, e.g., two, three or four, internal ribosome entry sites.

In certain embodiments, an arenavirus particle comprising a nucleotide sequence encoding a tumor antigen, tumor associated antigen or antigenic fragment thereof as provided herein further comprises at least one nucleotide sequence encoding at least one immunomodulatory peptide, polypeptide or protein. In certain embodiments, the immunomodulatory peptide, polypeptide or protein is Calreticulin (CRT), or a fragment thereof, Ubiquitin or a fragment thereof, Granulocyte-Macrophage Colony-Stimulating Factor (GM-CSF), or a fragment thereof, Invariant chain (CD74) or an antigenic fragment thereof; Mycobacterium tuberculosis Heat shock protein 70 or an antigenic fragment thereof, Herpes simplex virus 1 protein VP22 or an antigenic fragment thereof, CD40 ligand or an antigenic fragment thereof; or Fms-related tyrosine kinase 3 (Flt3) ligand or an antigenic fragment thereof.

In certain embodiments, an arenavirus particle provided herein comprises a genomic segment that a) has a removal or functional inactivation of an ORF that is present in the wild type form of the genomic segment; and b) encodes (either in sense or antisense): (i) one or more tumor antigen, tumor associated antigen or an antigenic fragment thereof provided herein, and (ii) one or more immunomodulatory peptide, polypeptide or protein provided herein.

In certain embodiments, the nucleotide sequence encoding the tumor antigen, tumor associated antigen or an antigenic fragment thereof provided herein, and the nucleotide sequence encoding the immunomodulatory peptide, polypeptide or protein provided herein, are on the same position of the viral genome. In certain embodiments, the nucleotide sequence encoding the tumor antigen, tumor associated antigen or an antigenic fragment thereof provided herein, and the nucleotide sequence encoding the immunomodulatory peptide, polypeptide or protein provided herein, are on different positions of the viral genome.

In certain embodiments, the nucleotide sequence encoding the tumor antigen, tumor associated antigen or an antigenic fragment thereof provided herein, and the nucleotide sequence encoding the immunomodulatory peptide, polypeptide or protein provided herein, are separated via a spacer sequence. In certain embodiments, the sequence encoding the tumor antigen, tumor associated antigen or an antigenic fragment thereof provided herein, and the nucleotide sequence encoding the immunomodulatory peptide, polypeptide or protein provided herein, are separated by an internal ribosome entry site, or a sequence encoding a protease cleavage site. In certain embodiments, the nucleotide sequence encoding the tumor antigen, tumor associated antigen or an antigenic fragment thereof provided herein, and the nucleotide sequence encoding the immunomodulatory peptide, polypeptide or protein provided herein, are separated by a nucleotide sequence encoding a linker or a self-cleaving peptide. Any linker peptide or self-cleaving peptide known to the skilled artisan can be used with the compositions and methods provided herein. A non-limiting example of a peptide linker is GSG. Non-limiting examples of a self-cleaving peptide are Porcine teschovirus-1 2A peptide, Those assign a virus 2A peptide, or Foot-and-mouth disease virus 2A peptide.

In certain embodiments, the tumor antigen, tumor associated antigen or an antigenic fragment thereof provided herein, and the immunomodulatory peptide, polypeptide or protein provided herein, are directly fused together. In certain embodiments, the tumor antigen, tumor associated antigen or an antigenic fragment thereof provided herein, and the immunomodulatory peptide, polypeptide or protein provided herein, are fused together via a peptide linker. In certain embodiments, the tumor antigen, tumor associated antigen or an antigenic fragment thereof provided herein, and the immunomodulatory peptide, polypeptide or protein provided herein are separated from each other via a self-cleaving peptide. A non-limiting example of a peptide linker is GSG. Non-limiting examples of a self-cleaving peptide are Porcine teschovirus-1 2A peptide, Those assign a virus 2A peptide, or Foot-and-mouth disease virus 2A peptide.

In certain embodiments, the tumor antigen, tumor associated antigen or an antigenic fragment thereof provided herein, and the immunomodulatory peptide, polypeptide or protein provided herein are expressed on the same arenavirus particle. In certain embodiments, the tumor antigen, tumor associated antigen or an antigenic fragment thereof provided herein, and the immunomodulatory peptide, polypeptide or protein provided herein are expressed on different areanavirus particles. In certain embodiments, the tumor antigen, tumor associated antigen or an antigenic fragment thereof provided herein, and the immunomodulatory peptide, polypeptide or protein provided herein are expressed on different viruses of the same strain. In certain embodiments, the tumor antigen, tumor associated antigen or an antigenic fragment thereof provided herein, and the immunomodulatory peptide, polypeptide or protein provided herein are expressed on different viruses of different strains.

In certain embodiments, an arenavirus particle generated to encode one or more tumor antigens, tumor associated antigens or antigenic fragments thereof comprises one or more nucleotide sequences encoding tumor antigens, tumor associated antigens or antigenic fragments thereof provided herein. In specific embodiments the tumor antigens, tumor associated antigens or antigenic fragments thereof provided herein are separated by various one or more linkers, spacers, or cleavage sites as described herein.

### (c) Generation of Infectious, Replication-Deficient Arenavirus Expressing a Tumor Antigen, Tumor Associated Antigen or Antigenic Fragment Thereof

Generally, arenavirus particles for use in the methods and compositions provided herein, such as combinations with a chemotherapeutic agent, can be recombinantly produced by standard reverse genetic techniques as described for LCMV (L. Flatz, A. Bergthaler, J. C. de la Torre, and D. D. Pinschewer, Proc Natl Acad Sci USA 103:4663-4668, 2006; A. B. Sanchez and J. C. de la Torre, Virology 350:370, 2006; E. Ortiz-Riano, B. Y. Cheng, J. C. de la Torre, L. Martinez-Sobrido. J Gen Virol. 94:1175-88, 2013). To generate infectious, replication-deficient arenaviruses for use with the present invention these techniques can be used, however, the genome of the rescued virus is modified as described herein. These modifications can be: i) one or more, e.g., two, three or four, of the four arenavirus ORFs (glycoprotein (GP); nucleoprotein (NP); the matrix protein Z; the RNA-dependent RNA polymerase L) are removed or functionally inactivated to prevent formation of infectious particles in normal cells albeit still allowing gene expression in arenavirus vector-infected host cells; and ii) nucleotides encoding for a tumor antigen, tumor associated antigen, or antigenic fragment thereof can be introduced. Infectious, replication-deficient viruses as described herein can be produced as described in International Patent Application Publication No. WO 2009/083210 (application number PCT/EP2008/010994) and International Patent Application Publication No. WO 2014/140301 (application number PCT/EP2014/055144), each of which is incorporated by reference herein in its entirety.

Once generated from cDNA, the infectious, replication-deficient arenaviruses provided herein can be propagated in complementing cells. Complementing cells are cells that provide the functionality that has been eliminated from the replication-deficient arenavirus by modification of its genome (e.g., if the ORF encoding the GP protein is deleted or functionally inactivated, a complementing cell does provide the GP protein).

Owing to the removal or functional inactivation of one or more of the viral genes in arenavirus vectors (here deletion of the glycoprotein, GP, will be taken as an example), arenavirus vectors can be generated and expanded in cells providing in trans the deleted viral gene(s), e.g., the GP in the present example. Such a complementing cell line, henceforth referred to as C-cells, is generated by transfecting a mammalian cell line such as BHK-21, HEK 293, VERO or other (here BHK-21 will be taken as an example) with one or more plasmid(s) for expression of the viral gene(s) of interest (complementation plasmid, referred to as C-plasmid). The C-plasmid(s) express the viral gene(s) deleted in the arenavirus vector to be generated under control of one or more expression cassettes suitable for expression in mammalian cells, e.g., a mammalian polymerase II promoter such as the CMV or EF 1alpha promoter with a polyadenylation signal. In addition, the complementation plasmid features a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Cells that can be used, e.g., BHK-21, HEK 293, MC57G or other, are kept in culture and are transfected with the complementation plasmid(s) using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing C-cell clones are identified using Western blot or flow cytometry procedures with antibodies directed against the viral protein(s) of interest. As an alternative to the use of stably transfected C-cells transient transfection of normal cells can complement the missing viral gene(s) in each of the steps where C-cells will be used below. In addition, a helper virus can be used to provide the missing functionality in trans.

Plasmids that can be used can be of two types: i) Two plasmids, referred to as TF-plasmids for expressing intracellularly in C-cells the minimal transacting factors of the arenavirus, is derived from e.g., NP and L proteins of LCMV in the present example; and ii) Plasmids, referred to as GS-plasmids, for expressing intracellularly in C-cells the arenavirus vector genome segments, e.g., the segments with designed modifications. TF-plasmids express the NP and L proteins of the respective arenavirus vector under control of an expression cassette suitable for protein expression in mammalian cells, typically e.g., a mammalian polymerase II promoter such as the CMV or EF 1alpha promoter, either one of them preferentially in combination with a polyadenylation signal. GS-plasmids express the small (S) and the large (L) genome segments of the vector. Typically, polymerase I-driven expression cassettes or T7 bacteriophage RNA polymerase (T7-) driven expression cassettes can be used, the latter preferentially with a 3′-terminal ribozyme for processing of the primary transcript to yield the correct end. In the case of using a T7-based system, expression of T7 in C-cells must be provided by either including in the recovery process an additional expression plasmid, constructed analogously to TF-plasmids, providing T7, or C-cells are constructed to additionally express T7 in a stable manner. In certain embodiments, TF and GS plasmids can be the same, i.e. the genome sequence and transacting factors can be transcribed by T7, poll and polII promoters from one plasmid.

For recovering of the arenavirus vector, the following procedures can be used. First day: C-cells, typically 80% confluent in M6-well plates, are transfected with a mixture of the two TF-plasmids plus the two GS-plasmids. In certain embodiments, the TF and GS plasmids can be the same, i.e. the genome sequence and transacting factors can be transcribed by T7, poll and polII promoters from one plasmid. For this one can exploit any of the commonly used strategies such as calcium-phosphate, liposome-based protocols or electroporation.

3-5 days later: The culture supernatant (arenavirus vector preparation) is harvested, aliquoted and stored at 4° C., −20° C. or −80° C. depending on how long the arenavirus vector should be stored prior to use. Then the arenavirus vector preparation's infectious titer is assessed by an immunofocus assay on C-cells.

The invention furthermore relates to expression of a tumor antigen, tumor associated antigen, or antigenic fragment thereof in a cell culture wherein the cell culture is infected with an infectious, replication-deficient arenavirus expressing a tumor antigen, tumor associated antigen, or antigenic fragment thereof. When used for expression of a tumor antigen, tumor associated antigen, or antigenic fragment thereof in cultured cells, the following two procedures can be used:
i) The cell type of interest is infected with the arenavirus vector preparation described herein at a multiplicity of infection (MOI) of one or more, e.g., two, three or four, resulting in production of the tumor antigen, tumor associated antigen, or antigenic fragment thereof in all cells already shortly after infection.
ii) Alternatively, a lower MOI can be used and individual cell clones can be selected for their level of virally driven expression of a tumor antigen, tumor associated antigen, or antigenic fragment thereof. Subsequently individual clones can be expanded infinitely owing to the non-cytolytic nature of arenavirus vectors. Irrespective of the approach, the tumor antigen, tumor associated antigen, or antigenic fragment thereof can subsequently be collected (and purified) either from the culture supernatant or from the cells themselves, depending on the properties of the tumor antigen, tumor associated antigen, or antigenic fragment thereof produced. However, the invention is not limited to these two strategies, and other ways of driving expression of a tumor antigen, tumor associated antigen, or antigenic fragment thereof using infectious, replication-deficient arenaviruses as vectors may be considered.

Alternatively, a rescue system consisting of three plasmids can be used: (1) the first plasmid expresses the protein NP by transcription via Polymerase II and subsequent translation in transfected cells; (2) the second plasmid gives rise to the (negative-stranded) L-Segment of the LCMV genome by transcription via Polymerase I as well as the L protein by transcription via Polymerase II from the same template in the opposite direction of the Polymerase I promoter; (3) the third plasmid gives rise to the S-segment of the LCMV genome (encoding the antigen coding sequence instead of the LCMV glycoprotein) via transcription by Polymerase I. 3 μg of each plasmid is used for electroporation of C-cells, followed by seeding of cells in 6-well plates and incubation at 37° C. After incubation, cells and supernatant from transfections are combined with freshly seeded C-cells, and vectors are harvested and cleared from cells & debris at a defined timepoint post infection. Once the vector has been generated, a nucleic acid encoding a tumor antigen, tumor associated antigen, or antigenic fragment thereof can be inserted into a plasmid from which a genomic segment of an infectious replication-deficient vector is transcribed by any technique known to the skilled artisan.

Owing to the removal or functional inactivation of one or more of the viral genes in arenavirus vectors (here deletion of the glycoprotein, GP, will be taken as an example) arenavirus vectors can be generated and expanded in cells that provide the deleted or functionally inactivated viral gene(s) (e.g., the GP) in trans. The resulting virus itself is infectious but is unable to produce further infectious progeny particles in non-complementing cells due to the lack of the deleted or functionally inactivated viral gene(s) (e.g., the GP). The complementing cell can provide the missing functionality either by stable transfection, transient transfection, or by infection with a helper virus that expresses the missing functionality.

In certain embodiments, the complementing cell provides the viral gene that has been deleted or functionally inactivated from the arenavirus vector genome. In a specific embodiment, the complementing cell provides the viral gene from a viral strain that is the same as the viral strain that was used to generate the genome of the arenavirus vector. In another embodiment, the complementing cell provides the viral gene from a viral strain that is different from the viral strain that was used to generate the genome of the arenavirus vector. For example, the viral gene provided in the complementing cell is obtained from the MP strain of LCMV. In another example, the viral gene provided in the complementing cell is obtained from the Clone 13 strain of LCMV. In another example, the viral gene provided in the complementing cell is obtained from the WE strain of LCMV.

In a specific embodiment, the complementing cell provides the GP of the MP strain of LCMV and the arenavirus vector comprises an ORF of a tumor antigen, tumor associated antigen, or antigenic fragment thereof as described herein in place of the ORF encoding the GP protein. In an even more specific embodiment, the complementing cell provides the GP of the MP strain of LCMV and the arenavirus vector is obtained from LCMV Clone 13 and comprises an ORF of a tumor antigen, tumor associated antigen, or antigenic fragment thereof as described herein in place of the ORF encoding the GP protein.

In a specific embodiment, the complementing cell provides the GP of the Clone 13 strain of LCMV and the arenavirus vector comprises an ORF of a tumor antigen, tumor associated antigen, or antigenic fragment thereof as described herein in place of the ORF encoding the GP protein. In an even more specific embodiment, the complementing cell provides the GP of the Clone 13 strain of LCMV and the arenavirus vector is obtained from LCMV MP strain and comprises an ORF of a tumor antigen, tumor associated antigen, or antigenic fragment thereof as described herein in place of the ORF encoding the GP protein.

In a specific embodiment, the complementing cell provides the GP of the WE strain of LCMV and the arenavirus vector comprises an ORF of a tumor antigen, tumor associated antigen, or antigenic fragment thereof as described herein in place of the ORF encoding the GP protein. In an even more specific embodiment, the complementing cell provides the GP of the WE strain of LCMV and the arenavirus vector is obtained from LCMV Clone 13 and comprises an ORF of a tumor antigen, tumor associated antigen, or antigenic fragment thereof as described herein in place of the ORF encoding the GP protein.

In a specific embodiment, the complementing cell provides the GP of the WE strain of LCMV and the arenavirus vector comprises an ORF of a tumor antigen, tumor associated antigen, or antigenic fragment thereof as described herein in place of the ORF encoding the GP protein. In an even more specific embodiment, the complementing cell provides the GP of the WE strain of LCMV and the arenavirus vector is obtained from LCMV MP strain and comprises an ORF of a tumor antigen, tumor associated antigen, or antigenic fragment thereof as described herein in place of the ORF encoding the GP protein.

### (d) Nucleic Acids, Vector Systems and Cell Lines

In one embodiment, described herein is a nucleic acid sequence which is the cDNA of the large genomic segment (L segment) of an infectious, replication-deficient arenavirus described herein, in which one ORF of the genomic segment is deleted or functionally inactivated, and the genomic segment comprises a nucleotide sequence encoding a tumor antigen, tumor associated antigen, or antigenic fragment thereof, which can be sued with the methods and compositions provided herein, such as combinations with a chemotherapeutic agent.

In one embodiment, described herein is a nucleic acid sequence that encodes the short genomic segment (S segment) of an infectious, replication-deficient arenavirus described herein, in which one ORF of the genomic segment is deleted or functionally inactivated and wherein the short genomic segment comprises a nucleotide sequence encoding a tumor antigen, tumor associated antigen, or antigenic fragment thereof. In another embodiment, described herein is a nucleic acid sequence that encodes the short genomic segment (S segment) of an infectious, replication-deficient arenavirus described herein, in which the ORF of the glycoprotein gene is deleted or functionally inactivated and wherein the short genomic segment comprises a nucleotide sequence encoding a tumor antigen, tumor associated antigen, or antigenic fragment thereof. In certain, more specific embodiments, the tumor antigen, tumor associated antigen, or antigenic fragment thereof is an antigen described in Section 5.1.(b).

In certain embodiments, the nucleic acid sequences provided herein can be derived from a particular strain of LCMV. Strains of LCMV include Clone 13, MP strain, Arm CA 1371, Arm E-250, WE, UBC, Traub, Pasteur, 810885, CH-5692, Marseille #12, HP65-2009, 200501927, 810362, 811316, 810316, 810366, 20112714, Douglas, GRO1, SN05, CABN and their derivatives. In specific embodiments, the nucleic acid is derived from LCMV Clone 13. In other specific embodiments, the nucleic acid is derived from LCMV MP strain.

In a more specific embodiment, provided herein is a nucleic acid that comprises an arenavirus genomic segment; and (ii) a nucleotide sequence encoding a tumor antigen, tumor associated antigen, or antigenic fragment thereof.

In one embodiment, described herein is a vector system comprising one or more vectors that together comprise the genome of an infectious, replication-deficient arenavirus particle described herein. Specifically, provided herein is a vector system wherein the one or more vectors comprise two arenavirus genomic segments, namely an L segment and an S segment, of an infectious, replication-deficient arenavirus described herein. Such a vector system can comprise (on one or more separate DNA molecules):
An arenavirus S genomic segment that is modified such that an arenavirus particle carrying this modified S genomic segment cannot produce infectious progeny virus particles and an arenavirus L genomic segment that comprises a nucleotide sequence encoding (in sense or antisense) a tumor antigen, tumor associated antigen, or antigenic fragment thereof,
An arenavirus L genomic segment that is modified such that an arenavirus particle carrying this modified L genomic segment cannot produce infectious progeny virus particles and an arenavirus S genomic segment that comprises a nucleotide sequence encoding (in sense or antisense) a tumor antigen, tumor associated antigen, or antigenic fragment thereof;
An arenavirus S genomic segment that is modified such that an arenavirus particle carrying this modified S genomic segment cannot produce infectious progeny virus particles and wherein the arenavirus S genomic segment comprises a nucleotide sequence encoding (in sense or antisense) a tumor antigen, tumor associated antigen, or antigenic fragment thereof and comprising a wild type arenavirus L genomic segment; or
An arenavirus L genomic segment that is modified such that an arenavirus particle carrying this modified L genomic segment cannot produce infectious progeny virus particles and wherein the arenavirus L genomic segment comprises a nucleotide sequence encoding (in sense or antisense) a tumor antigen, tumor associated antigen, or antigenic fragment thereof and comprising a wild type arenavirus S genomic segment.

In certain embodiments, described herein is a nucleic acid sequence comprising an arenavirus (e.g., LCMV) genomic segment in which the ORF encoding the GP of the S genomic segment is substituted with a nucleotide sequence encoding a tumor antigen, tumor associated antigen, or antigenic fragment thereof, which is selected from the group consisting of oncogenic viral antigens, cancer-testis antigens, oncofetal antigens, tissue differentiation antigens, mutant protein antigens, Adipophilin, AIM-2, ALDH1AI, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUCI, MUC5AC, p53 (non-mutant), PAX5, PBF, PRAME, PSMA, RAGE, RAGE-1, RGS5, RhoC, RNF43, RU2AS, secernin 1, SOXI0, STEAPI (six-transmembrane epithelial antigen of the prostate 1), survivinn, Telomerase, VEGF, WT1, EGF-R, CEA, CD20, CD33, CD52, gp 100 protein, MELANA/MART1, MART2,NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSXl or-SSX2 fusion protein, TGF-betaRII, Triosephosphate isomerase, ormdm-2, LMP2, HPV E6/E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP-1, CA-125, CA19-9, Calretinin, Epithelial membrane antigen (EMA), Epithelial tumor antigen (ETA), CD19, CD34, CD99, CD 117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29BCAA), CA 195, CA 242, CA 50, CAM 43, CEA, EBNA, EF2, Epstein-Barr virus antigen, HLA-A2, HLA-Ai i, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, pi80erbB-3, c-met, nm-23H1, TAG-72, TAG-72-4, CA-72-4, CAM 17.1, NuMa, 13-catenin, P16, TAGE, CT7, 43-9F,5T4, 791Tgp72, 13HCG, BCA225, BTAA, CD68KP1, CO-029, HTgp-175, M344, MG7-Ag, MOV18, NB70K, NY-CO-1, RCAS1, SDCCAGi6, TA-90, TAAL6, TLP, TPS, CD22, CD27, CD30, CD70, prostein, TARP (T cell receptor gamma alternate reading frame protein), Trp-p8, integrin avP3 (CD61), galactin, or Ral-B, CD123, CLL-1, CD38, CS-1, CD138, and ROR1.

In certain embodiments, described herein is a nucleic acid sequence comprising an arenavirus (e.g., LCMV) genomic segment in which the ORF encoding the GP of the S genomic segment is substituted with a nucleotide sequence encoding one or more a tumor antigen, tumor associated antigen, or antigenic fragment thereof (e.g., one or more of those listed in the above paragraph).

In another embodiment, provided herein is a cell wherein the cell comprises a nucleic acid or a vector system described above in this section. Cell lines derived from such cells, cultures comprising such cells, and methods of culturing such cells infected with nucleic acids or vector systems are also provided herein. In certain embodiments, provided herein is a cell wherein the cell comprises a nucleic acid comprising the large genomic segment (L segment) of an infectious, replication-deficient arenavirus described herein, in which one ORF of the genomic segment is deleted or functionally inactivated, and the genomic segment comprises a nucleotide sequence encoding a tumor antigen, tumor associated antigen, or antigenic fragment thereof.

In other embodiments, provided herein is a cell wherein the cell comprises a nucleic acid sequence that comprises the short genomic segment (S segment) of an infectious, replication-deficient arenavirus described herein, in which one ORF of the genomic segment is deleted or functionally inactivated and wherein the short genomic segment comprises a nucleotide sequence encoding a tumor antigen, tumor associated antigen, or antigenic fragment thereof.

In another embodiment, provided herein is a cell wherein the cell comprises two nucleic acids or vector systems described herein. Cell lines derived from such cells, cultures comprising such cells, and methods of culturing such cells infected with nucleic acids or vector systems are also provided herein.

### (e) Methods of Use

Vaccines have been successful for preventing and/or treating infectious diseases, such as those for polio virus and measles. However, therapeutic immunization in the setting of established, chronic disease, including cancer has been less successful. The ability to generate an arenavirus particle that is used in combination with a chemotherapeutic agent represents a new novel vaccine strategy.

In certain embodiments, provided herein are methods of treating a neoplastic disease in a subject. Such methods can include administering to a subject in need thereof an arenavirus particle provided herein and a chemotherapeutic agent provided herein. In certain embodiments, the arenavirus particle used in the methods is an infectious, replication-deficient arenavirus particle. Thus, in certain embodiments, the infectious, replication-deficient arenavirus particle used in the methods is engineered to contain a genome comprising a nucleotide sequence encoding a tumor antigen, tumor associated antigen or an antigenic fragment thereof, and the ability to amplify and express its genetic information in infected cells but unable to produce further infectious progeny particles in non-complementing cells.

In one embodiment, provided herein are methods of treating a neoplastic disease in a subject comprising administering to the subject one or more infectious, replication-deficient arenavirus particles expressing a tumor antigen, tumor associated antigen or an antigenic fragment thereof as provided herein or a composition thereof, and a chemotherapeutic agent provided herein. In a specific embodiment, a method for treating a neoplastic disease described herein comprises administering to a subject in need thereof a therapeutically effective amount of one or more infectious, replication-deficient arenavirus particles expressing a tumor antigen, tumor associated antigen or an antigenic fragment thereof provided herein or a composition thereof, and a chemotherapeutic agent provided herein. The subject can be a mammal, such as but not limited to a human, a mouse, a rat, a guinea pig, a domesticated animal, such as, but not limited to, a cow, a horse, a sheep, a pig, a goat, a cat, a dog, a hamster, a donkey. In a specific embodiment, the subject is a human.

In another embodiment, provided herein are methods for inducing an immune response against a neoplastic cell or tissue, such as a cancer cell or tumor, in a subject comprising administering to the subject an infectious, replication-deficient arenavirus particle expressing a tumor antigen, tumor associated antigen or an antigenic fragment thereof provided herein, or a composition thereof, and a chemotherapeutic agent provided herein.

In another embodiment, the subjects to whom an infectious, replication-deficient arenavirus particle expressing a tumor antigen, tumor associated antigen or an antigenic fragment thereof provided herein, or a composition thereof, and a chemotherapeutic agent provided herein is administered have, are susceptible to, or are at risk for a neoplastic disease.

In another embodiment, the subjects to whom an infectious, replication-deficient arenavirus particle expressing a tumor antigen, tumor associated antigen or an antigenic fragment thereof provided herein, or a composition thereof, and a chemotherapeutic agent provided herein is administered have, are susceptible to, or are at risk for development of a neoplastic disease, such as cancer, or exhibit a pre-cancerous tissue lesion. In another specific embodiment, the subjects to whom infectious, replication-deficient arenavirus particle expressing a tumor antigen, tumor associated antigen or an antigenic fragment thereof provided herein, or a composition thereof, and a chemotherapeutic agent provided herein is administered are diagnosed with a neoplastic disease, such as cancer, or exhibit a pre-cancerous tissue lesion.

In another embodiment, the subjects to whom an infectious, replication-deficient arenavirus particle expressing a tumor antigen, tumor associated antigen or an antigenic fragment thereof provided herein, or a composition thereof, and a chemotherapeutic agent provided herein is administered are suffering from, are susceptible to, or are at risk for, a neoplastic disease selected from, but not limited to, acute lymphoblastic leukemia; acute lymphoblastic lymphoma; acute lymphocytic leukaemia; acute myelogenous leukemia; acute myeloid leukemia (adult/childhood); adrenocortical carcinoma; AIDS-related cancers; AIDS-related lymphoma; anal cancer; appendix cancer; astrocytomas; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; ewing's sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; hodgkin lymphoma; hypopharyngeal cancer; hypothalamic and visual pathway glioma; intraocular melanoma; islet cell carcinoma (endocrine pancreas); Kaposi Sarcoma; kidney cancer (renal cell cancer); langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); merkel cell cancer; merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-hodgkin lymophoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; respiratory tract carcinoma involving the NUT gene on chromosome 15; retinoblastoma; rhabdomyosarcoma, childhood; salivary gland cancer; sarcoma, Ewing family of tumors; Sezary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sezary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor.

In another embodiment, an infectious, replication-deficient arenavirus particle expressing a tumor antigen, tumor associated antigen or an antigenic fragment thereof provided herein, or a composition thereof, and a chemotherapeutic agent provided herein is administered to a subject of any age group suffering from, are susceptible to, or are at risk for a neoplastic disease. In a specific embodiment, an infectious, replication-deficient arenavirus particle expressing a tumor antigen, tumor associated antigen or an antigenic fragment thereof provided herein, or a composition thereof, and a chemotherapeutic agent provided herein is administered to a subject with a compromised immune system, a pregnant subject, a subject undergoing an organ or bone marrow transplant, a subject taking immunosuppressive drugs, a subject undergoing hemodialysis, a subject who has cancer, or a subject who is suffering from, are susceptible to, or are at risk for a neoplastic disease. In a more specific embodiment, an infectious, replication-deficient arenavirus particle expressing a tumor antigen, tumor associated antigen or an antigenic fragment thereof provided herein, or a composition thereof, and a chemotherapeutic agent provided herein is administered to a subject who is a child of 0, 1, 2, 3, 4, 5, 6, 7, 8, 9, 10, 11, 12, 13, 14, 15, 16, or 17 years of age suffering from, are susceptible to, or are at risk for a neoplastic disease. In yet another specific embodiment, an infectious, replication-deficient arenavirus particle expressing a tumor antigen, tumor associated antigen or an antigenic fragment thereof provided herein, or a composition thereof, and a chemotherapeutic agent provided herein is administered to a subject who is an infant suffering from, is susceptible to, or is at risk for a neoplastic disease. In yet another specific embodiment, an infectious, replication-deficient arenavirus particle expressing a tumor antigen, tumor associated antigen or an antigenic fragment thereof provided herein, or a composition thereof, and a chemotherapeutic agent provided herein is administered to a subject who is an infant of 0, 1, 2, 3, 4, 5, 6, 7, 8, 9, 10, 11, or 12 months of age suffering from, is susceptible to, or is at risk for a neoplastic disease. In yet another specific embodiment, an infectious, replication-deficient arenavirus particle expressing a tumor antigen, tumor associated antigen or an antigenic fragment thereof provided herein, or a composition thereof, and a chemotherapeutic agent provided herein is administered to an elderly subject who is suffering from, is susceptible to, or is at risk for a neoplastic disease. In a more specific embodiment, an infectious, replication-deficient arenavirus particle expressing a tumor antigen, tumor associated antigen or an antigenic fragment thereof provided herein, or a composition thereof, and a chemotherapeutic agent provided herein is administered to a subject who is a senior subject of 65, 66, 67, 68, 69, 70, 71, 72, 73, 74, 75, 76, 77, 78, 79, 80, 81, 82, 83, 84, 85, 86, 87, 88, 89, or 90 years of age. Provided herein is a method for preventing a cancer in a subject susceptible to, or is at risk for a neoplastic disease.

In another embodiment, an infectious, replication-deficient arenavirus particle expressing a tumor antigen, tumor associated antigen or an antigenic fragment thereof provided herein, or a composition thereof, and a chemotherapeutic agent provided herein is administered to subjects with a heightened risk of cancer metastasis. In a specific embodiment, an infectious, replication-deficient arenavirus particle expressing a tumor antigen, tumor associated antigen or an antigenic fragment thereof provided herein, or a composition thereof, and a chemotherapeutic agent provided herein is administered to subjects in the neonatal period with a neonatal and therefore immature immune system.

In another embodiment, an infectious, replication-deficient arenavirus particle expressing a tumor antigen, tumor associated antigen or an antigenic fragment thereof provided herein, or a composition thereof, and a chemotherapeutic agent provided herein is administered to a subject having grade 0 (i.e., in situ neoplasm), grade 1, grade 2, grade 3 or grade 4 cancer or a subcategory thereof, such as grade 3A, 3B, or 3C, or an equivalent thereof.

In another embodiment, an infectious, replication-deficient arenavirus particle expressing a tumor antigen, tumor associated antigen or an antigenic fragment thereof provided herein, or a composition thereof, and a chemotherapeutic agent provided herein is administered to a subject having cancer at a Tumor, Node, Metastasis (TNM) stage of any combination selected from Tumor T1, T2, T3, and T4, and Node N0, N1, N2, or N3, and Metastasis M0 and Mi.

Successful treatment of a cancer patient can be assessed as prolongation of expected survival, induction of an anti-tumor immune response, or improvement of a particular characteristic of a cancer. Examples of characteristics of a cancer that might be improved include tumor size (e.g., T0, T is, or T1-4), state of metastasis (e.g., M0, M1), number of observable tumors, node involvement (e.g., NO, N1-4, Nx), grade (i.e., grades 1, 2, 3, or 4), stage (e.g., 0, I, II, III, or IV), presence or concentration of certain markers on the cells or in bodily fluids (e.g., AFP, B2M, beta-HCG, BTA, CA 15-3, CA 27.29, CA 125, CA 72.4, CA 19-9, calcitonin, CEA, chromgrainin A, EGFR, hormone receptors, HER2, HCG, immunoglobulins, NSE, NMP22, PSA, PAP, PSMA, 5-100, TA-90, and thyroglobulin), and/or associated pathologies (e.g., ascites or edema) or symptoms (e.g., cachexia, fever, anorexia, or pain). The improvement, if measureable by percent, can be at least 5, 10, 15, 20, 25, 30, 40, 50, 60, 70, 80, or 90% (e.g., survival, or volume or linear dimensions of a tumor).

In another embodiment, an infectious, replication-deficient arenavirus particle expressing a tumor antigen, tumor associated antigen or an antigenic fragment thereof provided herein, or a composition thereof, and a chemotherapeutic agent provided herein is administered to a subject having a dormant cancer (e.g., the subject is in remission). Thus, provided herein is a method for preventing reactivation of a cancer. Also provided herein are methods for reducing the frequency of reoccurence of a cancer.

In another embodiment, an infectious, replication-deficient arenavirus particle expressing a tumor antigen, tumor associated antigen or an antigenic fragment thereof provided herein, or a composition thereof, and a chemotherapeutic agent provided herein is administered to a subject having a recurrent a cancer.

In another embodiment, an infectious, replication-deficient arenavirus particle expressing a tumor antigen, tumor associated antigen or an antigenic fragment thereof provided herein, or a composition thereof, and a chemotherapeutic agent provided herein is administered to a subject with a genetic predisposition for a cancer. In another embodiment, an infectious, replication-deficient arenavirus particle expressing a tumor antigen, tumor associated antigen or an antigenic fragment thereof provided herein, or a composition thereof, and a chemotherapeutic agent provided herein is administered to a subject with risk factors. Exemplary risk factors include, aging, tobacco, sun exposure, radiation exposure, chemical exposure, family history, alcohol, poor diet, lack of physical activity, or being overweight.

In another embodiment, an infectious, replication-deficient arenavirus particle expressing a tumor antigen, tumor associated antigen or an antigenic fragment thereof provided herein, or a composition thereof, and a chemotherapeutic agent provided herein is administered to subjects who suffer from one or more types of cancers. In other embodiments, any type of neoplastic disease, such as cancer, that is susceptible to treatment with the compositions described herein might be targeted.

In another embodiment, administering an infectious, replication-deficient arenavirus particle expressing a tumor antigen, tumor associated antigen or an antigenic fragment thereof provided or a composition thereof to subjects confer cell-mediated immunity (CMI) against a neoplastic cell or tumor, such as a cancer cell or tumor. Without being bound by theory, in another embodiment, an infectious, replication-deficient arenavirus particle expressing a tumor antigen, tumor associated antigen or an antigenic fragment thereof provided or a composition thereof infects and expresses antigens of interest in antigen presenting cells (APC) of the host (e.g., macrophages) for direct presentation of antigens on Major Histocompatibility Complex (MHC) class I and II. In another embodiment, administering an infectious, replication-deficient arenavirus particle expressing a tumor antigen, tumor associated antigen or an antigenic fragment thereof provided herein, or a composition thereof, to subjects induces plurifunctional IFN-γ and TNF-α co-producing cancer-specific CD4+ and CD8+ T cell responses (IFN-γ is produced by CD4+ and CD8+ T cells and TNF-α is produced by CD4+ T cells) of high magnitude to treat a neoplastic disease.

In another embodiment, administering an infectious, replication-deficient arenavirus particle expressing a tumor antigen, tumor associated antigen or an antigenic fragment thereof provided herein, or a composition thereof, and a chemotherapeutic agent provided herein increases or improves one or more clinical outcome for cancer treatment. Non-limiting examples of such outcomes are overall survival, progression-free survival, time to progression, time to treatment failure, event-free survival, time to next treatment, overall response rate and duration of response. The increase or improvement in one or more of the clinical outcomes can be by at least about 10%, at least about 20%, at least about 25%, at least about 30%, at least about 35%, at least about 40%, at least about 50%, at least about 60%, at least about 70%, at least about 80%, at least about 90%, or more, compared to a patient or group of patients having the same neoplastic disease in the absence of such treatment.

Changes in cell-mediated immunity (CMI) response function against a neoplastic cell or tumor, including a cancer cell or tumor, induced by administering an infectious, replication-deficient arenavirus particle expressing a tumor antigen, tumor associated antigen or an antigenic fragment thereof provided, or a composition thereof, in subjects can be measured by any assay known to the skilled artisan including, but not limited to flow cytometry (see, e.g., Perfetto S. P. et al., Nat Rev Immun. 2004; 4(8):648-55), lymphocyte proliferation assays (see, e.g., Bonilla F. A. et al., Ann Allergy Asthma Immunol. 2008; 101:101-4; and Hicks M. J. et al., Am J Clin Pathol. 1983; 80:159-63), assays to measure lymphocyte activation including determining changes in surface marker expression following activation of measurement of cytokines of T lymphocytes (see, e.g., Caruso A. et al., Cytometry. 1997; 27:71-6), ELISPOT assays (see, e.g., Czerkinsky C. C. et al., J Immunol Methods. 1983; 65:109-121; and Hutchings P. R. Et al., J Immunol Methods. 1989; 120:1-8), or Natural killer cell cytotoxicity assays (see, e.g., Bonilla F. A. et al., Ann Allergy Asthma Immunol. 2005 May; 94(5 Suppl 1): S1-63).

Chemotherapeutic agents diclosed herein can be alkylating agents (e.g., cyclophosphamide), platinum-based therapeutics, antimetabolites, topoisomerase inhibitors, cytotoxic antibiotics, intercalating agents, mitosis inhibitors, taxanes, or combinations of two or more thereof. In certain embodiments, the alkylating agent is a nitrogen mustard, a nitrosourea, an alkyl sulfonate, a non-classical alkylating agent, or a triazene. In certain embodiments, the chemotherapeutic agent comprises one or more of cyclophosphamide, thiotepa, mechlorethamine (chlormethine/mustine), uramustine, melphalan, chlorambucil, ifosfamide, chlornaphazine, cholophosphamide, estramustine, novembichin, phenesterine, prednimustine, trofosfamide, uracil mustard, bendamustine, busulfan, improsulfan, piposulfan, carmustine, lomustine, chlorozotocin, fotemustine, nimustine, ranimustine, streptozucin, cisplatin, carboplatin, nedaplatin, oxaliplatin, satraplatin, triplatin tetranitrate, procarbazine, altretamine, dacarbazine, mitozolomide, temozolomide, paclitaxel, docetaxel, vinblastine, vincristine, vinorelbine, cabazitaxel, dactinomycin (actinomycin D), calicheamicin, dynemicin, amsacrine, doxarubicin, daunorubicin, epirubicin, mitoxantrone, idarubicin, pirarubicin, benzodopa, carboquone, meturedopa, uredopa, altretamine, triethylenemelamine, trietylenephosphoramide, triethiylenethiophosphoramide, trimethylolomelamine, bullatacin, bullatacinone, camptothecin, topotecan, bryostatin, callystatin, CC-1065, adozelesin, carzelesin, bizelesin, cryptophycin, dolastatin, duocarmycin, KW-2189, CB1-TM1, eleutherobin, pancratistatin, sarcodictyin, spongistatin, clodronate, esperamicin, neocarzinostatin chromophore, aclacinomysin, anthramycin, azaserine, bleomycin, cactinomycin, carabicin, carminomycin, carzinophilin, chromomycinis, detorubicin, 6-diazo-5-oxo-L-norleucine, esorubicin, idarubicin, marcellomycin, mitomycin, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, mitotane, trilostane, frolinic acid, aceglatone, aldophosphamide glycoside, aminolevulinic acid, eniluracil, bestrabucil, bisantrene, edatraxate, defofamine, demecolcine, diaziquone, elformithine, elliptinium acetate, etoglucid, gallium nitrate, hydroxyurea, lentinan, lonidainine, maytansine, ansamitocins, mitoguazone, mopidanmol, nitraerine, pentostatin, phenamet, pirarubicin, losoxantrone, podophyllinic acid, 2-ethylhydrazide, PSK polysaccharide complex, razoxane, rhizoxin, sizofiran, spirogermanium, tenuazonic acid, triaziquone, 2,2′,2″-trichlorotriethylamine; T-2 toxin, verracurin A, roridin A and anguidine, urethan, vindesine, mannomustine, mitobronitol, mitolactol, pipobroman, gacytosine, arabinoside (“Ara-C”), etoposide (VP-16), vinorelbine, novantrone, teniposide, edatrexate, aminopterin, xeloda, ibandronate, irinotecan (e.g., CPT-11), topoisomerase inhibitor RFS 2000, difluorometlhylornithine (DMFO), retinoic acid, capecitabine, plicomycin, gemcitabine, navelbine, transplatinum, and pharmaceutically acceptable salts, acids, or derivatives of any of the above. In specific embodiments, the chemotherapeutic agent comprises cyclophosphamide. In certain embodiments, the nitrogen mustard is mechlorethamine, cyclophosphamide, melphalan, chlorambucil, ifosfamide, or busulfan. In certain embodiments, the chemotherapeutic agent alkylates DNA. In certain embodiments, the chemotherapeutic agent alkylates DNA, resulting in the formation of interstrand cross-links (“ICLs”).

In certain embodiments, chemotherapeutic agents described herein are used in combination with an immune checkpoint inhibitor that inhibits, decreases or interferes with the activity of a negative checkpoint regulator. In certain embodiments, the negative checkpoint regulator is selected from the group consisting of Cytotoxic T-lymphocyte antigen-4 (CTLA-4), CD80, CD86, Programmed cell death 1 (PD-1), Programmed cell death ligand 1 (PD-L1), Programmed cell death ligand 2 (PD-L2), Lymphocyte activation gene-3 (LAG-3; also known as CD223), Galectin-3, B and T lymphocyte attenuator (BTLA), T-cell membrane protein 3 (TIM3), Galectin-9 (GAL9), B7-H1, B7-H3, B7-H4, T-Cell immunoreceptor with Ig and ITIM domains (TIGIT/Vstm3/WUCAM/VSIG9), V-domain Ig suppressor of T-Cell activation (VISTA), Glucocorticoid-induced tumor necrosis factor receptor-related (GITR) protein, Herpes Virus Entry Mediator (HVEM), OX40, CD27, CD28, CD137. CGEN-15001T, CGEN-15022, CGEN-15027, CGEN-15049, CGEN-15052, and CGEN-15092. In certain embodiments, the immune checkpoint inhibitor is an anti-PD-1 antibody.

In certain embodiments, an infectious, replication-deficient arenavirus particle expressing a tumor antigen, tumor associated antigen or an antigenic fragment thereof provided herein, or a composition thereof, and a chemotherapeutic agent provided herein is preferably administered in multiple injections (e.g., at least 2, 3, 4, 5, 6, 7, 8, 9, 10, 12, 14, 16, 18, 20, 25, 30, 40, 45, or 50 injections) or by continuous infusion (e.g., using a pump) at multiple sites (e.g., at least 2, 3, 4, 5, 6, 7, 8, 9, 10, 12, or 14 sites). In certain embodiments, the infectious, replication-deficient arenavirus particle expressing a tumor antigen, tumor associated antigen or an antigenic fragment thereof provided herein, or a composition thereof, is administered in two or more separate injections over a 6-month period, a 12-month period, a 24-month period, or a 48-month period. In certain embodiments, the infectious, replication-deficient arenavirus particle expressing a tumor antigen, tumor associated antigen or an antigenic fragment thereof provided herein, or a composition thereof, is administered with a first dose at an elected date, a second dose at least 2 months after the first dose, and a third does 6 months after the first dose.

In one example, cutaneous injections are performed at multiple body sites to reduce extent of local skin reactions. On a given vaccination day, the patient receives the assigned total dose administered from one syringe in 3 to 5 separate intradermal injections of the dose (e.g., at least 0.4 ml, 0.2 ml, or 0.1 ml) each in an extremity spaced at least about 5 cm (e.g., at least 4.5, 5, 6, 7, 8, 9, or cm) at needle entry from the nearest neighboring injection. On subsequent vaccination days, the injection sites are rotated to different limbs in a clockwise or counter-clockwise manner.

In certain embodiments, the methods further comprise co-administration of the arenavirus particle provided herein and a chemotherapeutic agent. In certain embodiments, the co-administration is simultaneous. In another embodiment, the arenavirus particle is administered prior to administration of the chemotherapeutic agent. In other embodiments, the arenavirus particle is administered after administration of the chemotherapeutic agent. In certain embodiments, the interval between administration of the arenavirus particle and the chemotherapeutic agent is about 1 hour, about 2 hours, about 3 hours, about 4 hours, about 5 hours, about 6 hours, about 7 hours, about 8 hours, about 9 hours, about 10 hours, about 11 hours, or about 12 hours. In certain embodiments, the interval between administration of the arenavirus particle and the chemotherapeutic agent is about 1 day, about 2 days, about 3 days, about 4 days, about 5 days, about 6 days, about 1 week, about 8 days, about 9 days, about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks. In certain embodiments, the interval between administration of the arenavirus particle and the chemotherapeutic agent is about 1 month, about 2 months, about 3 months, about 4 months, about 5 months, or about 6 months. In some embodiments, the method further includes administering at least one additional therapy.

In another embodiment, two infectious, replication-deficient arenavirus particles are administered in a treatment regime at molar ratios ranging from about 1:1 to 1:1000, in particular including: 1:1 ratio, 1:2 ratio, 1:5 ratio, 1:10 ratio, 1:20 ratio, 1:50 ratio, 1:100 ratio, 1:200 ratio, 1:300 ratio, 1:400 ratio, 1:500 ratio, 1:600 ratio, 1:700 ratio, 1:800 ratio, 1:900 ratio, 1:1000 ratio.

In certain embodiments, provided herein is a method of treating neoplastic disease wherein a first infectious, replication-deficient arenavirus particle is administered first as a “prime,” and a second infectious, replication-deficient arenavirus particle is administered as a “boost.” The first and the second infectious, replication-deficient arenavirus particles can express the same or different tumor antigens, tumor associated antigens or antigenic fragments thereof. Alternatively, or additionally, some certain embodiments, the “prime” and “boost” administration are performed with an infectious, replication-deficient arenavirus particle derived from different species. In certain specific embodiments, the “prime” administration is performed with an infectious, replication-deficient arenavirus particle derived from LCMV, and the “boost” is performed with an infectious, replication-deficient arenavirus particle derived from Junin virus. In certain specific embodiments, the “prime” administration is performed with an infectious, replication-deficient arenavirus particle derived from Junin virus, and the “boost” is performed with an infectious, replication-deficient arenavirus particle derived from LCMV. In certain embodiments, the “prime” administration is performed with an arenavirus particle derived from Pichinde virus, and the “boost” is performed with an arenavirus particle derived from LCMV. In certain embodiments, the “prime” administration is performed with an arenavirus particle derived from Pichinde virus, and the “boost” is performed with an arenavirus particle derived from Junin virus. In certain embodiments, the “prime” administration is performed with an arenavirus particle derived from LCMV, and the “boost” is performed with an arenavirus particle derived from Pichinde virus. In certain embodiments, the “prime” administration is performed with an arenavirus particle derived from Junin virus, and the “boost” is performed with an arenavirus particle derived from Pichinde virus. In certain embodiments, the “prime” administration and/or the “boost” administration are performed in combination with the administration of an immunomodulatory peptide, polypeptide, or protein. In certain embodiments, the “prime” administration and/or the “boost” administration are performed in combination with the administration of a chemotherapeutic agent.

In certain embodiments, administering a first infectious, replication-deficient arenavirus particle expressing a tumor antigen, tumor associated antigen or antigenic fragment thereof, followed by administering a second infectious, replication-deficient arenavirus particle expressing a tumor antigen, tumor associated antigen or antigenic fragment thereof results in a greater antigen specific CD8+ T cell response than administering a single infectious, replication-deficient arenavirus particle expressing a tumor antigen, tumor associated antigen or antigenic fragment thereof. In certain embodiments, the antigen specific CD8+ T cell count increases by 50%, 100%, 150% or 200% after the second administration compared to the first administration. In certain embodiments, administering a third infectious, replication-deficient arenavirus particle expressing a tumor antigen, tumor associated antigen or antigenic fragment thereof results in a greater antigen specific CD8+ T cell response than administering two consecutive infectious, replication-deficient arenavirus particles expressing a tumor antigen, tumor associated antigen or antigenic fragment thereof. In certain embodiments, the antigen specific CD8+ T cell count increases by about 50%, about 100%, about 150%, about 200% or about 250% after the third administration compared to the first administration.

In certain embodiments, provided herein are methods for treating a neoplastic disease comprising administering two or more arenavirus particles, wherein the two or more arenavirus particles are homologous, and wherein the time interval between each administration is about 1 week, about 2 weeks, about 3 week, about 4 weeks, about 5 weeks, about 6 weeks, about 7 weeks, about 8 weeks, about 3 months, about 4 months, about 5 months, about 6 months, about 7 months, about 8 months, about 9 months, about 10 months, about 11 months, about 12 months, about 18 months, or about 24 months.

In certain embodiments, administering a first infectious, replication-deficient arenavirus particle expressing a tumor antigen, tumor associated antigen or antigenic fragment thereof and a second, heterologous, infectious, replication-deficient arenavirus particle expressing a tumor antigen, tumor associated antigen or antigenic fragment thereof elicits a greater CD8+ T cell response than administering a first infectious, replication-deficient arenavirus particle expressing a tumor antigen, tumor associated antigen or antigenic fragment thereof and a second, homologous, infectious, replication-deficient arenavirus particle expressing a tumor antigen, tumor associated antigen or antigenic fragment thereof.

### (f) Compositions, Administration and Dosage

In certain embodiments, vaccines, immunogenic compositions (e.g., vaccine formulations), and pharmaceutical compositions comprising an arenavirus particle provided herein, can be used with the methods and compositions provided herein, such as combinations with a chemotherapeutic agent provided herein. Such vaccines, immunogenic compositions and pharmaceutical compositions can be formulated according to standard procedures in the art.

In another embodiment, provided herein are compositions comprising an infectious, replication-deficient arenavirus particle described herein, and, in certain embodiments, a chemotherapeutic agent provided herein. Such compositions can be used in methods of treating a neoplastic disease. In another specific embodiment, the immunogenic compositions provided herein can be used to induce an immune response in a host to whom the composition is administered. The immunogenic compositions described herein can be used as vaccines and can accordingly be formulated as pharmaceutical compositions. In a specific embodiment, the immunogenic compositions described herein are used in the treatment of a neoplastic disease a subject (e.g., human subject). In other embodiments, the vaccine, immunogenic composition or pharmaceutical composition are suitable for veterinary and/or human administration.

In certain embodiments, provided herein are immunogenic compositions comprising an arenavirus particle (or a combination of different arenavirus particles) as described herein. In certain embodiments, such an immunogenic composition further comprises a pharmaceutically acceptable excipient. In certain embodiments, such an immunogenic composition further comprises an adjuvant. The adjuvant for administration in combination with a composition described herein may be administered before, concomitantly with, or after administration of said composition. In some embodiments, the term “adjuvant” refers to a compound that when administered in conjunction with or as part of a composition described herein augments, enhances and/or boosts the immune response to an infectious, replication-deficient arenavirus particle, but when the compound is administered alone does not generate an immune response to the infectious, replication-deficient arenavirus particle. In some embodiments, the adjuvant generates an immune response to the infectious, replication-deficient arenavirus particle and does not produce an allergy or other adverse reaction. Adjuvants can enhance an immune response by several mechanisms including, e.g., lymphocyte recruitment, stimulation of B and/or T cells, and stimulation of macrophages. When a vaccine or immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., N. Engl. J. Med. 336, 86-91 (1997)).

The compositions comprise the infectious, replication-deficient arenavirus particles described herein alone or together with a pharmaceutically acceptable carrier and/or a chemotherapeutic agent. Suspensions or dispersions of genetically engineered arenavirus particles, especially isotonic aqueous suspensions or dispersions, can be used. The pharmaceutical compositions may be sterilized and/or may comprise excipients, e.g., preservatives, stabilizers, wetting agents and/or emulsifiers, solubilizers, salts for regulating osmotic pressure and/or buffers and are prepared in a manner known per se, for example by means of conventional dispersing and suspending processes. In certain embodiments, such dispersions or suspensions may comprise viscosity-regulating agents. The suspensions or dispersions are kept at temperatures around 2-8° C., or preferentially for longer storage may be frozen and then thawed shortly before use. For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

In certain embodiments, the compositions described herein additionally comprise a preservative, e.g., the mercury derivative thimerosal. In a specific embodiment, the pharmaceutical compositions described herein comprise 0.001% to 0.01% thimerosal. In other embodiments, the pharmaceutical compositions described herein do not comprise a preservative.

The pharmaceutical compositions comprise from about 10³ to about 10¹¹ focus forming units of the genetically engineered arenavirus particles. Unit dose forms for parenteral administration are, for example, ampoules or vials, e.g., vials containing from about 10³ to 10¹⁰ focus forming units or 10⁵ to 10¹⁵ physical particles of genetically engineered arenavirus particles.

In another embodiment, a vaccine or immunogenic composition provided herein is administered to a subject by, including but not limited to, oral, intradermal, intramuscular, intraperitoneal, intravenous, topical, subcutaneous, percutaneous, intranasal and inhalation routes, and via scarification (scratching through the top layers of skin, e.g., using a bifurcated needle). Specifically, subcutaneous, intramuscular or intravenous routes can be used.

For administration intranasally or by inhalation, the preparation for use according to the present invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflators may be formulated containing a powder mix of the compound and a suitable powder base such as lactose or starch.

The dosage of the active ingredient depends upon the type of vaccination and upon the subject, and their age, weight, individual condition, the individual pharmacokinetic data, and the mode of administration.

In certain embodiments, the compositions can be administered to the patient in a single dosage comprising a therapeutically effective amount of the arenavirus particle and/or a therapeutically effective amount of a chemotherapeutic agent. In some embodiments, the arenavirus particle can be administered to the patient in a single dose comprising an arenavirus particle and a chemotherapeutic agent, each in a therapeutically effective amount.

In certain embodiments, the composition is administered to the patient as a single dose followed by a second dose three to six weeks later. In accordance with these embodiments, the booster inoculations may be administered to the subjects at six to twelve month intervals following the second inoculation. In certain embodiments, the booster inoculations may utilize a different arenavirus particle or composition thereof. In some embodiments, the administration of the same composition as described herein may be repeated and separated by at least 1 day, 2 days, 3 days, 4 days, 5 days, 10 days, 15 days, 30 days, 45 days, 2 months, 75 days, 3 months, or at least 6 months.

Also provided are processes and uses of an arenavirus particle and a chemotherapeutic agent for the manufacture of vaccines in the form of pharmaceutical preparations, which comprise the arenavirus particle and the chemotherapeutic agent as an active ingredient. Still further provided is a combination of an arenavirus particle provided herein and a chemotherapeutic agent provided herein for use in the treatment of a neoplastic disease described herein. In certain embodiments, the combination is in the same pharmaceutical composition. In certain embodiments, the combination is not in the same pharmaceutical composition, such as when the arenavirus particle and the chemotherapeutic agent are to be separately administered. The pharmaceutical compositions of the present application are prepared in a manner known per se, for example by means of conventional mixing and/or dispersing processes.

Also provided herein are kits that can be used to perform the methods described herein. In certain embodiments, the kit provided herein can include one or more containers. These containers can hold for storage the compositions (e.g., pharmaceutical, immunogenic or vaccine composition) provided herein. Also included in the kit are instructions for use. These instructions describe, in sufficient detail, a treatment protocol for using the compositions contained therein. For example, the instructions can include dosing and administration instructions as provided herein for the methods of treating a neoplastic disease.

In certain embodiments, a kit provided herein includes containers that each contains the active ingredients for performing the methods described herein. Thus, in certain embodiments, the kit provided herein includes two or more containers and instructions for use, wherein one of the containers comprises an infectious, replication-deficient arenavirus particle provided herein and another container that comprises a chemotherapeutic agent provided herein.

In a specific embodiment, a kit provided herein includes two or more containers and instructions for use, wherein one of the containers comprises an infectious, replication-deficient arenavirus particle provided herein and another container that comprises a chemotherapeutic agent provided herein.

### (g) Assays

Assay for Measuring Arenavirus Vector Infectivity Any assay known to the skilled artisan can be used for measuring the infectivity of an arenavirus vector preparation. For example, determination of the virus/vector titer can be done by a “focus forming unit assay” (FFU assay). In brief, complementing cells, e.g. BEK 293 cells expressing LCMV GP protein, are plated and inoculated with different dilutions of a virus/vector sample. After an incubation period, to allow cells to form a monolayer and virus to attach to cells, the monolayer is covered with Methylcellulose. When the plates are further incubated, the original infected cells release viral progeny. Due to the Methylcellulose overlay the spread of the new viruses is restricted to neighboring cells. Consequently, each infectious particle produces a circular zone of infected cells called a Focus. Such Foci can be made visible and by that countable using antibodies against LCMV-NP and a HRP-based color reaction. The titer of a virus/vector can be calculated in focus-forming units per milliliter (FFU/mL).

To determine the infectious titer (FFU/mL) of transgene-carrying vectors this assay is modified by the use of the respective transgene-specific antibody instead of anti-LCMV-NP antibody.

Serum ELISA Determination of the humoral immune response upon vaccination of animals (e.g. mice, guinea pigs) can be done by antigen-specific serum ELISAs (enzyme-linked immunosorbent assays). In brief, plates are coated with antigen (e.g. recombinant protein), blocked to avoid unspecific binding of antibodies and incubated with serial dilutions of sera. After incubation, bound serum-antibodies can be detected, e.g., using an enzyme-coupled anti-species (e.g. mouse, guinea pig)-specific antibody (detecting total IgG or IgG subclasses) and subsequent color reaction. Antibody titers can be determined as, e.g., endpoint geometric mean titer.

Immunocapture ELISA (IC-ELISA) may also be performed (see Shanmugham et al., 2010, Clin. Vaccine Immunol. 17(8):1252-1260), wherein the capture agents are cross-linked to beads.

Immunocapture ELISA (IC-ELISA) may also be performed (see Shanmugham et al., 2010, Clin. Vaccine Immunol. 17(8):1252-1260), wherein the capture agents are cross-linked to beads.

Neutralizing Assay in ARPE-19 cells Determination of the neutralizing activity of induced antibodies in sera is performed with the following cell assay using ARPE-19 cells from ATCC and a GFP-tagged virus. In addition supplemental serum as a source of exogenous complement is used. The assay is started with seeding of 6.5×10³ cells/well (50 μl/well) in a 384 well plate one or two days before using for neutralization. The neutralization is done in 96-well sterile tissue culture plates without cells for 1 h at 37° C. After the neutralization incubation step the mixture is added to the cells and incubated for additional 4 days for GFP-detection with a plate reader. A positive neutralizing human sera is used as assay positive control on each plate to check the reliability of all results. Titers (EC50) are determined using a 4 parameter logistic curve fitting. As additional testing the wells are checked with a fluorescence microscope.

Plaque Reduction Assay In brief, plaque reduction (neutralization) assays for LCMV can be performed by use of a replication-deficient LCMV that is tagged with green fluorescent protein, 5% rabbit serum may be used as a source of exogenous complement, and plaques can be enumerated by fluorescence microscopy. Neutralization titers may be defined as the highest dilution of serum that results in a 50%, 75%, 90% or 95% reduction in plaques, compared with that in control (pre-immune) serum samples.

Neutralization Assay in guinea pig lung fibroblast (GPL) cells In brief, serial dilutions of test and control (pre-vaccination) sera were prepared in GPL complete media with supplemental rabbit serum (1%) as a source of exogenous complement. The dilution series spanned 1:40 through 1:5120. Serum dilutions were incubated with eGFP tagged virus (100-200 pfu per well) for 30 min at 37° C., and then transferred to 12-well plates containing confluent GPL cells. Samples were processed in triplicate. After 2 hours incubation at 37° C. the cells were washed with PBS, re-fed with GPL complete media and incubated at 37° C./5% C02 for 5 days. Plaques were visualized by fluorescence microscopy, counted, and compared to control wells. That serum dilution resulting in a 50% reduction in plaque number compared to controls was designated as the neutralizing titer.

qPCR LCMV RNA genomes are isolated using QIAamp Viral RNA mini Kit (QIAGEN), according to the protocol provided by the manufacturer. LCMV RNA genome equivalents are detected by quantitative PCR carried out on an StepOnePlus Real Time PCR System (Applied Biosystems) with SuperScript® III Platinum® One-Step qRT-PCR Kit (Invitrogen) and primers and probes (FAM reporter and NFQ-MGB Quencher) specific for part of the LCMV NP coding region. The temperature profile of the reaction is: 30 min at 60° C., 2 min at 95° C., followed by 45 cycles of 15 s at 95° C., 30 s at 56° C. RNA is quantified by comparison of the sample results to a standard curve prepared from a log 10 dilution series of a spectrophotometrically quantified, in vitro-transcribed RNA fragment, corresponding to a fragment of the LCMV NP coding sequence containing the primer and probe binding sites.

Neutralization Assay in guinea pig lung fibroblast (GPL) cells In brief, serial dilutions of test and control (pre-vaccination) sera were prepared in GPL complete media with supplemental rabbit serum (1%) as a source of exogenous complement. The dilution series spanned 1:40 through 1:5120. Serum dilutions were incubated with eGFP tagged virus (100-200 pfu per well) for 30 min at 37° C., and then transferred to 12-well plates containing confluent GPL cells. Samples were processed in triplicate. After 2 hours incubation at 37° C. the cells were washed with PBS, re-fed with GPL complete media and incubated at 37° C./5% CO₂ for 5 days. Plaques were visualized by fluorescence microscopy, counted, and compared to control wells. That serum dilution resulting in a 50% reduction in plaque number compared to controls was designated as the neutralizing titer.

Western Blotting Infected cells grown in tissue culture flasks or in suspension are lysed at indicated timepoints post infection using RIPA buffer (Thermo Scientific) or used directly without cell-lysis. Samples are heated to 99° C. for 10 minutes with reducing agent and NuPage LDS Sample buffer (NOVEX) and chilled to room temperature before loading on 4-12% SDS-gels for electrophoresis. Proteins are blotted onto membranes using Invitrogens iBlot Gel transfer Device and visualized by Ponceau staining. Finally, the preparations are probed with an primary antibodies directed against proteins of interest and alkaline phosphatase conjugated secondary antibodies followed by staining with 1-Step NBT/BCIP solution (INVITROGEN).

MHC-Peptide Multimer Staining Assay for Detection of Antigen-Specific CD8+ T-cell proliferation Any assay known to the skilled artisan can be used to test antigen-specific CD8+ T-cell responses. For example, the MHC-peptide tetramer staining assay can be used (see, e.g., Altman J. D. et al., Science. 1996; 274:94-96; and Murali-Krishna K. et al., Immunity. 1998; 8:177-187). Briefly, the assay comprises the following steps, a tetramer assay is used to detect the presence of antigen specific T-cells. In order for a T-cell to detect the peptide to which it is specific, it must both recognize the peptide and the tetramer of MHC molecules custom made for an antigen specific T-cell (typically fluorescently labeled). The tetramer is then detected by flow cytometry via the fluorescent label.

ELISPOT Assay for Detection of Antigen-Specific CD4+ T-cell Proliferation Any assay known to the skilled artisan can be used to test antigen-specific CD4+ T-cell responses. For example, the ELISPOT assay can be used (see, e.g., Czerkinsky C. C. et al., J Immunol Methods. 1983; 65:109-121; and Hutchings P. R. Et al., J Immunol Methods. 1989; 120:1-8). Briefly, the assay comprises the following steps: An immunospot plate is coated with an anti-cytokine antibody. Cells are incubated in the immunospot plate. Cells secrete cytokines and are then washed off. Plates are then coated with a second biotyinlated-anticytokine antibody and visualized with an avidin-HRP system.

Intracellular Cytokine Assay for Detection of Functionality of CD8+ and CD4+ T-cell Responses Any assay known to the skilled artisan can be used to test the functionality of CD8+ and CD4+ T cell responses. For example, the intracellular cytokine assay combined with flow cytometry can be used (see, e.g., Suni M. A. et al., J Immunol Methods. 1998; 212:89-98; Nomura L. E. et al., Cytometry. 2000; 40:60-68; and Ghanekar S. A. et al., Clinical and Diagnostic Laboratory Immunology. 2001; 8:628-63). Briefly, the assay comprises the following steps: activation of cells via specific peptides or protein, an inhibition of protein transport (e.g., brefeldin A) is added to retain the cytokines within the cell. After washing, antibodies to other cellular markers can be added to the cells. Cells are then fixed and permeabilized. The anti-cytokine antibody is added and the cells can be analyzed by flow cytometry.

Assay for Confirming Replication-Deficiency of Viral Vectors Any assay known to the skilled artisan that determines concentration of infectious and replication-competent virus particles can also be used as a to measure replication-deficient viral particles in a sample. For example, FFU assays with non-complementing cells can be used for this purpose.

Furthermore, plaque-based assays are the standard method used to determine virus concentration in terms of plaque forming units (PFU) in a virus sample. Specifically, a confluent monolayer of non-complementing host cells is infected with the virus at varying dilutions and covered with a semi-solid medium, such as agar to prevent the virus infection from spreading indiscriminately. A viral plaque is formed when a virus successfully infects and replicates itself in a cell within the fixed cell monolayer (see, e.g., Kaufmann, S. H.; Kabelitz, D. (2002). Methods in Microbiology Vol. 32:Immunology of Infection. Academic Press. ISBN 0-12-521532-0). Plaque formation can take 3-14 days, depending on the virus being analyzed. Plaques are generally counted manually and the results, in combination with the dilution factor used to prepare the plate, are used to calculate the number of plaque forming units per sample unit volume (PFU/mL). The PFU/mL result represents the number of infective replication-competent particles within the sample.

Assay for Expression of Viral Antigen Any assay known to the skilled artisan can be used for measuring expression of viral antigens. For example, FFU assayscan be performed. For detection, mono- or polyclonal antibody preparation(s) against respective viral antigens are used (transgene-specific FFU).

Animal Models The safety, tolerance and immunogenic effectiveness of vaccines comprising of an infectious, replication-deficient arenavirus expressing a tumor antigen, tumor associate antigen or antigenic fragment thereof described herein or a composition thereof can be tested in animals models. In certain embodiments, the animal models that can be used to test the safety, tolerance and immunogenic effectiveness of the vaccines and compositions thereof used herein include mouse, guinea pig, rat, monkey, and chimpanzee. In a preferred embodiment, the animal models that can be used to test the safety, tolerance and immunogenic effectiveness of the vaccines and compositions thereof used herein include mouse.

### Chemotherapeutic Agent Assays

A number of assays have been devised that are capable of assessing properties of proposed chemotherapeutic agents. Tumor models that can be used to test the methods and compositions disclosed herein include Colon26 (CT26), MC38 (mouse colon adenocarcinoma), B16F10 (B16), Lewis Lung (LLC), Madison109 (Mad 109), EMT-6 (murine breast cancer), 4T1 (4T1) (murine breast cancer), HCmel3 (murine melanoma), HgfxCDK4^{R24C/R24C} (murine melanoma), and (RENCA) (murine renal cancer).

In certain embodiments, in these model systems, “transplantable tumors” can be generated by subcutaneous (e.g., CT26, 4T1, MAD109, RENCA, LLC, or B16) or intracerebral (e.g., GL261, ONC26M4) inoculation of tumor cell lines into rodents, for example in adult female mice. Tumors can be developed over pre-determined time intervals, for example several days. These tumors are grown in syngeneic, immunocompetent rodent, e.g., mouse, strains. For example CT26, 4T1, MAD109, and RENCA can be grown in BALB/c mice, LLC, B16, and GL261 can be grown in C57BL/6 mice, and ONC26M4 can be grown in FVBN mice. “Spontaneous tumors” can be generated by intracerebral injection of DNA plasmids encoding a number (e.g., one, two, three or more) of oncogenes and encoding one or more reporter, e.g., firefly luciferase reporter, into neonatal C57BL/6 or FVBN mice to transform endogenous brain cells. Growth of gliomas can be monitored by techniques known in the art, e.g., bioluminescence imaging. Growth of subcutaneous tumors can be monitored by techniques known in the art, e.g., caliper measurements in three dimensions at specified time intervals.

### 5.2 Tri-Segmented Arenavirus Particles

In certain embodiments, tri-segmented arenavirus particles comprising a nucleotide sequence encoding a tumor antigen, tumor associated antigen or an antigenic fragment thereof in combination with a chemotherapeutic agent, can be used as immunotherapies for treating a neoplastic disease, such as cancer. The term “neoplastic” or “neoplasm” refers to an abnormal new growth of cells or tissue. This abnormal new growth can form a mass, also known as a tumor or neoplasia. A neoplasm includes a benign neoplasm, an in situ neoplasm, a malignant neoplasm, and a neoplasm of uncertain or unknown behavior. In certain embodiments, the neoplastic disease treated using the methods and compositions described herein is cancer.

Provided herein are combination treatments for the treatment and/or prevention of a neoplastic disease, such as cancer. Specifically, such combination treatments comprise administering arenavirus particles or viral vectors that comprise a nucleotide sequence encoding one or more tumor antigens, tumor associated antigens or antigenic fragments thereof, in combination with one or more chemotherapeutic agents. These genetically modified viruses can be administered to a subject for the treatment of a neoplastic disease, such as cancer. Detailed descriptions of the arenaviruses provided herein, including the nucleotide sequences encoding a tumor antigen, tumor associated antigen or antigenic fragment thereof can be found in Sections 5.2.(a), 5.2.(b), and 5.2.(c). Arenaviruses comprising an open reading frame at a non-natural position are described in Section 5.2.(a). Tri-segmented arenaviruses are described in Section 5.2.(b) Tumor antigens that can be used with the present methods and compositions can be found in Section 5.2.(c). Additionally, methods for generation of arenavirus particles or viral vectors for use in the methods and compositions described herein are described in more detail in Section 5.2. (d).

In addition to administering arenavirus particles or viral vectors to a subject, the immunotherapies for treating a neoplastic disease provided herein can include a chemotherapeutic agent. “Chemotherapeutic agents” are cytotoxic anti-cancer agents, and can be categorized by their mode of activity within a cell, for example, at what stage they affect the cell cycle (e.g., a mitosis inhibitor). Alternatively, chemotherapeutic agents can be characterized based on ability to cross-link DNA, to intercalate into DNA, or to induce chromosomal aberrations by affecting nucleic acid synthesis (e.g., alkylating agents), among other mechanisms of action. Chemotherapeutic agents can also be characterized based on chemical components or structure (e.g., platinum-based therapeutics). Thus, in certain embodiments, provided herein are methods and compositions for treating a neoplastic disease using an arenavirus particle or viral vector comprising a nucleotide sequence encoding a tumor antigen, tumor associated antigen or antigenic fragment thereof and a chemotherapeutic agent. Thus, in certain embodiments, provided herein are methods for treating a neoplastic disease using an arenavirus particle or viral vector comprising a nucleotide sequence encoding a tumor antigen, tumor associated antigen or antigenic fragment thereof, and a chemotherapeutic agent. Also, in certain embodiments, provided herein are compositions comprising an arenavirus particle or viral vector comprising a nucleotide sequence encoding a tumor antigen, tumor associated antigen or antigenic fragment thereof, and a chemotherapeutic agent. In certain embodiments, the arenavirus particle or viral vector provided herein is engineered to contain an arenavirus genomic segment having a nucleotide sequence encoding a tumor antigen, tumor associated antigen or antigenic fragment thereof and at least one arenavirus open reading frame (“ORF”) in a position other than the wild-type position of the ORF. In certain embodiments, the arenavirus particle or viral vector provided herein is an infectious, replication deficient arenavirus particle or viral vector. In other embodiments, the arenavirus particle provided herein is a tri-segmented arenavirus particle or viral vector, which can be replication-deficient or replication-competent. In still other embodiments, the tri-segmented arenavirus particle or viral vector provided herein, when propagated, does not result in a replication-competent bi-segmented viral particle. Methods and compositions for using an arenavirus particle or viral vector and a chemotherapeutic agent provided herein are described in more detail in Sections 5.2.(f) and 5.2.(g).

In addition to administering arenavirus particles or viral vectors to a subject in combination with a chemotherapeutic agent, the immunotherapies for treating a neoplastic disease provided herein can also include an immune checkpoint modulator. The term “immune checkpoint modulator” (also referred to as “checkpoint modulator” or as “checkpoint regulator”) refers to a molecule or to a compound that modulates (e.g., totally or partially reduces, inhibits, interferes with, activates, stimulates, increases, reinforces or supports) the function of one or more checkpoint molecules. Thus, an immune checkpoint modulator may be an immune checkpoint inhibitor or an immune checkpoint activator.

An “immune checkpoint inhibitor” refers to a molecule that inhibits, decreases or interferes with the activity of a negative checkpoint regulator. In certain embodiments, immune checkpoint inhibitors for use with the methods and compositions disclosed herein can inhibit the activity of a negative checkpoint regulator directly, or decrease the expression of a negative checkpoint regulator, or interfere with the interaction of a negative checkpoint regulator and a binding partner (e.g., a ligand). Immune checkpoint inhibitors for use with the methods and compositions disclosed herein include a protein, a polypeptide, a peptide, an antisense oligonucleotide, an antibody, an antibody fragment, or an inhibitory RNA molecule that targets the expression of a negative checkpoint regulator.

A “negative checkpoint regulator” refers to a molecule that down-regulates immune responses (e.g., T-cell activation) by delivery of a negative signal to T-cells following their engagement by ligands or counter-receptors. Exemplary functions of a negative-checkpoint regulator are to prevent out-of-proportion immune activation, minimize collateral damage, and/or maintain peripheral self-tolerance. In certain embodiments, a negative checkpoint regulator is a ligand or receptor expressed by an antigen presenting cell. In certain embodiments, a negative checkpoint regulator is a ligand or receptor expressed by a T-cell. In certain embodiments, a negative checkpoint regulator is a ligand or receptor expressed by both an antigen presenting cell and a T-cell.

(a) Arenaviruses with an Open Reading Frame in a Non-natural Position

In certain embodiments, arenaviruses with rearrangements of their ORFs and a nucleotide sequence encoding a tumor antigen, tumor associated antigen or an antigenic fragment thereof provided herein can be used with the methods and compositions provided herein, such as combinations with a chemotherapeutic agent. In certain embodiments, such arenaviruses are replication-competent and infectious. Thus, in certain embodiments, provided herein is an arenavirus genomic segment, wherein the arenavirus genomic segment is engineered to carry an arenavirus ORF in a position other than the position in which the respective gene is found in viruses isolated from the wild, such as LCMV-MP (referred to herein as “wild-type position”) of the ORF (i.e., a non-natural position) and a nucleotide sequence encoding a tumor antigen, tumor associated antigen or an antigenic fragment thereof provided herein.

The wild-type arenavirus genomic segments and ORFs are known in the art. In particular, the arenavirus genome consists of an S segment and an L segment. The S segment carries the ORFs encoding the GP and the NP. The L segment encodes the L protein and the Z protein. Both segments are flanked by the respective 5′ and 3′ UTRs.

In certain embodiments, an arenavirus genomic segment can be engineered to carry two or more arenavirus ORFs in a position other than the wild-type position. In other embodiments, the arenavirus genomic segment can be engineered to carry two arenavirus ORFs, or three arenavirus ORFs, or four arenavirus ORFs in a position other than the wild-type position.

In certain embodiments, an arenavirus genomic segment provided herein can be:
(xix) an arenavirus S segment, wherein the ORF encoding the NP is under control of an arenavirus 5′ UTR;
(xx) an arenavirus S segment, wherein the ORF encoding the Z protein is under control of an arenavirus 5′ UTR;
(xxi) an arenavirus S segment, wherein the ORF encoding the L protein is under control of an arenavirus 5′ UTR;
(xxii) an arenavirus S segment, wherein the ORF encoding the GP is under control of an arenavirus 3′ UTR;
(xxiii) an arenavirus S segment, wherein the ORF encoding the L protein is under control of an arenavirus 3′ UTR;
(xxiv) an arenavirus S segment, wherein the ORF encoding the Z protein is under control of an arenavirus 3′ UTR;
(xxv) an arenavirus L segment, wherein the ORF encoding the GP is under control of an arenavirus 5′ UTR;
(xxvi) an arenavirus L segment, wherein the ORF encoding the NP is under control of an arenavirus 5′ UTR;
(xxvii) an arenavirus L segment, wherein the ORF encoding the L protein is under control of an arenavirus 5′ UTR;
(xxviii) an arenavirus L segment, wherein the ORF encoding the GP is under control of an arenavirus 3′ UTR;
(xxix) an arenavirus L segment, wherein the ORF encoding the NP is under control of an arenavirus 3′ UTR; and
(xxx) an arenavirus L segment, wherein the ORF encoding the Z protein is under control of an arenavirus 3′ UTR.

In certain embodiments, the ORF that is in the non-natural position of the arenavirus genomic segment described herein can be under the control of an arenavirus 3′ UTR or an arenavirus 5′ UTR. In more specific embodiments, the arenavirus 3′ UTR is the 3′ UTR of the arenavirus S segment. In another specific embodiment, the arenavirus 3′ UTR is the 3′UTR of the arenavirus L segment. In more specific embodiments, the arenavirus 5′ UTR is the 5′ UTR of the arenavirus S segment. In other specific embodiments, the 5′ UTR is the 5′ UTR of the L segment.

In other embodiments, the ORF that is in the non-natural position of the arenavirus genomic segment described herein can be under the control of the arenavirus conserved terminal sequence element (the 5′- and 3-terminal 19-20-nt regions) (see e.g., Perez & de la Torre, 2003, J Virol. 77(2): 1184-1194).

In certain embodiments, the ORF that is in the non-natural position of the arenavirus genomic segment can be under the control of the promoter element of the 5′ UTR (see e.g., Albarino et al., 2011, J Virol., 85(8):4020-4). In another embodiment, the ORF that is in the non-natural position of the arenavirus genomic segment can be under the control of the promoter element of the 3′ UTR (see e.g., Albarino et al., 2011, J Virol., 85(8):4020-4). In more specific embodiments, the promoter element of the 5′ UTR is the 5′ UTR promoter element of the S segment or the L segment. In another specific embodiment, the promoter element of the 3′ UTR is the 3′ UTR the promoter element of the S segment or the L segment.

In certain embodiments, the ORF that is in the non-natural position of the arenavirus genomic segment can be under the control of a truncated arenavirus 3′ UTR or a truncated arenavirus 5′ UTR (see e.g., Perez & de la Torre, 2003, J Virol. 77(2): 1184-1194; Albarino et al., 2011, J Virol., 85(8):4020-4). In more specific embodiments, the truncated 3′ UTR is the 3′ UTR of the arenavirus S segment or L segment. In more specific embodiments, the truncated 5′ UTR is the 5′ UTR of the arenavirus S segment or L segment.

Also provided herein, is an arenavirus particle comprising a first genomic segment that has been engineered to carry an ORF in a position other than the wild-type position of the ORF and a second arenavirus genomic segment so that the arenavirus particle comprises an S segment and an L segment. In specific embodiments, the ORF in a position other than the wild-type position of the ORF is one of the arenavirus ORFs.

In certain specific embodiments, the arenavirus particle can comprise a full complement of all four arenavirus ORFs. In specific embodiments, the second arenavirus genomic segment has been engineered to carry an ORF in a position other than the wild-type position of the ORF. In another specific embodiment, the second arenavirus genomic segment can be the wild-type genomic segment (i.e., comprises the ORFs on the segment in the wild-type position).

In certain embodiments, the first arenavirus genomic segment is an L segment and the second arenavirus genomic segment is an S segment. In other embodiments, the first arenavirus genomic segment is an S segment and the second arenavirus genomic segment is an L segment.

Non-limiting examples of the arenavirus particle comprising a genomic segment with an ORF in a position other than the wild-type position of the ORF and a second genomic segment are illustrated in Table 1.

TABLE 1
Arenavirus particle

| Position 1 | Position 2 | Position 3 | Position 4 |
|---|---|---|---|
| GP | NP | L | Z |
| GP | Z | L | NP |
| GP | Z | NP | L |
| GP | L | NP | Z |
| GP | L | Z | NP |
| NP | GP | L | Z |
| NP | GP | Z | L |
| NP | L | GP | Z |
| NP | L | Z | GP |
| NP | Z | GP | L |
| NP | Z | L | GP |
| Z | GP | L | NP |
| Z | GP | NP | L |
| Z | NP | GP | L |
| Z | NP | L | GP |
| Z | L | NP | GP |
| Z | L | GP | NP |
| L | NP | GP | Z |
| L | NP | Z | GP |
| L | GP | Z | NP |
| L | GP | NP | Z |
| L | Z | NP | GP |
| L | Z | GP | NP |

*Position 1 is under the control of an arenavirus S segment 5′ UTR;
Position 2 is under the control of an arenavirus S segment 3′ UTR;
Position 3 is under the control of an arenavirus L segment 5′ UTR;
Position 4 is under the control of an arenavirus L segment 3′ UTR.

Also provided herein, is a cDNA of the arenavirus genomic segment engineered to carry an ORF in a position other than the wild-type position of the ORF and a nucleotide sequence encoding a tumor antigen, tumor associated antigen or an antigenic fragment thereof provided herein. In more specific embodiments, provided herein is a cDNA or a set of cDNAs of an arenavirus genome as set forth in Table 1.

In certain embodiments, a cDNA of the arenavirus genomic segment that is engineered to carry an ORF in a position other than the wild-type position of the ORF is part of or incorporated into a DNA expression vector. In a specific embodiment, a cDNA of the arenavirus genomic segment that is engineered to carry an ORF in a position other than the wild-type position of the ORF is part of or incorporated into a DNA expression vector that facilitates production of an arenavirus genomic segment as described herein. In another embodiment, a cDNA described herein can be incorporated into a plasmid. More detailed description of the cDNAs or nucleic acids and expression systems are provided is Section 5.2.(e). Techniques for the production of a cDNA are routine and conventional techniques of molecular biology and DNA manipulation and production. Any cloning technique known to the skilled artesian can be used. Such as techniques are well known and are available to the skilled artesian in laboratory manuals such as, Sambrook and Russell, Molecular Cloning: A laboratory Manual, 3^{rd} edition, Cold Spring Harbor Laboratory N. Y. (2001).

In certain embodiments, the cDNA of the arenavirus genomic segment that is engineered to carry an ORF in a position other than the wild-type position of the ORF and a nucleotide sequence encoding a tumor antigen, tumor associated antigen or an antigenic fragment thereof provided herein is introduced (e.g., transfected) into a host cell. Thus, in some embodiments provided herein, is a host cell comprising a cDNA of the arenavirus genomic segment that is engineered to carry an ORF in a position other than the wild-type position of the ORF (i.e., a cDNA of the genomic segment) and a nucleotide sequence encoding a tumor antigen, tumor associated antigen or an antigenic fragment thereof provided herein. In other embodiments, the cDNA described herein is part of or can be incorporated into a DNA expression vector and introduced into a host cell. Thus, in some embodiments provided herein is a host cell comprising a cDNA described herein that is incorporated into a vector. In other embodiments, the arenavirus genomic segment described herein is introduced into a host cell.

In certain embodiments, described herein is a method of producing the arenavirus genomic segment comprising a nucleotide sequence encoding a tumor antigen, tumor associated antigen or an antigenic fragment thereof provided herein, wherein the method comprises transcribing the cDNA of the arenavirus genomic segment. In certain embodiments, a viral polymerase protein can be present during transcription of the arenavirus genomic segment in vitro or in vivo.

In certain embodiments transcription of the arenavirus genomic segment is performed using a bi-directional promoter. In other embodiments, transcription of the arenavirus genomic segment is performed using a bi-directional expression cassette (see e.g., Ortiz-Riano et al., 2013, J Gen Virol., 94(Pt 6): 1175-1188). In more specific embodiments the bi-directional expression cassette comprises both a polymerase I and a polymerase II promoter reading from opposite sides into the two termini of the inserted arenavirus genomic segment, respectively. In yet more specific embodiments the bi-directional expression cassette with pol-I and pol-II promoters read from opposite sides into the L segment and S segment

In other embodiments, transcription of the cDNA of the arenavirus genomic segment described herein comprises a promoter. Specific examples of promoters include an RNA polymerase I promoter, an RNA polymerase II promoter, an RNA polymerase III promoter, a T7 promoter, an SP6 promote or a T3 promoter.

In certain embodiments, the method of producing the arenavirus genomic segment can further comprise introducing into a host cell the cDNA of the arenavirus genomic segment comprising a nucleotide sequence encoding a tumor antigen, tumor associated antigen or an antigenic fragment thereof provided herein. In certain embodiments, the method of producing the arenavirus genomic segment can further comprise introducing into a host cell the cDNA of the arenavirus genomic segment comprising a nucleotide sequence encoding a tumor antigen, tumor associated antigen or an antigenic fragment thereof provided herein, wherein the host cell expresses all other components for production of the arenavirus genomic segment; and purifying the arenavirus genomic segment from the supernatant of the host cell. Such methods are well-known to those skilled in the art.

Provided herein are cell lines, cultures and methods of culturing cells infected with nucleic acids, vectors, and compositions provided herein. More detailed description of nucleic acids, vector systems and cell lines described herein is provided in Section 5.2.(e).

In certain embodiments, the arenavirus particle as described herein results in an infectious and replication competent arenavirus particle. In specific embodiments, the arenavirus particle described herein is attenuated. In a particular embodiment, the arenavirus particle is attenuated such that the virus remains, at least partially, able to spread and can replicate in vivo, but can only generate low viral loads resulting in subclinical levels of infection that are non-pathogenic. Such attenuated viruses can be used as an immunogenic composition. Provided herein, are immunogenic compositions that comprise an arenavirus with an ORF in a non-natural position as described in Section (g).

(i) Replication-Defective Arenavirus Particle with an Open Reading Frame in a Non-Natural Position

In certain embodiments, provided herein is an arenavirus particle in which (i) an ORF is in a position other than the wild-type position of the ORF; and (ii) an ORF encoding GP, NP, Z protein, and L protein has been removed or functionally inactivated such that the resulting virus cannot produce further infectious progeny virus particles. An arenavirus particle comprising a genetically modified genome in which one or more ORFs has been deleted or functionally inactivated can be produced in complementing cells (i.e., cells that express the arenavirus ORF that has been deleted or functionally inactivated). The genetic material of the resulting arenavirus particle can be transferred upon infection of a host cell into the host cell, wherein the genetic material can be expressed and amplified. In addition, the genome of the genetically modified arenavirus particle described herein can encode a heterologous ORF from an organism other than an arenavirus particle.

In certain embodiments, an ORF of the arenavirus is deleted or functionally inactivated and replaced with a nucleotide sequence encoding a tumor antigen or tumor associated antigen as described herein. In a specific embodiment, the ORF that encodes the glycoprotein GP of the arenavirus is deleted or functionally inactivated. In certain embodiments, functional inactivation of a gene eliminates any translation product. In certain embodiments, functional inactivation refers to a genetic alteration that allows some translation, the translation product, however, is not longer functional and cannot replace the wild type protein.

In certain embodiments, at least one of the four ORFs encoding GP, NP, Z protein, and L protein is removed and replaced with a nucleotide sequence encoding a tumor antigen, tumor associated antigen or an antigenic fragment thereof provided herein. In another embodiment, at least one ORF, at least two ORFs, at least three ORFs, or at least four ORFs encoding GP, NP, Z protein and L protein can be removed and replaced with a nucleotide sequence encoding a tumor antigen, tumor associated antigen or an antigenic fragment thereof provided herein. In specific embodiments, only one of the four ORFs encoding GP, NP, Z protein, and L protein is removed and replaced with a nucleotide sequence encoding a tumor antigen, tumor associated antigen or an antigenic fragment thereof provided herein. In more specific embodiments, the ORF that encodes GP of the arenavirus genomic segment is removed. In another specific embodiment, the ORF that encodes the NP of the arenavirus genomic segment is removed. In more specific embodiments, the ORF that encodes the Z protein of the arenavirus genomic segment is removed. In yet another specific embodiment, the ORF encoding the L protein is removed.

Thus, in certain embodiments, the arenavirus particle provided herein comprises a genomic segment that (i) is engineered to carry an ORF in a non-natural position; (ii) an ORF encoding GP, NP, Z protein, or L protein is removed; (iii) the ORF that is removed is replaced with a nucleotide sequence encoding a tumor antigen, tumor associated antigen or an antigenic fragment thereof provided herein.

In certain embodiments, the fragment of the tumor antigen or tumor associated antigen is antigenic when it is capable of (i) eliciting an antibody immune response in a host (e.g., mouse, rabbit, goat, donkey or human) wherein the resulting antibodies bind specifically to an immunogenic protein expressed in or on a neoplastic cell (e.g., a cancer cell); and/or (ii) eliciting a specific T cell immune response.

In certain embodiments, the nucleotide sequence encoding an antigenic fragment provided herein is 8 to 100 nucleotides in length, 15 to 100 nucleotides in length, 25 to 100 nucleotides in length, 50 to 200 nucleotide in length, 50 to 400 nucleotide in length, 200 to 500 nucleotide in length, or 400 to 600 nucleotides in length, 500 to 800 nucleotide in length. In other embodiments, the nucleotide sequence encoding an antigenic fragment provided herein is 750 to 900 nucleotides in length, 800 to 100 nucleotides in length, 850 to 1000 nucleotides in length, 900 to 1200 nucleotides in length, 1000 to 1200 nucleotides in length, 1000 to 1500 nucleotides or 10 to 1500 nucleotides in length, 1500 to 2000 nucleotides in length, 1700 to 2000 nucleotides in length, 2000 to 2300 nucleotides in length, 2200 to 2500 nucleotides in length, 2500 to 3000 nucleotides in length, 3000 to 3200 nucleotides in length, 3000 to 3500 nucleotides in length, 3200 to 3600 nucleotides in length, 3300 to 3800 nucleotides in length, 4000 nucleotides to 4400 nucleotides in length, 4200 to 4700 nucleotides in length, 4800 to 5000 nucleotides in length, 5000 to 5200 nucleotides in length, 5200 to 5500 nucleotides in length, 5500 to 5800 nucleotides in length, 5800 to 6000 nucleotides in length, 6000 to 6400 nucleotides in length, 6200 to 6800 nucleotides in length, 6600 to 7000 nucleotides in length, 7000 to 7200 nucleotides in lengths, 7200 to 7500 nucleotides in length, or 7500 nucleotides in length. In some embodiments, the nucleotide sequence encodes a peptide or polypeptide that is 5 to 10 amino acids in length, 10 to 25 amino acids in length, 25 to 50 amino acids in length, 50 to 100 amino acids in length, 100 to 150 amino acids in length, 150 to 200 amino acids in length, 200 to 250 amino acids in length, 250 to 300 amino acids in length, 300 to 400 amino acids in length, 400 to 500 amino acids in length, 500 to 750 amino acids in length, 750 to 1000 amino acids in length, 1000 to 1250 amino acids in length, 1250 to 1500 amino acids in length, 1500 to 1750 amino acids in length, 1750 to 2000 amino acids in length, 2000 to 2500 amino acids in length, or more than 2500 or more amino acids in length. In some embodiments, the nucleotide sequence encodes a polypeptide that does not exceed 2500 amino acids in length. In specific embodiments the nucleotide sequence does not contain a stop codon. In certain embodiments, the nucleotide sequence is codon-optimized. In certain embodiments the nucleotide composition, nucleotide pair composition or both can be optimized. Techniques for such optimizations are known in the art and can be applied to optimize a nucleotide sequence encoding a tumor antigen, tumor associated antigen or an antigenic fragment thereof provided herein.

In certain embodiments, the growth and infectivity of the arenavirus particle is not affected by the nucleotide sequence encoding a tumor antigen, tumor associated antigen or an antigenic fragment thereof provided herein.

Techniques known to one skilled in the art may be used to produce an arenavirus particle comprising an arenavirus genomic segment engineered to carry an arenavirus ORF in a position other than the wild-type position and a nucleotide sequence encoding a tumor antigen, tumor associated antigen or an antigenic fragment thereof provided herein. For example, reverse genetics techniques may be used to generate such arenavirus particle. In other embodiments, the replication-defective arenavirus particle (i.e., the arenavirus genomic segment engineered to carry an arenavirus ORF in a position other than the wild-type position, wherein an ORF encoding GP, NP, Z protein, L protein, has been deleted) can be produced in a complementing cell.

In certain embodiments, an arenavirus particle or arenavirus genomic segment provided herein comprising a nucleotide sequence encoding a tumor antigen, tumor associated antigen or antigenic fragment thereof as provided herein further comprises at least one nucleotide sequence encoding at least one immunomodulatory peptide, polypeptide or protein. In certain embodiments, the immunomodulatory peptide, polypeptide or protein is Calreticulin (CRT), or a fragment thereof, Ubiquitin or a fragment thereof, Granulocyte-Macrophage Colony-Stimulating Factor (GM-CSF), or a fragment thereof, Invariant chain (CD74) or an antigenic fragment thereof; Mycobacterium tuberculosis Heat shock protein 70 or an antigenic fragment thereof; Herpes simplex virus 1 protein VP22 or an antigenic fragment thereof, CD40 ligand or an antigenic fragment thereof, or Fms-related tyrosine kinase 3 (Flt3) ligand or an antigenic fragment thereof.

In certain embodiments, the arenavirus genomic segment or the arenavirus particle used according to the present application can be Old World viruses, for example Lassa virus, Lymphocytic choriomeningitis virus (LCMV), Mobala virus, Mopeia virus, or Ippy virus, or New World viruses, for example Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Bear Canyon virus, or Whitewater Arroyo virus.

In certain embodiments, the arenavirus particle as described herein is suitable for use as a vaccine and methods of using such arenavirus particle in a vaccination and treatment for a neoplastic disease, for example, cancer, is provided. More detailed description of the methods of using the arenavirus particle described herein is provided in Section 5.2.(f)

In certain embodiments, the arenavirus particle as described herein is suitable for use as a pharmaceutical composition and methods of using such arenavirus particle in a vaccination and treatment for a neoplastic disease, for example, cancer, is provided. More detailed description of the methods of using the arenavirus particle described herein is provided in Section 5.2.(g).

### (b) Tri-Segmented Arenavirus Particle

In certain embodiments, tri-segmented arenavirus particles with rearrangements of their ORFs and a nucleotide sequence encoding a tumor antigen, tumor associated antigen or an antigenic fragment thereof provided herein can be used with the methods and compositions provided herein, such as combinations with a chemotherapeutic agent. In one aspect, provided herein is a tri-segmented arenavirus particle comprising one L segment and two S segments or two L segments and one S segment. In certain embodiments, the tri-segmented arenavirus particle does not recombine into a replication competent bi-segmented arenavirus particle. More specifically, in certain embodiments, two of the genomic segments (e.g., the two S segments or the two L segments, respectively) cannot recombine in a way to yield a single viral segment that could replace the two parent segments. In specific embodiments, the tri-segmented arenavirus particle comprises an ORF in a position other than the wild-type position of the ORF and a nucleotide sequence encoding a tumor antigen, tumor associated antigen or an antigenic fragment thereof provided herein. In yet another specific embodiment, the tri-segmented arenavirus particle comprises all four arenavirus ORFs. Thus, in certain embodiments, the tri-segmented arenavirus particle is replication competent and infectious. In other embodiments, the tri-segmented arenavirus particle lacks one of the four arenavirus ORFs. Thus, in certain embodiments, the tri-segmented arenavirus particle is infectious but unable to produce further infectious progeny in non-complementing cells.

In certain embodiments, the ORF encoding GP, NP, Z protein, or the L protein of the tri-segmented arenavirus particle described herein can be under the control of an arenavirus 3′ UTR or an arenavirus 5′ UTR. In more specific embodiments, the tri-segmented arenavirus 3′ UTR is the 3′ UTR of an arenavirus S segment(s). In another specific embodiment, the tri-segmented arenavirus 3′ UTR is the 3′ UTR of a tri-segmented arenavirus L segment(s). In more specific embodiments, the tri-segmented arenavirus 5′ UTR is the 5′ UTR of an arenavirus S segment(s). In other specific embodiments, the 5′ UTR is the 5′ UTR of the L segment(s).

In other embodiments, the ORF encoding GP, NP, Z protein, or the L protein of tri-segmented arenavirus particle described herein can be under the control of the arenavirus conserved terminal sequence element (the 5′- and 3-terminal 19-20-nt regions) (see e.g., Perez & de la Torre, 2003, J Virol. 77(2): 1184-1194).

In certain embodiments, the ORF encoding GP, NP, Z protein or the L protein of the tri-segmented arenavirus particle can be under the control of the promoter element of the 5′ UTR (see e.g., Albarino et al., 2011, J Virol., 85(8):4020-4). In another embodiment, the ORF encoding GP, NP Z protein, L protein of the tri-segmented arenavirus particle can be under the control of the promoter element of the 3′ UTR (see e.g., Albarino et al., 2011, J Virol., 85(8):4020-4). In more specific embodiments, the promoter element of the 5′ UTR is the 5′ UTR promoter element of the S segment(s) or the L segment(s). In another specific embodiment, the promoter element of the 3′ UTR is the 3′ UTR the promoter element of the S segment(s) or the L segment(s).

In certain embodiments, the ORF that encoding GP, NP, Z protein or the L protein of the tri-segmented arenavirus particle can be under the control of a truncated arenavirus 3′ UTR or a truncated arenavirus 5′ UTR (see e.g., Perez & de la Torre, 2003, J Virol. 77(2): 1184-1194; Albarino et al., 2011, J Virol., 85(8):4020-4). In more specific embodiments, the truncated 3′ UTR is the 3′ UTR of the arenavirus S segment or L segment. In more specific embodiments, the truncated 5′ UTR is the 5′ UTR of the arenavirus S segment(s) or L segment(s).

Also provided herein, is a cDNA of the tri-segmented arenavirus particle comprising a nucleotide sequence encoding a tumor antigen, tumor associated antigen or an antigenic fragment thereof provided herein. In more specific embodiments, provided herein is a DNA nucleotide sequence or a set of DNA nucleotide sequences encoding a tri-segmented arenavirus particle as set forth in Table 2 or Table 3.

In certain embodiments, the nucleic acids encoding the tri-segmented arenavirus genome are part of or incorporated into one or more DNA expression vectors. In a specific embodiment, nucleic acids encoding the genome of the tri-segmented arenavirus particle are part of or incorporated into one or more DNA expression vectors that facilitate production of a tri-segmented arenavirus particle as described herein. In another embodiment, a cDNA described herein can be incorporated into a plasmid. More detailed description of the cDNAs and expression systems are provided is Section 5.2.(e). Techniques for the production of a cDNA routine and conventional techniques of molecular biology and DNA manipulation and production. Any cloning technique known to the skilled artesian can be used. Such techniques are well known and are available to the skilled artesian in laboratory manuals such as, Sambrook and Russell, Molecular Cloning: A laboratory Manual, 3^{rd} edition, Cold Spring Harbor Laboratory N. Y. (2001).

In certain embodiments, the cDNA of the tri-segmented arenavirus comprising a nucleotide sequence encoding a tumor antigen, tumor associated antigen or an antigenic fragment thereof provided herein is introduced (e.g., transfected) into a host cell. Thus, in some embodiments provided herein, is a host cell comprising a cDNA of the tri-segmented arenavirus particle (i.e., a cDNA of the genomic segments of the tri-segmented arenavirus particle) and a nucleotide sequence encoding a tumor antigen, tumor associated antigen or an antigenic fragment thereof provided herein. In other embodiments, the cDNA described herein that is part of or can be incorporated into a DNA expression vector and introduced into a host cell. Thus, in some embodiments provided herein is a host cell comprising a cDNA described herein that is incorporated into a vector. In other embodiments, the tri-segmented arenavirus genomic segments (i.e., the L segment and/or S segment or segments) described herein is introduced into a host cell.

In certain embodiments, described herein is a method of producing the tri-segmented arenavirus particle, wherein the method comprises transcribing the cDNA of the tri-segmented arenavirus particle comprising a nucleotide sequence encoding a tumor antigen, tumor associated antigen or an antigenic fragment thereof provided herein. In certain embodiments, a viral polymerase protein can be present during transcription of the tri-segmented arenavirus particle in vitro or in vivo. In certain embodiments, transcription of the arenavirus genomic segment is performed using a bi-directional promoter.

In other embodiments, transcription of the arenavirus genomic segment is performed using a bi-directional expression cassette (see e.g., Ortiz-Riano et al., 2013, J Gen Virol., 94(Pt 6): 1175-1188). In more specific embodiments the bi-directional expression cassette comprises both a polymerase I and a polymerase II promoter reading from opposite sides into the two termini of the inserted arenavirus genomic segment, respectively.

In other embodiments, transcription of the cDNA of the arenavirus genomic segment described herein comprises a promoter. Specific examples of promoters include an RNA polymerase I promoter, an RNA polymerase II promoter, an RNA polymerase III promoter, a T7 promoter, an SP6 promoter or a T3 promoter.

In certain embodiments, the method of producing the tri-segmented arenavirus particle can further comprise introducing into a host cell the cDNA of the tri-segmented arenavirus particle comprising a nucleotide sequence encoding a tumor antigen, tumor associated antigen or an antigenic fragment thereof provided herein. In certain embodiments, the method of producing the tri-segmented arenavirus particle can further comprise introducing into a host cell the cDNA of the tri-segmented arenavirus particle that comprises a nucleotide sequence encoding a tumor antigen, tumor associated antigen or an antigenic fragment thereof provided herein, wherein the host cell expresses all other components for production of the tri-segmented arenavirus particle; and purifying the tri-segmented arenavirus particle from the supernatant of the host cell. Such methods are well-known to those skilled in the art.

Provided herein are cell lines, cultures and methods of culturing cells infected with nucleic acids, vectors, and compositions provided herein. More detailed description of nucleic acids, vector systems and cell lines described herein is provided in Section 5.2.(e).

In certain embodiments, the tri-segmented arenavirus particle as described herein results in a infectious and replication competent arenavirus particle. In specific embodiments, the arenavirus particle described herein is attenuated. In a particular embodiment, the tri-segmented arenavirus particle is attenuated such that the virus remains, at least partially, replication-competent and can replicate in vivo, but can only generate low viral loads resulting in subclinical levels of infection that are non-pathogenic. Such attenuated viruses can be used as an immunogenic composition.

In certain embodiments, the tri-segmented arenavirus particle has the same tropism as the bi-segmented arenavirus particle.

Also provided herein, are compositions that comprise the tri-segmented arenavirus particle as described in Section 5.2.(g).

### (i) Tri-Segmented Arenavirus Particle Comprising One L Segment and Two S Segments

Provided herein is a tri-segmented arenavirus particle that is replication competent. In certain specific embodiments, provided herein is a tri-segmented arenavirus particle that is replication defective. Tri-segmented arenavirus particles provided herein may be generated as described in International Publication No.: WO 2016/075250 A1 and International Patent Application No. PCT/EP2017/061865, which are herein incorporated in their entireties.

In one aspect, provided herein is a tri-segmented arenavirus particle comprising one L segment and two S segments. In certain embodiments, propagation of the tri-segmented arenavirus particle comprising one L segment and two S segments does not result in a replication-competent bi-segmented viral particle. In specific embodiments, propagation of the tri-segmented arenavirus particle comprising one L segment and two S segments does not result in a replication-competent bi-segmented viral particle after at least 10 days, at least 20 days, at least 30 days, at least 40 days, at least 50 days, at least 60 days, at least 70 days, at least 80 days, at least 90 days, or at least 100 days of persistent infection in mice lacking type I interferon receptor, type II interferon receptor and recombination activating gene (RAG1), and having been infected with 10⁴ PFU of the tri-segmented arenavirus particle (see Section 5.2.(h)(vii)). In other embodiments, propagation of the tri-segmented arenavirus particle comprising one L segment and two S segments does not result in a replication-competent bi-segmented viral particle after at least 10 passages, at least 20 passages, at least 30 passages, at least 40 passages, or at least 50 passages.

The tri-segmented arenavirus particle with all viral genes in their respective wild-type position is known in the art (e.g., Emonet et al., 2011 J. Virol., 85(4):1473; Popkin et al., 2011, J. Virol, 85(15):7928). In particular, the tri-segmented arenavirus genome consists of one L segment and two S segments, in which a nucleotide sequence encoding a tumor antigen, tumor associated antigen or an antigenic fragment thereof provided herein is inserted into one position on each S segment. More specifically, one S segment encodes GP and a tumor antigen, tumor associated antigen or an antigenic fragment thereof, respectively. The other S segment encodes a tumor antigen, a tumor associated antigen or an antigenic fragment thereof and NP, respectively. The L segment encodes the L protein and Z protein. All segments are flanked by the respective 5′ and 3′ UTRs.

In certain embodiments, inter-segmental recombination of the two S segments of the tri-segmented arenavirus particle, provided herein, that unities the two arenaviral ORFs on one instead of two separate segments results in a non functional promoter (i.e., a genomic segment of the structure: 5′ UTR-----------5′ UTR or a 3′ UTR------------3′ UTR), wherein each UTR forming one end of the genome is an inverted repeat sequence of the other end of the same genome.

In certain embodiments, the tri-segmented arenavirus particle comprising one L segment and two S segments has been engineered to carry an arenavirus ORF in a position other than the wild-type position of the ORF and a nucleotide sequence encoding a tumor antigen, tumor associated antigen or an antigenic fragment thereof provided herein. In other embodiments, the tri-segmented arenavirus particle comprising one L segment and two S segments has been engineered to carry two arenavirus ORFs, or three arenavirus ORFs, or four arenavirus ORFs, or five arenavirus ORFs, or six arenavirus ORFs in a position other than the wild-type position. In specific embodiments, the tri-segmented arenavirus particle comprising one L segment and two S segments comprises a full complement of all four arenavirus ORFs. Thus, in some embodiments, the tri-segmented arenavirus particle is an infectious and replication competent tri-segmented arenavirus particle. In specific embodiments, the two S segments of the tri-segmented arenavirus particle have been engineered to carry one of their ORFs in a position other than the wild-type position. In more specific embodiments, the two S segments comprise a full complement of the S segment ORF's. In certain specific embodiments, the L segment has been engineered to carry an ORF in a position other than the wild-type position or the L segment can be the wild-type genomic segment.

In certain embodiments, one of the two S segments can be:
(i) an arenavirus S segment, wherein the ORF encoding the Z protein is under control of an arenavirus 5′ UTR;
(ii) an arenavirus S segment, wherein the ORF encoding the L protein is under control of an arenavirus 5′ UTR;
(iii) an arenavirus S segment, wherein the ORF encoding the NP is under control of an arenavirus 5′ UTR;
(iv) an arenavirus S segment, wherein the ORF encoding the GP is under control of an arenavirus 3′ UTR;
(v) an arenavirus S segment, wherein the ORF encoding the L is under control of an arenavirus 3′ UTR; and
(vi) an arenavirus S segment, wherein the ORF encoding the Z protein is under control of an arenavirus 3′ UTR.

In certain embodiments, the tri-segmented arenavirus particle comprising one L segment and two S segments can comprise a duplicate ORF (i.e., two wild-type S segment ORFs e.g., GP or NP). In specific embodiments, the tri-segmented arenavirus particle comprising one L segment and two S segments can comprise one duplicate ORF (e.g., (GP, GP)) or two duplicate ORFs (e.g., (GP, GP) and (NP, NP)).

Table 2A, below, is an illustration of the genome organization of a tri-segmented arenavirus particle comprising one L segment and two S segments, wherein intersegmental recombination of the two S segments in the tri-segmented arenavirus genome does not result in a replication-competent bi-segmented viral particle and abrogates arenaviral promoter activity (i.e., the resulting recombined S segment is made up of two 3′UTRs instead of a 3′ UTR and a 5′ UTR).

TABLE 2A
Tri-segmented arenavirus particle
comprising one L segment and two S segments

| Position | Position | Position | Position | Position | Position |
|---|---|---|---|---|---|
| 1 | 2 | 3 | 4 | 5 | 6 |
| *ORF | GP | *ORF | NP | Z | L |
| *ORF | NP | *ORF | GP | Z | L |
| *ORF | NP | *ORF | GP | L | Z |
| *ORF | NP | *ORF | Z | L | GP |
| *ORF | NP | Z | GP | *ORF | Z |
| *ORF | NP | Z | GP | Z | *ORF |
| *ORF | NP | *ORF | L | Z | GP |
| *ORF | L | *ORF | NP | Z | GP |
| *ORF | L | Z | NP | *ORF | GP |
| *ORF | L | *ORF | GP | Z | NP |
| *ORF | L | Z | GP | *ORF | NP |
| *ORF | Z | L | NP | *ORF | GP |
| *ORF | Z | *ORF | GP | L | NP |
| *ORF | Z | L | GP | *ORF | NP |
| L | GP | *ORF | NP | *ORF | Z |
| L | GP | *ORF | *ORF | Z | NP |
| L | GP | *ORF | Z | *ORF | NP |
| L | *ORF | Z | GP | *ORF | NP |
| L | GP | *ORF | NP | *ORF | Z |
| L | GP | *ORF | Z | *ORF | NP |
| L | GP | Z | NP | *ORF | *ORF |
| L | GP | Z | NP | *ORF | *ORF |
| L | *ORF | Z | NP | *ORF | GP |
| L | NP | *ORF | Z | *ORF | GP |
| L | NP | Z | *ORF | GP | *ORF |
| L | *ORF | Z | *ORF | GP | NP |
| L | NP | Z | GP | *ORF | *ORF |
| L | NP | *ORF | Z | *ORF | GP |
| L | *ORF | Z | NP | *ORF | GP |
| L | Z | *ORF | GP | *ORF | NP |
| L | Z | *ORF | NP | *ORF | GP |
| Z | GP | *ORF | NP | *ORF | L |
| Z | GP | *ORF | *ORF | L | NP |
| Z | GP | *ORF | L | *ORF | NP |
| Z | *ORF | L | GP | *ORF | NP |
| Z | GP | *ORF | NP | *ORF | L |
| Z | GP | *ORF | L | *ORF | NP |
| Z | GP | L | NP | *ORF | *ORF |
| Z | GP | L | NP | *ORF | *ORF |
| Z | *ORF | L | NP | *ORF | GP |
| Z | NP | *ORF | *ORF | L | GP |
| Z | NP | *ORF | GP | *ORF | L |
| Z | NP | *ORF | *ORF | L | GP |
| Z | NP | *ORF | L | *ORF | GP |
| Z | NP | L | GP | *ORF | *ORF |
| Z | *ORF | L | GP | *ORF | NP |
| Z | NP | *ORF | GP | *ORF | L |
| Z | NP | *ORF | L | *ORF | GP |
| Z | *ORF | L | NP | *ORF | GP |
| Z | L | *ORF | GP | *ORF | NP |

Position 1 is under the control of an arenavirus S segment 5′ UTR;
Position 2 is under the control of an arenavirus S segment 3′ UTR;
Position 3 is under the control of an arenavirus S segment 5′ UTR;
Position 4 under the control of an arenavirus S segment 3′ UTR;
Position 5 is under the control of an arenavirus L segment 5′ UTR;
Position 6 is under the control of an arenavirus L segment 3′ UTR.
*ORF indicates that a nucleotide sequence encoding a tumor antigen, tumor associated antigen or an antigenic fragment thereof provided herein has been inserted.

In certain embodiments, the IGR between position one and position two can be an arenavirus S segment or L segment IGR; the IGR between position two and three can be an arenavirus S segment or L segment IGR; and the IGR between the position five and six can be an arenavirus L segment IGR. In a specific embodiment, the IGR between position one and position two can be an arenavirus S segment IGR; the IGR between position two and three can be an arenavirus S segment IGR; and the IGR between the position five and six can be an arenavirus L segment IGR. In certain embodiments, other combinations are also possible. For example, a tri-segmented arenavirus particle comprising one L segment and two S segments, wherein intersegmental recombination of the two S segments in the tri-segmented arenavirus genome does not result in a replication-competent bi-segmented viral particle and abrogates arenaviral promoter activity (i.e., the resulting recombined S segment is made up of two 5′UTRs instead of a 3′ UTR and a 5′ UTR).

In certain embodiments, intersegmental recombination of an S segment and an L segment in the tri-segmented arenavirus particle comprising one L segment and two S segments, restores a functional segment with two viral genes on only one segment instead of two separate segments. In other embodiments, intersegmental recombination of an S segment and an L segment in the tri-segmented arenavirus particle comprising one L segment and two S segments does not result in a replication-competent bi-segmented viral particle.

Table 2B, below, is an illustration of the genome organization of a tri-segmented arenavirus particle comprising one L segment and two S segments, wherein intersegmental recombination of an S segment and an L segment in the tri-segmented arenavirus genome does not result in a replication-competent bi-segmented viral particle and abrogates arenaviral promoter activity (i.e., the resulting recombined S segment is made up of two 3′UTRs instead of a 3′ UTR and a 5′ UTR).

TABLE 2B
Tri-segmented arenavirus particle
comprising one L segment and two S segments

| Position | Position | Position | Position | Position | Position |
|---|---|---|---|---|---|
| 1 | 2 | 3 | 4 | 5 | 6 |
| L | GP | *ORF | NP | Z | *ORF |
| L | GP | Z | *ORF | *ORF | NP |
| L | GP | *ORF | NP | Z | *ORF |
| L | GP | Z | *ORF | *ORF | NP |
| L | NP | *ORF | GP | Z | *ORF |
| L | NP | Z | *ORF | *ORF | GP |
| L | NP | *ORF | GP | Z | *ORF |
| L | NP | Z | *ORF | *ORF | GP |
| Z | GP | *ORF | NP | L | *ORF |
| Z | GP | L | *ORF | *ORF | NP |
| Z | GP | *ORF | NP | L | *ORF |
| Z | NP | L | *ORF | *ORF | GP |
| Z | NP | *ORF | GP | L | *ORF |
| Z | NP | L | *ORF | *ORF | GP |

Position 1 is under the control of an arenavirus S segment 5′ UTR;
Position 2 is under the control of an arenavirus S segment 3′ UTR;
Position 3 is under the control of an arenavirus S segment 5′ UTR;
Position 4 under the control of an arenavirus S segment 3′ UTR;
Position 5 is under the control of an arenavirus L segment 5′ UTR;
Position 6 is under the control of an arenavirus L segment 3′ UTR.
*ORF indicates that a nucleotide sequence encoding a tumor antigen, tumor associated antigen or an antigenic fragment thereof provided herein has been inserted.

In certain embodiments, the IGR between position one and position two can be an arenavirus S segment or L segment IGR; the IGR between position two and three can be an arenavirus S segment or L segment IGR; and the IGR between the position five and six can be an arenavirus L segment IGR. In a specific embodiment, the IGR between position one and position two can be an arenavirus S segment IGR; the IGR between position two and three can be an arenavirus S segment IGR; and the IGR between the position five and six can be an arenavirus L segment IGR. In certain embodiments, other combinations are also possible. For example, a tri-segmented arenavirus particle comprising one L segment and two S segments, wherein intersegmental recombination of the two S segments in the tri-segmented arenavirus genome does not result in a replication-competent bi-segmented viral particle and abrogates arenaviral promoter activity (i.e., the resulting recombined S segment is made up of two 5′UTRs instead of a 3′ UTR and a 5′ UTR).

In certain embodiments, one of skill in the art could construct an arenavirus genome with an organization as illustrated in Table 2A or 2B and as described herein, and then use an assay as described in Section 5.2.(h) to determine whether the tri-segmented arenavirus particle is genetically stable, i.e., does not result in a replication-competent bi-segmented viral particle as discussed herein.

### (ii) Tri-Segmented Arenavirus Particle Comprising Two L Segments and One S Segment

Provided herein is a tri-segmented arenavirus particle that is replication competent. In certain specific embodiments, provided herein is a tri-segmented arenavirus particle that is replication defective. Tri-segmented arenavirus particles provided herein may be generated as described in International Publication No.: WO 2016/075250 A1 and International Patent Application No. PCT/EP2017/061865, which are herein incorporated in their entireties.

In one aspect, provided herein is a tri-segmented arenavirus particle comprising two L segments and one S segment. In certain embodiments, propagation of the tri-segmented arenavirus particle comprising two L segments and one S segment does not result in a replication-competent bi-segmented viral particle. In specific embodiments, propagation of the tri-segmented arenavirus particle comprising two L segments and one S segment does not result in a replication-competent bi-segmented viral particle after at least 10 days, at least 20 days, at least 30 days, at least 40 days, or at least 50 days, at least 60 days, at least 70 days, at least 80 days, at least 90 days, at least 100 days of persistent in mice lacking type I interferon receptor, type II interferon receptor and recombination activating gene (RAG1), and having been infected with 10⁴ PFU of the tri-segmented arenavirus particle (see Section 5.2.(h)(vii)). In other embodiments, propagation of the tri-segmented arenavirus particle comprising two L segments and one S segment does not result in a replication-competent bi-segmented viral particle after at least 10 passages, 20 passages, 30 passages, 40 passages, or 50 passages.

In certain embodiments, inter-segmental recombination of the two L segments of the tri-segmented arenavirus particle, provided herein, that unities the two arenaviral ORFs on one instead of two separate segments results in a non functional promoter (i.e., a genomic segment of the structure: 5′ UTR-----------5′ UTR or a 3′ UTR------------3′ UTR), wherein each UTR forming one end of the genome is an inverted repeat sequence of the other end of the same genome.

In certain embodiments, the tri-segmented arenavirus particle comprising two L segments and one S segment has been engineered to carry an arenavirus ORF in a position other than the wild-type position of the ORF and a nucleotide sequence encoding a tumor antigen, tumor associated antigen or an antigenic fragment thereof provided herein. In other embodiments, the tri-segmented arenavirus particle comprising two L segments and one S segment has been engineered to carry two arenavirus ORFs, or three arenavirus ORFs, or four arenavirus ORFs, or five arenavirus ORFs, or six arenavirus ORFs in a position other than the wild-type position. In specific embodiments, the tri-segmented arenavirus particle comprising two L segments and one S segment comprises a full complement of all four arenavirus ORFs. Thus, in some embodiments, the tri-segmented arenavirus particle is an infectious and replication competent tri-segmented arenavirus particle. In specific embodiments, the two L segments of the tri-segmented arenavirus particle have been engineered to carry one of their ORFs in a position other than the wild-type position. In more specific embodiments, the two L segments comprise a full complement of the L segment ORF's. In certain specific embodiments, the S segment has been engineered to carry one of their ORFs in a position other than the wild-type position or the S segment can be the wild-type genomic segment.

In certain embodiments, one of the two L segments can be:
(xxxi) an L segment, wherein the ORF encoding the GP is under control of an arenavirus 5′ UTR;
(xxxii) an L segment, wherein the ORF encoding NP is under control of an arenavirus 5′ UTR;
(xxxiii) an L segment, wherein the ORF encoding the L protein is under control of an arenavirus 5′ UTR;
(xxxiv) an L segment, wherein the ORF encoding the GP is under control of an arenavirus 3′ UTR;
(xxxv) an L segment, wherein the ORF encoding the NP is under control of an arenavirus 3′ UTR; and
(xxxvi) an L segment, wherein the ORF encoding the Z protein is under control of an arenavirus 3′ UTR.

In certain embodiments, the tri-segmented arenavirus particle comprising one L segment and two S segments can comprise a duplicate ORF (i.e., two wild-type L segment ORFs e.g., Z protein or L protein). In specific embodiments, the tri-segmented arenavirus particle comprising two L segments and one S segment can comprise one duplicate ORF (e.g., (Z protein, Z protein)) or two duplicate ORFs (e.g., (Z protein, Z protein) and (L protein, L protein)).

Table 3, below, is an illustration of the genome organization of a tri-segmented arenavirus particle comprising two L segments and one S segment, wherein intersegmental recombination of the two L segments in the tri-segmented arenavirus genome does not result in a replication-competent bi-segmented viral particle and abrogates arenaviral promoter activity (i.e., the S segment is made up of two 3′UTRs instead of a 3′ UTR and a 5′ UTR). Based on Table 3 similar combinations could be predicted for generating an arenavirus particle made up of two 5′ UTRs instead of a 3′ UTR and a 5′ UTR.

TABLE 3
Tri-segmented arenavirus particle
comprising two L segments and one S segment

| Position | Position | Position | Position | Position | Position |
|---|---|---|---|---|---|
| 1 | 2 | 3 | 4 | 5 | 6 |
| ORF* | Z | ORF* | L | NP | GP |
| ORF* | Z | ORF* | L | GP | NP |
| ORF* | Z | GP | L | ORF* | NP |
| ORF* | Z | ORF* | GP | NP | L |
| ORF* | Z | GP | ORF* | NP | L |
| ORF* | Z | NP | ORF* | GP | L |
| ORF* | ORF* | NP | Z | GP | L |
| ORF* | Z | GP | NP | ORF* | L |
| ORF* | Z | NP | GP | ORF* | L |
| ORF* | L | ORF* | Z | NP | GP |
| ORF* | L | ORF* | Z | GP | NP |
| ORF* | L | ORF* | GP | NP | Z |
| ORF* | L | GP | Z | ORF* | NP |
| ORF* | L | ORF* | GP | NP | Z |
| ORF* | L | NP | Z | ORF* | GP |
| ORF* | L | GP | NP | ORF* | Z |
| ORF* | L | NP | GP | ORF* | Z |
| ORF* | GP | ORF* | L | NP | Z |
| ORF* | GP | NP | L | ORF* | Z |
| ORF* | GP | ORF* | Z | NP | L |
| ORF* | GP | NP | Z | ORF* | L |
| ORF* | NP | ORF* | L | GP | Z |
| ORF* | NP | GP | L | ORF* | Z |
| ORF* | NP | GP | Z | ORF* | L |
| ORF* | NP | ORF* | Z | GP | L |
| ORF* | L | ORF* | Z | NP | GP |
| ORF* | L | ORF* | Z | GP | NP |
| ORF* | L | ORF* | NP | GP | Z |
| ORF* | L | ORF* | GP | NP | Z |
| ORF* | L | NP | Z | ORF* | GP |
| ORF* | Z | ORF* | GP | NP | L |
| ORF* | Z | GP | L | ORF* | NP |
| ORF* | Z | NP | GP | ORF* | L |
| ORF* | Z | GP | NP | ORF* | L |
| ORF* | GP | ORF* | L | NP | Z |
| ORF* | GP | ORF* | L | Z | NP |
| ORF* | GP | ORF* | Z | GP | L |
| ORF* | GP | NP | L | ORF* | Z |
| GP | L | ORF* | Z | ORF* | NP |
| GP | L | ORF* | NP | ORF* | Z |
| GP | Z | ORF* | L | ORF* | NP |
| GP | Z | ORF* | L | ORF* | NP |
| GP | Z | ORF* | NP | ORF* | L |
| GP | NP | ORF* | Z | ORF* | L |
| NP | L | ORF* | Z | ORF* | GP |
| NP | L | ORF* | GP | ORF* | Z |
| NP | L | ORF* | Z | ORF* | GP |

*Position 1 is under the control of an arenavirus L segment 5′ UTR;
position 2 is under the control of an arenavirus L segment 3′ UTR;
position 3 is under the control of an arenavirus L segment 5′ UTR;
position 4 is under the control of an arenavirus L segment 3′ UTR;
position 5 is under the control of an arenavirus S segment 5′ UTR;
position 6 is under the control of an arenavirus S segment 3′ UTR.
*ORF indicates that a nucleotide sequence encoding a tumor antigen, tumor associated antigen or an antigenic fragment thereof provided herein has been inserted.

In certain embodiments, the IGR between position one and position two cab be an arenavirus S segment or L segment IGR; the IGR between position two and three can be an arenavirus S segment or L segment IGR; and the IGR between the position five and six can be an arenavirus L segment IGR. In a specific embodiment, the IGR between position one and position two can be an arenavirus L segment IGR; the IGR between position two and three can be an arenavirus L segment IGR; and the IGR between the position five and six can be an arenavirus S segment IGR. In certain embodiments, other combinations are also possible.

In certain embodiments, intersegmental recombination of an L segment and an S segment from the tri-segmented arenavirus particle comprising two L segments and one S segment restores a functional segment with two viral genes on only one segment instead of two separate segments. In other embodiments, intersegmental recombination of an L segment and an S segment in the tri-segmented arenavirus particle comprising two L segments and one S segment does not result in a replication-competent bi-segmented viral particle.

Table 3B, below, is an illustration of the genome organization of a tri-segmented arenavirus particle comprising two L segments and one S segment, wherein intersegmental recombination of an L segment and an S segment in the tri-segmented arenavirus genome does not result in a replication-competent bi-segmented viral particle and abrogates arenaviral promoter activity (i.e., the resulting recombined S segment is made up of two 3′UTRs instead of a 3′ UTR and a 5′ UTR).

TABLE 3B
Tri-segmented arenavirus particle
comprising two L segments and one S segment

| Position | Position | Position | Position | Position | Position |
|---|---|---|---|---|---|
| 1 | 2 | 3 | 4 | 5 | 6 |
| NP | Z | *ORF | GP | L | *ORF |
| NP | Z | GP | *ORF | *ORF | L |
| NP | Z | *ORF | GP | L | *ORF |
| NP | Z | GP | *ORF | *ORF | L |
| NP | L | *ORF | GP | Z | *ORF |
| NP | L | GP | *ORF | *ORF | Z |
| NP | L | *ORF | GP | Z | *ORF |
| NP | L | GP | *ORF | *ORF | Z |
| GP | Z | *ORF | NP | L | *ORF |
| GP | Z | NP | *ORF | *ORF | L |
| GP | Z | *ORF | NP | L | *ORF |
| GP | L | NP | *ORF | *ORF | Z |
| GP | L | *ORF | NP | Z | *ORF |
| GP | L | NP | *ORF | *ORF | Z |

*Position 1 is under the control of an arenavirus L segment 5′ UTR;
position 2 is under the control of an arenavirus L segment 3′ UTR;
position 3 is under the control of an arenavirus L segment 5′ UTR;
position 4 is under the control of an arenavirus L segment 3′ UTR;
position 5 is under the control of an arenavirus S segment 5′ UTR;
position 6 is under the control of an arenavirus S segment 3′ UTR.
*ORF indicates that a nucleotide sequence encoding a tumor antigen, tumor associated antigen or an antigenic fragment thereof provided herein has been inserted.

In certain embodiments, the IGR between position one and position two cab be an arenavirus S segment or L segment IGR; the IGR between position two and three can be an arenavirus S segment or L segment IGR; and the IGR between the position five and six can be an arenavirus L segment IGR. In a specific embodiment, the IGR between position one and position two can be an arenavirus L segment IGR; the IGR between position two and three can be an arenavirus L segment IGR; and the IGR between the position five and six can be an arenavirus S segment IGR. In certain embodiments, other combinations are also possible.

In certain embodiments, one of skill in the art could construct an arenavirus genome with an organization as illustrated in Table 3A or 3B and as described herein, and then use an assay as described in Section 5.2.(h) to determine whether the tri-segmented arenavirus particle is genetically stable, i.e., does not result in a replication-competent bi-segmented viral particle as discussed herein. (iii) Replication-Defective Tri-segmented Arenavirus Particle

In certain embodiments, provided herein is a tri-segmented arenavirus particle in which (i) an ORF is in a position other than the wild-type position of the ORF; and (ii) an ORF encoding GP, NP, Z protein, or L protein has been removed or functionally inactivated such that the resulting virus cannot produce further infectious progeny virus particles (i.e., is replication defective). In certain embodiments, the third arenavirus segment can be an S segment. In other embodiments, the third arenavirus segment can be an L segment. In more specific embodiments, the third arenavirus segment can be engineered to carry an ORF in a position other than the wild-type position of the ORF or the third arenavirus segment can be the wild-type arenavirus genomic segment. In yet more specific embodiments, the third arenavirus segment lacks an arenavirus ORF encoding GP, NP, Z protein, or the L protein.

In certain embodiments, a tri-segmented genomic segment could be a S or a L segment hybrid (i.e., a genomic segment that can be a combination of the S segment and the L segment). In other embodiments, the hybrid segment is an S segment comprising an L segment IGR. In another embodiment, the hybrid segment is an L segment comprising an S segment IGR. In other embodiments, the hybrid segment is an S segment UTR with and L segment IGR. In another embodiment, the hybrid segment is an L segment UTR with an S segment IGR. In specific embodiments, the hybrid segment is an S segment 5′ UTR with an L segment IGR or an S segment 3′ UTR with an L segment IGR. In other specific embodiments, the hybrid segment is an L segment 5′ UTR with an S segment IGR or an L segment 3′ UTR with an S segment IGR.

A tri-segmented arenavirus particle comprising a genetically modified genome in which one or more ORFs has been deleted or functionally inactivated can be produced in complementing cells (i.e., cells that express the arenavirus ORF that has been deleted or functionally inactivated). The genetic material of the resulting arenavirus particle can be transferred upon infection of a host cell into the host cell, wherein the genetic material can be expressed and amplified. In addition, the genome of the genetically modified arenavirus particle described herein can include a nucleotide sequence encoding a tumor antigen, tumor associated antigen or an antigenic fragment thereof provided herein.

In certain embodiments, at least one of the four ORFs encoding GP, NP, Z protein, and L protein is removed and replaced with a nucleotide sequence encoding a tumor antigen, tumor associated antigen or an antigenic fragment thereof provided herein. In another embodiment, at least one ORF, at least two ORFs, at least three ORFs, or at least four ORFs encoding GP, NP, Z protein and L protein can be removed and replaced with a nucleotide sequence encoding a tumor antigen, tumor associated antigen or an antigenic fragment thereof provided herein. In specific embodiments, only one of the four ORFs encoding GP, NP, Z protein, and L protein is removed and replaced with a nucleotide sequence encoding a tumor antigen, tumor associated antigen or an antigenic fragment thereof provided herein. In more specific embodiments, the ORF that encodes GP of the arenavirus genomic segment is removed. In another specific embodiment, the ORF that encodes the NP of the arenavirus genomic segment is removed. In more specific embodiments, the ORF that encodes the Z protein of the arenavirus genomic segment is removed. In yet another specific embodiment, the ORF encoding the L protein is removed.

In certain embodiments, provided herein is a tri-segmented arenavirus particle comprising one L segment and two S segments in which (i) an ORF is in a position other than the wild-type position of the ORF; and (ii) an ORF encoding GP or NP has been removed or functionally inactivated, such that the resulting virus is replication-defective and not infectious. In a specific embodiment, one ORF is removed and replaced with a nucleotide sequence encoding a tumor antigen, tumor associated antigen or an antigenic fragment thereof provided herein. In another specific embodiment, two ORFs are removed and replaced with a nucleotide sequence encoding a tumor antigen, tumor associated antigen or an antigenic fragment thereof provided herein. In other specific embodiments, three ORFs are removed and replaced with a nucleotide sequence encoding a tumor antigen, tumor associated antigen or an antigenic fragment thereof provided herein. In specific embodiments, the ORF encoding GP is removed and replaced with a nucleotide sequence encoding a tumor antigen, tumor associated antigen or an antigenic fragment thereof provided herein. In other specific embodiments, the ORF encoding NP is removed and replaced with a nucleotide sequence encoding a tumor antigen, tumor associated antigen or an antigenic fragment thereof provided herein. In yet more specific embodiments, the ORF encoding NP and the ORF encoding GP are removed and replaced with one or two nucleotide sequences encoding tumor antigens, tumor associated antigens or antigenic fragments thereof provided herein. Thus, in certain embodiments the tri-segmented arenavirus particle comprises (i) one L segment and two S segments; (ii) an ORF in a position other than the wild-type position of the ORF; (iii) one or more nucleotide sequences encoding tumor antigens, tumor associated antigens or an antigenic fragments thereof provided herein.

In certain embodiments, provided herein is a tri-segmented arenavirus particle comprising two L segments and one S segment in which (i) an ORF is in a position other than the wild-type position of the ORF; and (ii) an ORF encoding the Z protein, and/or the L protein has been removed or functionally inactivated, such that the resulting virus replication-defective and not infectious. In a specific embodiment, one ORF is removed and replaced with a nucleotide sequence encoding a tumor antigen, tumor associated antigen or an antigenic fragment thereof provided herein. In another specific embodiment, two ORFs are removed and replaced with a nucleotide sequence encoding a tumor antigen, tumor associated antigen or an antigenic fragment thereof provided herein. In specific embodiments, the ORF encoding the Z protein is removed and replaced with a nucleotide sequence encoding a tumor antigen, tumor associated antigen or an antigenic fragment thereof provided herein. In other specific embodiments, the ORF encoding the L protein is removed and replaced with a nucleotide sequence encoding a tumor antigen, tumor associated antigen or an antigenic fragment thereof provided herein. In yet more specific embodiments, the ORF encoding the Z protein and the ORF encoding the L protein is removed and replaced with a nucleotide sequence encoding a tumor antigen, tumor associated antigen or an antigenic fragment thereof provided herein. Thus, in certain embodiments the tri-segmented arenavirus particle comprises (i) two L segments and one S segment; (ii) an ORF in a position other than the wild-type position of the ORF; (iii) a nucleotide sequence encoding a tumor antigen, tumor associated antigen or an antigenic fragment thereof provided herein.

Thus, in certain embodiments, the tri-segmented arenavirus particle provided herein comprises a tri-segmented arenavirus particle (i.e., one L segment and two S segments or two L segments and one S segment) that i) is engineered to carry an ORF in a non-natural position; ii) an ORF encoding GP, NP, Z protein, or L protein is removed); iii) the ORF that is removed is replaced with one or more nucleotide sequences encoding tumor antigens, tumor associated antigens or antigenic fragments thereof provided herein.

In certain embodiments, the nucleotide sequence encoding an antigenic fragment provided herein is 8 to 100 nucleotides in length, 15 to 100 nucleotides in length, 25 to 100 nucleotides in length, 50 to 200 nucleotide in length, 50 to 400 nucleotide in length, 200 to 500 nucleotide in length, or 400 to 600 nucleotides in length, 500 to 800 nucleotide in length. In other embodiments, the nucleotide sequence encoding an antigenic fragment provided herein is 750 to 900 nucleotides in length, 800 to 100 nucleotides in length, 850 to 1000 nucleotides in length, 900 to 1200 nucleotides in length, 1000 to 1200 nucleotides in length, 1000 to 1500 nucleotides or 10 to 1500 nucleotides in length, 1500 to 2000 nucleotides in length, 1700 to 2000 nucleotides in length, 2000 to 2300 nucleotides in length, 2200 to 2500 nucleotides in length, 2500 to 3000 nucleotides in length, 3000 to 3200 nucleotides in length, 3000 to 3500 nucleotides in length, 3200 to 3600 nucleotides in length, 3300 to 3800 nucleotides in length, 4000 nucleotides to 4400 nucleotides in length, 4200 to 4700 nucleotides in length, 4800 to 5000 nucleotides in length, 5000 to 5200 nucleotides in length, 5200 to 5500 nucleotides in length, 5500 to 5800 nucleotides in length, 5800 to 6000 nucleotides in length, 6000 to 6400 nucleotides in length, 6200 to 6800 nucleotides in length, 6600 to 7000 nucleotides in length, 7000 to 7200 nucleotides in lengths, 7200 to 7500 nucleotides in length, or 7500 nucleotides in length. In some embodiments, the nucleotide sequence encodes a peptide or polypeptide that is 5 to 10 amino acids in length, 10 to 25 amino acids in length, 25 to 50 amino acids in length, 50 to 100 amino acids in length, 100 to 150 amino acids in length, 150 to 200 amino acids in length, 200 to 250 amino acids in length, 250 to 300 amino acids in length, 300 to 400 amino acids in length, 400 to 500 amino acids in length, 500 to 750 amino acids in length, 750 to 1000 amino acids in length, 1000 to 1250 amino acids in length, 1250 to 1500 amino acids in length, 1500 to 1750 amino acids in length, 1750 to 2000 amino acids in length, 2000 to 2500 amino acids in length, or more than 2500 or more amino acids in length. In some embodiments, the nucleotide sequence encodes a polypeptide that does not exceed 2500 amino acids in length. In specific embodiments the nucleotide sequence does not contain a stop codon. In certain embodiments, the nucleotide sequence is codon-optimized. In certain embodiments the nucleotide composition, nucleotide pair composition or both can be optimized. Techniques for such optimizations are known in the art and can be applied to optimize a nucleotide sequence encoding a tumor antigen, tumor associated antigen or an antigenic fragment thereof provided herein.

Any nucleotide sequence encoding a tumor antigen, tumor associated antigen or an antigenic fragment thereof provided herein may be included in the tri-segmented arenavirus particle. In one embodiment, the a nucleotide sequence encoding a tumor antigen, tumor associated antigen or an antigenic fragment thereof provided herein is capable of eliciting an immune response.

In certain embodiments, the growth and infectivity of the arenavirus particle is not affected by the nucleotide sequence encoding a tumor antigen, tumor associated antigen or an antigenic fragment thereof provided herein.

Techniques known to one skilled in the art may be used to produce an arenavirus particle comprising an arenavirus genomic segment engineered to carry an arenavirus ORF in a position other than the wild-type position and a nucleotide sequence encoding a tumor antigen, tumor associated antigen or an antigenic fragment thereof provided herein. For example, reverse genetics techniques may be used to generate such arenavirus particle. In other embodiments, the replication-defective arenavirus particle (i.e., the arenavirus genomic segment engineered to carry an arenavirus ORF in a position other than the wild-type position, wherein an ORF encoding GP, NP, Z protein, L protein, has been deleted) can be produced in a complementing cell.

In certain embodiments, a tri-segmented arenavirus particle provided herein comprising a nucleotide sequence encoding a tumor antigen, tumor associated antigen or antigenic fragment thereof as provided herein further comprises at least one nucleotide sequence encoding at least one immunomodulatory peptide, polypeptide or protein. In certain embodiments, the immunomodulatory peptide, polypeptide or protein is Calreticulin (CRT), or a fragment thereof, Ubiquitin or a fragment thereof, Granulocyte-Macrophage Colony-Stimulating Factor (GM-CSF), or a fragment thereof, Invariant chain (CD74) or an antigenic fragment thereof; Mycobacterium tuberculosis Heat shock protein 70 or an antigenic fragment thereof; Herpes simplex virus 1 protein VP22 or an antigenic fragment thereof, CD40 ligand or an antigenic fragment thereof, or Fms-related tyrosine kinase 3 (Flt3) ligand or an antigenic fragment thereof.

Arenaviruses for use with the methods and compositions provided herein can be Old World viruses, for example Lassa virus, Lymphocytic choriomeningitis virus (LCMV), Mobala virus, Mopeia virus, or Ippy virus, or New World viruses, for example Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Bear Canyon virus, or Whitewater Arroyo virus.

In certain embodiments, the tri-segmented arenavirus particle as described herein is suitable for use as a vaccine and methods of using such arenavirus particle in a vaccination and treatment for a neoplastic disease, for example, cancer, is provided. More detailed description of the methods of using the arenavirus particle described herein is provided in Section 5.2.(f)

In certain embodiments, the tri-segmented arenavirus particle as described herein is suitable for use as a pharmaceutical composition and methods of using such arenavirus particle in a vaccination and treatment for a neoplastic disease, for example, cancer, is provided. More detailed description of the methods of using the arenavirus particle described herein is provided in Section 5.2.(g). (c) Tumor Antigens, Tumor Associated Antigens and Antigenic Fragments

In certain embodiments, arenavirus particles with nucleotide sequence encoding a tumor antigen, tumor associated antigen or an antigenic fragment thereof provided herein can be used with the methods and compositions provided herein, such as combinations with a chemotherapeutic agent. In certain embodiments, a tumor antigen or tumor associated antigen for use with the methods and compositions described herein is an immunogenic protein expressed in or on a neoplastic cell or tumor, such as a cancer cell or malignant tumor. In certain embodiments, a tumor antigen or tumor associated antigen for use with the methods and compositions described herein is a non-specific, mutant, overexpressed or abnormally expressed protein, which can be present on both a neoplastic cell or tumor and a normal cell or tissue. In certain embodiments, a tumor antigen or tumor associated antigen for use with the methods and compositions described herein is a tumor-specific antigen which is restricted to tumor cells. In certain embodiments, a tumor antigen for use with the methods and compositions described herein is a cancer-specific antigen which is restricted to cancer cells.

In certain embodiments, a tumor antigen or tumor associated antigen can exhibit one, two, three, or more, including all, of the following characteristics: overexpressed/accumulated (i.e., expressed by both normal and neoplastic tissue, but highly expressed in neoplasia), oncofetal (i.e., usually only expressed in fetal tissues and in cancerous somatic cells), oncoviral or oncogenic viral (i.e., encoded by tumorigenic transforming viruses), cancer-testis (i.e., expressed only by cancer cells and adult reproductive tissues, e.g., the testis), lineage-restricted (i.e., expressed largely by a single cancer histotype), mutated (i.e., only expressed in neoplastic tissue as a result of genetic mutation or alteration in transcription), post-translationally altered (e.g., tumor-associated alterations in glycosylation), or idiotypic (i.e., developed from malignant clonal expansions of B or T lymphocytes).

In certain embodiments, the tumor antigen or tumor associated antigen for use with the methods and compositions described herein includes antigens from neoplastic diseases including acute lymphoblastic leukemia; acute lymphoblastic lymphoma; acute lymphocytic leukaemia; acute myelogenous leukemia; acute myeloid leukemia (adult/childhood); adrenocortical carcinoma; AIDS-related cancers; AIDS-related lymphoma; anal cancer; appendix cancer; astrocytomas; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; ewing's sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; hodgkin lymphoma; hypopharyngeal cancer; hypothalamic and visual pathway glioma; intraocular melanoma; islet cell carcinoma (endocrine pancreas); Kaposi Sarcoma; kidney cancer (renal cell cancer); langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); merkel cell cancer; merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-hodgkin lymophoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; respiratory tract carcinoma involving the NUT gene on chromosome 15; retinoblastoma; rhabdomyosarcoma, childhood; salivary gland cancer; sarcoma, Ewing family of tumors; Sezary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sezary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor.

In certain embodiments, the tumor antigen or tumor associated antigen for use with the methods and compositions disclosed herein includes oncogenic viral antigens, cancer-testis antigens, oncofetal antigens, tissue differentiation antigens, mutant protein antigens, Adipophilin, AIM-2, ALDH1AI, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUCI, MUC5AC, p53 (non-mutant), PAX5, PBF, PRAME, PSMA, RAGE, RAGE-1, RGS5, RhoC, RNF43, RU2AS, secernin 1, SOXI0, STEAPI (six-transmembrane epithelial antigen of the prostate 1), survivin, Telomerase, VEGF, WT1, EGF-R, CEA, CD20, CD33, CD52, MELANA/MART1, MART2,NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSXl or-SSX2 fusion protein, TGF-betaRII, Triosephosphate isomerase, ormdm-2, LMP2, HPV E6/E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP-1, GP100, CA-125, CA19-9, Calretinin, Epithelial membrane antigen (EMA), Epithelial tumor antigen (ETA), CD19, CD34, CD99, CD 117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXBI, SPA17, SSX, SYCP1, TPTE, Carbohydrate/ganglioside GM2 (oncofetal antigen-immunogenic-1 OFA-I-1), GM3, CA 15-3 (CA 27.29BCAA), CA 195, CA 242, CA 50, CAM 43, CEA, EBNA, EF2, Epstein-Barr virus antigen, HLA-A2, HLA-All, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, pi80erbB-3, c-met, nm-23H1, TAG-72, TAG-72-4, CA-72-4, CAM 17.1, NuMa, 13-catenin, P16, TAGE, CT7, 43-9F,5T4, 791Tgp72, 13HCG, BCA225, BTAA, CD68KP1, CO-029, HTgp-175, M344, MG7-Ag, MOV18, NB70K, NY-CO-1, RCAS1, SDCCAGi6, TA-90, TAAL6, TLP, TPS, CD22, CD27, CD30, CD70, prostein, TARP (T cell receptor gamma alternate reading frame protein), Trp-p8, integrin avP3 (CD61), galactin, or Ral-B, CD123, CLL-1, CD38, CS-1, CD138, and ROR1.

In certain embodiments, the tumor antigen or tumor associated antigen is a neoantigen. A “neoantigen,” as used herein, means an antigen that arises by mutation in a tumor cell and such an antigen is not generally expressed in normal cells or tissue. Without being bound by theory, because healthy tissues generally do not posses these antigens, neoantigens represent a preferred target. Additionally, without being bound by theory, in the context of the present invention, since the T cells that recognize the neoantigen may not have undergone negative thymic selection, such cells can have high avidity to the antigen and mount a strong immune response against tumors, while lacking the risk to induce destruction of normal tissue and autoimmune damage. In certain embodiments, the neoantigen is an MHC class I-restricted neoantigen. In certain embodiments, the neoantigen is an MHC class II-restricted neoantigen. In certain embodiments, a mutation in a tumor cell of the patient results in a novel protein that produces the neoantigen.

In certain embodiments, the tumor antigen or tumor associated antigen can be an antigen ortholog, e.g., a mammalian (i.e., non-human primate, pig, dog, cat, or horse) to a human tumor antigen or tumor associated antigen.

In certain embodiments, an antigenic fragment of a tumor antigen or tumor associated antigen described herein is encoded by the nucleotide sequence included within the arenavirus. In certain embodiments, a fragment is antigenic when it is capable of (i) eliciting an antibody immune response in a host (e.g., mouse, rabbit, goat, donkey or human) wherein the resulting antibodies bind specifically to an immunogenic protein expressed in or on a neoplastic cell (e.g., a cancer cell); and/or (ii) eliciting a specific T cell immune response.

In certain embodiments, the nucleotide sequence encoding antigenic fragment of a tumor antigen or tumor associated antigen is 8 to 100 nucleotides in length, 15 to 100 nucleotides in length, 25 to 100 nucleotides in length, 50 to 200 nucleotide in length, 50 to 400 nucleotide in length, 200 to 500 nucleotide in length, or 400 to 600 nucleotides in length, 500 to 800 nucleotide in length. In other embodiments, the heterologous ORF is 750 to 900 nucleotides in length, 800 to 100 nucleotides in length, 850 to 1000 nucleotides in length, 900 to 1200 nucleotides in length, 1000 to 1200 nucleotides in length, 1000 to 1500 nucleotides or 10 to 1500 nucleotides in length, 1500 to 2000 nucleotides in length, 1700 to 2000 nucleotides in length, 2000 to 2300 nucleotides in length, 2200 to 2500 nucleotides in length, 2500 to 3000 nucleotides in length, 3000 to 3200 nucleotides in length, 3000 to 3500 nucleotides in length, 3200 to 3600 nucleotides in length, 3300 to 3800 nucleotides in length, 4000 nucleotides to 4400 nucleotides in length, 4200 to 4700 nucleotides in length, 4800 to 5000 nucleotides in length, 5000 to 5200 nucleotides in length, 5200 to 5500 nucleotides in length, 5500 to 5800 nucleotides in length, 5800 to 6000 nucleotides in length, 6000 to 6400 nucleotides in length, 6200 to 6800 nucleotides in length, 6600 to 7000 nucleotides in length, 7000 to 7200 nucleotides in lengths, 7200 to 7500 nucleotides in length, or 7500 nucleotides in length. In some embodiments, the heterologous ORF encodes a peptide or polypeptide that is 5 to 10 amino acids in length, 10 to 25 amino acids in length, 25 to 50 amino acids in length, 50 to 100 amino acids in length, 100 to 150 amino acids in length, 150 to 200 amino acids in length, 200 to 250 amino acids in length, 250 to 300 amino acids in length, 300 to 400 amino acids in length, 400 to 500 amino acids in length, 500 to 750 amino acids in length, 750 to 1000 amino acids in length, 1000 to 1250 amino acids in length, 1250 to 1500 amino acids in length, 1500 to 1750 amino acids in length, 1750 to 2000 amino acids in length, 2000 to 2500 amino acids in length, or more than 2500 or more amino acids in length. In some embodiments, the nucleotide sequence encodes a polypeptide that does not exceed 2500 amino acids in length. In specific embodiments the nucleotide sequence does not contain a stop codon. In certain embodiments, the nucleotide sequence is codon-optimized. In certain embodiments the nucleotide composition, nucleotide pair composition or both can be optimized. Techniques for such optimizations are known in the art and can be applied to optimize a nucleotide sequence of a tumor antigen or tumor associated antigen.

In certain embodiments, the arenavirus genomic segment, the arenavirus particle or the tri-segmented arenavirus particle can comprise one or more nucleotide sequences encoding tumor antigens, tumor associated antigens, or antigenic fragments thereof. In other embodiments, the arenavirus genomic segment, the arenavirus particle or the tri-segmented arenavirus particle can comprise at least one nucleotide sequence encoding a tumor antigen, tumor associated antigen, or antigenic fragment thereof, at least two nucleotide sequences encoding tumor antigens, tumor associated antigens, or antigenic fragments thereof, at least three nucleotide sequences encoding tumor antigens, tumor associated antigens, or antigenic fragments thereof, or more nucleotide sequences encoding tumor antigens, tumor associated antigens, or antigenic fragments thereof.

In certain embodiments, an arenavirus particle comprising a nucleotide sequence encoding a tumor antigen, tumor associated antigen or antigenic fragment thereof as provided herein further comprises at least one nucleotide sequence encoding at least one immunomodulatory peptide, polypeptide or protein. In certain embodiments, the immunomodulatory peptide, polypeptide or protein is Calreticulin (CRT), or a fragment thereof, Ubiquitin or a fragment thereof, Granulocyte-Macrophage Colony-Stimulating Factor (GM-CSF), or a fragment thereof, Invariant chain (CD74) or an antigenic fragment thereof; Mycobacterium tuberculosis Heat shock protein 70 or an antigenic fragment thereof, Herpes simplex virus 1 protein VP22 or an antigenic fragment thereof, CD40 ligand or an antigenic fragment thereof; or Fms-related tyrosine kinase 3 (Flt3) ligand or an antigenic fragment thereof.

In certain embodiments, an arenavirus particle provided herein comprises a genomic segment that a) has a removal or functional inactivation of an ORF that is present in the wild type form of the genomic segment; and b) encodes (either in sense or antisense): (i) one or more tumor antigen, tumor associated antigen or an antigenic fragment thereof provided herein, and (ii) one or more immunomodulatory peptide, polypeptide or protein provided herein.

In certain embodiments, the nucleotide sequence encoding the tumor antigen, tumor associated antigen or an antigenic fragment thereof provided herein, and the nucleotide sequence encoding the immunomodulatory peptide, polypeptide or protein provided herein, are on the same position of the viral genome. In certain embodiments, the nucleotide sequence encoding the tumor antigen, tumor associated antigen or an antigenic fragment thereof provided herein, and the nucleotide sequence encoding the immunomodulatory peptide, polypeptide or protein provided herein, are on different positions of the viral genome.

In certain embodiments, the nucleotide sequence encoding the tumor antigen, tumor associated antigen or an antigenic fragment thereof provided herein, and the nucleotide sequence encoding the immunomodulatory peptide, polypeptide or protein provided herein, are separated via a spacer sequence. In certain embodiments, the sequence encoding the tumor antigen, tumor associated antigen or an antigenic fragment thereof provided herein, and the nucleotide sequence encoding the immunomodulatory peptide, polypeptide or protein provided herein, are separated by an internal ribosome entry site, or a sequence encoding a protease cleavage site. In certain embodiments, the nucleotide sequence encoding the tumor antigen, tumor associated antigen or an antigenic fragment thereof provided herein, and the nucleotide sequence encoding the immunomodulatory peptide, polypeptide or protein provided herein, are separated by a nucleotide sequence encoding a linker or a self-cleaving peptide. Any linker peptide or self-cleaving peptide known to the skilled artisan can be used with the compositions and methods provided herein. A non-limiting example of a peptide linker is GSG. Non-limiting examples of a self-cleaving peptide are Porcine teschovirus-1 2A peptide, Those assign a virus 2A peptide, or Foot-and-mouth disease virus 2A peptide.

In certain embodiments, the tumor antigen, tumor associated antigen or an antigenic fragment thereof provided herein, and the immunomodulatory peptide, polypeptide or protein provided herein, are directly fused together. In certain embodiments, the tumor antigen, tumor associated antigen or an antigenic fragment thereof provided herein, and the immunomodulatory peptide, polypeptide or protein provided herein, are fused together via a peptide linker. In certain embodiments, the tumor antigen, tumor associated antigen or an antigenic fragment thereof provided herein, and the immunomodulatory peptide, polypeptide or protein provided herein are separated from each other via a self-cleaving peptide. A non-limiting example of a peptide linker is GSG. Non-limiting examples of a self-cleaving peptide are Porcine teschovirus-1 2A peptide, Those assign a virus 2A peptide, or Foot-and-mouth disease virus 2A peptide.

In certain embodiments, the tumor antigen, tumor associated antigen or an antigenic fragment thereof provided herein, and the immunomodulatory peptide, polypeptide or protein provided herein are expressed on the same arenavirus particle. In certain embodiments, the tumor antigen, tumor associated antigen or an antigenic fragment thereof provided herein, and the immunomodulatory peptide, polypeptide or protein provided herein are expressed on different are an a virus particles. In certain embodiments, the tumor antigen, tumor associated antigen or an antigenic fragment thereof provided herein, and the immunomodulatory peptide, polypeptide or protein provided herein are expressed on different viruses of the same strain. In certain embodiments, the tumor antigen, tumor associated antigen or an antigenic fragment thereof provided herein, and the immunomodulatory peptide, polypeptide or protein provided herein are expressed on different viruses of different strains.

In certain embodiments, an arenavirus particle generated to encode one or more tumor antigens, tumor associated antigens or antigenic fragments thereof comprises one or more nucleotide sequences encoding tumor antigens, tumor associated antigens or antigenic fragments thereof provided herein. In specific embodiments the tumor antigens, tumor associated antigens or antigenic fragments thereof provided herein are separated by various one or more linkers, spacers, or cleavage sites as described herein.

### (d) Generation of an Arenavirus Particle and a Tri-Segmented Arenavirus Particle Expressing a Tumor Antigen, Tumor Associated Antigen or Antigenic Fragment Thereof

Generally, arenavirus particles for use in the methods and compositions provided herein, such as combinations with a chemotherapeutic agent, can be recombinantly produced by standard reverse genetic techniques as described for LCMV (see Flatz et al., 2006, Proc Natl Acad Sci USA 103:4663-4668; Sanchez et al., 2006, Virology 350:370; Ortiz-Riano et al., 2013, J Gen Virol. 94:1175-88, which are incorporated by reference herein). To generate the arenavirus particles provided herein, these techniques can be applied as described below. The genome of the viruses can be modified as described herein.

### (i) Non-Natural Position Open Reading Frame

The generation of an arenavirus particle comprising a genomic segment that has been engineered to carry a viral ORF in a position other than the wild-type position of the ORF and a nucleotide sequence encoding a tumor antigen, tumor associated antigen or antigenic fragment thereof can be recombinantly produced by any reverse genetic techniques known to one skilled in the art.

### (A) Infectious and Replication Competent Arenavirus Particle

In certain embodiments, the method of generating the arenavirus particle comprises (i) transfecting into a host cell the cDNA of the first arenavirus genomic segment; (ii) transfecting into a host cell the cDNA of the second arenavirus genomic segment; (iii) transfecting into a host cell plasmids expressing the arenavirus' minimal trans-acting factors NP and L; (iv) maintaining the host cell under conditions suitable for virus formation; and (v) harvesting the arenavirus particle. In certain more specific embodiments, the cDNA is comprised in a plasmid.

Once generated from cDNA, arenavirus particles (e.g., infectious and replication competent) can be propagated. In certain embodiments, the arenavirus particle can be propagated in any host cell that allows the virus to grow to titers that permit the uses of the virus as described herein. In one embodiment, the host cell allows the arenavirus particle to grow to titers comparable to those determined for the corresponding wild-type.

In certain embodiments, the arenavirus particle may be propagated in host cells. Specific examples of host cells that can be used include BHK-21, HEK 293, VERO or other. In a specific embodiment, the arenavirus particle may be propagated in a cell line.

In certain embodiments, the host cells are kept in culture and are transfected with one or more plasmid(s). The plasmid(s) express the arenavirus genomic segment(s) to be generated under control of one or more expression cassettes suitable for expression in mammalian cells, e.g., consisting of a polymerase I promoter and terminator.

Plasmids that can be used for the generation of the arenavirus particle can include: i) a plasmid encoding the S genomic segment e.g., pol-I S, ii) a plasmid encoding the L genomic segment e.g., pol-I L. In certain embodiments, the plasmid encoding an arenavirus polymerase that direct intracellular synthesis of the viral L and S segments can be incorporated into the transfection mixture. For example, a plasmid encoding the L protein and/or a plasmid encoding NP (pC-L and pC-NP, respectively) can be present. The L protein and NP are the minimal trans-acting factors necessary for viral RNA transcription and replication. Alternatively, intracellular synthesis of viral L and S segments, together with NP and L protein can be performed using an expression cassette with pol-I and pol-II promoters reading from opposite sides into the L and S segment cDNAs of two separate plasmids, respectively.

In certain embodiments, the arenavirus genomic segments are under the control of a promoter. Typically, RNA polymerase I-driven expression cassettes, RNA polymerase II-driven cassettes or T7 bacteriophage RNA polymerase driven cassettes can be used. In certain embodiments, the plasmid(s) encoding the arenavirus genomic segments can be the same, i.e., the genome sequence and transacting factors can be transcribed by a promoter from one plasmid. Specific examples of promoters include an RNA polymerase I promoter, an RNA polymerase II promoter, an RNA polymerase III promoter, a T7 promoter, an SP6 promoter or a T3 promoter.

In addition, the plasmid(s) can feature a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Transfection of a host cell with a plasmid(s) can be performed using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing clones are identified using Western blot or flow cytometry procedures with antibodies directed against the viral protein(s) of interest.

For recovering the arenavirus particle described herein, the following procedures are envisaged. First day: cells, typically 80% confluent in M6-well plates, are transfected with a mixture of the plasmids, as described above. For this one can exploit any commonly used strategies such as calcium-phosphate, liposome-based protocols or electroporation.

3-5 days later: The cultured supernatant (arenavirus vector preparation) is harvested, aliquoted and stored at 4° C., −20° C., or −80° C., depending on how long the arenavirus vector should be stored prior use. The arenavirus vector preparation's infectious titer is assessed by an immunofocus assay. Alternatively, the transfected cells and supernatant may be passaged to a larger vessel (e.g., a T75 tissue culture flask) on day 3-5 after transfection, and culture supernatant is harvested up to five days after passage.

The present application furthermore relates to expression of a heterologous ORF, wherein a plasmid encoding the genomic segment is modified to incorporated a heterologous ORF. The heterologous ORF can be incorporated into the plasmid using restriction enzymes.

### (B) Infectious, Replication-Defective Arenavirus Particle

Infectious, replication-defective arenavirus particles can be rescued as described above. However, once generated from cDNA, the infectious, replication-deficient arenaviruses provided herein can be propagated in complementing cells. Complementing cells are cells that provide the functionality that has been eliminated from the replication-deficient arenavirus by modification of its genome (e.g., if the ORF encoding the GP protein is deleted or functionally inactivated, a complementing cell does provide the GP protein).

Owing to the removal or functional inactivation of one or more of the ORFs in arenavirus vectors (here deletion of the glycoprotein, GP, will be taken as an example), arenavirus vectors can be generated and expanded in cells providing in trans the deleted viral gene(s), e.g., the GP in the present example. Such a complementing cell line, henceforth referred to as C-cells, is generated by transfecting a cell line such as BHK-21, HEK 293, VERO or other with one or more plasmid(s) for expression of the viral gene(s) of interest (complementation plasmid, referred to as C-plasmid). The C-plasmid(s) express the viral gene(s) deleted in the arenavirus vector to be generated under control of one or more expression cassettes suitable for expression in mammalian cells, e.g., a mammalian polymerase II promoter such as the EFlalpha promoter with a polyadenylation signal. In addition, the complementation plasmid features a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Cells that can be used, e.g., BHK-21, HEK 293, MC57G or other, are kept in culture and are transfected with the complementation plasmid(s) using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing C-cell clones are identified using Western blot or flow cytometry procedures with antibodies directed against the viral protein(s) of interest. As an alternative to the use of stably transfected C-cells transient transfection of normal cells can complement the missing viral gene(s) in each of the steps where C-cells will be used below. In addition, a helper virus can be used to provide the missing functionality in trans.

Plasmids can be of two types: i) two plasmids, referred to as TF-plasmids for expressing intracellularly in C-cells the minimal transacting factors of the arenavirus, is derived from e.g., NP and L proteins of LCMV in the present example; and ii) plasmids, referred to as GS-plasmids, for expressing intracellularly in C-cells the arenavirus vector genome segments, e.g., the segments with designed modifications. TF-plasmids express the NP and L proteins of the respective arenavirus vector under control of an expression cassette suitable for protein expression in mammalian cells, typically e.g., a mammalian polymerase II promoter such as the CMV or EF 1alpha promoter, either one of them preferentially in combination with a polyadenylation signal. GS-plasmids express the small (S) and the large (L) genome segments of the vector. Typically, polymerase I-driven expression cassettes or T7 bacteriophage RNA polymerase (T7-) driven expression cassettes can be used, the latter preferentially with a 3′-terminal ribozyme for processing of the primary transcript to yield the correct end. In the case of using a T7-based system, expression of T7 in C-cells must be provided by either including in the recovery process an additional expression plasmid, constructed analogously to TF-plasmids, providing T7, or C-cells are constructed to additionally express T7 in a stable manner. In certain embodiments, TF and GS plasmids can be the same, i.e., the genome sequence and transacting factors can be transcribed by T7, poll and polII promoters from one plasmid.

For recovering of the arenavirus vector, the following procedures can be used. First day: C-cells, typically 80% confluent in M6-well plates, are transfected with a mixture of the two TF-plasmids plus the two GS-plasmids. In certain embodiments, the TF and GS plasmids can be the same, i.e., the genome sequence and transacting factors can be transcribed by T7, poll and polII promoters from one plasmid. For this one can exploit any of the commonly used strategies such as calcium-phosphate, liposome-based protocols or electroporation.

3-5 days later: The culture supernatant (arenavirus vector preparation) is harvested, aliquoted and stored at 4° C., −20° C. or −80° C. depending on how long the arenavirus vector should be stored prior to use. Then the arenavirus vector preparation's infectious titer is assessed by an immunofocus assay on C-cells. Alternatively, the transfected cells and supernatant may be passaged to a larger vessel (e.g., a T75 tissue culture flask) on day 3-5 after transfection, and culture supernatant is harvested up to five days after passage.

The invention furthermore relates to expression of a antigen in a cell culture wherein the cell culture is infected with an infectious, replication-deficient arenavirus expressing an antigen. When used for expression of a antigen in cultured cells, the following two procedures can be used:
i) The cell type of interest is infected with the arenavirus vector preparation described herein at a multiplicity of infection (MOI) of one or more, e.g., two, three or four, resulting in production of the antigen in all cells already shortly after infection.
ii) Alternatively, a lower MOI can be used and individual cell clones can be selected for their level of virally driven antigen expression. Subsequently individual clones can be expanded infinitely owing to the non-cytolytic nature of arenavirus vectors. Irrespective of the approach, the antigen can subsequently be collected (and purified) either from the culture supernatant or from the cells themselves, depending on the properties of the antigen produced. However, the invention is not limited to these two strategies, and other ways of driving expression of antigen using infectious, replication-deficient arenaviruses as vectors may be considered.

### (ii) Generation of a Tri-Segmented Arenavirus Particle

A tri-segmented arenavirus particle comprising a nucleotide sequence encoding a tumor antigen, tumor associated antigen or antigenic fragment thereof can be recombinantly produced by reverse genetic techniques known in the art, for example as described by Emonet et al., 2008, PNAS, 106(9):3473-3478; Popkin et al., 2011, J. Virol., 85 (15):7928-7932, which are incorporated by reference herein. The generation of the tri-segmented arenavirus particle provided herein can be modified as described in Section 5.2(b).

### (A) Infectious and Replication Competent Tri-Segmented Arenavirus Particle

In certain embodiments, the method of generating the tri-segmented arenavirus particle comprises (i) transfecting into a host cell the cDNAs of the one L segment and two S segments or two L segments and one S segment; (ii) transfecting into a host cell plasmids expressing the arenavirus' minimal trans-acting factors NP and L; (iii) maintaining the host cell under conditions suitable for virus formation; and (iv) harvesting the arenavirus particle.

Once generated from cDNA, the tri-segmented arenavirus particle (i.e., infectious and replication competent) can be propagated. In certain embodiments tri-segmented arenavirus particle can be propagated in any host cell that allows the virus to grow to titers that permit the uses of the virus as described herein. In one embodiment, the host cell allows the tri-segmented arenavirus particle to grow to titers comparable to those determined for the corresponding wild-type.

In certain embodiments, the tri-segmented arenavirus particle may be propagated in host cells. Specific examples of host cells that can be used include BHK-21, HEK 293, VERO or other. In a specific embodiment, the tri-segmented arenavirus particle may be propagated in a cell line.

In certain embodiments, the host cells are kept in culture and are transfected with one or more plasmid(s). The plasmid(s) express the arenavirus genomic segment(s) to be generated under control of one or more expression cassettes suitable for expression in mammalian cells, e.g., consisting of a polymerase I promoter and terminator.

In specific embodiments, the host cells are kept in culture and are transfected with one or more plasmid(s). The plasmid(s) express the viral gene(s) to be generated under control of one or more expression cassettes suitable for expression in mammalian cells, e.g., consisting of a polymerase I promoter and terminator.

Plasmids that can be used for generating the tri-segmented arenavirus comprising one L segment and two S segments can include: i) two plasmids each encoding the S genome segment e.g., pol-I S, ii) a plasmid encoding the L genome segment e.g., pol-I L. Plasmids needed for the tri-segmented arenavirus comprising two L segments and one S segments are: i) two plasmids each encoding the L genome segment e.g., pol-L, ii) a plasmid encoding the S genome segment e.g., pol-I S.

In certain embodiments, plasmids encoding an arenavirus polymerase that direct intracellular synthesis of the viral L and S segments can be incorporated into the transfection mixture. For example, a plasmid encoding the L protein and a plasmid encoding NP (pC-L and pC-NP, respectively). The L protein and NP are the minimal trans-acting factors necessary for viral RNA transcription and replication. Alternatively, intracellular synthesis of viral L and S segments, together with NP and L protein can be performed using an expression cassette with pol-I and pol-II promoters reading from opposite sides into the L and S segment cDNAs of two separate plasmids, respectively.

In addition, the plasmid(s) features a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Transfection of BHK-21 cells with a plasmid(s) can be performed using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing clones are identified using Western blot or flow cytometry procedures with antibodies directed against the viral protein(s) of interest.

Typically, RNA polymerase I-driven expression cassettes, RNA polymerase II-driven cassettes or T7 bacteriophage RNA polymerase driven cassettes can be used, the latter preferentially with a 3′-terminal ribozyme for processing of the primary transcript to yield the correct end. In certain embodiments, the plasmids encoding the arenavirus genomic segments can be the same, i.e., the genome sequence and transacting factors can be transcribed by T7, polI and polII promoters from one plasmid.

For recovering the arenavirus the tri-segmented arenavirus vector, the following procedures are envisaged. First day: cells, typically 80% confluent in M6-well plates, are transfected with a mixture of the plasmids, as described above. For this one can exploit any commonly used strategies such as calcium-phosphate, liposome-based protocols or electroporation.

3-5 days later: The cultured supernatant (arenavirus vector preparation) is harvested, aliquoted and stored at 4° C., −20° C., or −80° C., depending on how long the arenavirus vector should be stored prior use. The arenavirus vector preparation's infectious titer is assessed by an immunofocus assay. Alternatively, the transfected cells and supernatant may be passaged to a larger vessel (e.g., a T75 tissue culture flask) on day 3-5 after transfection, and culture supernatant is harvested up to five days after passage.

In certain embodiments, expression of a nucleotide sequence encoding a tumor antigen, tumor associated antigen or antigenic fragment thereof is provided, wherein a plasmid encoding the genomic segment is modified to incorporated a nucleotide sequence encoding a tumor antigen, tumor associated antigen or antigenic fragment thereof. The nucleotide sequence encoding a tumor antigen, tumor associated antigen or antigenic fragment thereof can be incorporated into the plasmid using restriction enzymes.

### (B) Infectious, Replication-Defective Tri-Segmented Arenavirus Particle

Infectious, replication-defective tri-segmented arenavirus particles can be rescued as described above. However, once generated from cDNA, the infectious, replication-deficient arenaviruses provided herein can be propagated in complementing cells. Complementing cells are cells that provide the functionality that has been eliminated from the replication-deficient arenavirus by modification of its genome (e.g., if the ORF encoding the GP protein is deleted or functionally inactivated, a complementing cell does provide the GP protein).

Owing to the removal or functional inactivation of one or more of the ORFs in arenavirus vectors (here deletion of the glycoprotein, GP, will be taken as an example), arenavirus vectors can be generated and expanded in cells providing in trans the deleted viral gene(s), e.g., the GP in the present example. Such a complementing cell line, henceforth referred to as C-cells, is generated by transfecting a mammalian cell line such as BHK-21, HEK 293, VERO or other (here BHK-21 will be taken as an example) with one or more plasmid(s) for expression of the viral gene(s) of interest (complementation plasmid, referred to as C-plasmid). The C-plasmid(s) express the viral gene(s) deleted in the arenavirus vector to be generated under control of one or more expression cassettes suitable for expression in mammalian cells, e.g., a mammalian polymerase II promoter such as the CMV or EF 1alpha promoter with a polyadenylation signal. In addition, the complementation plasmid features a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Cells that can be used, e.g., BHK-21, HEK 293, MC57G or other, are kept in culture and are transfected with the complementation plasmid(s) using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing C-cell clones are identified using Western blot or flow cytometry procedures with antibodies directed against the viral protein(s) of interest. As an alternative to the use of stably transfected C-cells transient transfection of normal cells can complement the missing viral gene(s) in each of the steps where C-cells will be used below. In addition, a helper virus can be used to provide the missing functionality in trans.

Plasmids of two types can be used: i) two plasmids, referred to as TF-plasmids for expressing intracellularly in C-cells the minimal transacting factors of the arenavirus, is derived from e.g., NP and L proteins of LCMV in the present example; and ii) plasmids, referred to as GS-plasmids, for expressing intracellularly in C-cells the arenavirus vector genome segments, e.g., the segments with designed modifications. TF-plasmids express the NP and L proteins of the respective arenavirus vector under control of an expression cassette suitable for protein expression in mammalian cells, typically e.g., a mammalian polymerase II promoter such as the CMV or EF 1alpha promoter, either one of them preferentially in combination with a polyadenylation signal. GS-plasmids express the small (S) and the large (L) genome segments of the vector. Typically, polymerase I-driven expression cassettes or T7 bacteriophage RNA polymerase (T7-) driven expression cassettes can be used, the latter preferentially with a 3′-terminal ribozyme for processing of the primary transcript to yield the correct end. In the case of using a T7-based system, expression of T7 in C-cells must be provided by either including in the recovery process an additional expression plasmid, constructed analogously to TF-plasmids, providing T7, or C-cells are constructed to additionally express T7 in a stable manner. In certain embodiments, TF and GS plasmids can be the same, i.e., the genome sequence and transacting factors can be transcribed by T7, poll and polII promoters from one plasmid.

For recovering of the arenavirus vector, the following procedures can be used. First day: C-cells, typically 80% confluent in M6-well plates, are transfected with a mixture of the two TF-plasmids plus the two GS-plasmids. In certain embodiments, the TF and GS plasmids can be the same, i.e., the genome sequence and transacting factors can be transcribed by T7, polI and polII promoters from one plasmid. For this one can exploit any of the commonly used strategies such as calcium-phosphate, liposome-based protocols or electroporation.

3-5 days later: The culture supernatant (arenavirus vector preparation) is harvested, aliquoted and stored at 4° C., −20° C. or −80° C. depending on how long the arenavirus vector should be stored prior to use. Then the arenavirus vector preparation's infectious titer is assessed by an immunofocus assay on C-cells. Alternatively, the transfected cells and supernatant may be passaged to a larger vessel (e.g., a T75 tissue culture flask) on day 3-5 after transfection, and culture supernatant is harvested up to five days after passage.

The invention furthermore relates to expression of an antigen in a cell culture wherein the cell culture is infected with an infectious, replication-deficient tri-segmented arenavirus expressing a antigen. When used for expression of a CMV antigen in cultured cells, the following two procedures can be used:
i) The cell type of interest is infected with the arenavirus vector preparation described herein at a multiplicity of infection (MOI) of one or more, e.g., two, three or four, resulting in production of the tumor antigen, tumor associated antigen, or antigenic fragment thereof in all cells already shortly after infection.
ii) Alternatively, a lower MOI can be used and individual cell clones can be selected for their level of virally driven expression of a tumor antigen, tumor associated antigen or antigenic fragment thereof. Subsequently individual clones can be expanded infinitely owing to the non-cytolytic nature of arenavirus vectors. Irrespective of the approach, the tumor antigen, tumor associated antigen or antigenic fragment thereof can subsequently be collected (and purified) either from the culture supernatant or from the cells themselves, depending on the properties of the tumor antigen, tumor associated antigen or antigenic fragment produced. However, the invention is not limited to these two strategies, and other ways of driving expression of tumor antigen, tumor associated antigen or antigenic fragment thereof using infectious, replication-deficient arenaviruses as vectors may be considered.

### (e) Nucleic Acids, Vector Systems and Cell Lines

In certain embodiments, provided herein are cDNAs comprising or consisting of the arenavirus genomic segment or the tri-segmented arenavirus particle as described herein, which can be used with the methods and compositions provided herein, such as combinations with a chemotherapeutic agent.

### (i) Non-Natural Position Open Reading Frame

In one embodiment, provided herein are nucleic acids that encode an arenavirus genomic segment as described in Section 5.2.(a). In more specific embodiments, provided herein is a DNA nucleotide sequence or a set of DNA nucleotide sequences as set forth in Table 1. Host cells that comprise such nucleic acids are also provided Section 5.2.(a).

In specific embodiments, provided herein is a cDNA of the arenavirus genomic segment engineered to carry an ORF in a position other than the wild-type position of the ORF and a nucleotide sequence encoding a tumor antigen, tumor associated antigen or antigenic fragment thereof, wherein the arenavirus genomic segment encodes a heterologous ORF as described in Section 5.2(a)

In one embodiment, provided herein is a DNA expression vector system that encodes the arenavirus genomic segment engineered to carry an ORF in a position other than the wild-type position of the ORF and a nucleotide sequence encoding a tumor antigen, tumor associated antigen or antigenic fragment thereof. Specifically, provided herein is a DNA expression vector system wherein one or more vectors encodes two arenavirus genomic segments, namely, an L segment and an S segment, of an arenavirus particle described herein. Such a vector system can encode a nucleotide sequence encoding a tumor antigen, tumor associated antigen or antigenic fragment thereof.

In another embodiment, provided herein is a cDNA of the arenavirus S segment that has been engineered to carry an ORF in a position other than the wild-type position and a nucleotide sequence encoding a tumor antigen, tumor associated antigen or antigenic fragment thereof that is part of or incorporated into a DNA expression system. In other embodiments, provided herein is a cDNA of the arenavirus L segment that has been engineered to carry an ORF in a position other than the wild-type position and a nucleotide sequence encoding a tumor antigen, tumor associated antigen or antigenic fragment thereof that is part of or incorporated into a DNA expression system. In certain embodiments, is a cDNA of the arenavirus genomic segment that has been engineered to carry (i) an ORF in a position other than the wild-type position of the ORF; and (ii) and ORF encoding GP, NP, Z protein, or L protein has been removed and replaced with a nucleotide sequence encoding a tumor antigen, tumor associated antigen or antigenic fragment thereof.

In certain embodiments, the cDNA provided herein can be derived from a particular strain of LCMV. Strains of LCMV include Clone 13, MP strain, Arm CA 1371, Arm E-250, WE, UBC, Traub, Pasteur, 810885, CH-5692, Marseille #12, HP65-2009, 200501927, 810362, 811316, 810316, 810366, 20112714, Douglas, GRO1, SN05, CABN and their derivatives. In specific embodiments, the cDNA is derived from LCMV Clone 13. In other specific embodiments, the cDNA is derived from LCMV MP strain.

In certain embodiments, the vector generated to encode an arenavirus particle or a tri-segmented arenavirus particle as described herein may be based on a specific strain of LCMV. Strains of LCMV include Clone 13, MP strain, Arm CA 1371, Arm E-250, WE, UBC, Traub, Pasteur, 810885, CH-5692, Marseille #12, HP65-2009, 200501927, 810362, 811316, 810316, 810366, 20112714, Douglas, GRO1, SN05, CABN and their derivatives. In certain embodiments, an arenavirus particle or a tri-segmented arenavirus particle as described herein may be based on LCMV Clone 13. In other embodiments, the vector generated to encode an arenavirus particle or a tri-segmented arenavirus particle as described herein LCMV MP strain.

In another embodiment, provided herein is a cell, wherein the cell comprises a cDNA or a vector system described above in this section. Cell lines derived from such cells, cultures comprising such cells, methods of culturing such cells infected are also provided herein. In certain embodiments, provided herein is a cell, wherein the cell comprises a cDNA of the arenavirus genomic segment that has been engineered to carry an ORF in a position other than the wild-type position of the ORF and a nucleotide sequence encoding a tumor antigen, tumor associated antigen or antigenic fragment thereof. In some embodiments, the cell comprises the S segment and/or the L segment.

### (ii) Tri-Segmented Arenavirus Particle

In one embodiment, provided herein are nucleic acids that encode a tri-segmented arenavirus particle as described in Section 5.2.(b). In more specific embodiments, provided herein is a DNA nucleotide sequence or a set of DNA nucleotide sequences, for example, as set forth in Table 2 or Table 3. Host cells that comprise such nucleic acids are also provided Section 5.2(b).

In specific embodiments, provided herein is a cDNA consisting of a cDNA of the tri-segmented arenavirus particle that has been engineered to carry an ORF in a position other than the wild-type position of the ORF. In other embodiments, is a cDNA of the tri-segmented arenavirus particle that has been engineered to (i) carry an arenavirus ORF in a position other than the wild-type position of the ORF; and (ii) wherein the tri-segmented arenavirus particle encodes a heterologous ORF as described in Section 5.2(b).

In one embodiment, provided herein is a DNA expression vector system that together encode the tri-segmented arenavirus particle comprising a nucleotide sequence encoding a tumor antigen, tumor associated antigen or antigenic fragment thereof as described herein. Specifically, provided herein is a DNA expression vector system wherein one or more vectors encode three arenavirus genomic segments, namely, one L segment and two S segments or two L segments and one S segment of a tri-segmented arenavirus particle described herein. Such a vector system can encode a tumor antigen, tumor associated antigen or antigenic fragment thereof.

In another embodiment, provided herein is a cDNA of the arenavirus S segment(s) that has been engineered to carry an ORF in a position other than the wild-type position and a nucleotide sequence encoding a tumor antigen, tumor associated antigen or antigenic fragment thereof that is part of or incorporated into a DNA expression system. In other embodiments, a cDNA of the arenavirus L segment(s) that has been engineered to carry an ORF in a position other than the wild-type position and a nucleotide sequence encoding a tumor antigen, tumor associated antigen or antigenic fragment thereof that is part of or incorporated into a DNA expression system. In certain embodiments, is a cDNA of the tri-segmented arenavirus particle that has been engineered to carry (i) an ORF in a position other than the wild-type position of the ORF; and (ii) an ORF encoding GP, NP, Z protein, or L protein has been removed and replaced with a nucleotide sequence encoding a tumor antigen, tumor associated antigen or antigenic fragment thereof.

In certain embodiments, the cDNA provided herein can be derived from a particular strain of LCMV. Strains of LCMV include Clone 13, MP strain, Arm CA 1371, Arm E-250, WE, UBC, Traub, Pasteur, 810885, CH-5692, Marseille #12, HP65-2009, 200501927, 810362, 811316, 810316, 810366, 20112714, Douglas, GRO1, SN05, CABN and their derivatives. In specific embodiments, the cDNA is derived from LCMV Clone 13. In other specific embodiments, the cDNA is derived from LCMV MP strain.

In certain embodiments, the vector generated to encode an arenavirus particle or a tri-segmented arenavirus particle as described herein may be based on a specific strain of LCMV. Strains of LCMV include Clone 13, MP strain, Arm CA 1371, Arm E-250, WE, UBC, Traub, Pasteur, 810885, CH-5692, Marseille #12, HP65-2009, 200501927, 810362, 811316, 810316, 810366, 20112714, Douglas, GRO1, SN05, CABN and their derivatives. In certain embodiments, an arenavirus particle or a tri-segmented arenavirus particle as described herein may be based on LCMV Clone 13. In other embodiments, the vector generated to encode an arenavirus particle or a tri-segmented arenavirus particle as described herein LCMV MP strain.

In another embodiment, provided herein is a cell, wherein the cell comprises a cDNA or a vector system described above in this section. Cell lines derived from such cells, cultures comprising such cells, methods of culturing such cells infected are also provided herein. In certain embodiments, provided herein is a cell, wherein the cell comprises a cDNA of the tri-segmented arenavirus particle. In some embodiments, the cell comprises the S segment and/or the L segment.

### (f) Methods of Use

Vaccines have been successful for preventing and/or treating infectious diseases, such as those for polio virus and measles. However, therapeutic immunization in the setting of established, chronic disease, including cancer has been less successful. The ability to generate an arenavirus particle that is used in combination with a chemotherapeutic agent represents a new novel vaccine strategy.

In certain embodiments, provided herein are methods of treating a neoplastic disease in a subject. Such methods can include administering to a subject in need thereof an arenavirus particle provided herein and a chemotherapeutic agent provided herein. In certain embodiments, the arenavirus particle used in the methods is an infectious, replication-deficient arenavirus particle provided herein. In certain embodiments, the arenavirus particle used in the methods is a tri-segmented arenavirus particle provided herein, including an infectious, replication-deficient tri-segmented arenavirus particle or a replication-competent tri-segmented arenavirus particle. Thus, in certain embodiments, the arenavirus particle, including a tri-segmented arenavirus particle, used in the methods is replication-deficient, wherein the arenvirus particle is engineered to contain a genome comprising: (1) a nucleotide sequence encoding a tumor antigen, tumor associated antigen or an antigenic fragment thereof, and (2) the ability to amplify and express its genetic information in infected cells but unable to produce further infectious progeny particles in non-complementing cells. Moreover, in certain embodiments, a tri-segmented arenavirus particle used in the methods is replication-competent, wherein the arenvirus particle is engineered to contain a genome comprising: (1) a nucleotide sequence encoding a tumor antigen, tumor associated antigen or an antigenic fragment thereof, (2) the ability to amplify and express its genetic information in infected cells; and (3) the ability to produce further infectious progeny particles in normal, not genetically engineered cells.

In one embodiment, provided herein are methods of treating a neoplastic disease in a subject comprising administering to the subject one or more arenavirus particles expressing a tumor antigen, tumor associated antigen or an antigenic fragment thereof as provided herein or a composition thereof, and a chemotherapeutic agent provided herein. In a specific embodiment, a method for treating a neoplastic disease described herein comprises administering to a subject in need thereof a therapeutically effective amount of one or more arenavirus particles expressing a tumor antigen, tumor associated antigen or an antigenic fragment thereof provided herein or a composition thereof, and a chemotherapeutic agent provided herein. The subject can be a mammal, such as but not limited to a human, a mouse, a rat, a guinea pig, a domesticated animal, such as, but not limited to, a cow, a horse, a sheep, a pig, a goat, a cat, a dog, a hamster, a donkey. In a specific embodiment, the subject is a human.

In another embodiment, provided herein are methods for inducing an immune response against a neoplastic cell or tissue, such as a cancer cell or tumor, in a subject comprising administering to the subject an arenavirus particle expressing a tumor antigen, tumor associated antigen or an antigenic fragment thereof provided herein, or a composition thereof, and a chemotherapeutic agent provided herein.

In another embodiment, the subjects to whom an arenavirus particle expressing a tumor antigen, tumor associated antigen or an antigenic fragment thereof provided herein, or a composition thereof, and a chemotherapeutic agent provided herein is administered have, are susceptible to, or are at risk for a neoplastic disease.

In another embodiment, the subjects to whom an arenavirus particle expressing a tumor antigen, tumor associated antigen or an antigenic fragment thereof provided herein, or a composition thereof, and a chemotherapeutic agent provided herein is administered have, are susceptible to, or are at risk for development of a neoplastic disease, such as cancer, or exhibit a pre-cancerous tissue lesion. In another specific embodiment, the subjects to whom arenavirus particle expressing a tumor antigen, tumor associated antigen or an antigenic fragment thereof provided herein, or a composition thereof, and a chemotherapeutic agent provided herein is administered are diagnosed with a neoplastic disease, such as cancer, or exhibit a pre-cancerous tissue lesion.

In another embodiment, the subjects to whom an arenavirus particle expressing a tumor antigen, tumor associated antigen or an antigenic fragment thereof provided herein, or a composition thereof, and a chemotherapeutic agent provided herein is administered are suffering from, are susceptible to, or are at risk for, a neoplastic disease selected from, but not limited to, acute lymphoblastic leukemia; acute lymphoblastic lymphoma; acute lymphocytic leukaemia; acute myelogenous leukemia; acute myeloid leukemia (adult/childhood); adrenocortical carcinoma; AIDS-related cancers; AIDS-related lymphoma; anal cancer; appendix cancer; astrocytomas; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; ewing's sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; hodgkin lymphoma; hypopharyngeal cancer; hypothalamic and visual pathway glioma; intraocular melanoma; islet cell carcinoma (endocrine pancreas); Kaposi Sarcoma; kidney cancer (renal cell cancer); langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); merkel cell cancer; merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-hodgkin lymophoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; respiratory tract carcinoma involving the NUT gene on chromosome 15; retinoblastoma; rhabdomyosarcoma, childhood; salivary gland cancer; sarcoma, Ewing family of tumors; Sezary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sezary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor.

In another embodiment, an arenavirus particle expressing a tumor antigen, tumor associated antigen or an antigenic fragment thereof provided herein, or a composition thereof, and a chemotherapeutic agent provided herein is administered to a subject of any age group suffering from, are susceptible to, or are at risk for a neoplastic disease. In a specific embodiment, an arenavirus particle expressing a tumor antigen, tumor associated antigen or an antigenic fragment thereof provided herein, or a composition thereof, and a chemotherapeutic agent provided herein is administered to a subject with a compromised immune system, a pregnant subject, a subject undergoing an organ or bone marrow transplant, a subject taking immunosuppressive drugs, a subject undergoing hemodialysis, a subject who has cancer, or a subject who is suffering from, are susceptible to, or are at risk for a neoplastic disease. In a more specific embodiment, an arenavirus particle expressing a tumor antigen, tumor associated antigen or an antigenic fragment thereof provided herein, or a composition thereof, and a chemotherapeutic agent provided herein is administered to a subject who is a child of 0, 1, 2, 3, 4, 5, 6, 7, 8, 9, 10, 11, 12, 13, 14, 15, 16, or 17 years of age suffering from, are susceptible to, or are at risk for a neoplastic disease. In yet another specific embodiment, an arenavirus particle expressing a tumor antigen, tumor associated antigen or an antigenic fragment thereof provided herein, or a composition thereof, and a chemotherapeutic agent provided herein is administered to a subject who is an infant suffering from, is susceptible to, or is at risk for a neoplastic disease. In yet another specific embodiment, an arenavirus particle expressing a tumor antigen, tumor associated antigen or an antigenic fragment thereof provided herein, or a composition thereof, and a chemotherapeutic agent provided herein is administered to a subject who is an infant of 0, 1, 2, 3, 4, 5, 6, 7, 8, 9, 10, 11, or 12 months of age suffering from, is susceptible to, or is at risk for a neoplastic disease. In yet another specific embodiment, an arenavirus particle expressing a tumor antigen, tumor associated antigen or an antigenic fragment thereof provided herein, or a composition thereof, and a chemotherapeutic agent provided herein is administered to an elderly subject who is suffering from, is susceptible to, or is at risk for a neoplastic disease. In a more specific embodiment, an arenavirus particle expressing a tumor antigen, tumor associated antigen or an antigenic fragment thereof provided herein, or a composition thereof, and a chemotherapeutic agent provided herein is administered to a subject who is a senior subject of 65, 66, 67, 68, 69, 70, 71, 72, 73, 74, 75, 76, 77, 78, 79, 80, 81, 82, 83, 84, 85, 86, 87, 88, 89, or 90 years of age. Provided herein is a method for preventing a cancer in a subject susceptible to, or is at risk for a neoplastic disease.

In another embodiment, an arenavirus particle expressing a tumor antigen, tumor associated antigen or an antigenic fragment thereof provided herein, or a composition thereof, and a chemotherapeutic agent provided herein is administered to subjects with a heightened risk of cancer metastasis. In a specific embodiment, an arenavirus particle expressing a tumor antigen, tumor associated antigen or an antigenic fragment thereof provided herein, or a composition thereof, and a chemotherapeutic agent provided herein is administered to subjects in the neonatal period with a neonatal and therefore immature immune system.

In another embodiment, an arenavirus particle expressing a tumor antigen, tumor associated antigen or an antigenic fragment thereof provided herein, or a composition thereof, and a chemotherapeutic agent provided herein is administered to a subject having grade 0 (i.e., in situ neoplasm), grade 1, grade 2, grade 3 or grade 4 cancer or a subcategory thereof, such as grade 3A, 3B, or 3C, or an equivalent thereof.

In another embodiment, an arenavirus particle expressing a tumor antigen, tumor associated antigen or an antigenic fragment thereof provided herein, or a composition thereof, and a chemotherapeutic agent provided herein is administered to a subject having cancer at a Tumor, Node, Metastasis (TNM) stage of any combination selected from Tumor T1, T2, T3, and T4, and Node N0, N1, N2, or N3, and Metastasis M0 and M1.

Successful treatment of a cancer patient can be assessed as prolongation of expected survival, induction of an anti-tumor immune response, or improvement of a particular characteristic of a cancer. Examples of characteristics of a cancer that might be improved include tumor size (e.g., T0, T is, or T1-4), state of metastasis (e.g., M0, M1), number of observable tumors, node involvement (e.g., N0, N1-4, Nx), grade (i.e., grades 1, 2, 3, or 4), stage (e.g., 0, I, II, III, or IV), presence or concentration of certain markers on the cells or in bodily fluids (e.g., AFP, B2M, beta-HCG, BTA, CA 15-3, CA 27.29, CA 125, CA 72.4, CA 19-9, calcitonin, CEA, chromgrainin A, EGFR, hormone receptors, HER2, HCG, immunoglobulins, NSE, NMP22, PSA, PAP, PSMA, S-100, TA-90, and thyroglobulin), and/or associated pathologies (e.g., ascites or edema) or symptoms (e.g., cachexia, fever, anorexia, or pain). The improvement, if measureable by percent, can be at least 5, 10, 15, 20, 25, 30, 40, 50, 60, 70, 80, or 90% (e.g., survival, or volume or linear dimensions of a tumor).

In another embodiment, an arenavirus particle expressing a tumor antigen, tumor associated antigen or an antigenic fragment thereof provided herein, or a composition thereof, and a chemotherapeutic agent provided herein is administered to a subject having a dormant cancer (e.g., the subject is in remission). Thus, provided herein is a method for preventing reactivation of a cancer. Also provided herein are methods for reducing the frequency of reoccurence of a cancer.

In another embodiment, an arenavirus particle expressing a tumor antigen, tumor associated antigen or an antigenic fragment thereof provided herein, or a composition thereof, and a chemotherapeutic agent provided herein is administered to a subject having a recurrent a cancer.

In another embodiment, an arenavirus particle expressing a tumor antigen, tumor associated antigen or an antigenic fragment thereof provided herein, or a composition thereof, and a chemotherapeutic agent provided herein is administered to a subject with a genetic predisposition for a cancer. In another embodiment, an arenavirus particle expressing a tumor antigen, tumor associated antigen or an antigenic fragment thereof provided herein, or a composition thereof, and a chemotherapeutic agent provided herein is administered to a subject with risk factors. Exemplary risk factors include, aging, tobacco, sun exposure, radiation exposure, chemical exposure, family history, alcohol, poor diet, lack of physical activity, or being overweight.

In another embodiment, an arenavirus particle expressing a tumor antigen, tumor associated antigen or an antigenic fragment thereof provided herein, or a composition thereof, and a chemotherapeutic agent provided herein is administered to subjects who suffer from one or more types of cancers. In other embodiments, any type of neoplastic disease, such as cancer, that is susceptible to treatment with the compositions described herein might be targeted.

In another embodiment, administering an arenavirus particle expressing a tumor antigen, tumor associated antigen or an antigenic fragment thereof provided or a composition thereof to subjects confer cell-mediated immunity (CMI) against a neoplastic cell or tumor, such as a cancer cell or tumor. Without being bound by theory, in another embodiment, an arenavirus particle expressing a tumor antigen, tumor associated antigen or an antigenic fragment thereof provided or a composition thereof infects and expresses antigens of interest in antigen presenting cells (APC) of the host (e.g., macrophages) for direct presentation of antigens on Major Histocompatibility Complex (MHC) class I and II. In another embodiment, administering an arenavirus particle expressing a tumor antigen, tumor associated antigen or an antigenic fragment thereof provided herein, or a composition thereof, to subjects induces plurifunctional IFN-γ and TNF-α co-producing cancer-specific CD4+ and CD8+ T cell responses (IFN-γ is produced by CD4+ and CD8+ T cells and TNF-α is produced by CD4+ T cells) of high magnitude to treat a neoplastic disease.

In another embodiment, administering an arenavirus particle expressing a tumor antigen, tumor associated antigen or an antigenic fragment thereof provided herein, or a composition thereof, and a chemotherapeutic agent provided herein increases or improves one or more clinical outcome for cancer treatment. Non-limiting examples of such outcomes are overall survival, progression-free survival, time to progression, time to treatment failure, event-free survival, time to next treatment, overall response rate and duration of response. The increase or improvement in one or more of the clinical outcomes can be by at least about 10%, at least about 20%, at least about 25%, at least about 30%, at least about 35%, at least about 40%, at least about 50%, at least about 60%, at least about 70%, at least about 80%, at least about 90%, or more, compared to a patient or group of patients having the same neoplastic disease in the absence of such treatment.

Changes in cell-mediated immunity (CMI) response function against a neoplastic cell or tumor, including a cancer cell or tumor, induced by administering an arenavirus particle expressing a tumor antigen, tumor associated antigen or an antigenic fragment thereof provided, or a composition thereof, in subjects can be measured by any assay known to the skilled artisan including, but not limited to flow cytometry (see, e.g., Perfetto S. P. et al., Nat Rev Immun. 2004; 4(8):648-55), lymphocyte proliferation assays (see, e.g., Bonilla F. A. et al., Ann Allergy Asthma Immunol. 2008; 101:101-4; and Hicks M. J. et al., Am J Clin Pathol. 1983; 80:159-63), assays to measure lymphocyte activation including determining changes in surface marker expression following activation of measurement of cytokines of T lymphocytes (see, e.g., Caruso A. et al., Cytometry. 1997; 27:71-6), ELISPOT assays (see, e.g., Czerkinsky C. C. et al., J Immunol Methods. 1983; 65:109-121; and Hutchings P. R. Et al., J Immunol Methods. 1989; 120:1-8), or Natural killer cell cytotoxicity assays (see, e.g., Bonilla F. A. et al., Ann Allergy Asthma Immunol. 2005 May; 94(5 Suppl 1): S1-63).

Chemotherapeutic agents described herein can be alkylating agents (e.g., cyclophosphamide), platinum-based therapeutics, antimetabolites, topoisomerase inhibitors, cytotoxic antibiotics, intercalating agents, mitosis inhibitors, taxanes, or combinations of two or more thereof. In certain embodiments, the alkylating agent is a nitrogen mustard, a nitrosourea, an alkyl sulfonate, a non-classical alkylating agent, or a triazene. In certain embodiments, the chemotherapeutic agent comprises one or more of cyclophosphamide, thiotepa, mechlorethamine (chlormethine/mustine), uramustine, melphalan, chlorambucil, ifosfamide, chlornaphazine, cholophosphamide, estramustine, novembichin, phenesterine, prednimustine, trofosfamide, uracil mustard, bendamustine, busulfan, improsulfan, piposulfan, carmustine, lomustine, chlorozotocin, fotemustine, nimustine, ranimustine, streptozucin, cisplatin, carboplatin, nedaplatin, oxaliplatin, satraplatin, triplatin tetranitrate, procarbazine, altretamine, dacarbazine, mitozolomide, temozolomide, paclitaxel, docetaxel, vinblastine, vincristine, vinorelbine, cabazitaxel, dactinomycin (actinomycin D), calicheamicin, dynemicin, amsacrine, doxarubicin, daunorubicin, epirubicin, mitoxantrone, idarubicin, pirarubicin, benzodopa, carboquone, meturedopa, uredopa, altretamine, triethylenemelamine, trietylenephosphoramide, triethiylenethiophosphoramide, trimethylolomelamine, bullatacin, bullatacinone, camptothecin, topotecan, bryostatin, callystatin, CC-1065, adozelesin, carzelesin, bizelesin, cryptophycin, dolastatin, duocarmycin, KW-2189, CB1-TM1, eleutherobin, pancratistatin, sarcodictyin, spongistatin, clodronate, esperamicin, neocarzinostatin chromophore, aclacinomysin, anthramycin, azaserine, bleomycin, cactinomycin, carabicin, carminomycin, carzinophilin, chromomycinis, detorubicin, 6-diazo-5-oxo-L-norleucine, esorubicin, idarubicin, marcellomycin, mitomycin, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, mitotane, trilostane, frolinic acid, aceglatone, aldophosphamide glycoside, aminolevulinic acid, eniluracil, bestrabucil, bisantrene, edatraxate, defofamine, demecolcine, diaziquone, elformithine, elliptinium acetate, etoglucid, gallium nitrate, hydroxyurea, lentinan, lonidainine, maytansine, ansamitocins, mitoguazone, mopidanmol, nitraerine, pentostatin, phenamet, pirarubicin, losoxantrone, podophyllinic acid, 2-ethylhydrazide, PSK polysaccharide complex, razoxane, rhizoxin, sizofiran, spirogermanium, tenuazonic acid, triaziquone, 2,2′,2″-trichlorotriethylamine; T-2 toxin, verracurin A, roridin A and anguidine, urethan, vindesine, mannomustine, mitobronitol, mitolactol, pipobroman, gacytosine, arabinoside (“Ara-C”), etoposide (VP-16), vinorelbine, novantrone, teniposide, edatrexate, aminopterin, xeloda, ibandronate, irinotecan (e.g., CPT-11), topoisomerase inhibitor RFS 2000, difluorometlhylornithine (DMFO), retinoic acid, capecitabine, plicomycin, gemcitabine, navelbine, transplatinum, and pharmaceutically acceptable salts, acids, or derivatives of any of the above. In specific embodiments, the chemotherapeutic agent comprises cyclophosphamide. In certain embodiments, the nitrogen mustard is mechlorethamine, cyclophosphamide, melphalan, chlorambucil, ifosfamide, or busulfan. In certain embodiments, the chemotherapeutic agent alkylates DNA. In certain embodiments, the chemotherapeutic agent alkylates DNA, resulting in the formation of interstrand cross-links (“ICLs”).

In certain embodiments, chemotherapeutic agents described herein are used in combination with an immune checkpoint inhibitor that inhibits, decreases or interferes with the activity of a negative checkpoint regulator. In certain embodiments, the negative checkpoint regulator is selected from the group consisting of Cytotoxic T-lymphocyte antigen-4 (CTLA-4), CD80, CD86, Programmed cell death 1 (PD-1), Programmed cell death ligand 1 (PD-L1), Programmed cell death ligand 2 (PD-L2), Lymphocyte activation gene-3 (LAG-3; also known as CD223), Galectin-3, B and T lymphocyte attenuator (BTLA), T-cell membrane protein 3 (TIM3), Galectin-9 (GAL9), B7-H1, B7-H3, B7-H4, T-Cell immunoreceptor with Ig and ITIM domains (TIGIT/Vstm3/WUCAM/VSIG9), V-domain Ig suppressor of T-Cell activation (VISTA), Glucocorticoid-induced tumor necrosis factor receptor-related (GITR) protein, Herpes Virus Entry Mediator (HVEM), OX40, CD27, CD28, CD137. CGEN-15001T, CGEN-15022, CGEN-15027, CGEN-15049, CGEN-15052, and CGEN-15092. In certain embodiments, the immune checkpoint inhibitor is an anti-PD-1 antibody.

In certain embodiments, an arenavirus particle expressing a tumor antigen, tumor associated antigen or an antigenic fragment thereof provided herein, or a composition thereof, and a chemotherapeutic agent provided herein is preferably administered in multiple injections (e.g., at least 2, 3, 4, 5, 6, 7, 8, 9, 10, 12, 14, 16, 18, 20, 25, 30, 40, 45, or 50 injections) or by continuous infusion (e.g., using a pump) at multiple sites (e.g., at least 2, 3, 4, 5, 6, 7, 8, 9, 10, 12, or 14 sites). In certain embodiments, the arenavirus particle expressing a tumor antigen, tumor associated antigen or an antigenic fragment thereof provided herein, or a composition thereof, is administered in two or more separate injections over a 6-month period, a 12-month period, a 24-month period, or a 48-month period. In certain embodiments, the arenavirus particle expressing a tumor antigen, tumor associated antigen or an antigenic fragment thereof provided herein, or a composition thereof, is administered with a first dose at an elected date, a second dose at least 2 months after the first dose, and a third does 6 months after the first dose.

In one example, cutaneous injections are performed at multiple body sites to reduce extent of local skin reactions. On a given vaccination day, the patient receives the assigned total dose administered from one syringe in 3 to 5 separate intradermal injections of the dose (e.g., at least 0.4 ml, 0.2 ml, or 0.1 ml) each in an extremity spaced at least about 5 cm (e.g., at least 4.5, 5, 6, 7, 8, 9, or cm) at needle entry from the nearest neighboring injection. On subsequent vaccination days, the injection sites are rotated to different limbs in a clockwise or counter-clockwise manner.

In certain embodiments, the methods further comprise co-administration of the arenavirus particle provided herein and a chemotherapeutic agent. In certain embodiments, the co-administration is simultaneous. In another embodiment, the arenavirus particle is administered prior to administration of the chemotherapeutic agent. In other embodiments, the arenavirus particle is administered after administration of the chemotherapeutic agent. In certain embodiments, the interval between administration of the arenavirus particle and the chemotherapeutic agent is about 1 hour, about 2 hours, about 3 hours, about 4 hours, about 5 hours, about 6 hours, about 7 hours, about 8 hours, about 9 hours, about 10 hours, about 11 hours, or about 12 hours. In certain embodiments, the interval between administration of the arenavirus particle and the chemotherapeutic agent is about 1 day, about 2 days, about 3 days, about 4 days, about 5 days, about 6 days, about 1 week, about 8 days, about 9 days, about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks. In certain embodiments, the interval between administration of the arenavirus particle and the chemotherapeutic agent is about 1 month, about 2 months, about 3 months, about 4 months, about 5 months, or about 6 months. In some embodiments, the method further includes administering at least one additional therapy.

In another embodiment, two arenavirus particles are administered in a treatment regime at molar ratios ranging from about 1:1 to 1:1000, in particular including: 1:1 ratio, 1:2 ratio, 1:5 ratio, 1:10 ratio, 1:20 ratio, 1:50 ratio, 1:100 ratio, 1:200 ratio, 1:300 ratio, 1:400 ratio, 1:500 ratio, 1:600 ratio, 1:700 ratio, 1:800 ratio, 1:900 ratio, 1:1000 ratio.

In certain embodiments, provided herein is a method of treating neoplastic disease wherein a first arenavirus particle is administered first as a “prime,” and a second arenavirus particle is administered as a “boost.” The first and the second arenavirus particles can express the same or different tumor antigens, tumor associated antigens or antigenic fragments thereof. Alternatively, or additionally, some certain embodiments, the “prime” and “boost” administration are performed with an arenavirus particle derived from different species. In certain specific embodiments, the “prime” administration is performed with an arenavirus particle derived from LCMV, and the “boost” is performed with an arenavirus particle derived from Junin virus. In certain specific embodiments, the “prime” administration is performed with an arenavirus particle derived from Junin virus, and the “boost” is performed with an arenavirus particle derived from LCMV. In certain embodiments, the “prime” administration is performed with an arenavirus particle derived from Pichinde virus, and the “boost” is performed with an arenavirus particle derived from LCMV. In certain embodiments, the “prime” administration is performed with an arenavirus particle derived from Pichinde virus, and the “boost” is performed with an arenavirus particle derived from Junin virus. In certain embodiments, the “prime” administration is performed with an arenavirus particle derived from LCMV, and the “boost” is performed with an arenavirus particle derived from Pichinde virus. In certain embodiments, the “prime” administration is performed with an arenavirus particle derived from Junin virus, and the “boost” is performed with an arenavirus particle derived from Pichinde virus. In certain embodiments, the “prime” administration and/or the “boost” administration are performed in combination with the administration of an immunomodulatory peptide, polypeptide, or protein. In certain embodiments, the “prime” administration and/or the “boost” administration are performed in combination with the administration of a chemotherapeutic agent.

In certain embodiments, administering a first arenavirus particle expressing a tumor antigen, tumor associated antigen or antigenic fragment thereof, followed by administering a second arenavirus particle expressing a tumor antigen, tumor associated antigen or antigenic fragment thereof results in a greater antigen specific CD8+ T cell response than administering a single arenavirus particle expressing a tumor antigen, tumor associated antigen or antigenic fragment thereof. In certain embodiments, the antigen specific CD8+ T cell count increases by 50%, 100%, 150% or 200% after the second administration compared to the first administration. In certain embodiments, administering a third arenavirus particle expressing a tumor antigen, tumor associated antigen or antigenic fragment thereof results in a greater antigen specific CD8+ T cell response than administering two consecutive arenavirus particles expressing a tumor antigen, tumor associated antigen or antigenic fragment thereof. In certain embodiments, the antigen specific CD8+ T cell count increases by about 50%, about 100%, about 150%, about 200% or about 250% after the third administration compared to the first administration.

In certain embodiments, provided herein are methods for treating a neoplastic disease comprising administering two or more arenavirus particles, wherein the two or more arenavirus particles are homologous, and wherein the time interval between each administration is about 1 week, about 2 weeks, about 3 week, about 4 weeks, about 5 weeks, about 6 weeks, about 7 weeks, about 8 weeks, about 3 months, about 4 months, about 5 months, about 6 months, about 7 months, about 8 months, about 9 months, about 10 months, about 11 months, about 12 months, about 18 months, or about 24 months.

In certain embodiments, administering a first arenavirus particle expressing a tumor antigen, tumor associated antigen or antigenic fragment thereof and a second, heterologous, arenavirus particle expressing a tumor antigen, tumor associated antigen or antigenic fragment thereof elicits a greater CD8+ T cell response than administering a first arenavirus particle expressing a tumor antigen, tumor associated antigen or antigenic fragment thereof and a second, homologous, arenavirus particle expressing a tumor antigen, tumor associated antigen or antigenic fragment thereof.

### (g) Compositions, Administration, and Dosage

In certain embodiments, immunogenic compositions (e.g., vaccine formulations), and pharmaceutical compositions comprising an arenavirus particle provided herein can be used with the methods and compositions provided herein, such as combinations with a chemotherapeutic agent. Such vaccines, immunogenic compositions and pharmaceutical compositions can be formulated according to standard procedures in the art.

In another embodiment, provided herein are compositions comprising an infectious, replication-deficient arenavirus particle described herein, and, in certain embodiments, a chemotherapeutic agent provided herein. Such compositions can be used in methods of treating a neoplastic disease. In another specific embodiment, the immunogenic compositions provided herein can be used to induce an immune response in a host to whom the composition is administered. The immunogenic compositions described herein can be used as vaccines and can accordingly be formulated as pharmaceutical compositions. In a specific embodiment, the immunogenic compositions described herein are used in the treatment of a neoplastic disease a subject (e.g., human subject). In other embodiments, the vaccine, immunogenic composition or pharmaceutical composition are suitable for veterinary and/or human administration.

In certain embodiments, provided herein are immunogenic compositions comprising an arenavirus particle (or a combination of different arenavirus particles) as described herein. In certain embodiments, such an immunogenic composition further comprises a pharmaceutically acceptable excipient. In certain embodiments, such an immunogenic composition further comprises an adjuvant. The adjuvant for administration in combination with a composition described herein may be administered before, concomitantly with, or after administration of said composition. In some embodiments, the term “adjuvant” refers to a compound that when administered in conjunction with or as part of a composition described herein augments, enhances and/or boosts the immune response to an infectious, replication-deficient arenavirus particle, but when the compound is administered alone does not generate an immune response to the infectious, replication-deficient arenavirus particle. In some embodiments, the adjuvant generates an immune response to the infectious, replication-deficient arenavirus particle and does not produce an allergy or other adverse reaction. Adjuvants can enhance an immune response by several mechanisms including, e.g., lymphocyte recruitment, stimulation of B and/or T cells, and stimulation of macrophages. When a vaccine or immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., N. Engl. J. Med. 336, 86-91 (1997)).

The compositions comprise the infectious, replication-deficient arenavirus particles described herein alone or together with a pharmaceutically acceptable carrier and/or a chemotherapeutic agent. Suspensions or dispersions of genetically engineered arenavirus particles, especially isotonic aqueous suspensions or dispersions, can be used. The pharmaceutical compositions may be sterilized and/or may comprise excipients, e.g., preservatives, stabilizers, wetting agents and/or emulsifiers, solubilizers, salts for regulating osmotic pressure and/or buffers and are prepared in a manner known per se, for example by means of conventional dispersing and suspending processes. In certain embodiments, such dispersions or suspensions may comprise viscosity-regulating agents. The suspensions or dispersions are kept at temperatures around 2-8° C., or preferentially for longer storage may be frozen and then thawed shortly before use. For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

In certain embodiments, the compositions described herein additionally comprise a preservative, e.g., the mercury derivative thimerosal. In a specific embodiment, the pharmaceutical compositions described herein comprise 0.001% to 0.01% thimerosal. In other embodiments, the pharmaceutical compositions described herein do not comprise a preservative.

The pharmaceutical compositions comprise from about 10³ to about 10¹¹ focus forming units of the genetically engineered arenavirus particles. Unit dose forms for parenteral administration are, for example, ampoules or vials, e.g., vials containing from about 10³ to 10¹⁰ focus forming units or 10⁵ to 10¹⁵ physical particles of genetically engineered arenavirus particles.

In another embodiment, a vaccine or immunogenic composition provided herein is administered to a subject by, including but not limited to, oral, intradermal, intramuscular, intraperitoneal, intravenous, topical, subcutaneous, percutaneous, intranasal and inhalation routes, and via scarification (scratching through the top layers of skin, e.g., using a bifurcated needle). Specifically, subcutaneous, intramuscular or intravenous routes can be used.

For administration intranasally or by inhalation, the preparation for use according to the present invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflators may be formulated containing a powder mix of the compound and a suitable powder base such as lactose or starch.

The dosage of the active ingredient depends upon the type of vaccination and upon the subject, and their age, weight, individual condition, the individual pharmacokinetic data, and the mode of administration.

In certain embodiments, the compositions can be administered to the patient in a single dosage comprising a therapeutically effective amount of the arenavirus particle and/or a therapeutically effective amount of a chemotherapeutic agent. In some embodiments, the arenavirus particle can be administered to the patient in a single dose comprising an arenavirus particle and a chemotherapeutic agent, each in a therapeutically effective amount.

In certain embodiments, the composition is administered to the patient as a single dose followed by a second dose three to six weeks later. In accordance with these embodiments, the booster inoculations may be administered to the subjects at six to twelve month intervals following the second inoculation. In certain embodiments, the booster inoculations may utilize a different arenavirus particle or composition thereof. In some embodiments, the administration of the same composition as described herein may be repeated and separated by at least 1 day, 2 days, 3 days, 4 days, 5 days, 10 days, 15 days, 30 days, 45 days, 2 months, 75 days, 3 months, or at least 6 months.

In certain embodiments, the vaccine, immunogenic composition, or pharmaceutical composition comprising an arenavirus particle can be used as a live vaccination. Exemplary doses for a live arenavirus particle may vary from 10-100, or more, PFU of live virus per dose. In some embodiments, suitable dosages of an arenavirus particle or the tri-segmented arenavirus particle are 10², 5×10², 103, 5×103, 10⁴, 5×10⁴, 10⁵, 5×10⁵, 10⁶, 5×10⁶, 107, 5×107, 10⁸, 5×10⁸, 1×10⁹, 5×10⁹, 1×10¹⁰, 5×10¹⁰, 1×10¹¹, 5×10¹ or 10¹² pfu, and can be administered to a subject once, twice, three or more times with intervals as often as needed. In another embodiment, a live arenavirus is formulated such that a 0.2-mL dose contains 10⁶-5-10⁷⁵ fluorescent focal units of live arenavirus particle. In another embodiment, an inactivated vaccine is formulated such that it contains about 15 g to about 100 g, about 15 g to about 75 g, about 15 g to about 50 g, or about 15 g to about 30 g of an arenavirus

Also provided are processes and uses of an arenavirus particle and a chemotherapeutic agent for the manufacture of vaccines in the form of pharmaceutical preparations, which comprise the arenavirus particle and the chemotherapeutic agent as an active ingredient. Still further provided is a combination of an arenavirus particle provided herein and a chemotherapeutic agent provided herein for use in the treatment of a neoplastic disease described herein. In certain embodiments, the combination is in the same pharmaceutical composition. In certain embodiments, the combination is not in the same pharmaceutical composition, such as when the arenavirus particle and the chemotherapeutic agent are to be separately administered. The pharmaceutical compositions of the present application are prepared in a manner known per se, for example by means of conventional mixing and/or dispersing processes.

Also provided herein are kits that can be used to perform the methods described herein. In certain embodiments, the kit provided herein can include one or more containers. These containers can hold for storage the compositions (e.g., pharmaceutical, immunogenic or vaccine composition) provided herein. Also included in the kit are instructions for use. These instructions describe, in sufficient detail, a treatment protocol for using the compositions contained therein. For example, the instructions can include dosing and administration instructions as provided herein for the methods of treating a neoplastic disease.

In certain embodiments, a kit provided herein includes containers that each contains the active ingredients for performing the methods described herein. Thus, in certain embodiments, the kit provided herein includes two or more containers and instructions for use, wherein one of the containers comprises an infectious, replication-deficient arenavirus particle provided herein and another container that comprises a chemotherapeutic agent provided herein.

### (h) Assays

### (i) Arenavirus Detection Assays

The skilled artesian could detect an arenavirus genomic segment or tri-segmented arenavirus particle, as described herein using techniques known in the art. For example, RT-PCR can be used with primers that are specific to an arenavirus to detect and quantify an arenavirus genomic segment that has been engineered to carry an ORF in a position other than the wild-type position of the ORF or a tri-segmented arenavirus particle. Western blot, ELISA, radioimmunoassay, immuneprecipitation, immunecytochemistry, or immunocytochemistry in conjunction with FACS can be used to quantify the gene products of the arenavirus genomic segment or tri-segmented arenavirus particle.

### (ii) Assay to Measure Infectivity

Any assay known to the skilled artisan can be used for measuring the infectivity of an arenavirus vector preparation. For example, determination of the virus/vector titer can be done by a “focus forming unit assay” (FFU assay). In brief, complementing cells, e.g., MC57 cells are plated and inoculated with different dilutions of a virus/vector sample. After an incubation period, to allow cells to form a monolayer and virus to attach to cells, the monolayer is covered with Methylcellulose. When the plates are further incubated, the original infected cells release viral progeny. Due to the Methylcellulose overlay the spread of the new viruses is restricted to neighboring cells. Consequently, each infectious particle produces a circular zone of infected cells called a Focus. Such Foci can be made visible and by that countable using antibodies against LCMV-NP or another protein expressed by the arenavirus particle or the tri-segmented arenavirus particle and a HRP-based color reaction. The titer of a virus/vector can be calculated in focus-forming units per milliliter (FFU/mL).

(iii) Growth of an Arenavirus Particle

Growth of an arenavirus particle described herein can be assessed by any method known in the art or described herein (e.g., cell culture). Viral growth may be determined by inoculating serial dilutions of an arenavirus particle described herein into cell cultures (e.g., Vero cells or BHK-21 cells). After incubation of the virus for a specified time, the virus is isolated using standard methods.

### (iv) Serum ELISA

Determination of the humoral immune response upon vaccination of animals (e.g., mice, guinea pigs) can be done by antigen-specific serum ELISA's (enzyme-linked immunosorbent assays). In brief, plates are coated with antigen (e.g., recombinant protein), blocked to avoid unspecific binding of antibodies and incubated with serial dilutions of sera. After incubation, bound serum-antibodies can be detected, e.g., using an enzyme-coupled anti-species (e.g., mouse, guinea pig)-specific antibody (detecting total IgG or IgG subclasses) and subsequent color reaction. Antibody titers can be determined as, e.g., endpoint geometric mean titer.

Immunocapture ELISA (IC-ELISA) may also be performed (see Shanmugham et al., 2010, Clin. Vaccine Immunol. 17(8):1252-1260), wherein the capture agents are cross-linked to beads.

### (v) Assay to Measure the Neutralizing Activity of Induced Antibodies

Determination of the neutralizing antibodies in sera is performed with the following cell assay using ARPE-19 cells from ATCC and a GFP-tagged virus. In addition supplemental guinea pig serum as a source of exogenous complement is used. The assay is started with seeding of 6.5×10³ cells/well (50 μl/well) in a 384 well plate one or two days before using for neutralization. The neutralization is done in 96-well sterile tissue culture plates without cells for 1 h at 37° C. After the neutralization incubation step the mixture is added to the cells and incubated for additional 4 days for GFP-detection with a plate reader. A positive neutralizing human sera is used as assay positive control on each plate to check the reliability of all results. Titers (EC50) are determined using a 4 parameter logistic curve fitting. As additional testing the wells are checked with a fluorescence microscope.

### (A) Plaque Reduction Assay

In brief, plaque reduction (neutralization) assays for LCMV can be performed by use of a replication-competent or -deficient LCMV that is tagged with green fluorescent protein, 5% rabbit serum may be used as a source of exogenous complement, and plaques can be enumerated by fluorescence microscopy. Neutralization titers may be defined as the highest dilution of serum that results in a 50%, 75%, 90% or 95% reduction in plaques, compared with that in control (pre-immune) serum samples. qPCR LCMV RNA genomes are isolated using QIAamp Viral RNA mini Kit (QIAGEN), according to the protocol provided by the manufacturer. LCMV RNA genome equivalents are detected by quantitative PCR carried out on an StepOnePlus Real Time PCR System (Applied Biosystems) with SuperScript® III Platinum® One-Step qRT-PCR Kit (Invitrogen) and primers and probes (FAM reporter and NFQ-MGB Quencher) specific for part of the LCMV NP coding region or another genomic stretch of the arenavirus particle or the tri-segmented arenavirus particle. The temperature profile of the reaction may be: 30 min at 60° C., 2 min at 95° C., followed by 45 cycles of 15 s at 95° C., 30 s at 56° C. RNA can be quantified by comparison of the sample results to a standard curve prepared from a log 10 dilution series of a spectrophotometrically quantified, in vitro-transcribed RNA fragment, corresponding to a fragment of the LCMV NP coding sequence or another genomic stretch of the arenavirus particle or the tri-segmented arenavirus particle containing the primer and probe binding sites.

### (B) Neutralization Assay in Guinea Pig Lung Fibroblast (GPL) Cells

In brief, serial dilutions of test and control (pre-vaccination) sera were prepared in GPL complete media with supplemental rabbit serum (1%) as a source of exogenous complement. The dilution series spanned 1:40 through 1:5120. Serum dilutions were incubated with eGFP tagged virus (100-200 pfu per well) for 30 min at 37° C., and then transferred to 12-well plates containing confluent GPL cells. Samples were processed in triplicate. After 2 hours incubation at 37° C. the cells were washed with PBS, re-fed with GPL complete media and incubated at 37° C./5% CO₂ for 5 days. Plaques were visualized by fluorescence microscopy, counted, and compared to control wells. That serum dilution resulting in a 50% reduction in plaque number compared to controls was designated as the neutralizing titer.

### (C) Western Blotting

Infected cells grown in tissue culture flasks or in suspension are lysed at indicated timepoints post infection using RIPA buffer (Thermo Scientific) or used directly without cell-lysis. Samples are heated to 99° C. for 10 minutes with reducing agent and NuPage LDS Sample buffer (NOVEX) and chilled to room temperature before loading on 4-12% SDS-gels for electrophoresis. Proteins are blotted onto membranes using Invitrogens iBlot Gel transfer Device and visualized by Ponceau staining. Finally, the preparations are probed with a primary antibodies directed against proteins of interest and alkaline phosphatase conjugated secondary antibodies followed by staining with 1-Step NBT/BCIP solution (INVITROGEN).

### (D) MHC-Peptide Multimer Staining Assay for Detection of Antigen-Specific CD8+ T-Cell Proliferation

Any assay known to the skilled artisan can be used to test antigen-specific CD8+ T-cell responses. For example, the MHC-peptide tetramer staining assay can be used (see, e.g., Altman J. D. et al., Science. 1996; 274:94-96; and Murali-Krishna K. et al., Immunity. 1998; 8:177-187). Briefly, the assay comprises the following steps, a tetramer assay is used to detect the presence of antigen specific T-cells. In order for a T-cell to detect the peptide to which it is specific, it must both recognize the peptide and the tetramer of MHC molecules custom made for a defined antigen specificity and MHC haplotype of T-cells (typically fluorescently labeled). The tetramer is then detected by flow cytometry via the fluorescent label.

### (E) ELISPOT Assay for Detection of Antigen-Specific CD4+ T-Cell Proliferation.

Any assay known to the skilled artisan can be used to test antigen-specific CD4+ T-cell responses. For example, the ELISPOT assay can be used (see, e.g., Czerkinsky C. C. et al., J Immunol Methods. 1983; 65:109-121; and Hutchings P. R. et al., J Immunol Methods. 1989; 120:1-8). Briefly, the assay comprises the following steps: An immunospot plate is coated with an anti-cytokine antibody. Cells are incubated in the immunospot plate. Cells secrete cytokines and are then washed off. Plates are then coated with a second biotyinlated-anticytokine antibody and visualized with an avidin-HRP system.

### (F) Intracellular Cytokine Assay for Detection of Functionality of CD8+ and CD4+ T-Cell Responses.

Any assay known to the skilled artisan can be used to test the functionality of CD8+ and CD4+ T cell responses. For example, the intracellular cytokine assay combined with flow cytometry can be used (see, e.g., Suni M. A. et al., J Immunol Methods. 1998; 212:89-98; Nomura L. E. et al., Cytometry. 2000; 40:60-68; and Ghanekar S. A. et al., Clinical and Diagnostic Laboratory Immunology. 2001; 8:628-63). Briefly, the assay comprises the following steps: activation of cells via specific peptides or protein, an inhibition of protein transport (e.g., brefeldin A) is added to retain the cytokines within the cell. After a defined period of incubation, typically 5 hours, a washing steps follows, and antibodies to other cellular markers can be added to the cells. Cells are then fixed and permeabilized. The flurochrome-conjugated anti-cytokine antibodies are added and the cells can be analyzed by flow cytometry.

### (G) Assay for Confirming Replication-Deficiency of Viral Vectors

Any assay known to the skilled artisan that determines concentration of infectious and replication-competent virus particles can also be used as a to measure replication-deficient viral particles in a sample. For example, FFU assays with non-complementing cells can be used for this purpose.

Furthermore, plaque-based assays are the standard method used to determine virus concentration in terms of plaque forming units (PFU) in a virus sample. Specifically, a confluent monolayer of non-complementing host cells is infected with the virus at varying dilutions and covered with a semi-solid medium, such as agar to prevent the virus infection from spreading indiscriminately. A viral plaque is formed when a virus successfully infects and replicates itself in a cell within the fixed cell monolayer, and spreads to surrounding cells (see, e.g., Kaufmann, S. H.; Kabelitz, D. (2002). Methods in Microbiology Vol. 32:Immunology of Infection. Academic Press. ISBN 0-12-521532-0). Plaque formation can take 2-14 days, depending on the virus being analyzed. Plaques are generally counted manually and the results, in combination with the dilution factor used to prepare the plate, are used to calculate the number of plaque forming units per sample unit volume (PFU/mL). The PFU/mL result represents the number of infective replication-competent particles within the sample. When C-cells are used, the same assay can be used to titrate replication-deficient arenavirus particles or tri-segmented arenavirus particles.

### (vi) Assay for Expression of Viral Antigen

Any assay known to the skilled artisan can be used for measuring expression of viral antigens. For example, FFU assays can be performed. For detection, mono- or polyclonal antibody preparation(s) against the respective viral antigens are used (transgene-specific FFU).

(vii) Animal Models

To investigate recombination and infectivity of an arenavirus particle described herein in vivo animal models can be used. In certain embodiments, the animal models that can be used to investigate recombination and infectivity of a tri-segmented arenavirus particle include mouse, guinea pig, rabbit, and monkeys. In a preferred embodiment, the animal models that can be used to investigate recombination and infectivity of an arenavirus include mouse. In a more specific embodiment, the mice can be used to investigate recombination and infectivity of an arenavirus particle are triple-deficient for type I interferon receptor, type II interferon receptor and recombination activating gene 1 (RAG1).

In certain embodiments, the animal models can be used to determine arenavirus infectivity and transgene stability. In some embodiments, viral RNA can be isolated from the serum of the animal model. Techniques are readily known by those skilled in the art. The viral RNA can be reverse transcribed and the cDNA carrying the arenavirus ORFs can be PCR-amplified with gene-specific primers. Flow cytometry can also be used to investigate arenavirus infectivity and transgene stability.

### (A) Chemotherapeutic Agent Assays

A number of assays have been devised that are capable of assessing properties of proposed chemotherapeutic agents. Tumor models that can be used to test the methods and compositions disclosed herein include Colon26 (CT26), MC38 (mouse colon adenocarcinoma), B16F10 (B16), Lewis Lung (LLC), Madison109 (Mad 109), EMT-6 (murine breast cancer), 4T1 (4T1) (murine breast cancer), HCmel3 (murine melanoma), HgfxCDK4^{R24C/R24C} (murine melanoma), and (RENCA) (murine renal cancer).

In certain embodiments, in these model systems, “transplantable tumors” can be generated by subcutaneous (e.g., CT26, 4T1, MAD109, RENCA, LLC, or B16) or intracerebral (e.g., GL261, ONC26M4) inoculation of tumor cell lines into rodents, for example in adult female mice. Tumors can be developed over pre-determined time intervals, for example several days. These tumors are grown in syngeneic, immunocompetent rodent, e.g., mouse, strains. For example CT26, 4T1, MAD109, and RENCA can be grown in BALB/c mice, LLC, B16, and GL261 can be grown in C57BL/6 mice, and ONC26M4 can be grown in FVBN mice. “Spontaneous tumors” can be generated by intracerebral injection of DNA plasmids encoding a number (e.g., one, two, three or more) of oncogenes and encoding one or more reporter, e.g., firefly luciferase reporter, into neonatal C57BL/6 or FVBN mice to transform endogenous brain cells. Growth of gliomas can be monitored by techniques known in the art, e.g., bioluminescence imaging. Growth of subcutaneous tumors can be monitored by techniques known in the art, e.g., caliper measurements in three dimensions at specified time intervals.

### 5.3 Heterologous Prime Boost

In certain embodiments, provided herein are methods and compositions relating to a heterologous prime/boost using the replication-defective viruses or the tri-segmented, replication competent viruses described herein (see Sections 5.1 and 5.2). In specific embodiments, such heterologous prime/boost treatment regimens are conducted without concurrent chemotherapy or without concurrent treatment with immune checkpoint modulators. In other embodiments, chemotherapy and/or therapy with immune checkpoint modulators has been concluded prior to the initiation of the heterologous prime/boost regimen described in this section. In even other embodiments, a patient to be treated with the heterologous prime/boost regimen has not previously been treated for the present condition with chemotherapy and/or therapy with immune checkpoint modulators and is also not concurrently being treated with chemotherapy and/or therapy with immune checkpoint modulators. In certain embodiments, a patient is treated with multiple and/or successive heterologous primbe/boost regimens.

In certain embodiments, the heterologous prime/boost regimen comprises administering a first arenavirus-derived construct as described herein followed by administering a second arenavirus-derived construct as described herein. In a specific embodiment the first and the second arenavirus-based constructs comprise a nucleotide sequence encoding the same tumor antigen, tumor associated antigen or antigenic fragment thereof. The tumor antigen or tumor-associated antigen can be an antigen listed in Section 5.1.(a), 5.1.(b), 5.2.(a), 5.2.(b), or 5.2.(c). In a specific embodiment, both arenavirus-derived constructs comprise a nucleotide sequence encoding an antigen of an oncogenic virus, such as an HPV antigen, such as HPV16 E7/E6 fusion (eg, as described in WO2015/082570, which is incorporated herein in its entirety).

In certain embodiments, the first and the second arenavirus-based constructs are administered at least 1, 2, 3, 4, 5, 6, 7, 8, 9, 10, 11, 12, 13, or at least 14 days apart; at least 1, 2, 3, 4, 5, 6, 7, or at least 8 weeks apart; at least 1, 2, 3, 4, 5, 6, 7, 8, 9, 10, 11, or at least 12 months apart. In certain embodiments, the first and the second arenavirus-based constructs are administered at most 1, 2, 3, 4, 5, 6, 7, 8, 9, 10, 11, 12, 13, or at most 14 days apart; at most 1, 2, 3, 4, 5, 6, 7, or at most 8 weeks apart; at most 1, 2, 3, 4, 5, 6, 7, 8, 9, 10, 11, or at most 12 months apart.

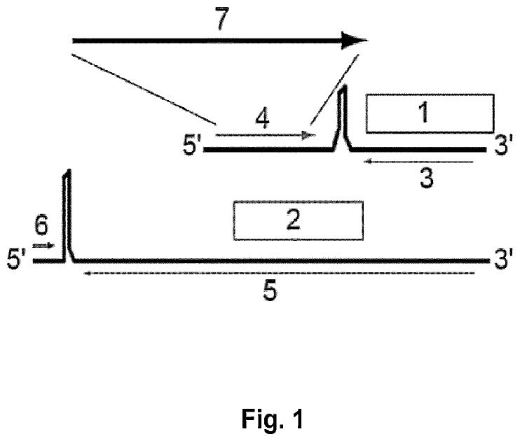

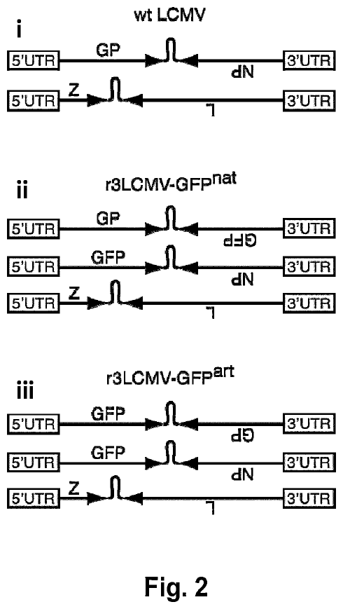

In certain embodiments, the first arenavirus-based construct has a genomic organization as shown in FIG. 1 (ie, the open reading frame for the GP protein is deleted or functionally inactivated and replaced with an open reading frame for the tumor antigen or tumor-associated antigen or antigen of a oncogenic virus) or as shown in FIG. 2 as outlined for r3LCMV-GFP^{arifcial} except that in place of the open reading frame encoding GFP, the virus has an open reading frame for a tumor antigen or tumor-associated antigen or antigen of a oncogenic virus. In certain embodiments, the second arenavirus-based construct has a genomic organization as shown in FIG. 1 (ie, the open reading frame for the GP protein is deleted or functionally inactivated and replaced with an open reading frame for the tumor antigen or tumor-associated antigen or antigen of a oncogenic virus) or as shown in FIG. 2 as outlined for r3LCMV-GFP^{arifcial} except that in place of the open reading frame encoding GFP, the virus has an open reading frame for a tumor antigen or tumor-associated antigen or antigen of a oncogenic virus. In a specific embodiment, the first and the second arenavirus-based constructs have a genomic organization as shown in FIG. 1 (ie, the open reading frame for the GP protein is deleted or functionally inactivated and replaced with an open reading frame for the tumor antigen or tumor-associated antigen or antigen of a oncogenic virus) or as shown in FIG. 2 as outlined for r3LCMV-GFP^{arifcial} except that in place of the open reading frame encoding GFP, the virus has an open reading frame for a tumor antigen or tumor-associated antigen or antigen of a oncogenic virus.

In a specific embodiment, the first and the second arenavirus-based constructs have a genomic organization as shown in FIG. 2 as outlined for r3LCMV-GFP^{arificial} except that in place of the open reading frame encoding GFP, the viruses have an open reading frame for a tumor antigen or tumor-associated antigen or antigen of a oncogenic virus (such as an HPV16 E7/E6 fusion protein). Further, the first arenavirus-based vaccine is derived from a Pichinde, Junin, or LCMV; and the second arenavirus-based vaccine is derived from a Pichinde, Junin, or LCMV (but different from the viral backbone of the first construct). In an even more specific embodiment, the first construct (prime) is derived from Pichinide virus and the second construct (boost) is derived from LCMV. The first and the second construct can be administered as viral particles as described herein.

In certain embodiments, provided herein are kits wherein the kit comprises two or more of the components of the treatment regimen provided herein. For example, in one embodiment, such a kit comprises (i) a container with a viral particle as described herein (eg, an arenvirus-based construct comprising an open reading frame encoding an antigen of interest); and (ii) a container with a chemotherapeutic agent. In another embodiment, such a kit comprises (i) a container with a first viral particle (for “prime”); and (ii) a container with a second viral construct (for “boost”); and optionally (iii) a container with a chemotherapeutic agent.

### 6. EQUIVALENTS

The viruses, nucleic acids, methods, host cells, and compositions disclosed herein are not to be limited in scope by the specific embodiments described herein. Indeed, various modifications of the viruses, nucleic acids, methods, host cells, and compositions in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

### 7. SEQUENCES

The sequences in Table 4 are illustrative amino acid sequences and nucleotide sequences that can be used with the methods and compositions described herein. In some instances a DNA sequence is used to describe the RNA sequence of a viral genomic segment. The RNA sequence can be readily deduced from the DNA sequence.

TABLE 4

| SEQ | | |
|---|---|---|
| ID | | |
| NO. | Description | Sequence |
| 1 | Lymphocytic | GCGCACCGGGGATCCTAGGCGTTTAGTTGCGCTGTTTGGTTGCACAACT |
| | choriomeningitis | TTCTTCGTGAGGCTGTCAGAAGTGGACCTGGCTGATAGCGATGGGTCAA |
| | virus clone 13 | GGCAAGTCCAGAGAGGAGAAAGGCACCAATAGTACAAACAGGGCCGAAA |
| | segment L, complete | TCCTACCAGATACCACCTATCTTGGCCCTTTAAGCTGCAAATCTTGCTG |
| | sequence (GenBank: | GCAGAAATTTGACAGCTTGGTAAGATGCCATGACCACTACCTTTGCAGG |
| | DQ361066.1) | CACTGTTTAAACCTTCTGCTGTCAGTATCCGACAGGTGTCCTCTTTGTA |
| | (The genomic | AATATCCATTACCAACCAGATTGAAGATATCAACAGCCCCAAGCTCTCC |
| | segment is RNA, the | ACCTCCCTACGAAGAGTAACACCGTCCGGCCCCGGCCCCGACAAACAGC |
| | sequence in SEQ ID | CCAGCACAAGGGAACCGCACGTCaCCCAACGCACACAGACACAGCACCC |
| | NO: 1 is shown for | AACACAGAACACGCACACACACACACACACACACCCACACGCACGCGCC |
| | DNA; however, | CCCACCACCGGGGGGCGCCCCCCCCCGGGGGGCGGCCCCCCGGGAGCCC |
| | exchanging all | GGGCGGAGCCCCACGGAGATGCCCATCAGTCGATGTCCTCGGCCACCGA |
| | thymidines (“T”) in | CCCGCCcAGCCAATCGTCGCAGGACCTCCCCTTGAGTCTAAACCTGCCC |
| | SEQ ID NO: 1 for | CCCACTgTTTCATACATCAAAGTGCTCCTAGATTTGCTAAAACAAAGTC |
| | uridines (“U”) | TGCAATCCTTAAAGGCGAACCAGTCTGGCAAAAGCGACAGTGGAATCAG |
| | provides the RNA | CAGAATAGATCTGTCTATACATAGTTCCTGGAGGATTACACTTATCTCT |
| | sequence.) | GAACCCAACAAATGTTCACCAGTTCTGAATCGATGCAGGAAGAGGTTCC |
| | | CAAGGACATCACTAATCTTTTCATAGCCCTCAAGTCCTGCTAGAAAGAC |
| | | TTTCATGTCCTTGGTCTCCAGCTTCACAATGATATTTTGGACAAGGTTT |
| | | CTTCCTTCAAAAAGGGCACCCATCTTTACAGTCAGTGGCACAGGCTCCC |
| | | ACTCAGGTCCAACTCTCTCAAAGTCAATAGATCTAATCCCATCCAGTAT |
| | | TCTTTTGGAGCCCAACAACTCAAGCTCAAGAGAATCACCAAGTATCAAG |
| | | GGATCTTCCATGTAATCCTCAAACTCTTCAGATCTGATATCAAAGACAC |
| | | CATCGTTCACCTTGAAGACAGAGTCTGTCCTCAGTAAGTGGAGGCATTC |
| | | ATCCAACATTCTTCTATCTATCTCACCCTTAAAGAGGTGAGAGCATGAT |
| | | AAAAGTTCAGCCACACCTGGATTCTGTAATTGGCACCTAACCAAGAATA |
| | | TCAATGAAAATTTCCTTAAACAGTCAGTATTATTCTGATTGTGCGTAAA |
| | | GTCCACTGAAATTGAAAACTCCAATACCCCTTTTGTGTAGTTGAGCATG |
| | | TAGTCCCACAGATCCTTTAAGGATTTAAATGCCTTTGGGTTTGTCAGGC |
| | | CCTGCCTAATCAACATGGCAGCATTACACACAACATCTCCCATTCGGTA |
| | | AGAGAACCACCCAAAACCAAACTGCAAATCATTCCTAAACATAGGCCTC |
| | | TCCACATTTTTGTTCACCACCTTTGAGACAAATGATTGAAAGGGGCCCA |
| | | GTGCCTCAGCACCATCTTCAGATGGCATCATTTCTTTATGAGGGAACCA |
| | | TGAAAAATTGCCTAATGTCCTGGTTGTTGCAACAAATTCTCGAACAAAT |
| | | GATTCAAAATACACCTGTTTTAAGAAGTTCTTGCAGACATCCCTCGTGC |
| | | TAACAACAAATTCATCAACCAGACTGGAGTCAGATCGCTGATGAGAATT |
| | | GGCAAGGTCAGAAAACAGAACAGTGTAATGTTCATCCCTTTTCCACTTA |
| | | ACAACATGAGAAATGAGTGACAAGGATTCTGAGTTAATATCAATTAAAA |
| | | CACAGAGGTCAAGGAATTTAATTCTGGGACTCCACCTCATGTTTTTTGA |
| | | GCTCATGTCAGACATAAATGGAAGAAGCTGATCCTCAAAGATCTTGGGA |
| | | TATAGCCGCCTCACAGATTGAATCACTTGGTTCAAATTCACTTTGTCCT |
| | | CCAGTAGCCTTGAGCTCTCAGGCTTTCTTGCTACATAATCACATGGGTT |
| | | TAAGTGCTTAAGAGTTAGGTTCTCACTGTTATTCTTCCCTTTGGTCGGT |
| | | TCTGCTAGGACCCAAACACCCAACTCAAAAGAGTTGCTCAATGAAATAC |
| | | AAATGTAGTCCCAAAGAAGAGGCCTTAAAAGGCATATATGATCACGGTG |
| | | GGCTTCTGGATGAGACTGTTTGTCACAAATGTACAGCGTTATACCATCC |
| | | CGATTGCAAACTCTTGTCACATGATCATCTGTGGTTAGATCCTCAAGCA |
| | | GCTTTTTGATATACAGATTTTCCCTATTTTTGTTTCTCACACACCTGCT |
| | | TCCTAGAGTTTTGCAAAGGCCTATAAAGCCAGATGAGATACAACTCTGG |
| | | AAAGCTGACTTGTTGATTGCTTCTGACAGCAGCTTCTGTGCACCCCTTG |
| | | TGAATTTACTACAAAGTTTGTTCTGGAGTGTCTTGATCAATGATGGGAT |
| | | TCTTTCCTCTTGGAAAGTCATCACTGATGGATAAACCACCTTTTGTCTT |
| | | AAAACCATCCTTAATGGGAACATTTCATTCAAATTCAACCAGTTAACAT |
| | | CTGCTAACTGATTCAGATCTTCTTCAAGACCGAGGAGGTCTCCCAATTG |
| | | AAGAATGGCCTCCtTTTTATCTCTGTTAAATAGGTCTAAGAAAAATTCT |
| | | TCATTAAATTCACCATTTTTGAGCTTATGATGCAGTTTCCTTACAAGCT |
| | | TTCTTACAACCTTTGTTTCATTAGGACACAGTTCCTCAATGAGTCTTTG |
| | | TATTCTGTAACCTCTAGAACCATCCAGCCAATCTTTCACATCAGTGTTG |
| | | GTATTCAGTAGAAATGGATCCAAAGGGAAATTGGCATACTTTAGGAGGT |
| | | CCAGTGTTCTCCTTTGGATACTATTAACTAGGGAGACTGGGACGCCATT |
| | | TGCGATGGCTTGATCTGCAATTGTATCTATTGTTTCACAAAGTTGATGT |
| | | GGCTCTTTACACTTGACATTGTGTAGCGCTGCAGATACAAACTTTGTGA |
| | | GAAGAGGGACTTCCTCCCCCCATACATAGAATCTAGATTTAAATTCTGC |
| | | AGCGAACCTCCCAGCCACACTTTTTGGGCTGATAAATTTGTTTAACAAG |
| | | CCGCTCAGATGAGATTGGAATTCCAACAGGACAAGGACTTCCTCCGGAT |
| | | CACTTACAACCAGGTCACTCAGCCTCCTATCAAATAAAGTGATCTGATC |
| | | ATCACTTGATGTGTAAGCCTCTGGTCTTTCGCCAAAGATAACACCAATG |
| | | CAGTAGTTGATGAACCTCTCGCTAAGCAAACCATAGAAGTCAGAAGCAT |
| | | TATGCAAGATTCCCTGCCCCATATCAATAAGGCTGGATATATGGGATGG |
| | | CACTATCCCCATTTCAAAATATTGTCTGAAAATTCTCTCAGTAACAGTT |
| | | GTTTCTGAACCCCTGAGAAGTTTTAGCTTCGACTTGACATATGATTTCA |
| | | TCATTGCATTCACAACAGGAAAGGGGACCTCGACAAGCTTATGCATGTG |
| | | CCAAGTTAACAAAGTGCTAACATGATCTTTCCCGGAACGCACATACTGG |
| | | TCATCACCTAGTTTGAGATTTTGTAGAAACATTAAGAACAAAAATGGGC |
| | | ACATCATTGGTCCCCATTTGCTGTGATCCATACTATAGTTTAAGAACCC |
| | | TTCCCGCACATTGATAGTCATTGACAAGATTGCATTTTCAAATTCCTTA |
| | | TCATTGTTTAAACAGGAGCCTGAAAAGAAACTTGAAAAAGACTCAAAAT |
| | | AATCTTCTATTAACCTTGTGAACATTTTTGTCCTCAAATCTCCAATATA |
| | | GAGTTCTCTATTTCCCCCAACCTGCTCTTTATAAGATAGTGCAAATTTC |
| | | AGCCTTCCAGAGTCAGGACCTACTGAGGTGTATGATGTTGGTGATTCTT |
| | | CTGAGTAGAAGCACAGATTTTTCAAAGCAGCACTCATACATTgTGTCAA |
| | | CGACAGAGCTTTACTAAGGGACTCAGAATTACTTTCCCTCTCACTGATT |
| | | CTCACGTCTTCTTCCAGTTTGTCCCAGTCAAATTTGAAATTCAAGCCTT |
| | | GCCTTTGCATATGCCTGTATTTCCCTGAGTACGCATTTGCATTCATTTG |
| | | CAACAGAATCATCTTCATGCAAGAAAACCAATCATTCTCAGAAAAGAAC |
| | | TTTCTACAAAGGTTTTTTGCCATCTCATCGAGGCCACACTGATCTTTAA |
| | | TGACTGAGGTGAAATACAAAGGTGACAGCTCTGTGGAACCCTCAACAGC |
| | | CTCACAGATAAATTTCATGTCATCATTGGTTAGACATGATGGGTCAAAG |
| | | TCTTCTACTAAATGGAAAGATATTTCTGACAAGATAACTTTTCTTAAGT |
| | | GAGCCATCTTCCCTGTTAGAATAAGCTGTAAATGATGTAGTCCTTTTGT |
| | | ATTTGTAAGTTTTTCTCCATCTCCTTTGTCATTGGCCCTCCTACCTCTT |
| | | CTGTACCGTGCTATTGTGGTGTTGACCTTTTCTTCGAGACTTTTGAAGA |
| | | AGCTTGTCTCTTCTTCTCCATCAAAACATATTTCTGCCAGGTTGTCTTC |
| | | CGATCTCCCTGTCTCTTCTCCCTTGGAACCGATGACCAATCTAGAGACT |
| | | AACTTGGAAACTTTATATTCATAGTCTGAGTGGCTCAACTTATACTTTT |
| | | GTTTTCTTACGAAACTCTCCGTAATTTGACTCACAGCACTAACAAGCAA |
| | | TTTGTTAAAGTCATATTCCAGAAGTCGTTCTCCATTTAGATGCTTATTA |
| | | ACCACCACACTTTTGTTACTAGCAAGATCTAATGCTGTCGCACATCCAG |
| | | AGTTAGTCATGGGATCTAGGCTGTTTAGCTTCTTCTCTCCTTTGAAAAT |
| | | TAAAGTGCCGTTGTTAAATGAAGACACCATTAGGCTAAAGGCTTCCAGA |
| | | TTAACACCTGGAGTTGTATGCTGACAGTCAATTTCTTTACTAGTGAATC |
| | | TCTTCATTTGCTCATAGAACACACATTCTTCCTCAGGAGTGATTGCTTC |
| | | CTTGGGGTTGACAAAAAAACCAAATTGACTTTTGGGCTCAAAGAACTTT |
| | | TCAAAACATTTTATCTGATCTGTTAGCCTGTCAGGGGTCTCCTTTGTGA |
| | | TCAAATGACACAGGTATGACACATTCAACATAAATTTAAATTTTGCACT |
| | | CAACAACACCTTCTCACCAGTACCAAAAATAGTTTTTATTAGGAATCTA |
| | | AGCAGCTTATACACCACCTTCTCAGCAGGTGTGATCAGATCCTCCCTCA |
| | | ACTTATCCATTAATGATGTAGATGAAAAATCTGACACTATTGCCATCAC |
| | | CAAATATCTGACACTCTGTACCTGCTTTTGATTTCTCTTTGTTGGGTTG |
| | | GTGAGCATTAGCAACAATAGGGTCCTCAGTGCAACCTCAATGTCGGTGA |
| | | GACAGTCTTTCAAATCAGGACATGATCTAATCCATGAAATCATGATGTC |
| | | TATCATATTGTATAAGACCTCATCTGAAAAAATTGGTAAAAAGAACCTT |
| | | TTAGGATCTGCATAGAAGGAAATTAAATGACCATCCGGGCCTTGTATGG |
| | | AGTAGCACCTTGAAGATTCTCCAGTCTTCTGGTATAATAGGTGGTATTC |
| | | TTCAGAGTCCAGTTTTATTACTTGGCAAAACACTTCTTTGCATTCTACC |
| | | ACTTGATATCTCACAGACCCTATTTGATTTTGCCTTAGTCTAGCAACTG |
| | | AGCTAGTTTTCATACTGTTTGTTAAGGCCAGACAAACAGATGATAATCT |
| | | TCTCAGGCTCTGTATGTTCTTCAGCTGCTCTGTGCTGGGTTGGAAATTG |
| | | TAATCTTCAAACTTCGTATAATACATTATCGGGTGAGCTCCAATTTTCA |
| | | TAAAGTTCTCAAATTCAGTGAATGGTATGTGGCATTCTTGCTCAAGGTG |
| | | TTCAGACAGTCCGTAATGCTCGAAACTCAGTCCCACCACTAACAGGCAT |
| | | TTTTGAATTTTTGCAATGAACTCACTAATAGAtGCCCTAAACAATTCCT |
| | | CAAAAGACACCTTTCTAAACACCTTTGACTTTTTTCTATTCCTCAAAAG |
| | | TCTAATGAACTCCTCTTTAGTGCTGTGAAAGCTTACCAGCCTATCATTC |
| | | ACACTACTATAGCAACAACCCACCCAGTGTTTATCATTTTTTAACCCTT |
| | | TGAATTTCGACTGTTTTATCAATGAGGAAAGACACAAAACATCCAGATT |
| | | TAACAACTGTCTCCTTCTAGTATTCAACAGTTTCAAACTCTTGACTTTG |
| | | TTTAACATAGAGAGGAGCCTCTCATATTCAGTGCTAGTCTCACTTCCCC |
| | | TTTCGTGCCCATGGGTCTCTGCAGTTATGAATCTCATCAAAGGACAGGA |
| | | TTCGACTGCCTCCCTGCTTAATGTTAAGATATCATCACTATCAGCAAGG |
| | | TTTTCATAGAGCTCAGAGAATTCCTTGATCAAGCCTTCAGGGTTTACTT |
| | | TCTGAAAGTTTCTCTTTAATTTCCCACTTTCTAAATCTCTTCTAAACCT |
| | | GCTGAAAAGAGAGTTTATTCCAAAAACCACATCATCACAGCTCATGTTG |
| | | GGGTTGATGCCTTCGTGGCACATCCTCATAATTTCATCATTGTGAGTTG |
| | | ACCTCGCATCTTTCAGAATTTTCATAGAGTCCATACCGGAGCGCTTGTC |
| | | GATAGTAGTCTTCAGGGACTCACAGAGTCTAAAATATTCAGACTCTTCA |
| | | AAGACTTTCTCATTTTGGTTAGAATACTCCAAAAGTTTGAATAAAAGGT |
| | | CTCTAAATTTGAAGTTTGCCCACTCTGGCATAAAACTATTATCATAATC |
| | | ACAACGACCATCTACTATTGGAACTAATGTGACACCCGCAACAGCAAGG |
| | | TCTTCCCTGATGCATGCCAATTTGTTAGTGTCCTCTATAAATTTCTTCT |
| | | CAAAACTGGCTGGaGtGCTCCTAACAAAACACTCAAGAAGAATGAGAGA |
| | | ATTGTCTATCAGCTTGTAACCATCAGGAATGATAAGTGGTAGTCCTGGG |
| | | CATACAATTCCAGACTCCACCAAAATTGTTTCCACAGACTTATCGTCGT |
| | | GGTTGTGTGTGCAGCCACTCTTGTCTGCACTGTCTATTTCAATGCAGCG |
| | | TGACAGCAACTTGAGTCCCTCAATCAGAACCATTCTGGGTTCCCTTTGT |
| | | CCCAGAAAGTTGAGTTTCTGCCTTGACAACCTCTCATCCTGTTCTATAT |
| | | AGTTTAAACATAACTCTCTCAATTCTGAGATGATTTCATCCATTGCGCA |
| | | TCAAAAAGCCTAGGATCCTCGGTGCG |
| 2 | Lymphocytic | CGCACCGGGGATCCTAGGCTTTTTGGATTGCGCTTTCCTC |
| | choriomeningitis | TAGATCAACTGGGTGTCAGGCCCTATCCTACAGAAGGATG |
| | virus segment S, | GGTCAGATTGTGACAATGTTTGAGGCTCTGCCTCACATCA |
| | complete sequence | TCGATGAGGTGATCAACATTGTCATTATTGTGCTTATCGT |
| | (The genomic | GATCACGGGTATCAAGGCTGTCTACAATTTTGCCACCTGT |
| | segment is RNA, the | GGGATATTCGCATTGATCAGTTTCCTACTTCTGGCTGGCA |
| | sequence in SEQ ID | GGTCCTGTGGCATGTACGGTCTTAAGGGACCCGACATTTA |
| | NO: 2 is shown for | CAAAGGAGTTTACCAATTTAAGTCAGTGGAGTTTGATATG |
| | DNA; however, | TCACATCTGAACCTGACCATGCCCAACGCATGTTCAGCCA |
| | exchanging all | ACAACTCCCACCATTACATCAGTATGGGGACTTCTGGACT |
| | thymidines (“T”) in | AGAATTGACCTTCACCAATGATTCCATCATCAGTCACAAC |
| | SEQ ID NO: 2 for | TTTTGCAATCTGACCTCTGCCTTCAACAAAAAGACCTTTG |
| | uridines (“U”) | ACCACACACTCATGAGTATAGTTTCGAGCCTACACCTCAG |
| | provides the RNA | TATCAGAGGGAACTCCAACTATAAGGCAGTATCCTGCGAC |
| | sequence.) | TTCAACAATGGCATAACCATCCAATACAACTTGACATTCT |
| | | CAGATCGACAAAGTGCTCAGAGCCAGTGTAGAACCTTCAG |
| | | AGGTAGAGTCCTAGATATGTTTAGAACTGCCTTCGGGGGG |
| | | AAATACATGAGGAGTGGCTGGGGCTGGACAGGCTCAGATG |
| | | GCAAGACCACCTGGTGTAGCCAGACGAGTTACCAATACCT |
| | | GATTATACAAAATAGAACCTGGGAAAACCACTGCACATAT |
| | | GCAGGTCCTTTTGGGATGTCCAGGATTCTCCTTTCCCAAG |
| | | AGAAGACTAAGTTCTTCACTAGGAGACTAGCGGGCACATT |
| | | CACCTGGACTTTGTCAGACTCTTCAGGGGTGGAGAATCCA |
| | | GGTGGTTATTGCCTGACCAAATGGATGATTCTTGCTGCAG |
| | | AGCTTAAGTGTTTCGGGAACACAGCAGTTGCGAAATGCAA |
| | | TGTAAATCATGATGCCGAATTCTGTGACATGCTGCGACTA |
| | | ATTGACTACAACAAGGCTGCTTTGAGTAAGTTCAAAGAGG |
| | | ACGTAGAATCTGCCTTGCACTTATTCAAAACAACAGTGAA |
| | | TTCTTTGATTTCAGATCAACTACTGATGAGGAACCACTTG |
| | | AGAGATCTGATGGGGGTGCCATATTGCAATTACTCAAAGT |
| | | TTTGGTACCTAGAACATGCAAAGACCGGCGAAACTAGTGT |
| | | CCCCAAGTGCTGGCTTGTCACCAATGGTTCTTACTTAAAT |
| | | GAGACCCACTTCAGTGATCAAATCGAACAGGAAGCCGATA |
| | | ACATGATTACAGAGATGTTGAGGAAGGATTACATAAAGAG |
| | | GCAGGGGAGTACCCCCCTAGCATTGATGGACCTTCTGATG |
| | | TTTTCCACATCTGCATATCTAGTCAGCATCTTCCTGCACC |
| | | TTGTCAAAATACCAACACACAGGCACATAAAAGGTGGCTC |
| | | ATGTCCAAAGCCACACCGATTAACCAACAAAGGAATTTGT |
| | | AGTTGTGGTGCATTTAAGGTGCCTGGTGTAAAAACCGTCT |
| | | GGAAAAGACGCTGAAGAACAGCGCCTCCCTGACTCTCCAC |
| | | CTCGAAAGAGGTGGAGAGTCAGGGAGGCCCAGAGGGTCTT |
| | | AGAGTGTCACAACATTTGGGCCTCTAAAAATTAGGTCATG |
| | | TGGCAGAATGTTGTGAACAGTTTTCAGATCTGGGAGCCTT |
| | | GCTTTGGAGGCGCTTTCAAAAATGATGCAGTCCATGAGTG |
| | | CACAGTGCGGGGTGATCTCTTTCTTCTTTTTGTCCCTTAC |
| | | TATTCCAGTATGCATCTTACACAACCAGCCATATTTGTCC |
| | | CACACTTTGTCTTCATACTCCCTCGAAGCTTCCCTGGTCA |
| | | TTTCAACATCGATAAGCTTAATGTCCTTCCTATTCTGTGA |
| | | GTCCAGAAGCTTTCTGATGTCATCGGAGCCTTGACAGCTT |
| | | AGAACCATCCCCTGCGGAAGAGCACCTATAACTGACGAGG |
| | | TCAACCCGGGTTGCGCATTGAAGAGGTCGGCAAGATCCAT |
| | | GCCGTGTGAGTACTTGGAATCTTGCTTGAATTGTTTTTGA |
| | | TCAACGGGTTCCCTGTAAAAGTGTATGAACTGCCCGTTCT |
| | | GTGGTTGGAAAATTGCTATTTCCACTGGATCATTAAATCT |
| | | ACCCTCAATGTCAATCCATGTAGGAGCGTTGGGGTCAATT |
| | | CCTCCCATGAGGTCTTTTAAAAGCATTGTCTGGCTGTAGC |
| | | TTAAGCCCACCTGAGGTGGACCTGCTGCTCCAGGCGCTGG |
| | | CCTGGGTGAATTGACTGCAGGTTTCTCGCTTGTGAGATCA |
| | | ATTGTTGTGTTTTCCCATGCTCTCCCCACAATCGATGTTC |
| | | TACAAGCTATGTATGGCCATCCTTCACCTGAAAGGCAAAC |
| | | TTTATAGAGGATGTTTTCATAAGGGTTCCTGTCCCCAACT |
| | | TGGTCTGAAACAAACATGTTGAGTTTTCTCTTGGCCCCGA |
| | | GAACTGCCTTCAAGAGGTCCTCGCTGTTGCTTGGCTTGAT |
| | | CAAAATTGACTCTAACATGTTACCCCCATCCAACAGGGCT |
| | | GCCCCTGCCTTCACGGCAGCACCAAGACTAAAGTTATAGC |
| | | CAGAAATGTTGATGCTGGACTGCTGTTCAGTGATGACCCC |
| | | CAGAACTGGGTGCTTGTCTTTCAGCCTTTCAAGATCATTA |
| | | AGATTTGGATACTTGACTGTGTAAAGCAAGCCAAGGTCTG |
| | | TGAGCGCTTGTACAACGTCATTGAGCGGAGTCTGTGACTG |
| | | TTTGGCCATACAAGCCATAGTTAGACTTGGCATTGTGCCA |
| | | AATTGATTGTTCAAAAGTGATGAGTCTTTCACATCCCAAA |
| | | CTCTTACCACACCACTTGCACCCTGCTGAGGCTTTCTCAT |
| | | CCCAACTATCTGTAGGATCTGAGATCTTTGGTCTAGTTGC |
| | | TGTGTTGTTAAGTTCCCCATATATACCCCTGAAGCCTGGG |
| | | GCCTTTCAGACCTCATGATCTTGGCCTTCAGCTTCTCAAG |
| | | GTCAGCCGCAAGAGACATCAGTTCTTCTGCACTGAGCCTC |
| | | CCCACTTTCAAAACATTCTTCTTTGATGTTGACTTTAAAT |
| | | CCACAAGAGAATGTACAGTCTGGTTGAGACTTCTGAGTCT |
| | | CTGTAGGTCTTTGTCATCTCTCTTTTCCTTCCTCATGATC |
| | | CTCTGAACATTGCTGACCTCAGAGAAGTCCAACCCATTCA |
| | | GAAGGTTGGTTGCATCCTTAATGACAGCAGCCTTCACATC |
| | | TGATGTGAAGCTCTGCAATTCTCTTCTCAATGCTTGCGTC |
| | | CATTGGAAGCTCTTAACTTCCTTAGACAAGGACATCTTGT |
| | | TGCTCAATGGTTTCTCAAGACAAATGCGCAATCAAATGCC |
| | | TAGGATCCACTGTGCG |
| 3 | Lymphocytic | GCGCACCGGGGATCCTAGGCTTTTTGGATTGCGCTTTCCT |
| | choriomeningitis | CTAGATCAACTGGGTGTCAGGCCCTATCCTACAGAAGGAT |
| | virus clone 13 | GGGTCAGATTGTGACAATGTTTGAGGCTCTGCCTCACATC |
| | segment S, complete | ATCGATGAGGTGATCAACATTGTCATTATTGTGCTTATCG |
| | sequence (GenBank: | TGATCACGGGTATCAAGGCTGTCTACAATTTTGCCACCTG |
| | DQ361065.2) | TGGGATATTCGCATTGATCAGTTTCCTACTTCTGGCTGGC |
| | (The genomic | AGGTCCTGTGGCATGTACGGTCTTAAGGGACCCGACATTT |
| | segment is RNA, the | ACAAAGGAGTTTACCAATTTAAGTCAGTGGAGTTTGATAT |
| | sequence in SEQ ID | GTCACATCTGAACCTGACCATGCCCAACGCATGTTCAGCC |
| | NO: 3 is shown for | AACAACTCCCACCATTACATCAGTATGGGGACTTCTGGAC |
| | DNA; however, | TAGAATTGACCTTCACCAATGATTCCATCATCAGTCACAA |
| | exchanging all | CTTTTGCAATCTGACCTCTGCCTTCAACAAAAAGACCTTT |
| | thymidines (“T”) in | GACCACACACTCATGAGTATAGTTTCGAGCCTACACCTCA |
| | SEQ ID NO: 3 for | GTATCAGAGGGAACTCCAACTATAAGGCAGTATCCTGCGA |
| | uridines (“U”) | CTTCAACAATGGCATAACCATCCAATACAACTTGACATTC |
| | provides the RNA | TCAGATGCACAAAGTGCTCAGAGCCAGTGTAGAACCTTCA |
| | sequence.) | GAGGTAGAGTCCTAGATATGTTTAGAACTGCCTTCGGGGG |
| | | GAAATACATGAGGAGTGGCTGGGGCTGGACAGGCTCAGAT |
| | | GGCAAGACCACCTGGTGTAGCCAGACGAGTTACCAATACC |
| | | TGATTATACAAAATAGAACCTGGGAAAACCACTGCACATA |
| | | TGCAGGTCCTTTTGGGATGTCCAGGATTCTCCTTTCCCAA |
| | | GAGAAGACTAAGTTCCTCACTAGGAGACTAGCGGGCACAT |
| | | TCACCTGGACTTTGTCAGACTCTTCAGGGGTGGAGAATCC |
| | | AGGTGGTTATTGCCTGACCAAATGGATGATTCTTGCTGCA |
| | | GAGCTTAAGTGTTTCGGGAACACAGCAGTTGCGAAATGCA |
| | | ATGTAAATCATGATGAAGAATTCTGTGACATGCTGCGACT |
| | | AATTGACTACAACAAGGCTGCTTTGAGTAAGTTCAAAGAG |
| | | GACGTAGAATCTGCCTTGCACTTATTCAAAACAACAGTGA |
| | | ATTCTTTGATTTCAGATCAACTACTGATGAGGAACCACTT |
| | | GAGAGATCTGATGGGGGTGCCATATTGCAATTACTCAAAG |
| | | TTTTGGTACCTAGAACATGCAAAGACCGGCGAAACTAGTG |
| | | TCCCCAAGTGCTGGCTTGTCACCAATGGTTCTTACTTAAA |
| | | TGAGACCCACTTCAGTGACCAAATCGAACAGGAAGCCGAT |
| | | AACATGATTACAGAGATGTTGAGGAAGGATTACATAAAGA |
| | | GGCAGGGGAGTACCCCCCTAGCATTGATGGACCTTCTGAT |
| | | GTTTTCCACATCTGCATATCTAGTCAGCATCTTCCTGCAC |
| | | CTTGTCAAAATACCAACACACAGGCACATAAAAGGTGGCT |
| | | CATGTCCAAAGCCACACCGATTAACCAACAAAGGAATTTG |
| | | TAGTTGTGGTGCATTTAAGGTGCCTGGTGTAAAAACCGTC |
| | | TGGAAAAGACGCTGAAGAACAGCGCCTCCCTGACTCTCCA |
| | | CCTCGAAAGAGGTGGAGAGTCAGGGAGGCCCAGAGGGTCT |
| | | TAGAGTGTCACAACATTTGGGCCTCTAAAAATTAGGTCAT |
| | | GTGGCAGAATGTTGTGAACAGTTTTCAGATCTGGGAGCCT |
| | | TGCTTTGGAGGCGCTTTCAAAAATGATGCAGTCCATGAGT |
| | | GCACAGTGCGGGGTGATCTCTTTCTTCTTTTTGTCCCTTA |
| | | CTATTCCAGTATGCATCTTACACAACCAGCCATATTTGTC |
| | | CCACACTTTGTCTTCATACTCCCTCGAAGCTTCCCTGGTC |
| | | ATTTCAACATCGATAAGCTTAATGTCCTTCCTATTCTGTG |
| | | AGTCCAGAAGCTTTCTGATGTCATCGGAGCCTTGACAGCT |
| | | TAGAACCATCCCCTGCGGAAGAGCACCTATAACTGACGAG |
| | | GTCAACCCGGGTTGCGCATTGAAGAGGTCGGCAAGATCCA |
| | | TGCCGTGTGAGTACTTGGAATCTTGCTTGAATTGTTTTTG |
| | | ATCAACGGGTTCCCTGTAAAAGTGTATGAACTGCCCGTTC |
| | | TGTGGTTGGAAAATTGCTATTTCCACTGGATCATTAAATC |
| | | TACCCTCAATGTCAATCCATGTAGGAGCGTTGGGGTCAAT |
| | | TCCTCCCATGAGGTCTTTTAAAAGCATTGTCTGGCTGTAG |
| | | CTTAAGCCCACCTGAGGTGGACCTGCTGCTCCAGGCGCTG |
| | | GCCTGGGTGAATTGACTGCAGGTTTCTCGCTTGTGAGATC |
| | | AATTGTTGTGTTTTCCCATGCTCTCCCCACAATCGATGTT |
| | | CTACAAGCTATGTATGGCCATCCTTCACCTGAAAGGCAAA |
| | | CTTTATAGAGGATGTTTTCATAAGGGTTCCTGTCCCCAAC |
| | | TTGGTCTGAAACAAACATGTTGAGTTTTCTCTTGGCCCCG |
| | | AGAACTGCCTTCAAGAGGTCCTCGCTGTTGCTTGGCTTGA |
| | | TCAAAATTGACTCTAACATGTTACCCCCATCCAACAGGGC |
| | | TGCCCCTGCCTTCACGGCAGCACCAAGACTAAAGTTATAG |
| | | CCAGAAATGTTGATGCTGGACTGCTGTTCAGTGATGACCC |
| | | CCAGAACTGGGTGCTTGTCTTTCAGCCTTTCAAGATCATT |
| | | AAGATTTGGATACTTGACTGTGTAAAGCAAGCCAAGGTCT |
| | | GTGAGCGCTTGTACAACGTCATTGAGCGGAGTCTGTGACT |
| | | GTTTGGCCATACAAGCCATAGTTAGACTTGGCATTGTGCC |
| | | AAATTGATTGTTCAAAAGTGATGAGTCTTTCACATCCCAA |
| | | ACTCTTACCACACCACTTGCACCCTGCTGAGGCTTTCTCA |
| | | TCCCAACTATCTGTAGGATCTGAGATCTTTGGTCTAGTTG |
| | | CTGTGTTGTTAAGTTCCCCATATATACCCCTGAAGCCTGG |
| | | GGCCTTTCAGACCTCATGATCTTGGCCTTCAGCTTCTCAA |
| | | GGTCAGCCGCAAGAGACATCAGTTCTTCTGCACTGAGCCT |
| | | CCCCACTTTCAAAACATTCTTCTTTGATGTTGACTTTAAA |
| | | TCCACAAGAGAATGTACAGTCTGGTTGAGACTTCTGAGTC |
| | | TCTGTAGGTCTTTGTCATCTCTCTTTTCCTTCCTCATGAT |
| | | CCTCTGAACATTGCTGACCTCAGAGAAGTCCAACCCATTC |
| | | AGAAGGTTGGTTGCATCCTTAATGACAGCAGCCTTCACAT |
| | | CTGATGTGAAGCTCTGCAATTCTCTTCTCAATGCTTGCGT |
| | | CCATTGGAAGCTCTTAACTTCCTTAGACAAGGACATCTTG |
| | | TTGCTCAATGGTTTCTCAAGACAAATGCGCAATCAAATGC |
| | | CTAGGATCCACTGTGCG |
| 4 | Lymphocytic | GCGCACCGGGGATCCTAGGCATTTTTGTTGCGCATTTTGT |
| | choriomeningitis | TGTGTTATTTGTTGCACAGCCCTTCATCGTGGGACCTTCA |
| | strain MP segment | CAAACAAACCAAACCACCAGCCATGGGCCAAGGCAAGTCC |
| | L, complete | AAAGAGGGAAGGGATGCCAGCAATACGAGCAGAGCTGAAA |
| | sequence | TTCTGCCAGACACCACCTATCTCGGACCTCTGAACTGCAA |
| | (The genomic | GTCATGCTGGCAGAGATTTGACAGTTTAGTCAGATGCCAT |
| | segment is RNA, the | GACCACTATCTCTGCAGACACTGCCTGAACCTCCTGCTGT |
| | sequence in SEQ ID | CAGTCTCCGACAGGTGCCCTCTCTGCAAACATCCATTGCC |
| | NO: 4 is shown for | AACCAAACTGAAAATATCCACGGCCCCAAGCTCTCCACCC |
| | DNA; however, | CCTTACGAGGAGTGACGCCCCGAGCCCCAACACCGACACA |
| | exchanging all | AGGAGGCCACCAACACAACGCCCAACACGGAACACACACA |
| | thymidines (“T”) in | CACACACCCACACACACATCCACACACACGCGCCCCCACA |
| | SEQ ID NO: 4 for | ACGGGGGCGCCCCCCCGGGGGTGGCCCCCCGGGTGCTCGG |
| | uridines (“U”) | GCGGAGCCCCACGGAGAGGCCAATTAGTCGATCTCCTCGA |
| | provides the RNA | CCACCGACTTGGTCAGCCAGTCATCACAGGACTTGCCCTT |
| | sequence.) | AAGTCTGTACTTGCCCACAACTGTTTCATACATCACCGTG |
| | | TTCTTTGACTTACTGAAACATAGCCTACAGTCTTTGAAAG |
| | | TGAACCAGTCAGGCACAAGTGACAGCGGTACCAGTAGAAT |
| | | GGATCTATCTATACACAACTCTTGGAGAATTGTGCTAATT |
| | | TCCGACCCCTGTAGATGCTCACCAGTTCTGAATCGATGTA |
| | | GAAGAAGGCTCCCAAGGACGTCATCAAAATTTCCATAACC |
| | | CTCGAGCTCTGCCAAGAAAACTCTCATATCCTTGGTCTCC |
| | | AGTTTCACAACGATGTTCTGAACAAGGCTTCTTCCCTCAA |
| | | AAAGAGCACCCATTCTCACAGTCAAGGGCACAGGCTCCCA |
| | | TTCAGGCCCAATCCTCTCAAAATCAAGGGATCTGATCCCG |
| | | TCCAGTATTTTCCTTGAGCCTATCAGCTCAAGCTCAAGAG |
| | | AGTCACCGAGTATCAGGGGGTCCTCCATATAGTCCTCAAA |
| | | CTCTTCAGACCTAATGTCAAAAACACCATCGTTCACCTTG |
| | | AAGATAGAGTCTGATCTCAACAGGTGGAGGCATTCGTCCA |
| | | AGAACCTTCTGTCCACCTCACCTTTAAAGAGGTGAGAGCA |
| | | TGATAGGAACTCAGCTACACCTGGACCTTGTAACTGGCAC |
| | | TTCACTAAAAAGATCAATGAAAACTTCCTCAAACAATCAG |
| | | TGTTATTCTGGTTGTGAGTGAAATCTACTGTAATTGAGAA |
| | | CTCTAGCACTCCCTCTGTATTATTTATCATGTAATCCCAC |
| | | AAGTTTCTCAAAGACTTGAATGCCTTTGGATTTGTCAAGC |
| | | CTTGTTTGATTAGCATGGCAGCATTGCACACAATATCTCC |
| | | CAATCGGTAAGAGAACCATCCAAATCCAAATTGCAAGTCA |
| | | TTCCTAAACATGGGCCTCTCCATATTTTTGTTCACTACTT |
| | | TTAAGATGAATGATTGGAAAGGCCCCAATGCTTCAGCGCC |
| | | ATCTTCAGATGGCATCATGTCTTTATGAGGGAACCATGAA |
| | | AAACTTCCTAGAGTTCTGCTTGTTGCTACAAATTCTCGTA |
| | | CAAATGACTCAAAATACACTTGTTTTAAAAAGTTTTTGCA |
| | | GACATCCCTTGTACTAACGACAAATTCATCAACAAGGCTT |
| | | GAGTCAGAGCGCTGATGGGAATTTACAAGATCAGAAAATA |
| | | GAACAGTGTAGTGTTCGTCCCTCTTCCACTTAACTACATG |
| | | AGAAATGAGCGATAAAGATTCTGAATTGATATCGATCAAT |
| | | ACGCAAAGGTCAAGGAATTTGATTCTGGGACTCCATCTCA |
| | | TGTTTTTTGAGCTCATATCAGACATGAAGGGAAGCAGCTG |
| | | ATCTTCATAGATTTTAGGGTACAATCGCCTCACAGATTGG |
| | | ATTACATGGTTTAAACTTATCTTGTCCTCCAGTAGCCTTG |
| | | AACTCTCAGGCTTCCTTGCTACATAATCACATGGGTTCAA |
| | | GTGCTTGAGGCTTGAGCTTCCCTCATTCTTCCCTTTCACA |
| | | GGTTCAGCTAAGACCCAAACACCCAACTCAAAGGAATTAC |
| | | TCAGTGAGATGCAAATATAGTCCCAAAGGAGGGGCCTCAA |
| | | GAGACTGATGTGGTCGCAGTGAGCTTCTGGATGACTTTGC |
| | | CTGTCACAAATGTACAACATTATGCCATCATGTCTGTGGA |
| | | TTGCTGTCACATGCGCATCCATAGCTAGATCCTCAAGCAC |
| | | TTTTCTAATGTATAGATTGTCCCTATTTTTATTTCTCACA |
| | | CATCTACTTCCCAAAGTTTTGCAAAGACCTATAAAGCCTG |
| | | ATGAGATGCAACTTTGAAAGGCTGACTTATTGATTGCTTC |
| | | TGACAGCAACTTCTGTGCACCTCTTGTGAACTTACTGCAG |
| | | AGCTTGTTCTGGAGTGTCTTGATTAATGATGGGATTCTTT |
| | | CCTCTTGGAAAGTCATTACTGATGGATAAACCACTTTCTG |
| | | CCTCAAGACCATTCTTAATGGGAACAACTCATTCAAATTC |
| | | AGCCAATTTATGTTTGCCAATTGACTTAGATCCTCTTCGA |
| | | GGCCAAGGATGTTTCCCAACTGAAGAATGGCTTCCTTTTT |
| | | ATCCCTATTGAAGAGGTCTAAGAAGAATTCTTCATTGAAC |
| | | TCACCATTCTTGAGCTTATGATGTAGTCTCCTTACAAGCC |
| | | TTCTCATGACCTTCGTTTCACTAGGACACAATTCTTCAAT |
| | | AAGCCTTTGGATTCTGTAACCTCTAGAGCCATCCAACCAA |
| | | TCCTTGACATCAGTATTAGTGTTAAGCAAAAATGGGTCCA |
| | | AGGGAAAGTTGGCATATTTTAAGAGGTCTAATGTTCTCTT |
| | | CTGGATGCAGTTTACCAATGAAACTGGAACACCATTTGCA |
| | | ACAGCTTGATCGGCAATTGTATCTATTGTTTCACAGAGTT |
| | | GGTGTGGCTCTTTACACTTAACGTTGTGTAATGCTGCTGA |
| | | CACAAATTTTGTTAAAAGTGGGACCTCTTCCCCCCACACA |
| | | TAAAATCTGGATTTAAATTCTGCAGCAAATCGCCCCACCA |
| | | CACTTTTCGGACTGATGAACTTGTTAAGCAAGCCACTCAA |
| | | ATGAGAATGAAATTCCAGCAATACAAGGACTTCCTCAGGG |
| | | TCACTATCAACCAGTTCACTCAATCTCCTATCAAATAAGG |
| | | TGATCTGATCATCACTTGATGTGTAAGATTCTGGTCTCTC |
| | | ACCAAAAATGACACCGATACAATAATTAATGAATCTCTCA |
| | | CTGATTAAGCCGTAAAAGTCAGAGGCATTATGTAAGATTC |
| | | CCTGTCCCATGTCAATGAGACTGCTTATATGGGAAGGCAC |
| | | TATTCCTAATTCAAAATATTCTCGAAAGATTCTTTCAGTC |
| | | ACAGTTGTCTCTGAACCCCTAAGAAGTTTCAGCTTTGATT |
| | | TGATATATGATTTCATCATTGCATTCACAACAGGAAAAGG |
| | | GACCTCAACAAGTTTGTGCATGTGCCAAGTTAATAAGGTG |
| | | CTGATATGATCCTTTCCGGAACGCACATACTGGTCATCAC |
| | | CCAGTTTGAGATTTTGAAGGAGCATTAAAAACAAAAATGG |
| | | GCACATCATTGGCCCCCATTTGCTATGATCCATACTGTAG |
| | | TTCAACAACCCCTCTCGCACATTGATGGTCATTGATAGAA |
| | | TTGCATTTTCAAATTCTTTGTCATTGTTTAAGCATGAACC |
| | | TGAGAAGAAGCTAGAAAAAGACTCAAAATAATCCTCTATC |
| | | AATCTTGTAAACATTTTTGTTCTCAAATCCCCAATATAAA |
| | | GTTCTCTGTTTCCTCCAACCTGCTCTTTGTATGATAACGC |
| | | AAACTTCAACCTTCCGGAATCAGGACCAACTGAAGTGTAT |
| | | GACGTTGGTGACTCCTCTGAGTAAAAACATAAATTCTTTA |
| | | AAGCAGCACTCATGCATTTTGTCAATGATAGAGCCTTACT |
| | | TAGAGACTCAGAATTACTTTCCCTTTCACTAATTCTAACA |
| | | TCTTCTTCTAGTTTGTCCCAGTCAAACTTGAAATTCAGAC |
| | | CTTGTCTTTGCATGTGCCTGTATTTCCCTGAGTATGCATT |
| | | TGCATTCATTTGCAGTAGAATCATTTTCATACACGAAAAC |
| | | CAATCACCCTCTGAAAAAAACTTCCTGCAGAGGTTTTTTG |
| | | CCATTTCATCCAGACCACATTGTTCTTTGACAGCTGAAGT |
| | | GAAATACAATGGTGACAGTTCTGTAGAAGTTTCAATAGCC |
| | | TCACAGATAAATTTCATGTCATCATTGGTGAGACAAGATG |
| | | GGTCAAAATCTTCCACAAGATGAAAAGAAATTTCTGATAA |
| | | GATGACCTTCCTTAAATATGCCATTTTACCTGACAATATA |
| | | GTCTGAAGGTGATGCAATCCTTTTGTATTTTCAAACCCCA |
| | | CCTCATTTTCCCCTTCATTGGTCTTCTTGCTTCTTTCATA |
| | | CCGCTTTATTGTGGAGTTGACCTTATCTTCTAAATTCTTG |
| | | AAGAAACTTGTCTCTTCTTCCCCATCAAAGCATATGTCTG |
| | | CTGAGTCACCTTCTAGTTTCCCAGCTTCTGTTTCTTTAGA |
| | | GCCGATAACCAATCTAGAGACCAACTTTGAAACCTTGTAC |
| | | TCGTAATCTGAGTGGTTCAATTTGTACTTCTGCTTTCTCA |
| | | TGAAGCTCTCTGTGATCTGACTCACAGCACTAACAAGCAA |
| | | TTTGTTAAAATCATACTCTAGGAGCCGTTCCCCATTTAAA |
| | | TGTTTGTTAACAACCACACTTTTGTTGCTGGCAAGGTCTA |
| | | ATGCTGTTGCACACCCAGAGTTAGTCATGGGATCCAAGCT |
| | | ATTGAGCCTCTTCTCCCCTTTGAAAATCAAAGTGCCATTG |
| | | TTGAATGAGGACACCATCATGCTAAAGGCCTCCAGATTGA |
| | | CACCTGGGGTTGTGCGCTGACAGTCAACTTCTTTCCCAGT |
| | | GAACTTCTTCATTTGGTCATAAAAAACACACTCTTCCTCA |
| | | GGGGTGATTGACTCTTTAGGGTTAACAAAGAAGCCAAACT |
| | | CACTTTTAGGCTCAAAGAATTTCTCAAAGCATTTAATTTG |
| | | ATCTGTCAGCCTATCAGGGGTTTCCTTTGTGATTAAATGA |
| | | CACAGGTATGACACATTCAACATGAACTTGAACTTTGCGC |
| | | TCAACAGTACCTTTTCACCAGTCCCAAAAACAGTTTTGAT |
| | | CAAAAATCTGAGCAATTTGTACACTACTTTCTCAGCAGGT |
| | | GTGATCAAATCCTCCTTCAACTTGTCCATCAATGATGTGG |
| | | ATGAGAAGTCTGAGACAATGGCCATCACTAAATACCTAAT |
| | | GTTTTGAACCTGTTTTTGATTCCTCTTTGTTGGGTTGGTG |
| | | AGCATGAGTAATAATAGGGTTCTCAATGCAATCTCAACAT |
| | | CATCAATGCTGTCCTTCAAGTCAGGACATGATCTGATCCA |
| | | TGAGATCATGGTGTCAATCATGTTGTGCAACACTTCATCT |
| | | GAGAAGATTGGTAAAAAGAACCTTTTTGGGTCTGCATAAA |
| | | AAGAGATTAGATGGCCATTGGGACCTTGTATAGAATAACA |
| | | CCTTGAGGATTCTCCAGTCTTTTGATACAGCAGGTGATAT |
| | | TCCTCAGAGTCCAATTTTATCACTTGGCAAAATACCTCTT |
| | | TACATTCCACCACTTGATACCTTACAGAGCCCAATTGGTT |
| | | TTGTCTTAATCTAGCAACTGAACTTGTTTTCATACTGTTT |
| | | GTCAAAGCTAGACAGACAGATGACAATCTTTTCAAACTAT |
| | | GCATGTTCCTTAATTGTTCCGTATTAGGCTGGAAATCATA |
| | | ATCTTCAAACTTTGTATAATACATTATAGGATGAGTTCCG |
| | | GACCTCATGAAATTCTCAAACTCAATAAATGGTATGTGGC |
| | | ACTCATGCTCAAGATGTTCAGACAGACCATAGTGCCCAAA |
| | | ACTAAGTCCCACCACTGACAAGCACCTTTGAACTTTTAAA |
| | | ATGAACTCATTTATGGATGTTCTAAACAAATCCTCAAGAG |
| | | ATACCTTTCTATACGCCTTTGACTTTCTCCTGTTCCTTAG |
| | | AAGTCTGATGAACTCTTCCTTGGTGCTATGAAAGCTCACC |
| | | AACCTATCATTCACACTCCCATAGCAACAACCAACCCAGT |
| | | GCTTATCATTTTTTGACCCTTTGAGTTTAGACTGTTTGAT |
| | | CAACGAAGAGAGACACAAGACATCCAAATTCAGTAACTGT |
| | | CTCCTTCTGGTGTTCAATAATTTTAAACTTTTAACTTTGT |
| | | TCAACATAGAGAGGAGCCTCTCATACTCAGTGCTAGTCTC |
| | | ACTTCCTCTCTCATAACCATGGGTATCTGCTGTGATAAAT |
| | | CTCATCAAAGGACAGGATTCAACTGCCTCCTTGCTTAGTG |
| | | CTGAAATGTCATCACTGTCAGCAAGAGTCTCATAAAGCTC |
| | | AGAGAATTCCTTAATTAAATTTCCGGGGTTGATTTTCTGA |
| | | AAACTCCTCTTGAGCTTCCCAGTTTCCAAGTCTCTTCTAA |
| | | ACCTGCTGTAAAGGGAGTTTATGCCAAGAACCACATCATC |
| | | GCAGTTCATGTTTGGGTTGACACCATCATGGCACATTTTC |
| | | ATAATTTCATCATTGTGAAATGATCTTGCATCTTTCAAGA |
| | | TTTTCATAGAGTCTATACCGGAACGCTTATCAACAGTGGT |
| | | CTTGAGAGATTCGCAAAGTCTGAAGTACTCAGATTCCTCA |
| | | AAGACTTTCTCATCTTGGCTAGAATACTCTAAAAGTTTAA |
| | | ACAGAAGGTCTCTGAACTTGAAATTCACCCACTCTGGCAT |
| | | AAAGCTGTTATCATAATCACACCGACCATCCACTATTGGG |
| | | ACCAATGTGATACCCGCAATGGCAAGGTCTTCTTTGATAC |
| | | AGGCTAGTTTATTGGTGTCCTCTATAAATTTCTTCTCAAA |
| | | ACTAGCTGGTGTGCTTCTAACGAAGCACTCAAGAAGAATG |
| | | AGGGAATTGTCAATCAGTTTATAACCATCAGGAATGATCA |
| | | AAGGCAGTCCCGGGCACACAATCCCAGACTCTATTAGAAT |
| | | TGCCTCAACAGATTTATCATCATGGTTGTGTATGCAGCCG |
| | | CTCTTGTCAGCACTGTCTATCTCTATACAACGCGACAAAA |
| | | GTTTGAGTCCCTCTATCAATACCATTCTGGGTTCTCTTTG |
| | | CCCTAAAAAGTTGAGCTTCTGCCTTGACAACCTCTCATCT |
| | | TGTTCTATGTGGTTTAAGCACAACTCTCTCAACTCCGAAA |
| | | TAGCCTCATCCATTGCGCATCAAAAAGCCTAGGATCCTCG |
| | | GTGCG |
| 5 | Lymphocytic | CGCACCGGGGATCCTAGGCTTTTTGGATTGCGCTTTCCTC |
| | choriomeningitis | AGCTCCGTCTTGTGGGAGAATGGGTCAAATTGTGACGATG |
| | strain MP segment | TTTGAGGCTCTGCCTCACATCATTGATGAGGTCATTAACA |
| | S, complete | TTGTCATTATCGTGCTTATTATCATCACGAGCATCAAAGC |
| | sequence | TGTGTACAATTTCGCCACCTGCGGGATACTTGCATTGATC |
| | (The genomic | AGCTTTCTTTTTCTGGCTGGCAGGTCCTGTGGAATGTATG |
| | segment is RNA, the | GTCTTGATGGGCCTGACATTTACAAAGGGGTTTACCGATT |
| | sequence in SEQ ID | CAAGTCAGTGGAGTTTGACATGTCTTACCTTAACCTGACG |
| | NO: 5 is shown for | ATGCCCAATGCATGTTCGGCAAACAACTCCCATCATTATA |
| | DNA; however, | TAAGTATGGGGACTTCTGGATTGGAGTTAACCTTCACAAA |
| | exchanging all | TGACTCCATCATCACCCACAACTTTTGTAATCTGACTTCC |
| | thymidines (“T”) in | GCCCTCAACAAGAGGACTTTTGACCACACACTTATGAGTA |
| | SEQ ID NO: 5 for | TAGTCTCAAGTCTGCACCTCAGCATTAGAGGGGTCCCCAG |
| | uridines (“U”) | CTACAAAGCAGTGTCCTGTGATTTTAACAATGGCATCACT |
| | provides the RNA | ATTCAATACAACCTGTCATTTTCTAATGCACAGAGCGCTC |
| | sequence.) | TGAGTCAATGTAAGACCTTCAGGGGGAGAGTCCTGGATAT |
| | | GTTCAGAACTGCTTTTGGAGGAAAGTACATGAGGAGTGGC |
| | | TGGGGCTGGACAGGTTCAGATGGCAAGACTACTTGGTGCA |
| | | GCCAGACAAACTACCAATATCTGATTATACAAAACAGGAC |
| | | TTGGGAAAACCACTGCAGGTACGCAGGCCCTTTCGGAATG |
| | | TCTAGAATTCTCTTCGCTCAAGAAAAGACAAGGTTTCTAA |
| | | CTAGAAGGCTTGCAGGCACATTCACTTGGACTTTATCAGA |
| | | CTCATCAGGAGTGGAGAATCCAGGTGGTTACTGCTTGACC |
| | | AAGTGGATGATCCTCGCTGCAGAGCTCAAGTGTTTTGGGA |
| | | ACACAGCTGTTGCAAAGTGCAATGTAAATCATGATGAAGA |
| | | GTTCTGTGATATGCTACGACTGATTGATTACAACAAGGCT |
| | | GCTTTGAGTAAATTCAAAGAAGATGTAGAATCCGCTCTAC |
| | | ATCTGTTCAAGACAACAGTGAATTCTTTGATTTCTGATCA |
| | | GCTTTTGATGAGAAATCACCTAAGAGACTTGATGGGAGTG |
| | | CCATACTGCAATTACTCGAAATTCTGGTATCTAGAGCATG |
| | | CAAAGACTGGTGAGACTAGTGTCCCCAAGTGCTGGCTTGT |
| | | CAGCAATGGTTCTTATTTGAATGAAACCCATTTCAGCGAC |
| | | CAAATTGAGCAGGAAGCAGATAATATGATCACAGAAATGC |
| | | TGAGAAAGGACTACATAAAAAGGCAAGGGAGTACCCCTCT |
| | | AGCCTTGATGGATCTATTGATGTTTTCTACATCAGCATAT |
| | | TTGATCAGCATCTTTCTGCATCTTGTGAGGATACCAACAC |
| | | ACAGACACATAAAGGGCGGCTCATGCCCAAAACCACATCG |
| | | GTTAACCAGCAAGGGAATCTGTAGTTGTGGTGCATTTAAA |
| | | GTACCAGGTGTGGAAACCACCTGGAAAAGACGCTGAACAG |
| | | CAGCGCCTCCCTGACTCACCACCTCGAAAGAGGTGGTGAG |
| | | TCAGGGAGGCCCAGAGGGTCTTAGAGTGTTACGACATTTG |
| | | GACCTCTGAAGATTAGGTCATGTGGTAGGATATTGTGGAC |
| | | AGTTTTCAGGTCGGGGAGCCTTGCCTTGGAGGCGCTTTCA |
| | | AAGATGATACAGTCCATGAGTGCACAGTGTGGGGTGACCT |
| | | CTTTCTTTTTCTTGTCCCTCACTATTCCAGTGTGCATCTT |
| | | GCATAGCCAGCCATATTTGTCCCAGACTTTGTCCTCATAT |
| | | TCTCTTGAAGCTTCTTTAGTCATCTCAACATCGATGAGCT |
| | | TAATGTCTCTTCTGTTTTGTGAATCTAGGAGTTTCCTGAT |
| | | GTCATCAGATCCCTGACAACTTAGGACCATTCCCTGTGGA |
| | | AGAGCACCTATTACTGAAGATGTCAGCCCAGGTTGTGCAT |
| | | TGAAGAGGTCAGCAAGGTCCATGCCATGTGAGTATTTGGA |
| | | GTCCTGCTTGAATTGTTTTTGATCAGTGGGTTCTCTATAG |
| | | AAATGTATGTACTGCCCATTCTGTGGCTGAAATATTGCTA |
| | | TTTCTACCGGGTCATTAAATCTGCCCTCAATGTCAATCCA |
| | | TGTAGGAGCGTTAGGGTCAATACCTCCCATGAGGTCCTTC |
| | | AGCAACATTGTTTGGCTGTAGCTTAAGCCCACCTGAGGTG |
| | | GGCCCGCTGCCCCAGGCGCTGGTTTGGGTGAGTTGGCCAT |
| | | AGGCCTCTCATTTGTCAGATCAATTGTTGTGTTCTCCCAT |
| | | GCTCTCCCTACAACTGATGTTCTACAAGCTATGTATGGCC |
| | | ACCCCTCCCCTGAAAGACAGACTTTGTAGAGGATGTTCTC |
| | | GTAAGGATTCCTGTCTCCAACCTGATCAGAAACAAACATG |
| | | TTGAGTTTCTTCTTGGCCCCAAGAACTGCTTTCAGGAGAT |
| | | CCTCACTGTTGCTTGGCTTAATTAAGATGGATTCCAACAT |
| | | GTTACCCCCATCTAACAAGGCTGCCCCTGCTTTCACAGCA |
| | | GCACCGAGACTGAAATTGTAGCCAGATATGTTGATGCTAG |
| | | ACTGCTGCTCAGTGATGACTCCCAAGACTGGGTGCTTGTC |
| | | TTTCAGCCTTTCAAGGTCACTTAGGTTCGGGTACTTGACT |
| | | GTGTAAAGCAGCCCAAGGTCTGTGAGTGCTTGCACAACGT |
| | | CATTGAGTGAGGTTTGTGATTGTTTGGCCATACAAGCCAT |
| | | TGTTAAGCTTGGCATTGTGCCGAATTGATTGTTCAGAAGT |
| | | GATGAGTCCTTCACATCCCAGACCCTCACCACACCATTTG |
| | | CACTCTGCTGAGGTCTCCTCATTCCAACCATTTGCAGAAT |
| | | CTGAGATCTTTGGTCAAGCTGTTGTGCTGTTAAGTTCCCC |
| | | ATGTAGACTCCAGAAGTTAGAGGCCTTTCAGACCTCATGA |
| | | TTTTAGCCTTCAGTTTTTCAAGGTCAGCTGCAAGGGACAT |
| | | CAGTTCTTCTGCACTAAGCCTCCCTACTTTTAGAACATTC |
| | | TTTTTTGATGTTGACTTTAGGTCCACAAGGGAATACACAG |
| | | TTTGGTTGAGGCTTCTGAGTCTCTGTAAATCTTTGTCATC |
| | | CCTCTTCTCTTTCCTCATGATCCTCTGAACATTGCTCACC |
| | | TCAGAGAAGTCTAATCCATTCAGAAGGCTGGTGGCATCCT |
| | | TGATCACAGCAGCTTTCACATCTGATGTGAAGCCTTGAAG |
| | | CTCTCTCCTCAATGCCTGGGTCCATTGAAAGCTTTTAACT |
| | | TCTTTGGACAGAGACATTTTGTCACTCAGTGGATTTCCAA |
| | | GTCAAATGCGCAATCAAAATGCCTAGGATCCACTGTGCG |
| 6 | Amino acid sequence | MSLSKEVKSFQWTQALRRELQGFTSDVKAAVIKDATSLLN |
| | of the NP protein | GLDFSEVSNVQRIMRKEKRDDKDLQRLRSLNQTVYSLVDL |
| | of the MP strain of | KSTSKKNVLKVGRLSAEELMSLAADLEKLKAKIMRSERPL |
| | LCMV | TSGVYMGNLTAQQLDQRSQILQMVGMRRPQQSANGVVRVW |
| | | DVKDSSLLNNQFGTMPSLTMACMAKQSQTSLNDVVQALTD |
| | | LGLLYTVKYPNLSDLERLKDKHPVLGVITEQQSSINISGY |
| | | NFSLGAAVKAGAALLDGGNMLESILIKPSNSEDLLKAVLG |
| | | AKKKLNMFVSDQVGDRNPYENILYKVCLSGEGWPYIACRT |
| | | SVVGRAWENTTIDLTNERPMANSPKPAPGAAGPPQVGLSY |
| | | SQTMLLKDLMGGIDPNAPTWIDIEGRFNDPVEIAIFQPQN |
| | | GQYIHFYREPTDQKQFKQDSKYSHGMDLADLFNAQPGLTS |
| | | SVIGALPQGMVLSCQGSDDIRKLLDSQNRRDIKLIDVEMT |
| | | KEASREYEDKVWDKYGWLCKMHTGIVRDKKKKEVTPHCAL |
| | | MDCIIFESASKARLPDLKTVHNILPHDLIFRGPNVVTL |
| 7 | Amino acid sequence | MGQIVTMFEALPHIIDEVINIVIIVLIIITSIKAVYNFAT |
| | of the GP protein | CGILALISFLFLAGRSCGMYGLDGPDIYKGVYRFKSVEFD |
| | of the MP strain of | MSYLNLTMPNACSANNSHHYISMGTSGLELTFTNDSIITH |
| | LCMV | NFCNLTSALNKRTFDHTLMSIVSSLHLSIRGVPSYKAVSC |
| | | DFNNGITIQYNLSFSNAQSALSQCKTFRGRVLDMFRTAFG |
| | | GKYMRSGWGWTGSDGKTTWCSQTNYQYLIIQNRTWENHCR |
| | | YAGPFGMSRILFAQEKTRFLTRRLAGTFTWTLSDSSGVEN |
| | | PGGYCLTKWMILAAELKCFGNTAVAKCNVNHDEEFCDMLR |
| | | LIDYNKAALSKFKEDVESALHLFKTTVNSLISDQLLMRNH |
| | | LRDLMGVPYCNYSKFWYLEHAKTGETSVPKCWLVSNGSYL |
| | | NETHFSDQIEQEADNMITEMLRKDYIKRQGSTPLALMDLL |
| | | MFSTSAYLISIFLHLVRIPTHRHIKGGSCPKPHRLTSKGI |
| | | CSCGAFKVPGVETTWKRR |
| 8 | amino acid sequence | MDEAISELRELCLNHIEQDERLSRQKLNFLGQREPRMVLI |
| | of the L protein of | EGLKLLSRCIEIDSADKSGCIHNHDDKSVEAILIESGIVC |
| | the MP strain of | PGLPLIIPDGYKLIDNSLILLECFVRSTPASFEKKFIEDT |
| | LCMV | NKLACIKEDLAIAGITLVPIVDGRCDYDNSFMPEWVNFKF |
| | | RDLLFKLLEYSSQDEKVFEESEYFRLCESLKTTVDKRSGI |
| | | DSMKILKDARSFHNDEIMKMCHDGVNPNMNCDDVVLGINS |
| | | LYSRFRRDLETGKLKRSFQKINPGNLIKEFSELYETLADS |
| | | DDISALSKEAVESCPLMRFITADTHGYERGSETSTEYERL |
| | | LSMLNKVKSLKLLNTRRRQLLNLDVLCLSSLIKQSKLKGS |
| | | KNDKHWVGCCYGSVNDRLVSFHSTKEEFIRLLRNRRKSKA |
| | | YRKVSLEDLFRTSINEFILKVQRCLSVVGLSFGHYGLSEH |
| | | LEHECHIPFIEFENFMRSGTHPIMYYTKFEDYDFQPNTEQ |
| | | LRNMHSLKRLSSVCLALTNSMKTSSVARLRQNQLGSVRYQ |
| | | VVECKEVFCQVIKLDSEEYHLLYQKTGESSRCYSIQGPNG |
| | | HLISFYADPKRFFLPIFSDEVLHNMIDTMISWIRSCPDLK |
| | | DSIDDVEIALRTLLLLMLTNPTKRNQKQVQNIRYLVMAIV |
| | | SDFSSTSLMDKLKEDLITPAEKVVYKLLRFLIKTVFGTGE |
| | | KVLLSAKFKFMLNVSYLCHLITKETPDRLTDQIKCFEKFF |
| | | EPKSEFGFFVNPKESITPEEECVFYDQMKKFTGKEVDCQR |
| | | TTPGVNLEAFSMMVSSFNNGTLIFKGEKRLNSLDPMTNSG |
| | | CATALDLASNKSVVVNKHLNGERLLEYDFNKLLVSAVSQI |
| | | TESFMRKQKYKLNHSDYEYKVSKLVSRLVIGSKETEAGKL |
| | | EGDSADICFDGEEETSFFKNLEDKVNSTIKRYERSKKTNE |
| | | GENEVGFENTKGLHHLQTILSGKMAYLRKVILSEISFHLV |
| | | EDFDPSCLTNDDMKFICEAIETSTELSPLYFTSAVKEQCG |
| | | LDEMAKNLCRKFFSEGDWFSCMKMILLQMNANAYSGKYRH |
| | | MQRQGLNFKFDWDKLEEDVRISERESNSESLSKALSLTKC |
| | | MSAALKNLCFYSEESPTSYTSVGPDSGRLKFALSYKEQVG |
| | | GNRELYIGDLRTKMFTRLIEDYFESFSSFFSGSCLNNDKE |
| | | FENAILSMTINVREGLLNYSMDHSKWGPMMCPFLFLMLLQ |
| | | NLKLGDDQYVRSGKDHISTLLTWHMHKLVEVPFPVVNAMM |
| | | KSYIKSKLKLLRGSETTVTERIFREYFELGIVPSHISSLI |
| | | DMGQGILHNASDFYGLISERFINYCIGVIFGERPESYTSS |
| | | DDQITLFDRRLSELVDSDPEEVLVLLEFHSHLSGLLNKFI |
| | | SPKSVVGRFAAEFKSRFYVWGEEVPLLTKFVSAALHNVKC |
| | | KEPHQLCETIDTIADQAVANGVPVSLVNCIQKRTLDLLKY |
| | | ANFPLDPFLLNTNTDVKDWLDGSRGYRIQRLIEELCPSET |
| | | KVMRRLVRRLHHKLKNGEFNEEFFLDLFNRDKKEAILQLG |
| | | NILGLEEDLSQLANINWLNLNELFPLRMVLRQKVVYPSVM |
| | | TFQEERIPSLIKTLQNKLCSKFTRGAQKLLSEAINKSAFQ |
| | | SCISSGFIGLCKTLGSRCVRNKNRDNLYIRKVLEDLAMDA |
| | | HVTAIHRHDGIMLYICDRQSHPEAHCDHISLLRPLLWDYI |
| | | CISLSNSFELGVWVLAEPVKGKNEGSSSLKHLNPCDYVAR |
| | | KPESSRLLEDKISLNHVIQSVRRLYPKIYEDQLLPFMSDM |
| | | SSKNMRWSPRIKFLDLCVLIDINSESLSLISHVVKWKRDE |
| | | HYTVLFSDLVNSHQRSDSSLVDEFVVSTRDVCKNFLKQVY |
| | | FESFVREFVATSRTLGSFSWFPHKDMMPSEDGAEALGPFQ |
| | | SFILKVVNKNMERPMFRNDLQFGFGWFSYRLGDIVCNAAM |
| | | LIKQGLTNPKAFKSLRNLWDYMINNTEGVLEFSITVDFTH |
| | | NQNNTDCLRKFSLIFLVKCQLQGPGVAEFLSCSHLFKGEV |
| | | DRRFLDECLHLLRSDSIFKVNDGVFDIRSEEFEDYMEDPL |
| | | ILGDSLELELIGSRKILDGIRSLDFERIGPEWEPVPLTVR |
| | | MGALFEGRSLVQNIVVKLETKDMRVFLAELEGYGNFDDVL |
| | | GSLLLHRFRTGEHLQGSEISTILQELCIDRSILLVPLSLV |
| | | PDWFTFKDCRLCFSKSKNTVMYETVVGKYRLKGKSCDDWL |
| | | TKSVVEEID |
| 9 | Amino acid sequence | MGQGKSKEGRDASNTSRAEILPDTTYLGPLNCKSCWQRFD |
| | of the Z protein of | SLVRCHDHYLCRHCLNLLLSVSDRCPLCKHPLPTKLKIST |
| | the MP strain of | APSSPPPYEE |
| | LCMV | |
| 10 | Junin virus | GCGCACCGGGGATCCTAGGCGTAACTTCATCATTAAAATCTCAGATTCT |
| | Candid#1 L segment | GCTCTGAGTGTGACTTACTGCGAAGAGGCAGACAAATGGGCAACTGCAA |
| | | CGGGGCATCCAAGTCTAACCAGCCAGACTCCTCAAGAGCCACACAGCCA |
| | | GCCGCAGAATTTAGGAGGGTAGCTCACAGCAGTCTATATGGTAGATATA |
| | | ACTGTAAGTGCTGCTGGTTTGCTGATACCAATTTGATAACCTGTAATGA |
| | | TCACTACCTTTGTTTAAGGTGCCATCAGGGTATGTTAAGGAATTCAGAT |
| | | CTCTGCAATATCTGCTGGAAGCCCCT |
| | | GCCCACCACAATCACAGTACCGGTGGAGCCAACAGCACCACCACCATAG |
| | | GCAGACTGCACAGGGTCAGACCCGACCCCCCGGGGGGCCCCCATGGGGA |
| | | CCCCCCGTGGGGGAACCCCGGGGGTGATGCGCCATTAGTCAATGTCTTT |
| | | GATCTCGACTTTGTGCTTCAGTGGCCTGCATGTCACCCCTTTCAATCTG |
| | | AACTGCCCTTGGGGATCTGATATCAGCAGGTCATTTAAAGATCT |
| | | GCTGAATGCCACCTTGAAATTTGAGAATTCCAACCAGTCACCAAATTTA |
| | | TCAAGTGAACGGATCAACTGCTCTTTGTGTA |
| | | GATCATAAACGAGGACAAAGTCCTCTTGCTGAAATAATATTGTTTGTGA |
| | | TGTTGTTTTTAGATAAGGCCATAGTTGGCTT |
| | | AATAAGGTTTCCACACTATCAATGTCCTCTAGTGCTCCAATTGCCTTGA |
| | | CTATGACATCCCCAGACAACTCAACTCTATA |
| | | TGTTGACAACCTTTCATTACCTCTGTAAAAGATACCCTCTTTCAAGACA |
| | | AGAGGTTCTCCTGGGTTATCTGGCCCAATGA |
| | | GGTCATATGCATACTTGTTACTTAGTTCAGAATAAAAGTCACCAAAGTT |
| | | GAACTTAACATGGCTCAGAATATTGTCATCA |
| | | TTTGTCGCAGCGTAGCCTGCATCAATAAACAAGCCAGCTAGGTCAAAGC |
| | | TCTCATGGCCTGTGAACAATGGTAGGCTAGC |
| | | GATAACCAGTGCACCATCCAACAATGAGTGGCTTCCCTCAGACCCAGAA |
| | | ACACATTGACTCATTGCATCCACATTCAGCT |
| | | CTAATTCAGGGGTACCGACATCATCCACTCCTAGTGAACTGACAATGGT |
| | | GTAACTGTACACCATCTTTCTTCTAAGTTTA |
| | | AATTTTGTCGAAACTCGTGTGTGTTCTACTTGAATGATCAATTTTAGTT |
| | | TCACAGCTTCTTGGCAAGCAACATTGCGCAA |
| | | CACAGTGTGCAGGTCCATCATGTCTTCCTGAGGCAACAAGGAGATGTTG |
| | | TCAACAGAGACACCCTCAAGGAAAACCTTGA |
| | | TATTATCAAAGCTAGAAACTACATAACCCATTGCAATGTCTTCAACAAA |
| | | CATTGCTCTTGATACTTTATTATTCCTAACT |
| | | GACAAGGTAAAATCTGTGAGTTCAGCTAGATCTACTTGACTGTCATCTT |
| | | CTAGATCTAGAACTTCATTGAACCAAAAGAA |
| | | GGATTTGAGACACGATGTTGACATGACTAGTGGGTTTATCATCGAAGAT |
| | | AAGACAACTTGCACCATGAAGTTCCTGCAAA |
| | | CTTGCTGTGGGCTGATGCCAACTTCCCAATTTGTATACTCTGACTGTCT |
| | | AACATGGGCTGAAGCGCAATCACTCTGTTTC |
| | | ACAATATAAACATTATTATCTCTTACTTTCAATAAGTGACTTATAATCC |
| | | CTAAGTTTTCATTCATCATGTCTAGAGCCAC |
| | | ACAGACATCTAGAAACTTGAGTCTTCCACTATCCAAAGATCTGTTCACT |
| | | TGAAGATCATTCATAAAGGGTGCCAAATGTT |
| | | CTTCAAATAGTTTGGGGTAATTTCTTCGTATAGAATGCAATACATGGTT |
| | | CATGCCTAATTGGTCTTCTATCTGTCGTACT |
| | | GCTTTGGGTTTAACAGCCCAGAAGAAATTCTTATTACATAAGACCAGAG |
| | | GGGCCTGTGGACTCTTAATAGCAGAAAACAC |
| | | CCACTCCCCTAACTCACAGGCATTTGTCAGCACCAAAGAGAAGTAATCC |
| | | CACAAAATTGGTTTAGAAAATTGGTTAACTT |
| | | CTTTAAGTGATTTTTGACAGTAAATAACTTTAGGCTTTCTCTCACAAAT |
| | | TCCACAAAGACATGGCATTATTCGAGTAAAT |
| | | ATGTCCTTTATATACAGAAATCCGCCTTTACCATCCCTAACACACTTAC |
| | | TCCCCATACTCTTACAAAACCCAATGAAGCC |
| | | TGAGGCAACAGAAGACTGAAATGCAGATTTGTTGATTGACTCTGCCAAG |
| | | ATCTTCTTCACGCCTTTTGTGAAATTTCTTG |
| | | ACAGCCTGGACTGTATTGTCCTTATCAATGTTGGCATCTCTTCTTTCTC |
| | | TAACACTCTTCGACTTGTCATGAGTTTGGTC |
| | | CTCAAGACCAACCTCAAGTCCCCAAAGCTCGCTAAATTGACCCATCTGT |
| | | AGTCTAGAGTTTGTCTGATTTCATCTTCACT |
| | | ACACCCGGCATATTGCAGGAATCCGGATAAAGCCTCATCCCCTCCCCTG |
| | | CTTATCAAGTTGATAAGGTTTTCCTCAAAGA |
| | | TTTTGCCTCTCTTAATGTCATTGAACACTTTCCTCGCGCAGTTCCTTAT |
| | | AAACATTGTCTCCTTATCATCAGAAAAAATA |
| | | GCTTCAATTTTCCTCTGTAGACGGTACCCTCTAGACCCATCAACCCAGT |
| | | CTTTGACATCTTGTTCTTCAATAGCTCCAAA |
| | | CGGAGTCTCTCTGTATCCAGAGTATCTAATCAATTGGTTGACTCTAATG |
| | | GAAATCTTTGACACTATATGAGTGCTAACCC |
| | | CATTAGCAATACATTGATCACAAATTGTGTCTATGGTCTCTGACAGTTG |
| | | TGTTGGAGTTTTACACTTAACGTTGTGTAGA |
| | | GCAGCAGACACAAACTTGGTGAGTAAAGGAGTCTCTTCACCCATGACAA |
| | | AAAATCTTGACTTAAACTCAGCAACAAAAGTTCCTATCACACTCTTTGG |
| | | GCTGATAAACTTGTTTAATTTAGAAGATAAGAATTCATGGAAGCACACC |
| | | ATTTCCAGCAGTT |
| | | CTGTCCTGTCTTGAAACTTTTCATCACTAAGGCAAGGAATTTTTATAAG |
| | | GCTAACCTGGTCATCGCTGGAGGTATAAGTG |
| | | ACAGGTATCACATCATACAATAAGTCAAGTGCATAACACAGAAATTGTT |
| | | CAGTAATTAGCCCATATAAATCTGATGTGTT |
| | | GTGCAAGATTCCCTGGCCCATGTCCAAGACAGACATTATATGGCTGGGG |
| | | ACCTGGTCCCTTGACTGCAGATACTGGTGAA |
| | | AAAACTCTTCACCAACACTAGTACAGTCACAACCCATTAAACCTAAAGA |
| | | TCTCTTCAATTTCCCTACACAGTAGGCTTCT |
| | | GCAACATTAATTGGAACTTCAACGACCTTATGAAGATGCCATTTGAGAA |
| | | TGTTCATTACTGGTTCAAGATTCACCTTTGT |
| | | TCTATCTCTGGGATTCTTCAATTCTAATGTGTACAAAAAAGAAAGGAAA |
| | | AGTGCTGGGCTCATAGTTGGTCCCCATTTGG |
| | | AGTGGTCATATGAACAGGACAAGTCACCATTGTTAACAGCCATTTTCAT |
| | | ATCACAGATTGCACGTTCGAATTCCTTTTCT |
| | | GAATTCAAGCATGTGTATTTCATTGAACTACCCACAGCTTCTGAGAAGT |
| | | CTTCAACTAACCTGGTCATCAGCTTAGTGTT |
| | | GAGGTCTCCCACATACAGTTCTCTATTTGAGCCAACCTGCTCCTTATAA |
| | | CTTAGTCCAAATTTCAAGTTCCCTGTATTTG |
| | | AGCTGATGCTTGTGAACTCTGTAGGAGAGTCGTCTGAATAGAAACATAA |
| | | ATTCCGTAGGGCTGCATTTGTAAAATAACTT |
| | | TTGTCTAGCTTATCAGCAATGGCTTCAGAATTGCTTTCCCTGGTACTAA |
| | | GCCGAACCTCATCCTTTAGTCTCAGAACTTC |
| | | ACTGGAAAAGCCCAATCTAGATCTACTTCTATGCTCATAACTACCCAAT |
| | | TTCTGATCATAATGTCCTTGAATTAAAAGAT |
| | | ACTTGAAGCATTCAAAGAATTCATCTTCTTGGTAGGCTATTGTTGTCAA |
| | | ATTTTTTAATAACAAACCCAAAGGGCAGATG |
| | | TCCTGCGGTGCTTCAAGAAAATAAGTCAATTTAAATGGAGATAGATAAA |
| | | CAGCATCACATAACTCTTTATACACATCAGA |
| | | CCTGAGCACATCTGGATCAAAATCCTTCACCTCATGCATTGACACCTCT |
| | | GCTTTAATCTCTCTCAACACTCCAAAAGGGG |
| | | CCCACAATGACTCAAGAGACTCTCGCTCATCAACAGATGGATTTTTTGA |
| | | TTTCAACTTGGTGATCTCAACTTTTGTCCCC |
| | | TCACTATTAGCCATCTTGGCTAGTGTCATTTGTACGTCATTTCTAATAC |
| | | CCTCAAAGGCCCTTACTTGATCCTCTGTTAA |
| | | ACTCTCATACATCACTGATAATTCTTCTTGATTGGTTCTGGTTCTTGAA |
| | | CCGGTGCTCACAAGACCTGTTAGATTTTTTA |
| | | ATATTAAGTAGTCCATGGAATCAGGATCAAGATTATACCTGCCTTTTGT |
| | | TTTAAACCTCTCAGCCATAGTAGAAACGCAT |
| | | GTTGAAACAAGTTTCTCCTTATCATAAACAGAAAGAATATTTCCAAGTT |
| | | CGTCGAGCTTGGGGATTACCACACTTTTATT |
| | | GCTTGACAGATCCAGAGCTGTGCTAGTGATGTTAGGCCTGTAGGGATTG |
| | | CTTTTCAGTTCACCTGTAACTTTAAGTCTTC |
| | | CTCTATTGAAGAGAGAAATGCAGAAGGACAAAATCTCTTTACACACTCC |
| | | TGGAATTTGAGTATCTGAGGAAGTCTTAGCC |
| | | TCTTTGGAAAAGAATCTGTCCAATCCTCTTATCATGGTGTCCTCTTGTT |
| | | CCAGTGTTAGACTCCCACTTAGAGGGGGGTT |
| | | TACAACAACACAATCAAACTTGACTTTGGGCTCAATAAACTTCTCAAAA |
| | | CACTTTATTTGATCTGTCAGGCGATCAGGTG |
| | | TCTCTTTGGTTACCAAGTGACACAGATAACTAACATTTAATAGATATTT |
| | | AAACCTTCTTGCAAAGTAAAGATCTGCATCT |
| | | TCCCCTTCACCCAAAATTGTCTGGAAAAGTTCCACAGCCATCCTCTGAA |
| | | TCAGCACCTCTGATCCAGACATGCAGTCGAC |
| | | CCTTAACTTTGACATCAAATCCACATGATGGATTTGATTTGCATATGCC |
| | | ATCAAGAAATATCTTAGACCTTGTAAAAATG |
| | | TCTGGTTCCTTTTGGAAGGGGAACAGAGTACAGCTAACACTAACAATCT |
| | | TAATATTGGCCTTGTCATTGTCATGAGTTCG |
| | | TGGCTAAAATCCAACCAGCTGGTCATTTCCTCACACATTTCAATTAACA |
| | | CATCCTCCGAAAATATAGGCAGGAAAAATCT |
| | | CTTTGGATCACAGTAAAAAGAGCCTTGTTCTTCCAATACCCCATTGATG |
| | | GATAGATAGATAGAATAGCACCTTGACTTCT |
| | | CACCTGTTTTTTGGTAAAACAAGAGACCAAATGTATTCTTTGTCAGATG |
| | | AAATCTTTGTACATAACACTCTCTTAGTCTA |
| | | ACATTCCCAAAATATCTAGAATACTCTCTTTCATTGATTAACAATCGGG |
| | | AGGAAAATGATGTCTTCATCGAGTTGACCAA |
| | | TGCAAGGGAAATGGAGGACAAAATCCTAAATAATTTCTTCTGCTCACCT |
| | | TCCACTAAGCTGCTGAATGGCTGATGTCTAC |
| | | AGATTTTCTCAAATTCCTTGTTAATAGTATATCTCATCACTGGTCTGTC |
| | | AGAAACAAGTGCCTGAGCTAAAATCATCAAG |
| | | CTATCCATATCAGGGTGTTTTATTAGTTTTTCCAGCTGTGACCAGAGAT |
| | | CTTGATGAGAGTTCTTCAATGTTCTGGAACA |
| | | CGCTTGAACCCACTTGGGGCTGGTCATCAATTTCTTCCTTATTAGTTTA |
| | | ATCGCCTCCAGAATATCTAGAAGTCTGTCAT |
| | | TGACTAACATTAACATTTGTCCAACAACTATTCCCGCATTTCTTAACCT |
| | | TACAATTGCATCATCATGCGTTTTGAAAAGA |
| | | TCACAAAGTAAATTGAGTAAAACTAAGTCCAGAAACAGTAAAGTGTTTC |
| | | TCCTGGTGTTGAAAACTTTTAGACCTTTCAC |
| | | TTTGTTACACACGGAAAGGGCTTGAAGATAACACCTCTCTACAGCATCA |
| | | ATAGATATAGAATTCTCATCTGACTGGCTTT |
| | | CCATGTTGACTTCATCTATTGGATGCAATGCGATAGAGTAGACTACATC |
| | | CATCAACTTGTTTGCACAAAAAGGGCAGCTG |
| | | GGCACATCACTGTCTTTGTGGCTTCCTAATAAGATCAAGTCATTTATAA |
| | | GCTTAGACTTTTGTGAAAATTTGAATTTCCC |
| | | CAACTGCTTGTCAAAAATCTCCTTCTTAAACCAAAACCTTAACTTTATG |
| | | AGTTCTTCTCTTATGACAGATTCTCTAATGT |
| | | CTCCTCTAACCCCAACAAAGAGGGATTCATTTAACCTCTCATCATAACC |
| | | CAAAGAATTCTTTTTCAAGCATTCGATGTTT |
| | | TCTAATCCCAAGCTCTGGTTTTTTGTGTTGGACAAACTATGGATCAATC |
| | | GCTGGTATTCTTGTTCTTCAATATTAATCTC |
| | | TTGCATAAATTTTGATTTCTTTAGGATGTCGATCAGCAACCACCGAACT |
| | | CTTTCAACAACCCAATCAGCAAGGAATCTAT |
| | | TGCTGTAGCTAGATCTGCCATCAACCACAGGAACCAACGTAATCCCTGC |
| | | CCTTAGTAGGTCGGACTTTAGGTTTAAGAGC |
| | | TTTGACATGTCACTCTTCCATTTTCTCTCAAACTCATCAGGATTGACCC |
| | | TAACAAAGGTTTCCAATAGGATGAGTGTTTT |
| | | CCCTGTGAGTTTGAAGCCATCCGGAATGACTTTTGGAAGGGTGGGACAT |
| | | AGTATGCCATAGTCAGACAGGATCACATCAA |
| | | CAAACTTCTGATCTGAATTGATCTGACAGGCGTGTGCCTCACAGGACTC |
| | | AAGCTCTACTAAACTTGACAGAAGTTTGAAC |
| | | CCTTCCAACAACAGAGAGCTGGGGTGATGTTGAGATAAAAAGATGTCCC |
| | | TTTGGTATGCTAGCTCCTGTCTTTCTGGAAA |
| | | ATGCTTTCTAATAAGGCTTTTTATTTCATTTACTGATTCCTCCATGCTC |
| | | AAGTGCCGCCTAGGATCCTCGGTGCG |
| 11 | Junin virus | GCGCACCGGGGATCCTAGGCGATTTTGGTTACGCTATAATTGTAACTGT |
| | Candid#1 S segment | TTTCTGTTTGGACAACATCAAAAACATCCATTGCACAATGGGGCAGTTC |
| | | ATTAGCTTCATGCAAGAAATACCAACCTTTTTGCAGGAGGCTCTGAACA |
| | | TTGCTCTTGTTGC |
| | | AGTCAGTCTCATTGCCATCATTAAGGGTATAGTGAACTTGTACAAAAGT |
| | | GGTTTATTCCAATTCTTTGTATTCCTAGCGC |
| | | TTGCAGGAAGATCCTGCACAGAAGAAGCTTTCAAAATCGGACTGCACAC |
| | | TGAGTTCCAGACTGTGTCCTTCTCAATGGTG |
| | | GGTCTCTTTTCCAACAATCCACATGACCTACCTTTGTTGTGTACCTTAA |
| | | ACAAGAGCCATCTTTACATTAAGGGGGGCAA |
| | | TGCTTCATTTCAGATCAGCTTTGATGATATTGCAGTATTGTTGCCACAG |
| | | TATGATGTTATAATACAACATCCAGCAGATA |
| | | TGAGCTGGTGTTCCAAAAGTGATGATCAAATTTGGTTGTCTCAGTGGTT |
| | | CATGAATGCTGTGGGACATGATTGGCATCTA |
| | | GACCCACCATTTCTGTGTAGGAACCGTGCAAAGACAGAAGGCTTCATCT |
| | | TTCAAGTCAACACCTCCAAGACTGGTGTCAA |
| | | TGGAAATTATGCTAAGAAGTTTAAGACTGGCATGCATCATTTATATAGA |
| | | GAATATCCTGACCCTTGCTTGAATGGCAAAC |
| | | TGTGCTTAATGAAGGCACAACCTACCAGTTGGCCTCTCCAATGTCCACT |
| | | CGACCACGTTAACACATTACACTTCCTTACA |
| | | AGAGGTAAAAACATTCAACTTCCAAGGAGGTCCTTGAAAGCATTCTTCT |
| | | CCTGGTCTTTGACAGACTCATCCGGCAAGGA |
| | | TACCCCTGGAGGCTATTGTCTAGAAGAGTGGATGCTCGTAGCAGCCAAA |
| | | ATGAAGTGTTTTGGCAATACTGCTGTAGCAA |
| | | AATGCAATTTGAATCATGACTCTGAATTCTGTGACATGTTGAGGCTCTT |
| | | TGATTACAACAAAAATGCTATCAAAACCCTA |
| | | AATGATGAAACTAAGAAACAAGTAAATCTGATGGGGCAGACAATCAATG |
| | | CCCTGATATCTGACAATTTATTGATGAAAAA |
| | | CAAAATTAGGGAACTGATGAGTGTCCCTTACTGCAATTACACAAAATTT |
| | | TGGTATGTCAACCACACACTTTCAGGACAAC |
| | | ACTCATTACCAAGGTGCTGGTTAATAAAAAACAACAGCTATTTGAACAT |
| | | CTCTGACTTCCGTAATGACTGGATATTAGAA |
| | | AGTGACTTCTTAATTTCTGAAATGCTAAGCAAAGAGTATTCGGACAGGC |
| | | AGGGTAAAACTCCTTTGACTTTAGTTGACAT |
| | | CTGTATTTGGAGCACAGTATTCTTCACAGCGTCACTCTTCCTTCACTTG |
| | | GTGGGTATACCCTCCCACAGACACATCAGGG |
| | | GCGAAGCATGCCCTTTGCCACACAGGTTGAACAGCTTGGGTGGTTGCAG |
| | | ATGTGGTAAGTACCCCAATCTAAAGAAACCA |
| | | ACAGTTTGGCGTAGAGGACACTAAGACCTCCTGAGGGTCCCCACCAGCC |
| | | CGGGCACTGCCCGGGCTGGTGTGGCCCCCCAGTCCGCGGCCTGGCCGCG |
| | | GACTGGGGAGGCACTGCTTACAGTGCATAGGCTGCCTTCGGGAGGAACA |
| | | GCAAGCTCGGTGGTAATAGAGGTGTAGGTTCCTCCTCATAGAGCTTCCC |
| | | ATCTAGCACTGACTGAAACATTATGCAGTCTAGCAGAGCACAGTGTGGT |
| | | TCACTGGAGGCCAACTTGAAGGGAGTATCCTTTTCCCTCTTTTTCTTAT |
| | | TGACAACCACTCCATTGTGATATTTG |
| | | CATAAGTGACCATATTTCTCCCAGACCTGTTGATCAAACTGCCTGGCTT |
| | | GTTCAGATGTGAGCTTAACATCAACCAGTTT |
| | | AAGATCTCTTCTTCCATGGAGGTCAAACAACTTCCTGATGTCATCGGAT |
| | | CCTTGAGTAGTCACAACCATGTCTGGAGGCA |
| | | GCAAGCCGATCACGTAACTAAGAACTCCTGGCATTGCATCTTCTATGTC |
| | | CTTCATTAAGATGCCGTGAGAGTGTCTGCTA |
| | | CCATTTTTAAACCCTTTCTCATCATGTGGTTTTCTGAAGCAGTGAATGT |
| | | ACTGCTTACCTGCAGGTTGGAATAATGCCAT |
| | | CTCAACAGGGTCAGTGGCTGGTCCTTCAATGTCGAGCCAAAGGGTGTTG |
| | | GTGGGGTCGAGTTTCCCCACTGCCTCTCTGA |
| | | TGACAGCTTCTTGTATCTCTGTCAAGTTAGCCAATCTCAAATTCTGACC |
| | | GTTTTTTTCCGGCTGTCTAGGACCAGCAACT |
| | | GGTTTCCTTGTCAGATCAATACTTGTGTTGTCCCATGACCTGCCTGTGA |
| | | TTTGTGATCTAGAACCAATATAAGGCCAACC |
| | | ATCGCCAGAAAGACAAAGTTTGTACAAAAGGTTTTCATAAGGATTTCTA |
| | | TTGCCTGGTTTCTCATCAATAAACATGCCTT |
| | | CTCTTCGTTTAACCTGAATGGTTGATTTTATGAGGGAAGAGAAGTTTTC |
| | | TGGGGTGACTCTGATTGTTTCCAACATGTTT |
| | | CCACCATCAAGAATAGATGCTCCAGCCTTTACTGCAGCTGAAAGACTGA |
| | | AGTTGTAACCAGAAATATTGATGGAGCTTTC |
| | | ATCTTTAGTCACAATCTGAAGGCAGTCATGTTCCTGAGTCAGTCTGTCA |
| | | AGGTCACTTAAGTTTGGATACTTCACAGTGT |
| | | ATAGAAGCCCAAGTGAGGTTAAAGCTTGTATGACACTGTTCATTGTCTC |
| | | ACCTCCTTGAACAGTCATGCATGCAATTGTC |
| | | AATGCAGGAACAGAGCCAAACTGATTGTTTAGCTTTGAAGGGTCTTTAA |
| | | CATCCCATATCCTCACCACACCATTTCCCCC |
| | | AGTCCCTTGCTGTTGAAATCCCAGTGTTCTCAATATCTCTGATCTTTTA |
| | | GCAAGTTGTGACTGGGACAAGTTACCCATGT |
| | | AAACCCCCTGAGAGCCTGTCTCTGCTCTTCTTATCTTGTTTTTTAATTT |
| | | CTCAAGGTCAGACGCCAACTCCATCAGTTCA |
| | | TCCCTCCCCAGATCTCCCACCTTGAAAACTGTGTTTCGTTGAACACTCC |
| | | TCATGGACATGAGTCTGTCAACCTCTTTATT |
| | | CAGGTCCCTCAACTTGTTGAGGTCTTCTTCCCCCTTTTTAGTCTTTCTG |
| | | AGTGCCCGCTGCACCTGTGCCACTTGGTTGA |
| | | AGTCGATGCTGTCAGCAATTAGCTTGGCGTCCTTCAAAACATCTGACTT |
| | | GACAGTCTGAGTGAATTGGCTCAAACCTCTC |
| | | CTTAAGGACTGAGTCCATCTAAAGCTTGGAACCTCCTTGGAGTGTGCCA |
| | | TGCCAGAAGTTCTGGTGATTTTGATCTAGAA |
| | | TAGAGTTGCTCAGTGAAAGTGTTAGACACTATGCCTAGGATCCACTGTG |
| | | CG |
| 12 | Amino acid sequence | MSLSKEVKSFQWTQALRRELQSFTSDVKAAVIKDATNLLNGLDFSEVSN |
| | of the NP protein | VQRIMRKEKRDDKDLQRLRSLNQTVHSLVDLKSTSKKNVLKVGRLSAEE |
| | of the Clone 13 | LMSLAADLEKLKAKIMRSERPQASGVYMGNLTTQQLDQRSQILQIVGMR |
| | strain of LCMV | KPQQGASGVVRVWDVKDSSLLNNQFGTMPSLTMACMAKQSQTPLNDVVQ |
| | (GenBank Accession | ALTDLGLLYTVKYPNLNDLERLKDKHPVLGVITEQQSSINISGYNFSLG |
| | No. ABC96002.1; | AAVKAGAALLDGGNMLESILIKPSNSEDLLKAVLGAKRKLNMFVSDQVG |
| | GI: 86440166) | DRNPYENILYKVCLSGEGWPYIACRTSIVGRAWENTTIDLTSEKPAVNS |
| | | PRPAPGAAGPPQVGLSYSQTMLLKDLMGGIDPNAPTWIDIEGRFNDPVE |
| | | IAIFQPQNGQFIHFYREPVDQKQFKQDSKYSHGMDLADLFNAQPGLTSS |
| | | VIGALPQGMVLSCQGSDDIRKLLDSQNRKDIKLIDVEMTREASREYEDK |
| | | VWDKYGWLCKMHTGIVRDKKKKEITPHCALMDCIIFESASKARLPDLKT |
| | | VHNILPHDLIFRGPNVVTL |
| 13 | Amino acid sequence | MGQIVTMFEALPHIIDEVINIVIIVLIVITGIKAVYNFATCGIFALISF |
| | of the GP protein | LLLAGRSCGMYGLKGPDIYKGVYQFKSVEFDMSHLNLTMPNACSANNSH |
| | of the Clone 13 | HYISMGTSGLELTFTNDSIISHNFCNLTSAFNKKTFDHTLMSIVSSLHL |
| | strain of LCMV | SIRGNSNYKAVSCDFNNGITIQYNLTFSDAQSAQSQCRTFRGRVLDMFR |
| | (GenBank Accession | TAFGGKYMRSGWGWTGSDGKTTWCSQTSYQYLIIQNRTWENHCTYAGPF |
| | No. ABC96001.2; | GMSRILLSQEKTKFLTRRLAGTFTWTLSDSSGVENPGGYCLTKWMILAA |
| | GI: 116563462) | ELKCFGNTAVAKCNVNHDEEFCDMLRLIDYNKAALSKFKEDVESALHLF |
| | | KTTVNSLISDQLLMRNHLRDLMGVPYCNYSKFWYLEHAKTGETSVPKCW |
| | | LVTNGSYLNETHFSDQIEQEADNMITEMLRKDYIKRQGSTPLALMDLLM |
| | | FSTSAYLVSIFLHLVKIPTHRHIKGGSCPKPHRLTNKGICSCGAFKVPG |
| | | VKTVWKRR |
| 14 | amino acid sequence | MDEIISELRELCLNYIEQDERLSRQKLNFLGQREPRMVLIEGLKLLSRC |
| | of the L protein of | IEIDSADKSGCTHNHDDKSVETILVESGIVCPGLPLIIPDGYKLIDNSL |
| | the Clone 13 strain | ILLECFVRSTPASFEKKFIEDTNKLACIREDLAVAGVTLVPIVDGRCDY |
| | of LCMV | DNSFMPEWANFKFRDLLFKLLEYSNQNEKVFEESEYFRLCESLKTTIDK |
| | (GenBank Accession | RSGMDSMKILKDARSTHNDEIMRMCHEGINPNMSCDDVVFGINSLFSRF |
| | No. ABC96004.1; | RRDLESGKLKRNFQKVNPEGLIKEFSELYENLADSDDILTLSREAVESC |
| | GI: 86440169) | PLMRFITAETHGHERGSETSTEYERLLSMLNKVKSLKLLNTRRRQLLNL |
| | | DVLCLSSLIKQSKFKGLKNDKHWVGCCYSSVNDRLVSFHSTKEEFIRLL |
| | | RNRKKSKVFRKVSFEELFRASISEFIAKIQKCLLVVGLSFEHYGLSEHL |
| | | EQECHIPFTEFENFMKIGAHPIMYYTKFEDYNFQPSTEQLKNIQSLRRL |
| | | SSVCLALTNSMKTSSVARLRQNQIGSVRYQVVECKEVFCQVIKLDSEEY |
| | | HLLYQKTGESSRCYSIQGPDGHLISFYADPKRFFLPIFSDEVLYNMIDI |
| | | MISWIRSCPDLKDCLTDIEVALRTLLLLMLTNPTKRNQKQVQSVRYLVM |
| | | AIVSDFSSTSLMDKLREDLITPAEKVVYKLLRFLIKTIFGTGEKVLLSA |
| | | KFKFMLNVSYLCHLITKETPDRLTDQIKCFEKFFEPKSQFGFFVNPKEA |
| | | ITPEEECVFYEQMKRFTSKEIDCQHTTPGVNLEAFSLMVSSFNNGTLIF |
| | | KGEKKLNSLDPMTNSGCATALDLASNKSVVVNKHLNGERLLEYDFNKLL |
| | | VSAVSQITESFVRKQKYKLSHSDYEYKVSKLVSRLVIGSKGEETGRSED |
| | | NLAEICFDGEEETSFFKSLEEKVNTTIARYRRGRRANDKGDGEKLTNTK |
| | | GLHHLQLILTGKMAHLRKVILSEISFHLVEDFDPSCLTNDDMKFICEAV |
| | | EGSTELSPLYFTSVIKDQCGLDEMAKNLCRKFFSENDWFSCMKMILLQM |
| | | NANAYSGKYRHMQRQGLNFKFDWDKLEEDVRISERESNSESLSKALSLT |
| | | QCMSAALKNLCFYSEESPTSYTSVGPDSGRLKFALSYKEQVGGNRELYI |
| | | GDLRTKMFTRLIEDYFESFSSFFSGSCLNNDKEFENAILSMTINVREGF |
| | | LNYSMDHSKWGPMMCPFLFLMFLQNLKLGDDQYVRSGKDHVSTLLTWHM |
| | | HKLVEVPFPVVNAMMKSYVKSKLKLLRGSETTVTERIFRQYFEMGIVPS |
| | | HISSLIDMGQGILHNASDFYGLLSERFINYCIGVIFGERPEAYTSSDDQ |
| | | ITLFDRRLSDLVVSDPEEVLVLLEFQSHLSGLLNKFISPKSVAGRFAAE |
| | | FKSRFYVWGEEVPLLTKFVSAALHNVKCKEPHQLCETIDTIADQAIANG |
| | | VPVSLVNSIQRRTLDLLKYANFPLDPFLLNTNTDVKDWLDGSRGYRIQR |
| | | LIEELCPNETKVVRKLVRKLHHKLKNGEFNEEFFLDLFNRDKKEAILQL |
| | | GDLLGLEEDLNQLADVNWLNLNEMFPLRMVLRQKVVYPSVMTFQEERIP |
| | | SLIKTLQNKLCSKFTRGAQKLLSEAINKSAFQSCISSGFIGLCKTLGSR |
| | | CVRNKNRENLYIKKLLEDLTTDDHVTRVCNRDGITLYICDKQSHPEAHR |
| | | DHICLLRPLLWDYICISLSNSFELGVWVLAEPTKGKNNSENLTLKHLNP |
| | | CDYVARKPESSRLLEDKVNLNQVIQSVRRLYPKIFEDQLLPFMSDMSSK |
| | | NMRWSPRIKFLDLCVLIDINSESLSLISHVVKWKRDEHYTVLFSDLANS |
| | | HQRSDSSLVDEFVVSTRDVCKNFLKQVYFESFVREFVATTRTLGNFSWF |
| | | PHKEMMPSEDGAEALGPFQSFVSKVVNKNVERPMFRNDLQFGFGWFSYR |
| | | MGDVVCNAAMLIRQGLTNPKAFKSLKDLWDYMLNYTKGVLEFSISVDFT |
| | | HNQNNTDCLRKFSLIFLVRCQLQNPGVAELLSCSHLFKGEIDRRMLDEC |
| | | LHLLRTDSVFKVNDGVFDIRSEEFEDYMEDPLILGDSLELELLGSKRIL |
| | | DGIRSIDFERVGPEWEPVPLTVKMGALFEGRNLVQNIIVKLETKDMKVF |
| | | LAGLEGYEKISDVLGNLFLHRFRTGEHLLGSEISVILQELCIDRSILLI |
| | | PLSLLPDWFAFKDCRLCFSKSRSTLMYETVGGRFRLKGRSCDDWLGGSV |
| | | AEDID |
| 15 | Amino acid | MGQGKSREEKGTNSTNRAEILPDTTYLGPLSCKSCWQKFDSLVRCHDHY |
| | sequence of the Z | LCRHCLNLLLSVSDRCPLCKYPLPTRLKISTAPSSPPPYEE |
| | protein of the | |
| | Clone 13 strain of | |
| | LCMV | |
| | (GenBank Accession | |
| | No. ABC96003.1; | |
| | GI: 86440168) | |
| 16 | Amino acid sequence | MGQIVTMFEALPHIIDEVINIVIIVLIIITSIKAVYNFATCGILALVSF |
| | of the GP protein | LFLAGRSCGMYGLNGPDIYKGVYQFKSVEFDMSHLNLTMPNACSANNSH |
| | of the WE strain of | HYISMGSSGLELTFTNDSILNHNFCNLTSAFNKKTFDHTLMSIVSSLHL |
| | LCMV | SIRGNSNHKAVSCDFNNGITIQYNLSFSDPQSAISQCRTFRGRVLDMFR |
| | | TAFGGKYMRSGWGWAGSDGKTTWCSQTSYQYLIIQNRTWENHCRYAGPF |
| | | GMSRILFAQEKTKFLTRRLAGTFTWTLSDSSGVENPGGYCLTKWMILAA |
| | | ELKCFGNTAVAKCNVNHDEEFCDMLRLIDYNKAALSKFKQDVESALHVF |
| | | KTTVNSLISDQLLMRNHLRDLMGVPYCNYSKFWYLEHAKTGETSVPKCW |
| | | LVTNGSYLNETHFSDQIEQEADNMITEMLRKDYIKRQGSTPLALMDLLM |
| | | FSTSAYLISIFLHLVKIPTHRHIKGGSCPKPHRLTNKGICSCGAFKVPG |
| | | VKTIWKRR |
| 17 | WE specific primer | 5′AATCGTCTCTAAGGATGGGTCAGATTGTGACAATG-3′ |
| 18 | WE specific fusion- | 5′AATCGTCTCTAAGGATGGGTCAGATTGTGACAATG-3′ |
| | primer carrying an | |
| | overhang | |
| | complementary to | |
| | the WET-specific | |
| | primer | |
| 19 | WE specific primer | 5′CTCGGTGATCATGTTATCTGCTTCTTGTTCGATTTGA-3′ |
| 20 | WE specific fusion- | 5′AATCGTCTCTTTCTTTATCTCCTCTTCCAGATGG-3′ |
| | primer | |
| | complementary to | |
| | the WE-sequence | |
| 21 | Primer specific for | 5′-GGCTCCCAGATCTGAAAACTGTT-3′ |
| | LCMV NP | |
| 22 | NP- and GP-specific | 5′-GCTGGCTTGTCACTAATGGCTC-3′ |
| | primers; NP- | |
| | specific: same as | |
| | in RT reaction, GP- | |
| | specific: 5′ | |

### 8. EXAMPLES

### 8.1 Example 1: Effect Between r3LCMV Treatment and Chemotherapy

A potential synergistic effect between r3LCMV treatment and low-dose chemotherapy (cyclophosphamide treatment) was evaluated in the B16F10 mouse melanoma model.

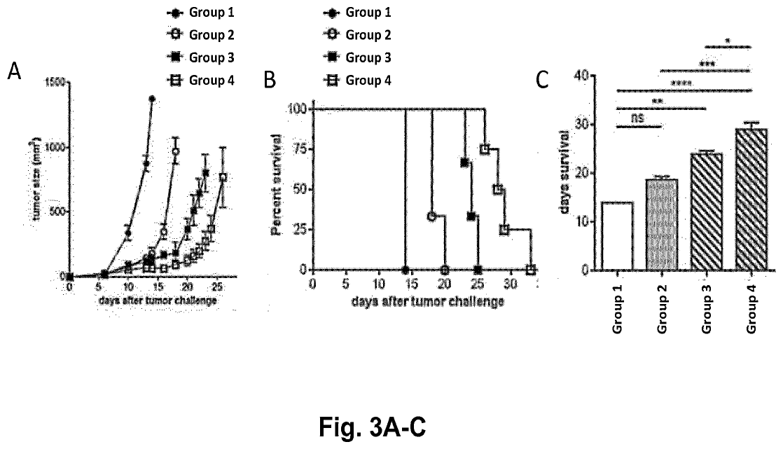

1×10⁵ B16F10 tumor cells were implanted subcutaneously into C57BL/6 mice on day 0. Mice were subsequently left untreated (group 1), treated intraperitoneally with 2 mg cyclophosphamide on day 6 (group 2), injected intravenously with 2.1×10⁵ PFU (in total) of a vector mix (7×10⁴ PFU of each r3LCMV-GP100, r3LCMV-Trp1 and r3LCMV-Trp2) on day 7 (group 3), or treated with a combination of cyclophosphamide (day 6) and r3LCMV-vector mix (day 7) (group 4). The genomic organization of the r3LCMV constructs is essentially as shown for r3LCMV-GFP^{art} in FIG. 2 except that in place of the GFP open reading frame the constructs have the open reading frame encoding for the antigen of interest, i.e. GP100, Trp1 and Trp2. Tumor growth after tumor challenge (FIG. 3A) as well as animal survival (FIGS. 3B and C) were monitored. Symbols represent the mean±SEM of three mice (groups 1-3) or four mice (group 4) per group. Treatment with the r3LCMV vector mix had a larger effect on tumor growth than chemotherapy alone. Best tumor control was achieved by combination of chemotherapy and treatment with the r3LCMV vector mix, indicating that the two combined treatments showed a synergistic effect.

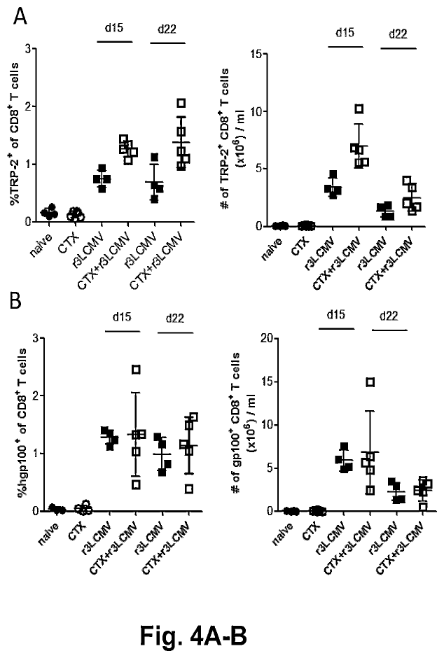

T cell frequencies in the blood of test animals were analyzed by tetramer staining and flow cytometric analysis on days 15 and 22 of the experiment. Results indicate that considerably higher relative (FIG. 4A, left panel) and absolute (FIG. 4A, right panel) numbers of Trp2-specific CD8+ T cells were induced in mice treated with a combination of cyclophosphamide and r3LCMV-vectors compared to animals treated with r3LCMV vectors only. This synergistic effect could not be observed in this experiment for GP100-specific CD8+ T cells (FIG. 4B).

### 8.2 Example 2: Effect Between r3LCMV Treatment and Chemotherapy in HCmel3 Model

A potential synergistic effect between r3LCMV treatment and low-dose chemotherapy (cyclophosphamide treatment) is evaluated in the HCmel3 mouse melanoma model. HCmel3 tumor cells are derived from a primary Hgf-Cdk4^{R24C} melanoma.

HCmel3 tumor cells (4×10⁵ cells) are implanted subcutaneously into C57BL/6 mice on day 0. On day 15, when all tumors are palpable, mice in groups 3 and 4 are treated intraperitoneally with 2 mg cyclophosphamide (CTX). On day 16 mice in groups 2 and 3 are injected intravenously with 7×10⁴ RCV FFU r3LCMV-Trp2. Mice in group 4 are immunized intravenously with 1×10⁵ RCV FFU r3PICV-Trp2.

The genomic organization of the r3LCMV constructs is essentially as shown for r3LCMV-GFP^{art} in FIG. 2 except that in place of the GFP open reading frame the constructs have the open reading frame encoding for the antigen of interest, i.e. Trp2. Tumor growth after tumor challenge is monitored.

Trp2-specific CD8+ T cell frequencies in the blood of test animals are analyzed by tetramer staining.

### 8.3 Example 3: Effect Between r3LCMV Treatment and Chemotherapy in HgfxCDK4^{R24C/R24C} Mouse

The HgfxCDK4^{R24C/R24C} model is a syngeneic model where mice develop spontaneous tumors which show some similarities to human melanomas (Landsberg et al., Autochthonous primary and metastatic melanomas in Hgf-Cdk4 R24C mice evade T-cell-mediated immune surveillance. 2010; Bald et al., Immune cell-Poor Melanomas benefit from PD-1 Blockade after targeted type I IFN activation, 2014.).

A potential synergistic effect between r3LCMV treatment and low-dose chemotherapy (cyclophosphamide treatment) is evaluated in the HgfxCDK4^{R24C/R24C} mouse model. Mice are left untreated (group 1), treated intraperitoneally with 2 mg cyclophosphamide when tumors are palpable (around day 60) (group 2), injected intravenously with a vector mix (r3LCMV-GP100, r3LCMV-Trp1 and r3LCMV-Trp2) when tumors are palpable (around day 60) (group 3), or treated with a combination of cyclophosphamide and r3LCMV-vector mix (group 4). The genomic organization of the r3LCMV constructs is essentially as shown for r3LCMV-GFP^{a}n in FIG. 2 except that in place of the GFP open reading frame the constructs have the open reading frame encoding for the antigen of interest, i.e. GP100, Trp1 and Trp2. Tumor growth as well as animal survival are monitored.

T cell frequencies in the blood of test animals are analyzed by tetramer staining and flow cytometric analysis on days 15 and 22 of the experiment.

### 8.4 Example 4: Effect Between r3LCMV Treatment and Chemotherapy with Heterologous Prime Boost

The experiments in Examples 1 and 2 (both the B16F10 and HCmel3 mouse models) are repeated to determine immune responses after heterologous prime boost vaccination using the following combinations of vectors with chemotherapy (cyclophosphamide): r3LCMV/r3LCMV, r3JUNV/r3LCMV, and r3PICV/r3LCMV.

### 8.5 Example 5: Heterologous Prime Boost

To investigate the immunogenicity of homologous versus heterologous prime-boost immunization, the induction of antigen-specific CD8+ T cell responses was compared between (i) mice treated with two administrations of r3LCMV-E7E6 (replication-competent LCMV vector expressing the antigens E7 and E6 from human papillomavirus type 16 (HPV16)) in a homologous prime-boost setting and (ii) animals primed with r3PICV-E7E6 (replication-competent Pichinde virus vector expressing E7 and E6 antigens) and boosted with r3LCMV-E7E6 in a heterologous prime-boost setting.

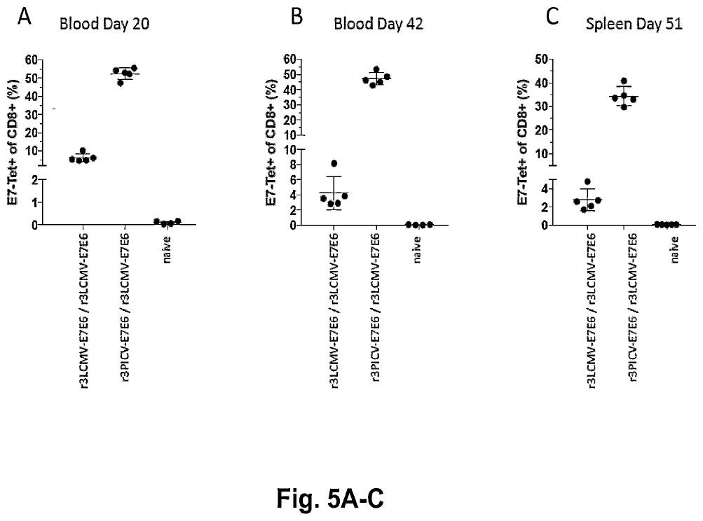

Results of this experiment are depicted in FIG. 5A-C: C57BL/6 mice (5 mice per group) were immunized intravenously on day 0 with 10⁵ RCV FFU of r3LCMV-E7E6 (group 1) or 10⁵ RCV FFU of r3PICV-E7E6 (group 2) or were left untreated (group 3). The genomic organization of the r3LCMV constructs is essentially as shown for r3LCMV-GFP^{art} in FIG. 2 except that in place of the GFP open reading frame the constructs have the open reading frame encoding for the antigen of interest, i.e. E7E6 (which is a fusion protein of the E6 and E7 proteins of HPV. On day 13 mice in groups 1 and 2 were boosted with 10⁵ RCV FFU of r3LCMV-E7E6. Mice of group 3 were again left untreated. E7-specific CD8+ T cell frequencies were subsequently analyzed by tetramer staining (Db-E7 (49-57)-Tetramer) on days 20 and 42 in the blood, and on day 51 in the spleen of test animals.

Respective results indicated that potent and durable, antigen-specific CD8+ T cell responses were induced in animals of test groups 1 and 2, treated with replication-competent arenavirus vectors expressing the E7 antigen. Significantly higher CD8+ T cell frequencies were induced by heterologous prime-boost combinations using r3PICV-E7E6 in combination with r3LCMV-E7E6 (group 2) compared to homologous immunizations using r3LCMV-E7E6 only (group 1).

Homologous and heterologous prime-boost vaccination regimens were further analyzed and compared in regard to their anti-tumor efficacy in the TC-1 mouse tumor model (Lin et al, 1996, Cancer Res.; 56(1):21-6). The level of tumor growth inhibition after administration of (i) two doses of r3LCMV-E7E6 (homologous prime-boost) or (ii) one dose of r3PICV-E7E6 followed by one dose of r3LCMV-E7E6 (heterologous prime-boost) was compared in TC-1 tumor bearing mice.

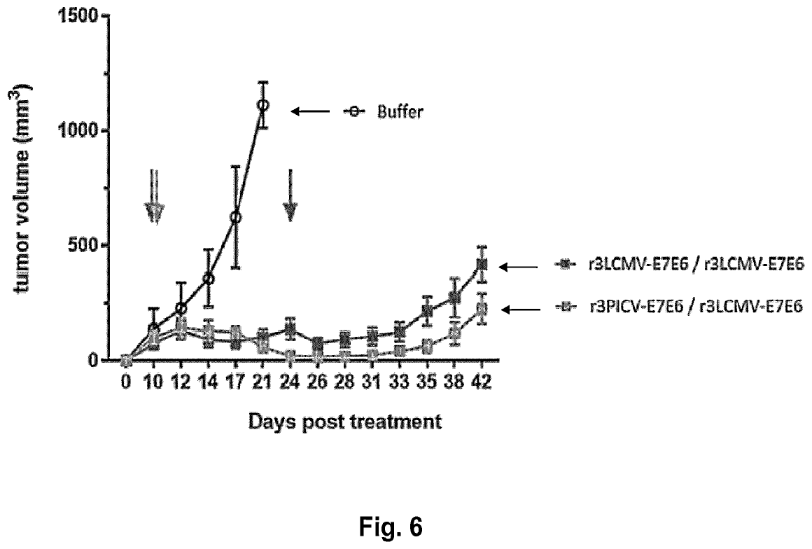

Results of these experiment are depicted in FIG. 6: On day 0 of the experiment female C57BL/6 mice (n=5 or n=3 animals per group for experimental groups and buffer group, respectively) were challenged subcutaneously with 1×10⁵ TC-1 tumor cells, derived from mouse primary epithelial cells, co-transformed with HPV16 E6 and E7 and c-Ha-ras oncogenes. Ten days later (day 10 of the experiment) mice were immunized intravenously with either buffer (group 1) or 10⁵ RCV FFU r3LCMV-E7E6 (group 2) or 10⁵ RCV FFU r3PICV-E7E6 (group 3). 14 days post prime (day 24 of the experiment) mice in groups 2 and 3 received a boost administration of 10⁵ RCV FFU r3LCMV-E7E6. Tumor growth was subsequently monitored over time. Arithmetic means+/−SEM are shown. Arrows indicate time points of vaccination.

Respective results indicate that compared to the control group tumor growth was significantly delayed in all groups treated with replication-competent arenavirus vectors expressing the HPV E7 and E6 antigens. Higher levels of tumor growth control was observed in the test group treated with r3PICV-E7E6 in combination with r3LCMV-E7E6 in a heterologous prime-boost fashion.

### 8.6 Example 6: Effect Between rLCMV Treatment, Chemotherapy and Immune Checkpoint Inhibitor Treatment in the B16F10 Mouse Melanoma Model

A potential synergistic effect between rLCMV treatment, low-dose chemotherapy (cyclophosphamide treatment) and immune checkpoint inhibitor (anti PD-1) treatment is evaluated in the B16F10 mouse melanoma model.

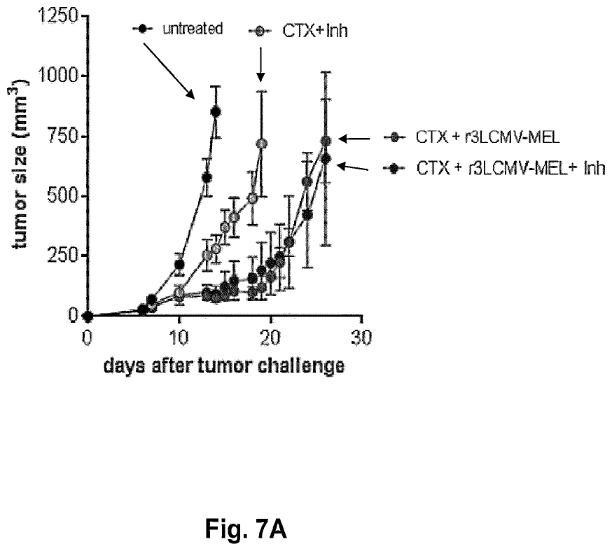

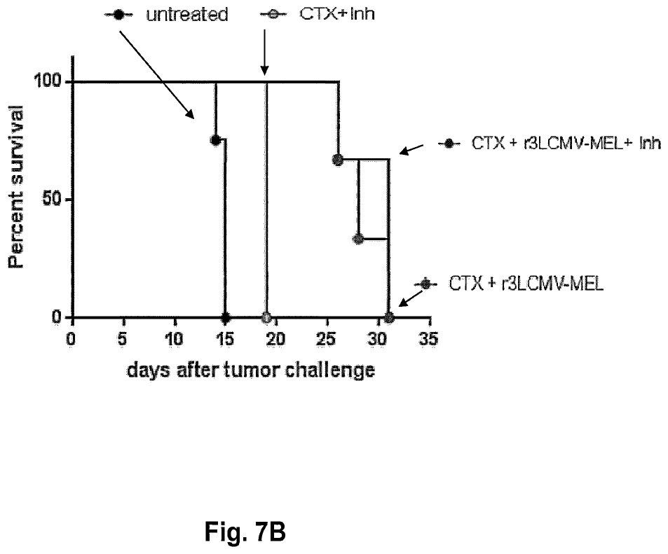

Results of the experiment are depicted in FIG. 7A-B. 1×10⁵ B16F10 tumor cells were implanted subcutaneously into C57BL/6 mice on day 0. Mice were subsequently left untreated (group 1), treated intraperitoneally with 2 mg cyclophosphamide (CTX) on day 6 and 200 μg each of anti PD-1 and anti-CTLA-4 on days 10, 13, 16, 19 and 22 (group 2), treated intraperitoneally with 2 mg cyclophosphamide on day 6 and injected intravenously with 1.2×10⁵ FFU (in total) of a r3LCMV vector mix (r3LCMV-GP100, r3LCMV-Trp1 and r3LCMV-Trp2) on day 7 (group 3), or treated with cyclophosphamide on day 6, an r3LCMV-vector mix on day 7 and anti PD-1 and anti-CTLA-4 on days 10, 13, 16, 19 and 22 (group 4).

Respective results indicated that no additional effect on tumor growth inhibition could be achieved by combining the checkpoint inhibitor treatment with the combination of chemotherapy and r3LCMV.

1-221. (canceled)

222. A method for treating a neoplastic disease in a subject in need thereof, wherein the method comprises administering to the subject an arenavirus particle and a chemotherapeutic agent, wherein the arenavirus particle is a tri-segmented arenavirus particle comprising one L segment and two S segments, and wherein the arenavirus particle is engineered to contain an arenavirus genomic segment comprising:
(i) a nucleotide sequence encoding a tumor antigen, a tumor associated antigen, or an antigenic fragment thereof; and
(ii) at least one arenavirus open reading frame (“ORF”) in a position other than the wild-type position of the ORF, wherein the ORF encodes the glycoprotein (“GP”), the nucleoprotein (“NP”), the matrix protein Z (“Z protein”) or the RNA dependent RNA polymerase L (“L protein”) of the arenavirus particle.

223. The method of claim 222, wherein the chemotherapeutic agent is a platinum-based chemotherapy, a taxane, or a combination thereof.

224. The method of claim 222, wherein the tumor associated antigen is a human papillomavirus (HPV)-associated antigen.

225. The method of claim 222, wherein the neoplastic disease is a human papillomavirus (HPV) induced cancer.

226. The method of claim 222, wherein the neoplastic disease is anal cancer; cervical cancer; head and neck cancer; mouth cancer; oral cancer; oral cavity cancer; penile cancer; throat cancer; vaginal cancer; vulvar cancer, or a combination thereof.

227. The method of claim 224, wherein the HPV-associated antigen is HPV E7 and E6 fusion protein, HPV E6, or HPV E7.

228. The method of claim 222, wherein:
(i) the arenavirus particle and the chemotherapeutic agent are co-administered simultaneously;
(ii) the arenavirus particle and the chemotherapeutic agent are administered on the same day;
(iii) the arenavirus particle is administered prior to administration of the chemotherapeutic agent; or
(iv) the arenavirus particle is administered after administration of the chemotherapeutic agent.

229. The method of claim 228, wherein the interval between administration of the arenavirus particle and the chemotherapeutic agent is about 1 hour, about 2 hours, about 3 hours, about 4 hours, about 5 hours, about 6 hours, about 7 hours, about 8 hours, about 9 hours, about 10 hours, about 11 hours, about 12 hours, about 1 day, about 2 days, about 3 days, about 4 days, about 5 days, about 6 days, about 1 week, about 8 days, about 9 days, about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, or more.

230. The method of claim 222, wherein the arenavirus particle and the chemotherapeutic agent are administered in a therapeutically effective amount.

231. The method of claim 222, wherein the method further comprises administering to the subject a second arenavirus particle, after a period of time from the first administration of the arenavirus particle.

232. The method of claim 231, wherein the second arenavirus particle is derived from a different arenavirus species as the arenavirus particle, and the second arenavirus particle is engineered to contain an arenavirus genomic segment comprising the same tumor antigen, tumor associated antigen, or antigenic fragment thereof as those in the arenavirus particle.

233. The method of claim 232, wherein the second arenavirus particle is derived from lymphocytic choriomeningitis virus (“LCMV”) and the arenavirus particle is derived from Pichinde virus (“PICV”).

234. The method of claim 222, wherein:
(i) propagation of the tri-segmented arenavirus particle does not result in a replication-competent bi-segmented viral particle;
(ii) propagation of the tri-segmented arenavirus particle does not result in a replication-competent bi-segmented viral particle after 70 days of persistent infection in mice lacking type I interferon receptor, type II interferon receptor and recombination activating gene 1 (RAG1) and having been infected with 10⁴ PFU of the tri-segmented arenavirus particle; and/or
(iii) inter-segmental recombination of two S segments, uniting two arenavirus ORFs on only one instead of two separate segments, abrogates viral promoter activity.

235. The method of claim 222, wherein one of the two S segments is selected from the group consisting of:
(i) an S segment, wherein the ORF encoding the NP is under control of an arenavirus 5′ UTR;
(ii) an S segment, wherein the ORF encoding the Z protein is under control of an arenavirus 5′ UTR;
(iii) an S segment, wherein the ORF encoding the L protein is under control of an arenavirus 5′ UTR;
(iv) an S segment, wherein the ORF encoding the GP is under control of an arenavirus 3′ UTR;
(v) an S segment, wherein the ORF encoding the L protein is under control of an arenavirus 3′ UTR; and
(vi) an S segment, wherein the ORF encoding the Z protein is under control of an arenavirus 3′ UTR.

236. The method of claim 222, wherein the ORF encoding the GP is under control of an arenavirus 3′ UTR.

237. The method of claim 222, wherein each of the two S segments comprises a nucleotide sequence encoding the tumor antigen, tumor associated antigen, or antigenic fragment thereof.

238. The method of claim 222, wherein the arenavirus particle is derived from lymphocytic choriomeningitis virus (“LCMV”) or Pichinde virus (“PICV”).

239. The method of claim 238, wherein:
(i) the LCMV is MP strain, WE strain, Armstrong strain, or Armstrong Clone 13 strain; or
(ii) the PICV is strain Munchique CoAn4763 isolate P18, or P2 strain.

240. The method of claim 222, wherein the method further comprises administering an immune checkpoint inhibitor to the subject.

241. The method of claim 240, wherein the immune checkpoint inhibitor is an anti-PD-1 antibody.

242. A method for treating a human papillomavirus (HIPV) induced cancer in a subject in need thereof, wherein the method comprises administering to the subject an arenavirus particle and a chemotherapeutic agent, wherein the chemotherapeutic agent is a platinum-based chemotherapy, a taxane, or a combination thereof, wherein the arenavirus particle is derived from lymphocytic choriomeningitis virus (“LCMV”) or Pichinde virus (“PICV”) and is a tri-segmented arenavirus particle comprising one L segment, a first S segment, and a second S segment, and wherein:
(i) the first S segment comprises an open reading frame encoding the arenaviral glycoprotein GP in a position under control of a genomic 3′ untranslated region and an open reading frame encoding a HPV E7 and E6 fusion protein in a position under control of a genomic 5′ untranslated region;
(ii) the second S segment comprises an open reading frame encoding the arenaviral nucleoprotein NP in a position under control of a genomic 3′ untranslated region and an open reading frame encoding a HPV E7 and E6 fusion protein in a position under control of a genomic 5′ untranslated region; and
(iii) the L segment comprises an open reading frame encoding ribonucleic acid (RNA) dependent RNA polymerase L in a position under control of a genomic 3′ untranslated region and an open reading frame encoding the matrix protein Z in a position under control of a genomic 5′ untranslated region.
